(19) United States
(12) Patent Application Publication
(10) Pub. No.: US 2023/0149531 A1
(43) Pub. Date: May 18, 2023

(54) Suprastructure Comprising Modified Influenza Hemagglutinin With Reduced Interaction With Sialic Acid

(71) Applicants: Hilary E. Hendin, Montreal (CA); Brian J. Ward, Montreal (CA); Medicago Inc., Quebec (CA)

(72) Inventors: Pierre-Olivier Lavoie, Quebec (CA); Hilary E. Hendin, Montreal (CA); Brian J. Ward, Montreal (CA); Nathalie Landry, St-Jean-Chrysostome (CA); Marc-Andre D'Aoust, Quebec (CA); Mikael Bedard, Quebec (CA); Pooja Saxena, Montreal (CA)

(21) Appl. No.: 17/920,625

(22) PCT Filed: Apr. 22, 2021
(86) PCT No.: PCT/CA2021/050554

§ 371 Date: Oct. 21, 2022

Related U.S. Application Data
(60) Provisional application No. 63/014,008, filed on Apr. 22, 2020.

(51) Int. Cl. A61K 39/145; C12N 7/00; A61P 31/16; C07K 14/005

(52) U.S. Cl. CPC A61K 39/145; C12N 7/00; A61P 31/16; C07K 14/005; C12N 2760/16122; C12N 2760/16123; C12N 2760/16134; C12N 2760/16152; A61K 2039/5258

(57) ABSTRACT

A suprastructure comprising a modified influenza hemagglutinin (HA) is provided. The modified HA may comprise one or more than one alteration that reduces non-cognate binding of the modified HA to sialic acid (SA) on the surface of a cell, while maintaining cognate interaction with the cell, such as a B cell. A composition comprising the suprastructure and modified HA and a pharmaceutically acceptable carrier is also described. A method of increasing an immunological response or inducing immunity in response to a vaccine comprising the suprastructure and modified HA is also provided.

### FIELD OF INVENTION

The present invention relates to suprastructures that comprise modified influenza hemagglutinin (HA) protein. The modified HA protein comprises one or more than one alteration that reduces non-cognate interaction of the modified HA to sialic acid (SA).

### BACKGROUND OF THE INVENTION

Influenza viruses are members of the Orthomyxoviridae family (single-stranded, negative-sense RNA) that cause acute respiratory infection in humans. Seasonal outbreaks of influenza are responsible for approximately 250,000-500,000 deaths worldwide each year. Antigenic variants of influenza arise through inter-species genetic reassortment and pose a significant pandemic threat. Public vaccination programs help to minimize the morbidity and mortality associated with influenza infection, however current vaccine formulations are only effective in 50-60% of healthy adults and significant strain-to-strain variation in immunogenicity is evident. For example, vaccines targeting avian strains of influenza generally elicit poor antibody responses compared to those targeting mammalian (i.e.: seasonal) strains. As a result, pandemic vaccines often require higher doses of antigen and/or the addition of adjuvants to achieve reasonable levels of seroconversion.

A universal vaccine is one that elicits broadly neutralizing antibodies at protective titers when administered to a subject. The development of a universal influenza vaccine would be useful to diminish the threat posed by influenza virus.

There are four types of influenza virus: A, B, C and D, of which influenza A and B are the causative organism for seasonal disease epidemics in humans. Influenza A viruses are further divided based on the expression of hemagglutinin (HA) and neuraminidase (NA) glycoprotein subtypes on the surface of the virus. There are 18 different HA subtypes (H1-H18).

HA is a trimeric lectin that facilitates binding of the influenza virus particle to sialic acid-containing proteins on the surface of target cells and mediates release of the viral genome into the target cell. HA proteins comprise two structural elements: the head, which is the primary target of seroprotective antibodies; and the stalk. HA is translated as a single polypeptide, HA0 (assembled as trimers), that must be cleaved by a serine endoprotease between the HA1 (˜40 kDa) and HA2 (˜20 kDa) subdomains. After cleavage, the two disulfide-bonded protein domains adopt the requisite conformation necessary for viral infectivity. HA1 forms the globular head domain containing the receptor-binding site (RBS), and is the least conserved segment of the influenza virus genome. HA2 is a single-pass integral membrane protein with fusion peptide (FP), soluble ectodomain (SE), transmembrane (TM), and cytoplasmic tail (CT) with respective lengths of approximately 25, 160, 25, and 10 residues. HA2 together with the N and C terminal HA1 residues forms a stalk domain, which includes the transmembrane region, and is relatively conserved.

Suprastructures (protein suprastructures), for example, virus-like particles (VLPs) may be used in immunogenic compositions. VLPs closely resemble mature virions, but they do not contain viral genomic material, and they are non-replicative which make them safe for administration as a vaccine. In addition, VLPs can be engineered to express viral glycoproteins on the surface of the VLP, which is their most native physiological configuration. Since VLPs resemble intact virions and are multivalent particulate structures, VLPs may be more effective in inducing neutralizing antibodies to the glycoprotein than soluble envelope protein antigens.

VLPs have been produced in plants (WO2009/076778; WO2009/009876; WO 2009/076778; WO 2010/003225; WO 2010/003235; WO2010/006452; WO2011/03522; WO 2010/148511; and WO2014153674, which are incorporated herein by reference). For example, WO2009/009876 and WO 2009/076778 disclose the production of virus-like particles (VLP) comprising influenza hemagglutinin (HA) in plants. Such plant produced VLPs closely resemble influenza viruses, and vaccines made from plant made VLPs elicit good antibody titers and strong cellular responses making them a promising alternative to current vaccine formulations (Landry, N. et. al. 2014 Clin Immun (Orlando Fla.) Aug. 17, 2014).

Humoral immunity (antibody-mediated immunity), is an adaptative immunity mediated by antibodies secreted by B cells. The antibodies produced by the B cells may then be used to neutralize an antigen or pathogen. Humoral immunity involves B-cell activation arising from the B cell binding a foreign antigen or pathogen. Activated B cells interact closely with helper T cells to form a complex that results in proliferation of the B-cells to produce plasma cells and memory B cells. When the memory cells encounter the antigen (pathogen) they can divide to form plasma cells. Plasma cells produce large numbers of antibodies which then bind the antigen (pathogen). Antibodies produced by plasma B cells neutralize viruses and toxins released by bacteria; kill organisms by activating the complement system; coat the antigen (opsonization) or form an antigen-antibody complex to stimulate phagocytosis; and prevent the antigen from adhering to its receptor, for example on host target cells.

Cell-mediated immunity (CMI) is mediated by antigen-specific CD4 and CD8 T cells and there is no antibody involvement. CMI responses are initiated when antigen presenting cells (APCs) including macrophages, dendritic cells, and in some circumstances, B cells internalize a microbial organism or parts thereof. The whole organism or material of microbial origin is then broken down into small antigenic peptides, which are presented on MHC molecules on the surface of the APC. Naïve CD4 and CD8 T cells that recognize specific microbial peptides on the surface of APCs become activated and release cytokines to promote antigen-specific T cell proliferation and differentiation into various effector and memory subsets. The main mediators of anti-viral CMI are type 1 CD4+ helper T cells (Th1) which activate macrophages to promote microbial clearance and cytotoxic CD8 T cells which directly kill infected target cells. Memory T cells are reactivated upon subsequent exposure to the pathogen and provide long-lived immunity.

Influenza hemagglutinin (HA) initiates infection by binding to sialic acid (SA) residues on the surface of respiratory epithelial cells. HA binds SA via a conserved region at the receptor binding site located on the globular head region of the HA molecule (Whittle, J. R., et al., 2014, J Virol, 88(8): p. 4047-57). The specificity and affinity of this interaction is strain-dependent, with mammalian influenza strains (e.g. H1NI) preferentially binding to α(2,6)-linked SA and avian influenza strains (e.g. H5N1 or H7N9) typically binding to α(2,3)-linked SA (Ramos I., et. al., 2013 J. Gen. Virol. 94:2417-2423). The receptor specificity of influenza and the distribution of SA receptors in the human respiratory tract greatly contribute to the severity and transmissibility of infection. α(2,6)-linked SA are densely expressed in the upper respiratory tract resulting in relatively mild but highly transmissible infections with mammalian influenza strains (e.g. H1N1). However, α(2,3)-linked SA predominate in the lower respiratory tract resulting in reduced transmission of avian influenza strains (e.g. H5N1, H7N9) but considerably higher severity and mortality.

Sialic acid (SA) residues are expressed throughout the body including on the surface of immune cells. As a result, HA in vaccines binds to SA-expressing host cells. Additionally, there are differences in the pattern of α(2,6)-linked SA and α(2,3)-linked SA on human immune cells. VLP vaccine candidates bearing H1 or H5 interact with distinct subsets of human peripheral blood mononuclear cells (PBMC) in an HA-dependent manner to induce strain-specific innate immune responses (Hendin H. E., et. al., 2017 Vaccine 35:2592-2599). Early events in the infection pathway may influence subsequent adaptive responses and HA binding properties may be a factor contributing to vaccine immunogenicity and efficacy.

Meisner, J., et al., (2008, J. Virol. 82, 5079-83) generated a Y98F H3 (A/Aichi/2/68) virus using reverse genetics. The Y98F mutation reduced binding 20-fold. Three months post-infection, mice infected with Y98F or native/wild type virus had similar HAI titers. Analysis of viral plaques isolated from lungs of Y98F-infected mice indicated reversion, in that 13 out of 18 isolates had acquired other mutations that restored HA binding.

Y98F HA has been used as a probe, for example Villar et. al. (2016, Sci Rep, 6: p. 36298) prepared nanoparticles using self-associating ferritin to create 8-mers of HA to increase valency of the probe. Zost et al (2019, Cell Rep. 29:4460-4470) expressed Y98F H3 on the surface of 293F cells to measure neutralizing antibodies in human sera. Tan, H.-X. X. et al. (2019, J Clin. Invest. 129, 850-862) prepared Y98F HA for use as a probe to identify HA-specific antibody responses and antigen-specific B cells. Tan also reports vaccinating with Y98F HA and an HA stem and found that the immunogenicity of the Y98F HA protein was comparable to that of the control HA stem. Whittle et al. (2014, J Virol, 88(8): p. 4047-57) describe H1 HA comprising a Y98F mutation in the amino acid sequence of H1 that inhibits SA binding while permitting host-cell binding. Since native HA proteins bind to SA on B cells and cause a high level of background ‘noise’ in studies that focus on binding between the B cell receptor and its cognate antigen, Whittle describes the use of the Y98F-HA as a probe to detect HA-specific B cell receptor interaction in patients that have previously been vaccinated with an H5 influenza virus.

WO2015183969 describes nanoparticle-based vaccine consisting of a novel HA stabilized stem (SS) without the variable immunodominant head region genetically fused to the surface of nanoparticles (Gen6 HA-SS np, also referred to as Hl-SS-np). WO2015183969 found that Hl-SS-np induced effective signaling through wild-type B cell receptor. However, nanoparticle with full-length HA containing Y98F mutation to abolish nonspecific binding to sialic acid (HA-np), induced wild-type B cell receptor to a lesser extent, suggesting a reduced immune response to HA with Y98F mutation.

The receptor binding site is located on the globular head of HA and amino acid 98 is at the base of the receptor binding site. The phenol side chain of Y98 forms a hydrogen bond with sialic acid to facilitate binding. Phenylalanine has a similar structure to tyrosine so that the shape of the binding pocket and antigenicity is maintained by the Y98F mutation. However, phenylalanine lacks a hydroxyl group on the side chain and therefore cannot form hydrogen bonds with sialic acid. While the Y98F substitution prevents HA binding to SA, the overall structure and conformation of HA remains intact (Zost S. J., et. al., 2019, Cell Rep. 29:4460-4470).

The potential role of cognate and non-cognate interactions between HA and host cells on influenza vaccine outcomes using suprastructures, for example, protein complexes, or VLPs comprising a modified HA that reduces binding of the modified HA to sialic acid (SA) is described herein.

### SUMMARY OF THE INVENTION

The present invention relates to suprastructures or virus like particles (VLPs) that comprise modified influenza hemagglutinin (HA) protein. The modified HA protein comprises one or more than one alteration that reduces interaction of the modified HA to sialic acid (SA), the interaction might be a non-cognate interaction.

According to the present invention there is provided a suprastructure comprising modified influenza hemagglutinin (HA), the modified HA comprising one or more than one alteration that reduces non-cognate interaction of the modified HA to sialic acid (SA) of a target, while maintaining cognate interaction, with the target. Furthermore, it is provided a suprastructure comprising modified influenza hemagglutinin (HA), the modified HA comprising one or more than one alteration that reduces non-cognate interaction of the modified HA to sialic acid (SA) of a protein on the surface of a cell, while maintaining cognate interaction with the cell.

For example, the modified HA may comprise one or more than one alteration that reduces binding of the modified HA to sialic acid (SA), while maintaining cognate interactions, with a target or a cell. Non-limiting examples of the target may include a B cell receptor, and/or one or more targets comprising a B cell surface receptor that comprises SA. Non-limiting examples of a cell may include B cell and non-limiting examples of protein on the surface of the cell may include B cell surface receptor.

The alteration that reduces binding of the modified HA to SA may comprise a substitution, deletion or insertion of one or more amino acids within the modified HA. Furthermore, the suprastructure may be a virus like particle (VLP). A composition comprising the suprastructure or VLP, and a pharmaceutically acceptable carrier, a vaccine comprising the composition, and a vaccine comprising the composition in combination with an adjuvant are also described.

Also provided herein is a plant or portion of a plant comprising a suprastructure or VLP comprising modified influenza hemagglutinin (HA), the modified HA comprising one or more than one alteration that reduces binding of the modified HA to sialic acid (SA) to a target or protein on the surface of a cell, while maintaining cognate interactions with the target or cell. Non-limiting examples of the target may include a B cell receptor, and/or one or more targets comprising a B cell surface receptor that comprises SA. Non-limiting examples of a cell may include B cell and non-limiting examples of the protein on the surface of the cell may include B cell surface receptor.

A nucleic acid encoding a modified HA comprising modified influenza hemagglutinin (HA), the modified HA comprising one or more than one alteration that reduces binding of the modified HA to sialic acid (SA), while maintaining cognate interactions, with a target or protein on the surface of a cell is also described. Non-limiting examples of the target may include a B cell receptor, and/or a B cell surface receptor that comprises SA. Furthermore, a plant or portion of a plant comprising the nucleic acid is provided herein.

Also disclosed is a method of inducing immunity to an influenza virus infection in an animal or subject in need thereof, comprising administering a vaccine, the vaccine comprising:
a suprastructure or VLP comprising a modified influenza hemagglutinin (HA), the modified HA comprising one or more than one alteration that reduces binding of the modified HA to sialic acid (SA) while maintaining cognate interactions with a target for example a protein on the surface of a cell, such as a B cell receptor, a B cell surface receptor that comprises SA, or a combination thereof, and
a pharmaceutical carrier to the animal or subject.
   The vaccine may be administered to the animal or the subject orally, intradermally, intranasally, intramuscularly, intraperitoneally, intravenously, or subcutaneously.

Described herein a method of improving an immunological response of a (first) animal or a subject in response to an antigen challenge comprising,

i) administering to the animal or the subject a first vaccine, the first vaccine comprising a vaccine comprising a suprastructure or VLP comprising a modified influenza hemagglutinin (HA), the modified HA comprising one or more than one alteration that reduces binding of the modified HA to sialic acid (SA) while maintaining cognate interactions with a target, for example a protein on the surface of a cell, such as a B cell receptor or a B cell surface receptor that comprises SA, and a pharmaceutical carrier, to the animal or subject and determining the immunological response;

ii) administering to a second animal or second subject a second vaccine comprising a composition comprising a suprastructure or virus like particle comprising a corresponding parent HA and determining a second immunological response;

iii) comparing the immunological response with the second immunological response, thereby determining the improvement in immunological response; wherein, the immunological response is a cellular immunological response, a humoral immunological response, and both the cellular immunological response and the humoral immunological response.

A method of increasing a magnitude or quality of, or improving, an immunological response of an animal or a subject in response to an antigen challenge is also provided. This method comprises administering a first vaccine, the first vaccine comprising a suprastructure or VLP comprising a modified influenza hemagglutinin (HA), the modified HA comprising one or more than one alteration that reduces binding of the modified HA to sialic acid (SA) while maintaining cognate interactions with a target, for example a protein on the surface of a cell, such as a B cell receptor or a B cell surface receptor that comprises SA; and a pharmaceutical carrier, to the animal or subject and determining the immunological response, wherein the immunological response is a cellular immunological response, a humoral immunological response, and both the cellular immunological response and the humoral immunological response, and wherein the immunological response is increased or improved when compared with a second immunological response obtained following administration of a second vaccine comprising virus like particles comprising a corresponding parent HA to a second subject.

Also provided is a method of producing a suprastructure or virus like particle (VLP) in a host comprising expressing a nucleic acid encoding a modified HA comprising modified influenza hemagglutinin (HA), the modified HA comprising one or more than one alteration that reduces binding of the modified HA to sialic acid (SA) while maintaining cognate interactions with a target, for example a protein on the surface of a cell, such as a B cell receptor or a B cell surface receptor that comprises SA, within the host under conditions that result in the expression of the nucleic acid and production of the suprastructure or VLP. The host may include, but is not limited to a eukaryotic host, a eukaryotic cell, a mammalian host, a mammalian cell, an avian host, an avian cell, an insect host, an insect cell, a baculovirus cell, or a plant host, a plant or a portion of a plant, a plant cell. If desired, the suprastructure or VLP may be obtained or extracted from the host and purified.

A method of producing the suprastructure or the VLP comprising the modified HA in a plant or portion of a plant comprising is also provided, The method comprises introducing the nucleic acid as just defined within the plant or portion of the plant, and growing the plant or portion of the plant under conditions that result in the expression of the nucleic acid and production of the suprastructure or the VLP is disclosed. A method of producing a suprastructure comprising modified HA in a plant or portion of a plant may also comprise, growing a plant, or portion of a plant that comprises the nucleic acid as just defined, under conditions that result in the expression of the nucleic acid and production of the suprastructure or VLP. If desired, in any of these methods, the plant or portion of the plant may be harvested and the suprastructure or VLP purified.

A composition comprising a suprastructure comprising a modified HA, and a pharmaceutically acceptable carrier is also described. The modified HA of the suprastructure comprises one or more than one alteration that reduces binding of the modified HA to sialic acid (SA) while maintaining cognate interactions with a target. Non-limiting examples of the target may include a B cell receptor, and/or one or more targets comprising a B cell surface receptor that comprises SA. Also disclosed is the composition (as just described) comprising the suprastructure or a VLP comprising the modified HA with one or more than one alteration as just described, wherein, the modified HA is selected from:
i) a modified H1 HA, wherein the one or more than one alteration is Y9TF; wherein the numbering of the alteration corresponds to the position of reference sequence with SEQ ID NO: 203;
ii) a modified H3 HA, wherein the one or more than one alteration is selected from Y98F, S136D; Y98F, S136N; Y98F, S137N; Y98F, D190G; Y98F, D190K; Y98F, R222W; Y98F, S228N; Y98F, S228Q; S136D; S136N; D190K; S228N; and S228Q; wherein the numbering of the alteration corresponds to position of reference sequence with SEQ ID NO: 204.
iii) a modified H5 HA, wherein the one or more than one alteration is Y91F; wherein the numbering of the alteration corresponds to position of reference sequence with SEQ ID NO: 205.
iv) a modified H7 HA, wherein the one or more than one alteration is Y88F; wherein the numbering of the alteration corresponds to position of reference sequence with SEQ ID NO: 206;
v) a modified B HA, wherein the one or more than one alteration is selected from S140A; S142A; G138A; L203A; D195G; and L203W; wherein the numbering of the alteration corresponds to position of reference sequence with SEQ ID NO: 207; or
vi) a combination thereof.

A modified influenza H1 hemagglutinin (HA) comprising one or more than one alteration that reduces binding of the modified H1 HA to sialic acid (SA), while maintaining cognate interactions, with a target, for example a B cell receptor, and/or one or more targets comprising a B cell surface receptor that comprises SA is described. The modified H1 HA may comprise plant-specific N-glycans or modified N-glycans. A virus like particle (VLP) comprising the modified H1 HA as just defined is also described. Furthermore, the VLP may comprise one or more than one lipid derived from a plant.

Also disclosed is a modified influenza H3 hemagglutinin (HA) comprising one or more than one alteration that reduces binding of the modified H3 HA to sialic acid (SA), while maintaining cognate interactions with a target, for example a B cell receptor, and/or one or more targets comprising a B cell surface receptor that comprises SA. The modified H3 HA may comprise plant-specific N-glycans or modified N-glycans. A virus like particle (VLP) comprising the modified H3 HA as just defined is also described. Furthermore, the VLP may comprise one or more than one lipid derived from a plant.

A modified influenza H7 hemagglutinin (HA) comprising one or more than one alteration that reduces binding of the modified H7 HA to sialic acid (SA), while maintaining cognate interactions with a target, for example a B cell receptor, and/or one or more targets comprising a B cell surface receptor that comprises SA, is also described. The modified H7 HA may comprise plant-specific N-glycans or modified N-glycans. A virus like particle (VLP) comprising the modified H7 HA as just defined is also described. Furthermore, the VLP may comprise one or more than one lipid derived from a plant.

Also disclosed is a modified influenza H5 hemagglutinin (HA) comprising one or more than one alteration that reduces binding of the modified H5 HA to sialic acid (SA), while maintaining cognate interactions, with a target, for example a B cell receptor, and/or one or more targets comprising a B cell surface receptor that comprises SA. The modified H5 HA may comprise plant-specific N-glycans or modified N-glycans. A virus like particle (VLP) comprising the modified B HA as just defined is also described. Furthermore, the VLP may comprise one or more than one lipid derived from a plant.

Further disclosed is a suprastructure comprising modified influenza hemagglutinin (HA), the modified HA comprising one or more than one alteration, the modified HA being selected from:
i) a modified H1 HA, wherein the one or more than one alteration is Y91F; wherein the numbering of the alteration corresponds to the position of reference sequence with SEQ ID NO: 203;
ii) a modified H3 HA, wherein the one or more than one alteration is selected from Y98F, S136D; Y98F, S136N; Y98F, S137N; Y98F, D190G; Y98F, D190K; Y98F, R222W; Y98F, S228N; Y98F, S228Q; S136D; S136N; D190K; S228N; and S228Q; wherein the numbering of the alteration corresponds to position of reference sequence with SEQ ID NO: 204.
iii) a modified H5 HA, wherein the one or more than one alteration is Y91F; wherein the numbering of the alteration corresponds to position of reference sequence with SEQ ID NO: 205.
iv) a modified H7 HA, wherein the one or more than one alteration is Y88F; wherein the numbering of the alteration corresponds to position of reference sequence with SEQ ID NO: 206;
v) a modified B HA, wherein the one or more than one alteration is selected from S140A; S142A; G138A; L203A; D195G; and L203W; wherein the numbering of the alteration corresponds to position of reference sequence with SEQ ID NO: 207; or
vi) a combination thereof.

In the suprastructure as described above, the modified HA reduces non-cognate interaction of the modified HA to sialic acid (SA) of a protein on the surface of a cell, while maintaining cognate interaction, with the cell. The suprastructure and/or the modified HA comprised within the suprastructure may increases an immunological response of an animal or a subject in response to an antigen challenge.

Also disclosed is a modified influenza B hemagglutinin (HA) comprising one or more than one alteration that reduces binding of the modified B HA to sialic acid (SA), while maintaining cognate interactions, with a target, for example a B cell receptor, and/or one or more targets comprising a B cell surface receptor that comprises SA. The modified B HA may comprise plant-specific N-glycans or modified N-glycans. A virus like particle (VLP) comprising the modified B HA as just defined is also described. Furthermore, the VLP may comprise one or more than one lipid derived from a plant.

A method of increasing a magnitude or quality of, or improving, an immunological response of an animal or a subject in response to an antigen challenge is also provided. The method comprises administering a first vaccine, the first vaccine comprising the vaccine as defined above to the animal or subject and determining the immunological response, wherein the immunological response is a cellular immunological response, a humoral immunological response, and both the cellular immunological response and the humoral immunological response, and wherein the immunological response is increased or improved when compared with a second immunological response obtained following administration, to a second animal or subject, of a second vaccine comprising a composition comprising virus like particles comprising a corresponding wild type HA.

As described herein, use of a modified HA protein, a suprastructure (protein suprastructure), or VLP comprising the modified HA protein, as an influenza vaccine was observed to increase immunogenicity and efficacy when compared to the immunogenicity and efficacy of an influenza vaccine comprising a corresponding parent HA that does not comprise the modification that results in reduced, non-detectable, or no non-cognate interaction with SA, for example, reduced, non-detectable, or no SA binding. The parent HA that does not comprise the modification that results in reduced, non-detectable, or no non-cognate interaction with SA may include a non-modified HA, a wild type influenza HA, an HA comprising a sequence that is altered, but the alteration is not associated with SA binding, a suprastructure or VLP comprising the parent HA, a wild type influenza HA, or the HA comprising a sequence that is altered, but the alteration is not associated with SA binding.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1A shows a sequence alignment of the amino acid sequences of hemagglutinin (HA) of A/California/7/09 (H1N1) (SEQ ID NO:2); A/Idaho/7/18 (H1N1) (SEQ ID NO:101); A/Brisbane/02/18 (H1N1) (SEQ ID NO: 195); A/Kansas/14/17 (H3N2) (SEQ ID NO: 61); A/Minnesota/41/19 (H3N2) (SEQ ID NO: 13). A/Indonesia/5/2005 (H5N1) (SEQ ID NO: 14); A/Egypt/NO4915/14 (H5N1) (SEQ ID NO:108; A/Shanghai/2/2013 (H7N9) (SEQ ID NO: 21); A/Hangzhou/1/13 (H7N9) (SEQ ID NO: 109); Outlined residues align with amino acids Y98 of HA from influenza H3 strains, for example A/Kansas/14/17 (H3N2) (SEQ ID NO: 61). Signal peptides have been removed for clarity. FIG. 1B shows a sequence alignment of the amino acid sequences of hemagglutinin (HA) of B/Phuket/3703/13 (Yamagata lineage) (SEQ ID NO:28); B/Singapore/INFKK-16-0569/16 (Yamagata lineage) (SEQ ID NO:14); B/Maryland/15/16 (Victoria lineage) (SEQ ID NO:15); B/Victoria/705/18 (Victoria lineage) (SEQ ID NO:16); B/Washington/12/19 (Victoria lineage) (SEQ ID NO:17); B/Darwin/8/19 (Victoria lineage) (SEQ ID NO:18); B/Darwin/20/19 (Victoria lineage) (SEQ ID NO:19). Signal peptides have been removed for clarity. FIG. 1C shows the production of virus-like particle (VLP) comprising either HA that bind to sialic acid (binding VLP) or HA that do not bind to sialic acid (non-binding VLP) using HA from the four types of seasonal influenza: influenza type A H1 (H1/Brisbane), influenza type A H3 (H3/Kansas), influenza B/Yamagata (B/Phuket), and influenza B/Victoria (B/Maryland). The production of VLPs was also confirmed for H1/California, H1/Idaho, B/Singapore and B/Washington (data not shown).

FIG. 2A shows the relative yields (fold-change) of VLPs comprising a H1 A/Idaho/07/2018 (parent H1; set to “1”), and a VLP comprising modified Y91F H1 A/Idaho/07/2018 derived from the parent H1 (n=6). FIG. 2B shows the hemagglutination titers of VLPs comprising H1 A/Idaho/07/2018 (parent H1), and a VLP comprising modified Y91F H1 A/Idaho/07/2018, derived from the parent H1 (n=6). FIG. 2C shows the relative yields (fold-change) of VLPs comprising a H1 A/Brisbane/02/2018 (parent H1; set to “1”), and a VLP comprising modified Y91F H1 A/Brisbane/02/2018 derived from the parent H1 (n=6). FIG. 2D shows the hemagglutination titers of VLPs comprising H1 A/Brisbane/02/2018 (parent H1), and a VLP comprising modified Y91F H1 A/Brisbane/02/2018, derived from the parent H1 (n=6).

FIG. 3A shows the relative yields (fold-change) of VLPs comprising H3 Kansas/14/2017 (parent H3; construct 7281; left hand bar) and VLPs comprising Y98F H3 Kansas/14/2017 (construct 8179; derived from the parent H3); Y98F, S136D H3 Kansas/14/2017 (construct 8384; derived from the parent H3); Y98F, S136N H3 Kansas/14/2017 (construct 8385; derived from the parent H3); Y98F, S137N H3 Kansas/14/2017 (construct 8387; derived from the parent H3); Y98F, D190G H3 Kansas/14/2017 (construct 8388; derived from the parent H3); Y98F, D190K H3 Kansas/14/2017 (construct 8389; derived from the parent H3); Y98F, R222W H3 Kansas/14/2017 (construct 8391; derived from the parent H3); Y98F, S228N H3 Kansas/14/2017 (construct 8392; derived from the parent H3); Y98F, S228Q H3 Kansas/14/2017 (construct 8393; derived from the parent H3), (n=6). FIG. 3B shows the hemagglutination titers of VLPs comprising H3 Kansas/14/2017 (parent H3; construct 7281; left hand bar), and VLPs comprising Y98F H3 Kansas/14/2017 (construct 8179; derived from the parent H3); Y98F, S136D H3 Kansas/14/2017 (construct 8384; derived from the parent H3); Y98F, S136N H3 Kansas/14/2017 (construct 8385; derived from the parent H3); Y98F, S137N H3 Kansas/14/2017 (construct 8387; derived from the parent H3); Y98F, D190G H3 Kansas/14/2017 (construct 8388; derived from the parent H3); Y98F, D190K H3 Kansas/14/2017 (construct 8389; derived from the parent H3); Y98F, R222W H3 Kansas/14/2017 (construct 8391; derived from the parent H3); Y98F, S228N H3 Kansas/14/2017 (construct 8392; derived from the parent H3); Y98F, S228Q H3 Kansas/14/2017 (construct 8393; derived from the parent H3), (n=6). FIG. 3C shows the relative yields (fold-change) of VLPs comprising H3 Kansas/14/2017 (parent H3; construct 7281; left hand bar) and VLPs comprising S136D H3 Kansas/14/2017 (construct 8477; derived from the parent H3); S136N H3 Kansas/14/2017 (construct 8478; derived from the parent H3); D190K H3 Kansas/14/2017 (construct 8481; derived from the parent H3); R222W H3 Kansas/14/2017 (construct 8482; derived from the parent H3); S228N H3 Kansas/14/2017 (construct 8483; derived from the parent H3); S228Q H3 Kansas/14/2017 (construct 8484; derived from the parent H3), (n=6). FIG. 3D shows the hemagglutination titers of VLPs comprising H3 Kansas/14/2017 (parent H3; construct 7281; left hand bar) and VLPs comprising S136D H3 Kansas/14/2017 (construct 8477; derived from the parent H3); S136N H3 Kansas/14/2017 (construct 8478; derived from the parent H3); D190K H3 Kansas/14/2017 (construct 8481; derived from the parent H3); R222W H3 Kansas/14/2017 (construct 8482; derived from the parent H3); S228N H3 Kansas/14/2017 (construct 8483; derived from the parent H3); S228Q H3 Kansas/14/2017 (construct 8484; derived from the parent H3), (n=6).

FIG. 4A shows the relative yields (fold-change) of VLPs comprising B/Phuket/3073/2013 (parent B; construct 2835; left hand bar, set to “1”), and VLPs comprising S140A B/Phuket/3073/2013 (construct 8352; derived from the parent B); S142A B/Phuket/3073/2013 (construct 8354; derived from the parent B HA); G138A B/Phuket/3073/2013 (construct 8358; derived from the parent B HA); L203A B/Phuket/3073/2013 (construct 8363; derived from the parent B HA); D195G B/Phuket/3073/2013 (construct 8376; derived from the parent B HA); L203W B/Phuket/3073/2013 (construct 8382; derived from the parent B HA), (n=6). FIG. 4B shows the hemagglutination titers of VLPs comprising B/Phuket/3073/2013 (parent B HA; construct 2835; left hand bar), and VLPs comprising S140A B/Phuket/3073/2013 (construct 8352; derived from the parent B HA); S142A B/Phuket/3073/2013 (construct 8354; derived from the parent B HA); G138A B/Phuket/3073/2013 (construct 8358; derived from the parent B HA); L203A B/Phuket/3073/2013 (construct 8363; derived from the parent B HA); D195G B/Phuket/3073/2013 (construct 8376; derived from the parent B HA); L203W B/Phuket/3073/2013 (construct 8382; derived from the parent B HA), (n=6). FIG. 4C shows the relative yields (fold-change) of VLPs comprising B/Singapore/INFKK-16-0569/2016 (parent B; construct 2879; left hand bar, set to “1”), and VLPs comprising G138A B/Singapore/INFKK-16-0569/2016 (construct 8485; derived from the parent B HA); S140A B/Singapore/INFKK-16-0569/2016 (construct 8486; derived from the parent B HA); S142A B/Singapore/INFKK-16-0569/2016 (construct 8487; derived from the parent B HA); D195G B/Singapore/INFKK-16-0569/2016 (construct 8488; derived from the parent B HA); L203A B/Singapore/INFKK-16-0569/2016 (construct 8489; derived from the parent B HA); L203W B/Singapore/INFKK-16-0569/2016 (construct 8490; derived from the parent B HA), (n=6). FIG. 4D shows the hemagglutination titers of VLPs comprising B/Singapore/INFKK-16-0569/2016 (parent B; construct 2879; left hand bar, set to “1”), and VLPs comprising G138A B/Singapore/INFKK-16-0569/2016 (construct 8485; derived from the parent B HA); S140A B/Singapore/INFKK-16-0569/2016 (construct 8486; derived from the parent B HA); S142A B/Singapore/INFKK-16-0569/2016 (construct 8487; derived from the parent B HA); D195G B/Singapore/INFKK-16-0569/2016 (construct 8488; derived from the parent B HA); L203A B/Singapore/INFKK-16-0569/2016 (construct 8489; derived from the parent B HA); L203W B/Singapore/INFKK-16-0569/2016 (construct 8490; derived from the parent B HA), (n=6). FIG. 4E shows the relative yields (fold-change) of VLPs comprising B/Maryland/15/2016 (parent B; construct 6791; left hand bar, set to “1”), and VLPs comprising G138A B/Maryland/15/2016 (construct 8434; derived from the parent B HA); S140A B/Maryland/15/2016 (construct 8435; derived from the parent B HA); S142A B/Maryland/15/2016 (construct 8436; derived from the parent B HA); D194G B/Maryland/15/2016 (construct 8437; derived from the parent B HA); L202A B/Maryland/15/2016 (construct 8438; derived from the parent B HA); L202W B/Maryland/15/2016 (construct 8439; derived from the parent B HA), (n=6). FIG. 4F shows the hemagglutination titers of VLPs comprising B/Maryland/15/2016 (parent B; construct 6791; left hand bar, set to “1”), and VLPs comprising G138A B/Maryland/15/2016 (construct 8434; derived from the parent B HA); S140A B/Maryland/15/2016 (construct 8435; derived from the parent B HA); S142A B/Maryland/15/2016 (construct 8436; derived from the parent B HA); D194G B/Maryland/15/2016 (construct 8437; derived from the parent B HA); L202A B/Maryland/15/2016 (construct 8438; derived from the parent B HA); L202W B/Maryland/15/2016 (construct 8439; derived from the parent B HA), (n=6). FIG. 4G shows the relative yields (fold-change) of VLPs comprising B/Washington/02/2019 (parent B; construct 7679; left hand bar, set to “1”), and VLPs comprising G138A B/Washington/02/2019 (construct 8440; derived from the parent B HA); S140A B/Washington/02/2019 (construct 8441; derived from the parent B HA); S142A B/Washington/02/2019 (construct 8442; derived from the parent B HA); D193G B/Washington/02/2019 (construct 8443; derived from the parent B HA); L201A B/Washington/02/2019 (construct 8444; derived from the parent B HA); L201W B/Washington/02/2019 (construct 8445; derived from the parent B HA), (n=6). FIG. 4H shows the hemagglutination titers of VLPs comprising B/Washington/02/2019 (parent B; construct 7679; left hand bar, set to “1”), and VLPs comprising G138A B/Washington/02/2019 (construct 8440; derived from the parent B HA); S140A B/Washington/02/2019 (construct 8441; derived from the parent B HA); S142A B/Washington/02/2019 (construct 8442; derived from the parent B HA); D193G B/Washington/02/2019 (construct 8443; derived from the parent B HA); L201A B/Washington/02/2019 (construct 8444; derived from the parent B HA); L201W B/Washington/02/2019 (construct 8445; derived from the parent B HA), (n=6). FIG. 4I shows the relative yields (fold-change) of VLPs comprising B/Darwin/20/2019 (parent B; construct 8333; left hand bar, set to “1”), and VLPs comprising G138A B/Darwin/20/2019 (construct 8458; derived from the parent B HA); S140A B/Darwin/20/2019 (construct 8459; derived from the parent B HA); S142A B/Darwin/20/2019 (construct 8460; derived from the parent B HA); D193G B/Darwin/20/2019 (construct 8461; derived from the parent B HA); L201A B/Darwin/20/2019 (construct 8462; derived from the parent B HA); L201W B/Darwin/20/2019 (construct 8463; derived from the parent B HA), (n=6). FIG. 4J shows the hemagglutination titers of VLPs comprising B/Darwin/20/2019 (parent B; construct 8333; left hand bar, set to “1”), and VLPs comprising G138A B/Darwin/20/2019 (construct 8458; derived from the parent B HA); S140A B/Darwin/20/2019 (construct 8459; derived from the parent B HA); S142A B/Darwin/20/2019 (construct 8460; derived from the parent B HA); D193G B/Darwin/20/2019 (construct 8461; derived from the parent B HA); L201A B/Darwin/20/2019 (construct 8462; derived from the parent B HA); L201W B/Darwin/20/2019 (construct 8463; derived from the parent B HA), (n=6). FIG. 4K shows the relative yields (fold-change) of VLPs comprising B/Victoria/705/2018 (parent B; construct 8150; left hand bar, set to “1”), and VLPs comprising G138A B/Victoria/705/2018 (construct 8446; derived from the parent B HA); S140A B/Victoria/705/2018 (construct 8447; derived from the parent B HA); S142A B/Victoria/705/2018 (construct 8448; derived from the parent B HA); D193G B/Victoria/705/2018 (construct 8450; derived from the parent B HA); L201A B/Victoria/705/2018 (construct 8449; derived from the parent B HA); L201W B/Victoria/705/2018 (construct 8451; derived from the parent B HA), (n=6). FIG. 4L shows the hemagglutination titers of VLPs comprising B/Victoria/705/2018 (parent B; construct 8150; left hand bar, set to “1”), and VLPs comprising G138A B/Victoria/705/2018 (construct 8446; derived from the parent B HA); S140A B/Victoria/705/2018 (construct 8447; derived from the parent B HA); S142A B/Victoria/705/2018 (construct 8448; derived from the parent B HA); D193G B/Victoria/705/2018 (construct 8450; derived from the parent B HA); L201A B/Victoria/705/2018 (construct 8449; derived from the parent B HA); L201W B/Victoria/705/2018 (construct 8451; derived from the parent B HA), (n=6). FIG. 4M shows the hemagglutination titers of VLPs comprising H5 A/Indonesia/5/05 (parent H5; construct 2295; left hand bar, set to “1”), and VLPs comprising modified HA Y91F H5 A/Indonesia/5/05 (construct 6101; derived from the parent H5 HA). FIG. 4N shows the hemagglutination titers of VLPs comprising H7 A/Shanghai/2/2013 (parent H7; construct 6102; left hand bar, set to “1”), and VLPs comprising modified HA Y88F H7 A/Shanghai/2/2013 (construct 6103; derived from the parent H7 HA);

FIG. 5A shows that Y91F H1-VLP is unable to agglutinate cells. Human PBMC (1×10⁶) incubated with VLP (5 μg/mL) for 30 min (37° C., 5% CO₂). Left hand panel shows PBMC incubated with cRPMI medium (control) with no agglutination observed; Middle panel shows agglutination following incubation of PBMC with parent H1 VLP (wild type/non-modified H1 A/California/07/2009 VLP); Right hand panel shows no agglutination when PBMC were incubated with Y91F H1 A/California/07/2009 VLP. FIG. 5B shows that Y91F H1 A/California/07/2009 VLP is unable to agglutinate cells. Hemagglutination of 0.5% turkey erythrocytes incubated for 2h with H1 A/California/07/2009 VLP (parent H1), or Y91F H1 A/California/07/2009 VLP (2-fold serial dilution). Upper panel shows agglutination in the presence of parent H1 VLP; Lower panel shows no agglutination in the presence of Y91F H1 A/California/07/2009 VLP. FIG. 5C shows that Y91F H1 A/California/07/2009 VLP does not bind glycans comprising sialic acid, determined using SPR; Control: parent H1 A/California/07/2009 VLP. Left panel: total protein from H1 A/California/07/2009 VLP and Y91F H1 A/California/07/2009 VLP; Right panel H1 A/California/07/2009 VLP and Y91F A/California/07/2009 VLP binding with sialic acid; BLQ signifies “below limit of quantification”. FIG. 5D shows that Y98F H3 A/Kansas/14/17 VLP binds glycans comprising sialic acid, determined using SPR; Control: parent H3 A/Kansas/14/17. Left panel: total protein from parent H3 A/Kansas/14/17 VLP and Y98F A/Kansas/14/17 VLP; Right panel: parent H3 A/Kansas/14/17 VLP and Y98F A/Kansas/14/17 VLP binding with sialic acid.

FIG. 6 shows HA-SA interactions influence human PBMC activation. 1×10⁶ PBMC were stimulated with wild type/non-modified H1 A/California/07/2009 VLP (parent H1) or Y91F H1 A/California/07/2009 VLP for 6h (37° C., 5% CO₂) and CD69 was detected by flow cytometry. Data are presented as the proportion of CD69⁺ cells within each PBMC sub-population. Left panel: B cells; middle panel: CD4⁺ cells; Right panel: CD8⁺ cells. Error bars represent the standard error of the mean (SEM), (n=3).

FIG. 7A shows that Y91F H1-VLP elicits a stronger neutralizing antibody response than native H1 A/California/07/2009 VLP (wild type/non-modified; parent H1). BALB/c mice (8-10 weeks) were vaccinated IM (intermuscular) with 3 μg H1 A/California/07/2009 VLP or Y91F H1 A/California/07/2009 VLP, or an equivalent volume of PBS. Serum was collected 21 days post-vaccination and the H1-specific neutralizing antibody response was characterized by hemagglutination inhibition assay (HAI; left panel) and microneutralization assay (MN; right panel). Sample (n=9). Error bars for HAI and MN represent 95% confidence intervals of the geometric mean. Statistical significance was determined by Mann-Whitney test (*P<0.033, **P<0.01, ***P<0.001). FIG. 7B shows a time course of H1-specific IgG titers by ELISA up to 8 weeks post vaccination. BALB/c mice (8-10 weeks) were vaccinated IM with 3 μg H1 A/California/07/2009 VLP (parent H1) or Y91F H1 A/California/07/2009 VLP, or an equivalent volume of PBS. Serum was collected at the times indicated. Error bars represent standard error of the mean (SEM). FIG. 7C shows a time course of the avidity index of H1-specific IgG at 8 weeks post vaccination (% bound after treatment with indicated concentration of urea). BALB/c mice (8-10 weeks) were vaccinated IM with 3 μg H1 A/California/07/2009 VLP (parent H1) or Y91F H1 A/California/07/2009 VLP, or an equivalent volume of PBS. Serum was collected at the times indicated. Error bars represent standard error of the mean (SEM). FIG. 7D shows a time course of H7 IgG Titers up to 8 weeks post vaccination (3 μg). BALB/c mice (8-10 weeks) were vaccinated IM with 3 μg H7 A/Shanghai/2/2013 VLP (parent H7) or Y88F H7 A/Shanghai/2/2013 VLP, or an equivalent volume of PBS, and serum was collected at the indicated times. H7-specific IgG titers were determined by ELISA. FIG. 7E shows a time course of the avidity index of H7-specific IgG up to 2 months post vaccination. BALB/c mice (8-10 weeks) were vaccinated IM with 3 μg H7 A/Shanghai/2/2013 VLP (parent H7) or Y88F H7 A/Shanghai/2/2013 VLP, or an equivalent volume of PBS. Serum was collected at the indicated times. Avidity Index: % bound after treatment at 6M and 8M urea. Error bars represent SEM. FIG. 7F shows long term maintenance of IgG avidity. Y91F H1 A/California/07/2009 VLP results in the production of higher avidity IgG compared to the native H1 A/California/07/2009 VLP (parent H1). Avidity is maintained in both groups for at least 7 months. BALB/c mice (8-10 weeks) were vaccinated IM with 3 μg wild type/non-modified H1 A/California/07/2009 VLP or Y91F H1 A/California/07/2009 VLP, or an equivalent volume of PBS, and serum was collected at the time intervals indicated.

FIGS. 7G and 7H show that the non-binding H1 A/California/07/2009 VLP resulted in higher HI and MN titers at 7 months post-vaccination and improved durability of HI titers. Mice (n=7-8/group) were vaccinated (IM) with H1-VLP or Y91F H1-VLP (3 μg/dose). Sera were collected on a monthly basis to measure HI titers (7G) and MN titers (7H). Statistical significance was determined by multiple t tests corrected for multiple comparisons using the Holm-Sidak method (*p<0.033, **p<0.01). FIG. 7I shows hemagglutination inhibition (HI) titers following vaccination with H1 A/Idaho/07/2018 VLP or Y91F A/Idaho/07/2018 VLP. Mice (n=8/group) were vaccinated with 1 μg binding or non-binding (Y91F) H1-VLP (A/Idaho/07/2018) and boosted with 1 μg at day 21. Sera were collected and HI titers were measured 21d post-boost. Statistical significance was evaluated using the Mann-Whitney test. FIG. 7J shows IgG titers by ELISA with H1 A/Idaho/07/2018 VLP or Y91F A/Idaho/07/2018 VLP following a single vaccine dose (D21) and post-boost (D42). Mice (n=8/group) were vaccinated with 1 μg binding or non-binding (Y91F) H1-VLP (A/Idaho/07/2018) and boosted with 1 μg at day 21. Sera were collected and H1-specific IgG was measured by ELISA 21d post-prime and 21d post-boost (d42). FIG. 7K shows IgG titers by ELISA following vaccination with H1 A/Brisbane/02/2018 HA trimers or Y91F A/Brisbane/02/2018 HA trimers following a single vaccine dose (D21) and post-boost (D42). Mice (n=18/group) were vaccinated with 0.5 μg binding or non-binding recombinant H1 (A/Brisbane/02/2018) HA and boosted with 0.5 μg at day 21. Sera were collected and H1-specific IgG was measured by ELISA 21d post-prime and 21d post-boost (d42). FIG. 7L shows the avidity index of H1-specific IgG with H1 A/Brisbane/02/2018 HA or Y91F A/Brisbane/02/2018 HA. IgG avidity was assessed using an avidity ELISA. Bound serum samples were treated with 4-6M Urea and the avidity index represents the proportion of IgG that remains bound after the urea incubation ([IgG titer 2-10M urea]/[IgG titer 0M urea]). Statistical significance was determined by Mann-Whitney test (*p<0.033, ***p<0.001). FIG. 7M shows no change in hemagglutination inhibition (HI) titers following vaccination with parent B/Phuket/3073/2013 and non-binding (NB) D195G B/Phuket/3073/2013 VLP (left panel). Mice (n=7-8/group) were vaccinated with 1 μg binding B/Phuket/3073/2013 VLP or non-binding (NB) D195G B/Phuket/3073/2013 VLP and boosted with 1 μg at day 21. Sera were collected and HI titers were measured 21d post-boost. Microneutralization (MN) titers were lower following vaccination with non-binding (NB) D195G B/Phuket/3073/2013 VLP as compared to binding B/Phuket/3073/2013 VLP but the difference was not statistically significant (right panel). FIG. 7N shows that binding HA B/Phuket/3073/2013 VLP or non-binding (NB) D195G HA B/Phuket/3073/2013 VLP resulted in similar amounts of HA-specific IgG but there is a slight increase in IgG avidity among mice vaccinated with the non-binding D195G B/Phuket/3073/2013 VLP. Mice (n=7-8/group) were vaccinated with 1 μg binding or non-binding D195G B/Phuket/3073/2013 VLP and boosted with 1 μg at day 21. Sera were collected and B-specific IgG was measured by ELISA 21d post-prime and 21d post-boost (d42) (right panel). FIG. 7O shows IgG avidity assessed using an avidity ELISA. Bound serum samples were treated with 4-6M Urea and the avidity index represents the proportion of IgG that remains bound after the urea incubation ([IgG titer 2-10M urea]/[IgG titer 0M urea]). Differences in avidity were not statistically significant between binding HA B/Phuket/3073/2013 VLP or non-binding (NB) D195G HA B/Phuket/3073/2013 VLP.

FIG. 8A shows increase in memory B cells following vaccination with Y91F H1-BLP. BALB/c mice (8-10 weeks) were vaccinated IM (intermuscular) on Day 0 and Day 21 with 3 μg or 0.5 μg wild type/non-modified H1 A/California/07/2009 VLP (parent H1) or Y91F H1 A/California/07/2009 VLP, or an equivalent volume of PBS. H1-specific memory B cells were measured in the spleen and bone marrow by IgG ELISpot 4 weeks post-boost. Cells were stimulated for 72h with R848 and recIL-2 to identify memory B cells and were evaluated immediately following isolation for in vivo activated ASCs. Spots were counted and measured using the ImmunoSpot plate reader (Cellular Technology Limited). Error bars: standard error of the mean (SEM). Statistical significance was determined Kruskal Wallis test (*P<0.033, **P<0.01). FIG. 8B shows in vivo activated ASCs were measured in the spleen and bone marrow by IgG ELISpot 4 weeks post-boost. Cells were evaluated immediately following isolation for in vivo activated ASCs. Spots were counted and measured as indicated in FIG. 8A. FIG. 8C shows in vivo activated ASCs measured in the spleen (left) and bone marrow (right) by IgG ELISpot 4 weeks post-boost. IgG ELISpot assay was carried out (as per FIG. 8B) to identify in vivo activated ASCs and pictures were obtained using the ImmunoSpot plate reader (Cellular Technology Limited). FIG. 8D shows that the non-binding H1-VLP resulted in slightly increased bone marrow plasma cells (BMPC) at 7 months post-vaccination and correlated with maintenance of MN titers. Mice (n=7-8/group) were vaccinated (IM) with H1-VLP or Y91F H1-VLP (3 μg/dose). Mice were euthanized at 7 mpv and BM was collected to quantify H1-specific plasma cells (PC) in the bone marrow by ELISpot. Representative wells from each group are shown on the right. All mice that had >10 BMPC/1×10⁶ cells maintained their MN titers between 3 and 7 months post-vaccination. All mice with <10 BMPC/1×10⁶ cells had a decline in MN titers after 3 months.

FIG. 9A shows the proliferative response in mice vaccinated with wild type/non-modified H1 A/California/07/2009 VLP (parent H1) or Y91F H1 A/California/07/2009 VLP. FIG. 9B shows the proliferative response in mice vaccinated with a series of peptides obtained from parent H1 A/California/07/2009 VLP (left hand bar) and Y91F H1 A/California/07/2009 VLP (right hand bar). BALB/c mice (8-10 weeks) were vaccinated IM with 3 μg parent (wild type/non-modified) H1 A/California/07/2009 VLP or Y91F H1 A/California/07/2009 VLP, or an equivalent volume of PBS. Four weeks post-vaccination, mice were euthanized and spleens were harvested. Splenocytes (2.5×10⁵) were stimulated with parent (wild type/non-modified) H1 A/California/07/2009 VLP (FIG. 9A), or pools of 20 overlapping peptides (15aa each) spanning the entire parent H1 HA sequence (2 μg/mL; FIG. 9B) for 72h (37° C., 5% CO₂). Proliferative responses were measured on the basis of bromodeoxyuridine (BrdU) incorporation and data are presented as a ratio of proliferation compared to unstimulated cells. Error bars represent standard error of the mean (SEM), n=8.

FIG. 10A shows that cell mediated immune response is maintained upon vaccination with Y91F H1 A/California/07/2009 VLP. BALB/c mice (8-10 weeks) were vaccinated IM with 3 μg wild type/non-modified H1 A/California/07/2009 VLP (parent H1) or Y91F H1 A/California/07/2009 VLP, or an equivalent volume of PBS. Four weeks post-vaccination, or post-boost at day 28, mice were euthanized and spleens were harvested. Splenocytes (1×10⁶) were stimulated with wild type/non-modified H1 A/California/07/2009 VLP or Y91F H1 A/California/07/2009 VLP (2 μg/mL) for 18h (37° C., 5% CO₂). Intracellular IL-2, TNFα, and IFNγ were measured by flow cytometry. Data are presented as total proportion of CD4⁺ T cells producing at least one of the measured cytokines. Left bar: PBS; Middle bar parent H1-VLP; right bar: Y91F H1 VLP. FIG. 10B shows monofunctional CD4⁺ T cell populations (methods as per FIG. 10A). Left bar: PBS; Middle bar: parent H1 HA VLP; right bar: Y91F H1 VLP. FIG. 10C shows polyfunctional CD4⁺ T cell populations (methods as per FIG. 10A). All values are background subtracted using unstimulated cells from the same animal. Left bar: PBS; Middle bar: parent H1 HA VLP; right bar: Y91F H1 VLP. Error bars represent standard error of the mean (SEM), n=10-16. Statistical significance was determined by Brown-Forsythe and Welch one-way ANOVA(*P<0.033). FIG. 10D shows the data from FIGS. 10A-10C in a different format as follows: Left Panel: frequency of CD4+ T cells expressing CD44 (antigen specific) and at least one of IL-2, TNFα or IFNγ. Background values obtained from non-stimulated samples were subtracted from values obtained following stimulation with H1-VLP. Right panel: individual cytokine signatures for each mouse obtained by Boolean analysis. Background values obtained from non-stimulated samples were subtracted from values obtained following stimulation with H1-VLP. The bar graph shows the frequency of each of the populations and the pie charts show the prevalence of each responding population among total responding cells. FIG. 10E shows that the frequency of IL-2⁺TNFα⁺IFNγ⁻ CD4⁺ T cells in the BM correlate with HI titer. Mice vaccinated with the non-binding HI-VLP had a significant increase in the frequency of IL-2⁺TNFα⁺IFNγ⁺ CD4⁺ T cells in the BM (see FIG. 10D) which correlated with increased HI titers in these mice. Rank correlation technique was applied to evaluate the relationship between the frequency of IL-2⁺TNFα⁺IFNγ⁻ CD4⁺ T cells in the BM and HAI titer. Mice vaccinated with Y91F HI-VLP are shown in outlined white circle and H1-VLP are shown in solid dark. FIGS. 10F and 10G show that total splenic CD4 T cell responses were maintained upon introduction of the non-binding mutation (1 week post-boost). Mice (n=8/group) were vaccinated with 1 μg binding or non-binding (Y91F) HI-VLP (A/Idaho/07/2018) and boosted with 1 μg at day 21. Mice were euthanized 1 week post-boost and spleens were harvested to measure antigen-specific (CD44+) CD4 T cells by flow cytometry. Both vaccines resulted in similar frequencies of responding cells (10F) with similar frequencies of polyfunctional CD4 T cells (10G). Statistical significance was determined by Kruskal-Wallis test with Dunn's multiple comparisons (10F) or two-way ANOVA with Tukey's multiple comparisons (10G). *p<0.033, **p<0.01, ***p<0.001. FIGS. 10H and 10I show that fewer CD4 T cells expressed IFNγ upon vaccination with non-binding H1-VLP (3 weeks post-boost). Mice (n=8/group) were vaccinated with 1 μg binding or non-binding (Y91F) H1-VLP (A/Idaho/07/2018) and boosted with 1 μg at day 21. Mice were euthanized 3 weeks post-boost and spleens were harvested to measure antigen-specific (CD44+) CD4 T cells by flow cytometry. The frequency of total responding CD4 T cells was reduced following vaccination with Y91F H1-VLP but this difference was not significant (10H). Similar to H1 California, the IL-2⁺TNFα⁺IFNγ⁻ population dominated the response to Y91F H1-VLP (10G). However, most IFNγ⁺ populations were reduced in mice vaccinated with Y91F H1-VLP. Statistical significance was determined by Kruskal-Wallis test with Dunn's multiple comparisons (10H) or two-way ANOVA with Tukey's multiple comparisons (10I) *p<0.033, **p<0.01, ***p<0.001.

FIG. 11A shows percent survival following vaccination over a 12 day period. Female BALB/c mice were challenged with H1N1 A/California/07/09 (1.58×10³ TCID₅₀) 28 days post-vaccination with 3 μg H1 A/California/07/2009 VLP (parent H1), 3 μg Y91F H1 A/California/07/2009 VLP, or an equivalent volume of PBS. Mice were closely monitored for weight loss and were euthanized if they lost >20% of their initial weight. Error bars represent standard error of the mean (SEM), n=12. FIG. 11B show percent weight loss each day following infection over the 12-day period following the challenge with H1N1 A/California/07/09 (1.58×10³ TCID₅₀) 28 days post-vaccination with 3 μg H1 A/California/07/2009 VLP (parent H1), 3 μg Y91F H1 A/California/07/2009 VLP, or an equivalent volume of PBS. Error bars represent SEM, n=12. FIG. 11C shows that Y91F H1 A/California/07/2009 VLP promotes enhanced viral clearance following challenge with H1N1 A/California/07/09 (1.58×10³ TCID₅₀) 28 days post-vaccination with 3 μg wild type/non-modified H1 A/California/07/2009 VLP (parent H1), 3 μg Y91F H1 A/California/07/2009 VLP, or an equivalent volume of PBS. At 3 and 5 dpi a subset of the mice were euthanized and lungs were collected and homogenized to measure viral load by TCID₅₀. Viral titers were calculated using the Karber method. Error bars represent SEM, n=9. FIG. 11D shows the cytokine profiles of mock-infected and infected lungs at 3 dpi and 5 dpi (days post infection). Mice were challenged with 1.6×10³ TCID₅₀ of H1N1 (A/California/07/09) 28 days post-vaccination and a subset of mice were mock infected with an equivalent volume of media. A subset of the mice (n=9/group/time point) were euthanized at 3 (left) and 5 (right) days post infection (dpi) to evaluate pulmonary inflammation. Concentrations of cytokines and chemokines in the supernatant of lung homogenates were measured by multiplex ELISA (Quansys). At 3 dpi both vaccine groups had reduced inflammatory cytokines compared to the placebo group but there were no differences between vaccines. By 5 dpi the lungs of mice vaccinated with the non-binding Y91F H1-VLP had markedly less inflammatory cytokines typically associated with lung pathology. IFNγ neared baseline levels in these mice. FIG. 11E shows H&E stains of lung tissue at 10× magnification. Mice were challenged with 1.6×10³ TCID₅₀ of H1N1 (A/California/07/09) 28 days post-vaccination and a subset of mice were mock infected with an equivalent volume of media. A subset of the mice were euthanized at 4 days post infection (dpi) to evaluate lung pathology. Mice vaccinated with Y91F H1-VLP had decreased pulmonary inflammation compared to H1-VLP-vaccinated mice and more closely resembled the mock-infected mice.

FIG. 12A shows a schematic representation of construct 1190 (2X35S/CPMV 160/NOS-based expression cassette; left hand side), and construct 3637 (2X35S/CPMV 160/NOS-based expression cassette; right hand side). FIG. 12B shows a schematic representation of construct 2530 (2X35S/CPMV 160/NOS-based expression cassette, left hand side), and construct 4499 (2X35S/CPMV 160/NOS-based expression cassette, right hand side). FIG. 12C shows a schematic representation of construct 1314, encoding HA0 H1 A-Cal-7-09, and construct 6100, encoding HA0 H1 A-Cal-7-09 with a Y91F mutation. FIG. 12D shows a schematic representation of construct 1314, encoding HA0 H1 A-Idaho-07-2018, and construct 8177, encoding HA0 H1 A-Idaho-07-2018 with a Y91F mutation. FIG. 12E shows a schematic representation of construct 6722, encoding HA0 H1 A-Brisbane-02-2018, and construct 8433, encoding HA0 H1 A-Brisbane-02-2018 with a Y91F mutation. FIG. 12F shows a schematic representation of construct 7281, encoding HA0 H3 A-Kansas-14-2017, and construct 8179, encoding HA0 H3 A-Kansas-14-2017 with a Y98F mutation. FIG. 12G shows a schematic representation of construct 8384, encoding HA0 H3 A-Kansas-14-2017 with a Y98F mutation and a S136D mutation, and construct 8385, encoding HA0 H3 A-Kansas-14-2017 with a Y98F mutation and a S136N mutation. FIG. 12H shows a schematic representation of construct 8387, encoding HA0 H3 A-Kansas-14-2017 with a Y98F mutation and a S137N mutation, and construct 8388, encoding HA0 H3 A-Kansas-14-2017 with a Y98F mutation and a D190G mutation. FIG. 12I shows a schematic representation of construct 8389, encoding HA0 H3 A-Kansas-14-2017 with a Y98F mutation and a D190K mutation, and construct 8391, encoding HA0 H3 A-Kansas-14-2017 with a Y98F mutation and a R222W mutation. FIG. 12J shows a schematic representation of construct 8392, encoding HA0 H3 A-Kansas-14-2017 with a Y98F mutation and a S228N mutation, and construct 8393, encoding HA0 H3 A-Kansas-14-2017 with a Y98F mutation and a S228Q mutation. FIG. 12K shows a schematic representation of construct 8477, encoding HA0 H3 A-Kansas-14-2017 with a S136D mutation, and construct 8478, encoding HA0 H3 A-Kansas-14-2017 with a S136N mutation. FIG. 12L shows a schematic representation of construct 8481, encoding HA0 H3 A-Kansas-14-2017 with a D190K mutation, and construct 8482, encoding HA0 H3 A-Kansas-14-2017 with a R222W mutation. FIG. 12M shows a schematic representation of construct 8483, encoding HA0 H3 A-Kansas-14-2017 with a S228N mutation, and construct 8484, encoding HA0 H3 A-Kansas-14-2017 with a S228Q mutation. FIG. 12N shows a schematic representation of construct 2295, encoding HA0 H5 A-Indo-5-05, and construct 6101, encoding HA0 H5 A-Indo-5-05 with a Y91F mutation. FIG. 12O shows a schematic representation of construct 6102, encoding HA0 H7 A-Shanghai-2-13, and construct 6103, encoding HA0 H7 A-Shanghai-2-13 with a Y88F mutation. FIG. 12P shows a schematic representation of construct 2835, encoding HA0 HA B-Phuket-3073-13, and construct 8352, encoding HA0 HA B-Phuket-3073-13 with a S140A mutation. FIG. 12Q shows a schematic representation of construct 8354, encoding HA0 HA B-Phuket-3073-13 with a S142A mutation, and construct 8358, encoding HA0 HA B-Phuket-3073-13 with a G138A mutation. FIG. 12R shows a schematic representation of construct 8363, encoding HA0 HA B-Phuket-3073-13 with a L203A mutation, and construct 8376, encoding HA0 HA B-Phuket-3073-13 with a D195G mutation. FIG. 12S shows a schematic representation of construct 8382, encoding HA0 HA B-Phuket-3073-13 with a L203W mutation. FIG. 12T shows a schematic representation of construct 2879, encoding HA0 HA B/Sing/INFKK-16-0569/16, and construct 8485, encoding HA0 HA B/Sing/INFKK-16-0569/16 with a G138A mutation. FIG. 12U shows a schematic representation of construct 8486, encoding HA0 HA B/Sing/INFKK-16-0569/16 with a S140A mutation, and construct 8487, encoding HA0 HA B/Sing/INFKK-16-0569/16 with a S142A mutation. FIG. 12V shows a schematic representation of construct 8488, encoding HA0 HA B/Sing/INFKK-16-0569/16 with a D195G mutation, and construct 8489, encoding HA0 HA B/Sing/INFKK-16-0569/16 with a L203A mutation. FIG. 12W shows a schematic representation of construct 8490, encoding HA0 HA B/Sing/INFKK-16-0569/16 with a L203W mutation. FIG. 12X shows a schematic representation of construct 6791, encoding HA0 B-Maryland-15-2016, and construct 8434, encoding HA0 B-Maryland-15-2016 with a G138A mutation. FIG. 12Y shows a schematic representation of construct 8435, encoding HA0 B-Maryland-15-2016 with a S140A mutation, and construct 8436, encoding HA0 B-Maryland-15-2016 with a S142A mutation. FIG. 12Z shows a schematic representation of construct 8437, encoding HA0 B-Maryland-15-2016 with a D194G mutation, and construct 8438, encoding HA0 B-Maryland-15-2016 with a L202A mutation. FIG. 12AA shows a schematic representation of construct 8439, encoding HA0 B-Maryland-15-2016 with a L202W mutation. FIG. 12AB shows a schematic representation of construct 7679, encoding HA0 B-Wash-02-2019, and construct 8440, encoding HA0 B-Wash-02-2019 with a G138A mutation. FIG. 12AC shows a schematic representation of construct 8441, encoding HA0 B-Wash-02-2019 with a S140A mutation, and construct 8442, encoding HA0 B-Wash-02-2019 with a S142A mutation. FIG. 12AD shows a schematic representation of construct 8443, encoding HA0 B-Wash-02-2019 with a D193G mutation, and construct 8444, encoding HA0 B-Wash-02-2019 with a L201A mutation. FIG. 12AE shows a schematic representation of construct 8445, encoding HA0 B-Wash-02-2019 with a L201W mutation. FIG. 12AF shows a schematic representation of construct 8333, encoding HA0 B-Darwin-20-2019, and construct 8458, encoding HA0 B-Darwin-20-2019 with a G138A mutation. FIG. 12AG shows a schematic representation of construct 8459, encoding HA0 B-Darwin-20-2019 with a S140A mutation, and construct 8460, encoding HA0 B-Darwin-20-2019 with a S142A mutation. FIG. 12AH shows a schematic representation of construct 8461, encoding HA0 B-Darwin-20-2019 with a D193G mutation, and construct 8462, encoding HA0 B-Darwin-20-2019 with a L201A mutation. FIG. 12AI shows a schematic representation of construct 8463, encoding HA0 B-Darwin-20-2019 with a L201W mutation. FIG. 12AJ shows a schematic representation of construct 8150, encoding HA0 B-Victoria-705-2018, and construct 8446, encoding HA0 B-Victoria-705-2018 with a G138A mutation. FIG. 12AK shows a schematic representation of construct 8447, encoding HA0 B-Victoria-705-2018 with S140A mutation, and construct 8448, encoding HA0 B-Victoria-705-2018 with a S142A mutation. FIG. 12AL shows a schematic representation of construct 8449, encoding HA0 B-Victoria-705-2018 with D193G mutation, and construct 8450, encoding HA0 B-Victoria-705-2018 with a L201A mutation. FIG. 12AM shows a schematic representation of construct 8451, encoding HA0 B-Victoria-705-2018 with L201W mutation.

FIG. 13A shows the nucleic acid sequence of PDI-H1A/California/7/2009 (SEQ ID NO: 1); FIG. 13B shows the amino acid sequence of PDI-H1 A/California/7/2009 (SEQ ID NO: 2); FIG. 13C shows the nucleic acid sequence of PDI-H1 A/California/7/2009 Y91F (SEQ ID NO: 11); FIG. 13D shows the amino acid sequence of PDI-H1 A/California/7/2009 Y91F (SEQ ID NO:12). FIG. 13E shows the nucleic acid sequence of PDI-H1 A/Idaho/7/18 (SEQ ID NO: 100); FIG. 13F shows the amino acid sequence of PDI-H1 A/Idaho/7/18 (SEQ ID NO: 101); FIG. 13G shows the nucleic acid sequence of PDI-H1 A/Idaho/7/18 Y91F (SEQ ID NO: 104); FIG. 13H shows the amino acid sequence of PDI-H1 A/Idaho/7/18 Y91F (SEQ ID NO:105); FIG. 13I shows the nucleic acid sequence of PDI-H1 A/Brisbane/02/2018 (SEQ ID NO: 194); FIG. 13J shows the amino acid sequence of PDI-H1 A/Brisbane/02/2018 (SEQ ID NO: 195); FIG. 13K shows the nucleic acid sequence of PDI-H1 A/Brisbane/02/2018 Y98F (SEQ ID NO: 196). FIG. 13L shows the amino acid sequence of PDI-H1 A/Brisbane/02/2018 Y98F (SEQ ID NO: 197).

FIG. 14A shows the nucleic acid sequence of PDI-H3 A/Kansas/14/2017 (SEQ ID NO: 60); FIG. 14B shows the amino acid sequence of PDI-H3 A/Kansas/14/2017 (SEQ ID NO: 61); FIG. 14C shows the nucleic acid sequence of PDI-H3 A/Kansas/14/2017 Y98F (SEQ ID NO: 64); FIG. 14D shows the amino acid sequence of PDI-H3 A/Kansas/14/2017 Y98F (SEQ ID NO: 65); FIG. 14E shows the nucleic acid sequence of PDI-H3 A/Kansas/14/2017 Y98F, S136D (SEQ ID NO: 68); FIG. 14F shows the amino acid sequence of PDI-H3 A/Kansas/14/2017 Y98F, S136D (SEQ ID NO: 69); FIG. 14G shows the nucleic acid sequence of PDI-H3 A/Kansas/14/2017 Y98F, S136N (SEQ ID NO: 72); FIG. 14H shows the amino acid sequence of PDI-H3 A/Kansas/14/2017 Y98F, S136N (SEQ ID NO: 73); FIG. 14I shows the nucleic acid sequence of PDI-H3 A/Kansas/14/2017 Y98F, S137N (SEQ ID NO: 76); FIG. 14J shows the amino acid sequence of PDI-H3 A/Kansas/14/2017 Y98F, S137N (SEQ ID NO: 77); FIG. 14K shows the nucleic acid sequence of PDI-H3 A/Kansas/14/2017 Y98F, D190G (SEQ ID NO: 80); FIG. 14L shows the amino acid sequence of PDI-H3 A/Kansas/14/2017 Y98F, D190G (SEQ ID NO: 81); FIG. 14M shows the nucleic acid sequence of PDI-H3 A/Kansas/14/2017 Y98F, D190K (SEQ ID NO: 84); FIG. 14N shows the amino acid sequence of PDI-H3 A/Kansas/14/2017 Y98F, D190K (SEQ ID NO: 85); FIG. 14O shows the nucleic acid sequence of PDI-H3 A/Kansas/14/2017 Y98F, R222W (SEQ ID NO: 88); FIG. 14P shows the amino acid sequence of PDI-H3 A/Kansas/14/2017 Y98F, R222W (SEQ ID NO: 89); FIG. 14Q shows the nucleic acid sequence of PDI-H3 A/Kansas/14/2017 Y98F, S228N (SEQ ID NO: 92); FIG. 14R shows the amino acid sequence of PDI-H3 A/Kansas/14/2017 Y98F, S228N (SEQ ID NO: 93); FIG. 14S shows the nucleic acid sequence of PDI-H3 A/Kansas/14/2017 Y98F, S228Q (SEQ ID NO: 96); FIG. 14T shows the amino acid sequence of PDI-H3 A/Kansas/14/2017 Y98F, S228Q (SEQ ID NO: 97); FIG. 14U shows the nucleic acid sequence of PDI-H3 A/Kansas/14/2017 S136D (SEQ ID NO: 111); FIG. 14V shows the amino acid sequence of PDI-H3 A/Kansas/14/2017 S136D (SEQ ID NO: 112); FIG. 14W shows the nucleic acid sequence of PDI-H3 A/Kansas/14/2017 S136N (SEQ ID NO: 113); FIG. 14X shows the amino acid sequence of PDI-H3 A/Kansas/14/2017 S136N (SEQ ID NO: 114); FIG. 14Y shows the nucleic acid sequence of PDI-H3 A/Kansas/14/2017 D190K (SEQ ID NO: 115); FIG. 14Z shows the amino acid sequence of PDI-H3 A/Kansas/14/2017 D190K (SEQ ID NO: 116); FIG. 14AA shows the nucleic acid sequence of PDI-H3 A/Kansas/14/2017 R222W (SEQ ID NO: 117); FIG. 14AB shows the amino acid sequence of PDI-H3 A/Kansas/14/2017 R222W (SEQ ID NO: 118); FIG. 14AC shows the nucleic acid sequence of PDI-H3 A/Kansas/14/2017 S228N (SEQ ID NO: 119); FIG. 14AD shows the amino acid sequence of PDI-H3 A/Kansas/14/2017 S228N (SEQ ID NO: 120); FIG. 14AE shows the nucleic acid sequence of PDI-H3 A/Kansas/14/2017 S228Q (SEQ ID NO: 121); FIG. 14AF shows the amino acid sequence of PDI-H3 A/Kansas/14/2017 S228Q (SEQ ID NO: 122).

FIG. 15A shows the nucleic acid sequence of PDI H7 A/Shanghai/2/2013 (SEQ ID NO:20); FIG. 15B shows the amino acid sequence of PDI H7 A/Shanghai/2/2013 (SEQ ID NO:21); FIG. 15C shows the nucleic acid sequence of PDI H7 A/Shanghai/2/2013 Y88F (SEQ ID NO:25); FIG. 15D shows the amino acid sequence of PDI H7 A/Shanghai/2/2013 Y88F (SEQ ID NO:26); FIG. 15E shows the nucleic acid sequence of PDI H5 A/Indonesia/5/2005 (SEQ ID NO:198); FIG. 15F shows the amino acid sequence of PDI H5 A/Indonesia/5/2005 (SEQ ID NO:199); FIG. 15G shows the nucleic acid sequence of a primer IF-H5ITMCT.s1-4r (SEQ ID NO:200); FIG. 15H shows the nucleic acid sequence of PDI H5 A/Indonesia/5/2005 Y91F (SEQ ID NO:201); FIG. 15I shows the amino acid sequence of PDI H5 A/Indonesia/5/2005 Y91F (SEQ ID NO:202);

FIG. 16A shows the nucleic acid sequence of PDI B/Phuket/3073/2013 (Prl-) (SEQ ID NO:27); FIG. 16B shows the amino acid sequence of PDI B/Phuket/3073/2013 (Prl-) (SEQ ID NO:28); FIG. 16C shows the nucleic acid sequence of PDI B/Phuket/3073/2013 S140A (Prl-) (SEQ ID NO:32); FIG. 16D shows the amino acid sequence of PDI B/Phuket/3073/2013 S140A (Prl-) (SEQ ID NO:33); FIG. 16E shows the nucleic acid sequence of PDI B/Phuket/3073/2013 S142A (Prl-) (SEQ ID NO:36); FIG. 16F shows the amino acid sequence of PDI B/Phuket/3073/2013 S142A (Prl-) (SEQ ID NO:37); FIG. 16G shows the nucleic acid sequence of PDI B/Phuket/3073/2013 G138A (Prl-) (SEQ ID NO:40); FIG. 16H shows the amino acid sequence of PDI B/Phuket/3073/2013 G138A (Prl-) (SEQ ID NO:41); FIG. 16I shows the nucleic acid sequence of PDI B/Phuket/3073/2013 L203A (Prl-) (SEQ ID NO:44); FIG. 16J shows the amino acid sequence of PDI B/Phuket/3073/2013 L203A (Prl-) (SEQ ID NO:45); FIG. 16K shows the nucleic acid sequence of PDI B/Phuket/3073/2013 D195G (Prl-) (SEQ ID NO:48); FIG. 16L shows the amino acid sequence of PDI B/Phuket/3073/2013 D195G (Prl-) (SEQ ID NO:49); FIG. 16M shows the nucleic acid sequence of PDI B/Phuket/3073/2013 L203W (Prl-) (SEQ ID NO:52); FIG. 16N shows the amino acid sequence of PDI B/Phuket/3073/2013 L203W (Prl-) (SEQ ID NO:53); FIG. 16O shows the nucleic acid sequence of PDI-B/Singapore/INFKK-16-0569/2016 (Prl-) DNA (SEQ ID NO:123); FIG. 16P shows the amino acid sequence of PDI-B/Singapore/INFKK-16-0569/2016 (Prl-) AA (SEQ ID NO:124); FIG. 16Q shows the nucleic acid sequence of PDI-B/Singapore/INFKK-16-0569/2016-G138A (Prl-) DNA (SEQ ID NO:125); FIG. 16R shows the amino acid sequence of PDI-B/Singapore/INFKK-16-0569/2016-G138A (Prl-) AA (SEQ ID NO:126); FIG. 16S shows the nucleic acid sequence of PDI-B/Singapore/INFKK-16-0569/2016-S140A (Prl-) DNA (SEQ ID NO:127); FIG. 16T shows the amino acid sequence of PDI-B/Singapore/INFKK-16-0569/2016-S140A (Prl-) AA (SEQ ID NO:128); FIG. 16U shows the nucleic acid sequence of PDI-B/Singapore/INFKK-16-0569/2016-S142A (Prl-) DNA (SEQ ID NO:129); FIG. 16V shows the amino acid sequence of PDI-B/Singapore/INFKK-16-0569/2016-S142A (Prl-) AA (SEQ ID NO:130); FIG. 16W shows the nucleic acid sequence of PDI-B/Singapore/INFKK-16-0569/2016-D195G (Prl-) DNA (SEQ ID NO:131); FIG. 16X shows the amino acid sequence of PDI-B/Singapore/INFKK-16-0569/2016-D195G (Prl-) AA (SEQ ID NO:132); FIG. 16Y shows the nucleic acid sequence of PDI-B/Singapore/INFKK-16-0569/2016-L203A (Prl-) DNA (SEQ ID NO:133); FIG. 16Z shows the amino acid sequence of PDI-B/Singapore/INFKK-16-0569/2016-L203A (Prl-) AA (SEQ ID NO:134); FIG. 16AA shows the nucleic acid sequence of PDI-B/Singapore/INFKK-16-0569/2016-L203W (Prl-) DNA (SEQ ID NO:135); FIG. 16AB shows the amino acid sequence of PDI-B/Singapore/INFKK-16-0569/2016-L203W (Prl-) AA (SEQ ID NO:136); FIG. 16AC shows the nucleic acid sequence of PDI-B/Maryland/15/2016 (Prl-) DNA (SEQ ID NO:137); FIG. 16AD shows the amino acid sequence of PDI-B/Maryland/15/2016 (Prl-) AA (SEQ ID NO:138); FIG. 16AE shows the nucleic acid sequence of a primer IF-B-Bris(nat).c (SEQ ID NO:139); FIG. 16AF shows the nucleic acid sequence of PDI-B/Maryland/15/2016-G138A (Prl-) DNA (SEQ ID NO:140); FIG. 16AG shows the amino acid sequence of PDI-B/Maryland/15/2016-G138A (Prl-) AA (SEQ ID NO:141); FIG. 16AH shows the nucleic acid sequence of PDI-B/Maryland/15/2016-S140A (Prl-) DNA (SEQ ID NO:142); FIG. 16AI shows the amino acid sequence of PDI-B/Maryland/15/2016-S140A (Prl-) AA (SEQ ID NO:143); FIG. 16AJ shows the nucleic acid sequence of PDI-B/Maryland/15/2016-S142A (Prl-) DNA (SEQ ID NO:144); FIG. 16AK shows the amino acid sequence of PDI-B/Maryland/15/2016-S142A (Prl-) AA (SEQ ID NO:145); FIG. 16AL shows the nucleic acid sequence of PDI-B/Maryland/15/2016-D194G (Prl-) DNA (SEQ ID NO:146); FIG. 16AM shows the amino acid sequence of PDI-B/Maryland/15/2016-D194G (Prl-) AA (SEQ ID NO:147); FIG. 16AN shows the nucleic acid sequence of PDI-B/Maryland/15/2016-L202A (Prl-) DNA (SEQ ID NO: 148); FIG. 16AO shows the amino acid sequence of PDI-B/Maryland/15/2016-L202A (Prl-) AA (SEQ ID NO:149); FIG. 16AP shows the nucleic acid sequence of PDI-B/Maryland/15/2016-L202W (Prl-) DNA (SEQ ID NO:150); FIG. 16AQ shows the amino acid sequence of PDI-B/Maryland/15/2016-L202W (Prl-) AA (SEQ ID NO:151); FIG. 16AR shows the nucleic acid sequence of PDI-B/Washington/02/2019 (Prl-) DNA (SEQ ID NO:152); FIG. 16AS shows the amino acid sequence of PDI-B/Washington/02/2019 (Prl-) AA (SEQ ID NO:153); FIG. 16AT shows the nucleic acid sequence of PDI-B/Washington/02/2019-G138A (Prl-) DNA (SEQ ID NO:154); FIG. 16AU shows the amino acid sequence of PDI-B/Washington/02/2019-G138A (Prl-) AA (SEQ ID NO:155); FIG. 16AV shows the nucleic acid sequence of PDI-B/Washington/02/2019-S140A (Prl-) DNA (SEQ ID NO:156); FIG. 16AW shows the amino acid sequence of PDI-B/Washington/02/2019-S140A (Prl-) AA (SEQ ID NO:157); FIG. 16AX shows the nucleic acid sequence of PDI-B/Washington/02/2019-S142A (Prl-) DNA (SEQ ID NO:158); FIG. 16AY shows the amino acid sequence of PDI-B/Washington/02/2019-5142A (Prl-) AA (SEQ ID NO:159); FIG. 16AZ shows the nucleic acid sequence of PDI-B/Washington/02/2019-D193G (Prl-) DNA (SEQ ID NO:160); FIG. 16BA shows the amino acid sequence of PDI-B/Washington/02/2019-D193G (Prl-) AA (SEQ ID NO:161); FIG. 16BB shows the nucleic acid sequence of PDI-B/Washington/02/2019-L201A (Prl-) DNA (SEQ ID NO:162); FIG. 16BC shows the amino acid sequence of PDI-B/Washington/02/2019-L201A (Prl-) AA (SEQ ID NO:163); FIG. 16BD shows the nucleic acid sequence of PDI-B/Washington/02/2019-L201W (Prl-) DNA (SEQ ID NO:164); FIG. 16BE shows the amino acid sequence of PDI-B/Washington/02/2019-L201W (Prl-) AA (SEQ ID NO:165); FIG. 16BF shows the nucleic acid sequence of PDI-B/Victoria/705/2018 (Prl-) DNA (SEQ ID NO:180); FIG. 16BG shows the amino acid sequence of PDI-B/Victoria/705/2018 (Prl-) AA (SEQ ID NO:181); FIG. 16BH shows the nucleic acid sequence of PDI-B/Victoria/705/2018-G138A (Prl-) DNA (SEQ ID NO:182); FIG. 16BI shows the amino acid sequence of PDI-B/Victoria/705/2018-G138A (Prl-) AA (SEQ ID NO:183); FIG. 16BJ shows the nucleic acid sequence of PDI-B/Victoria/705/2018-S140A (Prl-) DNA (SEQ ID NO:184); FIG. 16BK shows the amino acid sequence of PDI-B/Victoria/705/2018-S140A (Prl-) AA (SEQ ID NO:185); FIG. 16BL shows the nucleic acid sequence of PDI-B/Victoria/705/2018-S142A (Prl-) DNA (SEQ ID NO:186); FIG. 16BM shows the amino acid sequence of PDI-B/Victoria/705/2018-S142A (Prl-) AA (SEQ ID NO:187); FIG. 16BN shows the nucleic acid sequence of PDI-B/Victoria/705/2018-D193G (Prl-) DNA (SEQ ID NO:188); FIG. 16BO shows the amino acid sequence of PDI-B/Victoria/705/2018-D193G (Prl-) AA (SEQ ID NO:189); FIG. 16BP shows the nucleic acid sequence of PDI-B/Victoria/705/2018-L201A (Prl-) DNA (SEQ ID NO:190); FIG. 16BQ shows the amino acid sequence of PDI-B/Victoria/705/2018-L201A (Prl-) AA (SEQ ID NO:191); FIG. 16BR shows the nucleic acid sequence of PDI-B/Victoria/705/2018-L201W (Prl-) DNA (SEQ ID NO:192); FIG. 16BS shows the amino acid sequence of PDI-B/Victoria/705/2018-L201W (Prl-) AA (SEQ ID NO:193); FIG. 16BT shows the amino acid sequence of HA H1 A/California/07/2009 (SEQ ID NO:203); FIG. 16BU shows the amino acid sequence of HA H3 A/Kansas/14/2017 (SEQ ID NO:204); FIG. 16BV shows the amino acid sequence of HA H5 A/Indonesia/05/2005 (SEQ ID NO:205); FIG. 16BW shows the amino acid sequence of HA H7 A/Shanghai/2/2013 (SEQ ID NO:206); FIG. 16BX shows the amino acid sequence of HA B B/Phuket/3073/2013 (SEQ ID NO:207); FIG. 16BY shows the amino acid sequence of HA B B/Maryland/15/2016 (SEQ ID NO:208); FIG. 16BZ shows the amino acid sequence of HA B B/Victoria/705/2018 (SEQ ID NO:209).

FIG. 17A shows the nucleic acid sequence for cloning vector 1190 from left to right T-DNA (SEQ ID NO: 5); FIG. 17B shows the nucleic acid sequence for construct 1314 from 2X35S prom to NOS term (SEQ ID NO: 6); FIG. 17C shows the nucleic acid sequence for cloning vector 3637 from left to right T-DNA (SEQ ID NO: 9) FIG. 17D shows the nucleic acid sequence for construct 6100 from 2X35S prom to NOS term (SEQ ID NO: 10); FIG. 17E shows the nucleic acid sequence for cloning vector 2530 from left to right T-DNA (SEQ ID NO: 54); FIG. 17F shows the nucleic acid sequence for construct 2835 from 2X35S prom to NOS term (SEQ ID NO: 55)); FIG. 17G shows the nucleic acid sequence for Cloning vector 4499 from left to right T-DNA (SEQ ID NO: 56); FIG. 17H shows the nucleic acid sequence for construct 8352 from 2X35S prom to NOS term (SEQ ID NO: 57). FIG. 17I shows the nucleic acid sequence for construct 7281 from 2X35S prom to NOS term (SEQ ID NO: 58). FIG. 17J shows the nucleic acid sequence for construct 8179 from 2X35S prom to NOS term (SEQ ID NO: 59).

FIGS. 18A and B shows that total splenic CD4 T cell responses were maintained upon introduction of the alteration from Y91F. Mice (n=10/group) were vaccinated with 3 μg binding or non-binding (Y91F) H5-VLP and boosted with 3 μg at 8 weeks. Mice were euthanized 5 weeks post-boost and spleens were harvested to measure antigen-specific (CD44+) CD4 T cells by flow cytometry. Both vaccines resulted in similar frequencies of responding cells (18A) with similar frequencies of polyfunctional CD4 T cells (18B). However, Y91F H5-VLP resulted in fewer IFNγ single positive cells. (triple positive) CD4 T cells (18B). Statistical significance was determined by Kruskal-Wallis test with Dunn's multiple comparisons (18A) or two-way ANOVA with Tukey's multiple comparisons (18B). *p<0.033, **p<0.01, ***p<0.001

FIGS. 18C and D show that splenic CD8 T cell responses were reduced upon introduction of the non-binding mutation. Mice (n=10/group) were vaccinated with 3 μg binding or non-binding (Y91F) H5-VLP and boosted with 3 μg at 8 weeks. Mice were euthanized 5 weeks post-boost and spleens were harvested to measure antigen-specific (CD44+) CD8 T cells by flow cytometry. Both VLPs resulted in a significant increase in total responding cells compared to the placebo group but the response was considerably stronger in mice that received the WT H5-VLP (18C). This increase was driven by an increase in IFNγ single-positive cells and IL-2⁺IFNγ⁺ cells (18D). Statistical significance was determined by Kruskal-Wallis test with Dunn's multiple comparisons (18C) or two-way ANOVA with Tukey's multiple comparisons (18D). *p<0.033, **p<0.01, ***p<0.001. FIG. 18E shows that non-binding H5-VLP results in increased H5-specific bone marrow plasma cells (BMPC). Mice (n=10/group) were vaccinated with 3 μg binding or non-binding (Y91F) H5-VLP and boosted with 3 μg at 8 weeks. Mice were euthanized 5 weeks post-boost and bone marrow (BM) was harvested to measure H5-specific BMPC by ELISpot assay. Images of representative wells are shown on the right. Statistical significance was evaluated using the Mann-Whitney test. FIGS. 18F and 18G shows that non-binding H5-VLP results in increased antigen-specific CD4 T cells in the bone marrow (BM). Mice (n=10/group) were vaccinated with 3 μg binding or non-binding (Y91F) H5-VLP and boosted with 3 μg at 8 weeks. Mice were euthanized 5 weeks post-boost and BM harvested to measure antigen-specific (CD44+) CD4 T cells by flow cytometry. Only Y91F H5-VLP resulted in a significant increase in responding CD4 T cells compared to the placebo group (18F). Y91F H1-VLP also resulted in a significant increase in IL-2⁺TNFα⁺IFNγ⁻ CD4 T cells compared to the WT H5-VLP (18G). Statistical significance was determined by Kruskal-Wallis test with Dunn's multiple comparisons (18F) or two-way ANOVA with Tukey's multiple comparisons (18G). *p<0.033, **p<0.01, ***p<0.001

FIG. 19A shows that the non-binding H7-VLP results in significantly higher hemagglutination inhibition (HI) titers at all time points measured. Mice (n=10/group) were vaccinated with 3 μg binding or non-binding (Y88F) H7-VLP and boosted with 3 μg at 8 weeks. Sera were collected and HI titers were measured at weeks 4, 8 and 13. Statistical significance was determined by multiple T-tests with Holm-Sidak's multiple comparisons. *p<0.033, **p<0.01, ***p<0.001. FIG. 19B shows that binding and non-binding (Y88F) H7-VLP result in similar total H7-specific IgG titers. FIG. 19C shows that the non-binding H7-VLP results in enhanced IgG avidity maturation. Bound serum samples were treated with 0-10M Urea and the avidity index represents the proportion of IgG that remains bound after the urea incubation ([IgG titer 2-10M urea]/[IgG titer 0M urea]). The left panel shows avidity indices at week 13. The right panel shows changes in avidity over time (8M urea). Statistical significance was determined by multiple T-tests with Holm-Sidak's multiple comparisons. *p<0.033, **p<0.01. FIG. 19D shows that non-binding H7-VLP results in increased H7-specific bone marrow plasma cells (BMPC). Mice (n=10/group) were vaccinated with 3 μg binding or non-binding (Y88F) H7-VLP and boosted with 3 μg at 8 weeks. Mice were euthanized 5 weeks post-boost and bone marrow (BM) was harvested to measure H7-specific BMPC by ELISpot assay. Images of representative wells are shown on the right. Statistical significance was evaluated using the Mann-Whitney test. FIGS. 19E and 19F shows that splenic CD4 T cell responses were maintained upon introduction of the non-binding mutation. Mice (n=10/group) were vaccinated with 3 μg binding or non-binding (Y88F) H7-VLP and boosted with 3 μg at 8 weeks. Mice were euthanized 5 weeks post-boost and spleens were harvested to measure antigen-specific (CD44+) CD4 T cells by flow cytometry. Both vaccines resulted in similar frequencies of responding cells (19E) with similar frequencies of IL-2⁺TNFα⁺IFNγ⁺ (triple positive) CD4 T cells (19F). The Y88F H7-VLP resulted in increased IL-2 single positive cells. Statistical significance was determined by Kruskal-Wallis test with Dunn's multiple comparisons (19E) or two-way ANOVA with Tukey's multiple comparisons (19F). *p<0.033, **p<0.01, ***p<0.001. FIGS. 19G and 19H shows that splenic CD8 T cell responses were similar between vaccine groups. Mice (n=10/group) were vaccinated with 3 μg binding or non-binding (Y88F) H7-VLP and boosted with 3 μg at 8 weeks. Mice were euthanized 5 weeks post-boost and spleens were harvested to measure antigen-specific (CD44+) CD8 T cells by flow cytometry. In general, CD8 T cell responses were weak. Only the WT H7-VLP resulted in a significant increase in total responding cells (19G), driven by an increase in IFNγ single-positive cells (19H). Polyfunctional CD8 T cell signatures were similar in both vaccine groups with a significant increase in IL-2⁺IFNγ⁺ cells. Statistical significance was determined by Kruskal-Wallis test with Dunn's multiple comparisons (19G) or two-way ANOVA with Tukey's multiple comparisons (19H). *p<0.033, **p<0.01, ***p<0.001

FIGS. 20A and 20B shows that fewer CD4 T cells expressing IFNγ upon vaccination with non-binding B-VLP (3 weeks post-boost). Mice (n=8/group) were vaccinated with 1 μg binding or non-binding (NB) B-VLP (D195G B/Phuket/3073/2013) and boosted with 1 μg at day 21. Mice were euthanized 3 weeks post-boost and spleens were harvested to measure antigen-specific (CD44+) CD4 T cells by flow cytometry. The frequency of total responding CD4 T cells was similar between vaccine groups (20A). Similar to other non-binding VLPs, the IL-2⁺ populations dominated the response to the NB B-VLP (20B). However, IFNγ⁺ cells were reduced in mice vaccinated with NB B-VLP. Statistical significance was determined by Kruskal-Wallis test with Dunn's multiple comparisons (20A) or two-way ANOVA with Tukey's multiple comparisons (20B). *p<0.033, **p<0.01, ***p<0.001.

### DETAILED DESCRIPTION

The following description is of a preferred embodiment.

As used herein, the terms “comprising”, “having”, “including”, “containing”, and grammatical variations thereof, are inclusive or open-ended and do not exclude additional, un-recited elements and/or method steps. The term “consisting essentially of” when used herein in connection with a product, use or method, denotes that additional elements and/or method steps may be present, but that these additions do not materially affect the manner in which the recited method or use functions. The term “consisting of” when used herein in connection with a product, use or method, excludes the presence of additional elements and/or method steps. A product, use or method described herein as comprising certain elements and/or steps may also, in certain embodiments, consist essentially of those elements and/or steps, and in other embodiments consist of those elements and/or steps, whether or not these embodiments are specifically referred to. In addition, the use of the singular includes the plural, and “or” means “and/or” unless otherwise stated. Unless otherwise defined herein, all technical and scientific terms used herein have the same meaning as commonly understood by one of ordinary skill in the art. As used herein, the term “about” refers to an approximately +/−10% variation from a given value. It is to be understood that such a variation is always included in any given value provided herein, whether or not it is specifically referred to. The use of the word “a” or “an” when used herein in conjunction with the term “comprising” may mean “one,” but it is also consistent with the meaning of “one or more,” “at least one” and “one or more than one.”

As used herein the abbreviations “CMI” refers to cell-mediated immunity; “HA” refers to hemagglutinin; “HAI” refers to hemagglutination inhibition; “MN” refers to microneutralization; “PBMC” refers to peripheral blood mononuclear cells; “tRBC” refers to turkey red blood cell; “SA” refers to sialic acid; “SPR” refers to surface plasmon resonance; “UIV” refers to universal influenza vaccine; “VLP” refers to virus-like particle.

The term host as used herein may comprise any suitable eukaryotic host as would be known to one of skill in the art, for example but not limited to, a eukaryotic cell, a eukaryotic cell culture, a mammalian cell culture, an insect cell, an insect cell culture, a baculovirus cell, an avian cell, an egg cell, a plant cell, a plant, or a portion of a plant.

The term “portion of a plant”, “plant portion”, “plant matter”, “plant biomass”, “plant material” as used herein, refers to any part of the plant including but not limited to leaves, stem, root, flowers, fruits, a plant cell obtained from leaves, stem, root, flowers, fruits, a plant extract obtained from leaves, stem, root, flowers, fruits, or a combination thereof. The term “plant extract”, as used herein, refers to a plant-derived product that is obtained following treating a plant, a portion of a plant, a plant cell, or a combination thereof, physically (for example by freezing followed by extraction in a suitable buffer), mechanically (for example by grinding or homogenizing the plant or portion of the plant followed by extraction in a suitable buffer), enzymatically (for example using cell wall degrading enzymes), chemically (for example using one or more chelators or buffers), or a combination thereof. A plant extract may comprise plant tissue, cells, or any fraction thereof, intracellular plant components, extracellular plant components, liquid or solid extracts of plants, or a combination thereof.

A plant extract may be further processed to remove undesired plant components for example cell wall debris. A plant extract may be obtained to assist in the recovery of one or more components from the plant, portion of the plant or plant cell, for example suprastructures, nucleic acids, lipids, carbohydrates, or a combination thereof, from the plant, portion of the plant, or plant cell.

“Suprastructures” (protein suprastructures) include, but are not limited to, multimeric proteins such for example dimeric proteins, trimeric proteins, polymeric proteins, rosettes comprising proteins, metaproteins, protein complexes, protein-lipid complexes, VLPs, or a combination thereof.

Furthermore, the suprastructures may be a scaffold comprising protein or multimeric proteins. For example the suprastructures may be nanoparticles, nanostructures, protein nanostructures, polymer such as for example sugar polymer, micelles, vesicles, membranes or membrane fragments comprising protein or multimeric proteins. In an non-limiting example, the suprastructure may have a size range from about 10 nm to about 350 nm, or any amount therebetween.

If the plant extract comprises proteins, then it may be referred to as a protein extract. A protein extract (or a suprastructure extract) may be a crude plant extract, a partially purified plant or protein extract, or a purified product, that comprises one or more suprastructures, dimeric proteins, trimeric proteins, polymeric proteins, rosettes comprising proteins, metaproteins, protein complexes, protein-lipid complexes, VLPs, or a combination thereof, from the plant tissue. If desired a suprastructure extract, for example a protein extract, or a plant extract, may be partially purified using techniques known to one of skill in the art, for example, the extract may be subjected to salt or pH precipitation, centrifugation, gradient density centrifugation, filtration, chromatography, for example, size exclusion chromatography, ion exchange chromatography, affinity chromatography, or a combination thereof. A suprastructure or protein extract may also be purified, using techniques that are known to one of skill in the art.

The term “construct”, “vector” or “expression vector”, as used herein, refers to a recombinant nucleic acid for transferring exogenous nucleic acid sequences into host cells (e.g. plant cells) and directing expression of the exogenous nucleic acid sequences in the host cells. “Expression cassette” refers to a nucleotide sequence comprising a nucleic acid of interest under the control of, and operably (or operatively) linked to, an appropriate promoter or other regulatory elements for transcription of the nucleic acid of interest in a host cell. As one of skill in the art would appreciate, the expression cassette may comprise a termination (terminator) sequence that is any sequence that is active the plant host. For example, the termination sequence may be derived from the RNA-2 genome segment of a bipartite RNA virus, e.g. a comovirus, the termination sequence may be a NOS terminator, or terminator sequence may be obtained from the 3′UTR of the alfalfa plastocyanin gene.

The constructs of the present disclosure may further comprise a 3′ untranslated region (UTR). A 3′ untranslated region contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. Non-limiting examples of suitable 3′ regions are the 3′ transcribed non-translated regions containing a polyadenylation signal of Agrobacterium tumor inducing (Ti) plasmid genes, such as the nopaline synthase (Nos gene) and plant genes such as the soybean storage protein genes, the small subunit of the ribulose-1, 5-bisphosphate carboxylase gene (ssRUBISCO; U.S. Pat. No. 4,962,028; which is incorporated herein by reference), the promoter used in regulating plastocyanin expression.

By “regulatory region” “regulatory element” or “promoter” it is meant a portion of nucleic acid typically, but not always, upstream of the protein coding region of a gene, which may be comprised of either DNA or RNA, or both DNA and RNA. When a regulatory region is active, and in operative association, or operatively linked, with a nucleotide sequence of interest, this may result in expression of the nucleotide sequence of interest. A regulatory element may be capable of mediating organ specificity or controlling developmental or temporal gene activation. A “regulatory region” includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element may comprise a basal promoter element, responsible for the initiation of transcription, as well as other regulatory elements that modify gene expression.

There are several types of regulatory regions, including those that are developmentally regulated, inducible or constitutive. A regulatory region that is developmentally regulated or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well. Examples of tissue-specific regulatory regions, for example see-specific a regulatory region, include the napin promoter, and the cruciferin promoter (Rask et al., 1998, J. Plant Physiol. 152: 595-599; Bilodeau et al., 1994, Plant Cell 14: 125-130). An example of a leaf-specific promoter includes the plastocyanin promoter (see U.S. Pat. No. 7,125,978, which is incorporated herein by reference).

An inducible regulatory region is one that is capable of directly or indirectly activating transcription of one or more DNA sequences or genes in response to an inducer. In the absence of an inducer the DNA sequences or genes will not be transcribed. Typically, the protein factor that binds specifically to an inducible regulatory region to activate transcription may be present in an inactive form, which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, I. R. P., 1998, Trends Plant Sci. 3, 352-358). Examples, of potential inducible promoters include, but not limited to, tetracycline-inducible promoter (Gatz, C., 1997, Ann. Rev. Plant Physiol. Plant Mol. Biol. 48, 89-108), steroid inducible promoter (Aoyama, T. and Chua, N.H., 1997, Plant J. 2, 397-404) and ethanol-inducible promoter (Salter, M. G., et al, 1998, Plant Journal 16, 127-132; Caddick, M. X., et al, 1998, Nature Biotech. 16, 177-180) cytokinin inducible IB6 and CKI1 genes (Brandstatter, I. and Kieber, J. J., 1998, Plant Cell 10, 1009-1019; Kakimoto, T., 1996, Science 274, 982-985) and the auxin inducible element, DR5 (Ulmasov, T., et al., 1997, Plant Cell 9, 1963-1971).

A constitutive regulatory region directs the expression of a gene throughout the various parts of a plant and continuously throughout plant development. Examples of known constitutive regulatory elements include promoters associated with the CaMV 35S transcript. (p35S; Odell et al., 1985, Nature, 313: 810-812; which is incorporated herein by reference), the rice actin 1 (Zhang et al, 1991, Plant Cell, 3: 1155-1165), actin 2 (An et al., 1996, Plant J., 10: 107-121), or tms 2 (U.S. Pat. No. 5,428,147), and triosephosphate isomerase 1 (Xu et. al., 1994, Plant Physiol. 106: 459-467) genes, the maize ubiquitin 1 gene (Comejo et al, 1993, Plant Mol. Biol. 29: 637-646), the Arabidopsis ubiquitin 1 and 6 genes (Holtorf et al, 1995, Plant Mol. Biol. 29: 637-646), the tobacco translational initiation factor 4A gene (Mandel et al, 1995 Plant Mol. Biol. 29: 995-1004), the Cassava Vein Mosaic Virus promoter, pCAS, (Verdaguer et al., 1996); the promoter of the small subunit of ribulose biphosphate carboxylase, pRbcS: (Outchkourov et al., 2003), the pUbi (for monocots and dicots).

The term “constitutive” as used herein does not necessarily indicate that a nucleotide sequence under control of the constitutive regulatory region is expressed at the same level in all cell types, but that the sequence is expressed in a wide range of cell types even though variation in abundance is often observed.

A nucleic acid comprising encoding a modified HA protein as described herein may further comprise sequences that enhance expression of the modified HA protein in the desired host, for example a plant, portion of the plant, or plant cell.

The term “plant-derived expression enhancer”, as used herein, refers to a nucleotide sequence obtained from a plant, the nucleotide sequence encoding a 5′UTR. Examples of a plant derived expression enhancer are described in WO2019/173924 and PCT/CA2019/050319 (both of which are incorporated herein by reference) or in Diamos A. G. et. al. (2016, Front Plt Sci. 7:1-15; which is incorporated herein by reference). The plant-derived expression enhancer may also be selected from nbMT78, nbATL75, nbDJ46, nbCHP79, nbEN42, atHSP69, atGRP62, atPK65, atRP46, nb30S72, nbGT61, nbPV55, nbPPI43, nbPM64, nbH2A86 as described in PCT/CA2019/050319 (which is incorporated herein by reference), and nbEPI42, nbSNS46, nbCSY65, nbHEL40, nbSEP44 as described in PCT/CA/2019/050319 (which is incorporated herein by reference).

The plant derived expression enhancer may be used within a plant expression system comprising a regulatory region that is operatively linked with the plant-derived expression enhancer sequence and a nucleotide sequence of interest.

Sequences that enhance expression may also include a CPMV enhancer element. The term “CPMV enhancer element”, as used herein, refers to a nucleotide sequence encoding the 5′UTR regulating the Cowpea Mosaic Virus (CPMV) RNA2 polypeptide or a modified CPMV sequence as is known in the art. For example, a CPMV enhancer element or a CPMV expression enhancer, includes a nucleotide sequence as described in WO2015/14367; WO2015/103704; WO2007/135480; WO2009/087391; Sainsbury F., and Lomonossoff G. P., (2008, Plant Physiol. 148: pp. 1212-1218), each of which is incorporated herein by reference. A CPMV enhancer sequence can enhance expression of a downstream heterologous open reading frame (ORF) to which they are attached. The CPMV expression enhancer may include CPMV HT, CPMVX (where X=160, 155, 150, 114), for example CPMV 160, CPMVX+(where X=160, 155, 150, 114), for example CPMV 160+, CPMV-HT+, CPMV HT+[WT115], or CPMV HT+[511] (WO2015/143567; WO2015/103704 which are incorporated herein by reference). The CPMV expression enhancer may be used within a plant expression system comprising a regulatory region that is operatively linked with the CPMV expression enhancer sequence and a nucleotide sequence of interest.

The term “5′UTR” or “5′ untranslated region” or “5′ leader sequence” refers to regions of an mRNA that are not translated. The 5′UTR typically begins at the transcription start site and ends just before the translation initiation site or start codon of the coding region. The 5′ UTR may modulate the stability and/or translation of an mRNA transcript.

By “operatively linked” it is meant that the particular sequences interact either directly or indirectly to carry out an intended function, such as mediation or modulation of expression of a nucleic acid sequence. The interaction of operatively linked sequences may, for example, be mediated by proteins that interact with the operatively linked sequences.

Post-transcriptional gene silencing (PTGS) may be involved in limiting expression of transgenes in plants, and co-expression of a suppressor of silencing from the potato virus Y (HcPro) may be used to counteract the specific degradation of transgene mRNAs (Brigneti et al., 1998). Alternate suppressors of silencing are well known in the art and may be used as described herein (Chiba et al., 2006, Virology 346:7-14; which is incorporated herein by reference), for example but not limited to, TEV-p1/HC-Pro (Tobacco etch virus-p1/HC-Pro), BYV-p21, p19 of Tomato bushy stunt virus (TBSV p19), capsid protein of Tomato crinkle virus (TCV-CP), 2b of Cucumber mosaic virus; CMV-2b), p25 of Potato virus X (PVX-p25), p11 of Potato virus M (PVM-p11), p11 of Potato virus S (PVS-p11), p16 of Blueberry scorch virus, (BScV-p16), p23 of Citrus tristexa virus (CTV-p23), p24 of Grapevine leafroll-associated virus-2, (GLRaV-2 p24), p10 of Grapevine virus A, (GVA-p10), p14 of Grapevine virus B (GVB-p14), p10 of Heracleum latent virus (HLV-p10), or p16 of Garlic common latent virus (GCLV-p16). Therefore, a suppressor of silencing, for example, but not limited to, HcPro, TEV-p1/HC-Pro, BYV-p21, TBSV p19, TCV-CP, CMV-2b, PVX-p25, PVM-p11, PVS-p11, BScV-p16, CTV-p23, GLRaV-2 p24, GBV-p14, HLV-p10, GCLV-p16 or GVA-p10, may be co-expressed along with the nucleic acid sequence encoding the protein of interest to further ensure high levels of protein production within a plant.

The expression constructs as described above may be present in a vector. The vector may comprise border sequences which permit the transfer and integration of the expression cassette into the genome of the organism or host. For example, the construct may be a plant binary vector, for example a binary transformation vector based on pPZP (Hajdukiewicz, et al. 1994). Other example constructs include pBin19 (see Frisch, D. A., L. W. Harris-Haller, et al. 1995, Plant Molecular Biology 27: 405-409).

The constructs of the present invention can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. D T. Dennis, D H Turpin, D D Lefebrve, D B Layzell (eds), Addison Wesly, Langmans Ltd. London, pp. 561-579 (1997). Other methods include direct DNA uptake, the use of liposomes, electroporation, for example using protoplasts, micro-injection, microprojectiles or whiskers, and vacuum infiltration. See, for example, Bilang, et al. (Gene 100: 247-250 (1991), Scheid et al. (Mol. Gen. Genet. 228: 104-112, 1991), Guerche et al. (Plant Science 52: 111-116, 1987), Neuhause et al. (Theor. Appl Genet. 75: 30-36, 1987), Klein et al., Nature 327: 70-73 (1987); Howell et al. (Science 208: 1265, 1980), Horsch et al. (Science 227: 1229-1231, 1985), DeBlock et al., Plant Physiology 91: 694-701, 1989), Methods for Plant Molecular Biology (Weissbach and Weissbach, eds., Academic Press Inc., 1988), Methods in Plant Molecular Biology (Schuler and Zielinski, eds., Academic Press Inc., 1989), Liu and Lomonossoff (J. Virol Meth, 105:343-348, 2002,), U.S. Pat. Nos. 4,945,050; 5,036,006; and 5,100,792, U.S. patent application Ser. No. 08/438,666, filed May 10, 1995, and Ser. No. 07/951,715, filed Sep. 25, 1992, (all of which are hereby incorporated by reference).

Transient expression methods may be used to express the constructs of the present invention (see Liu and Lomonossoff, 2002, Journal of Virological Methods, 105:343-348; which is incorporated herein by reference). Alternatively, a vacuum-based transient expression method, as described by Kapila et al. 1997 (incorporated herein by reference) may be used. These methods may include, for example, but are not limited to, a method of Agro-inoculation or Agro-infiltration, however, other transient methods may also be used as noted above. With either Agro-inoculation or Agro-infiltration, a mixture of Agrobacteria comprising the desired nucleic acid enter the intercellular spaces of a tissue, for example the leaves, aerial portion of the plant (including stem, leaves and flower), other portion of the plant (stem, root, flower), or the whole plant. After crossing the epidermis the Agrobacterium infect and transfer t-DNA copies into the cells. The t-DNA is episomally transcribed and the mRNA translated, leading to the production of the protein of interest in infected cells, however, the passage of t-DNA inside the nucleus is transient.

The term “wild type”, “native”, “native protein” or “native domain”, as used herein, refers to a protein or domain having a primary amino acid sequence identical to wildtype. Native proteins or domains may be encoded by nucleotide sequences having 100% sequence similarity to the wildtype sequence. A native amino acid sequence may also be encoded by a human codon (hCod) optimized nucleotide sequence or a nucleotide sequence comprising an increased GC content when compared to the wild type nucleotide sequence provided that the amino acid sequence encoded by the hCod-nucleotide sequence exhibits 100% sequence identity with the native amino acid sequence.

By a nucleotide sequence that is “human codon optimized” or a “hCod” nucleotide sequence, it is meant the selection of appropriate DNA nucleotides for the synthesis of an oligonucleotide sequence or fragment thereof that approaches the codon usage generally found within an oligonucleotide sequence of a human nucleotide sequence. By “increased GC content” it is meant the selection of appropriate DNA nucleotides for the synthesis of an oligonucleotide sequence or fragment thereof in order to approach codon usage that, when compared to the corresponding native oligonucleotide sequence, comprises an increase of GC content, for example, from about 1 to about 30%, or any amount therebetween, over the length of the coding portion of the oligonucleotide sequence. For example, from about 1, 2, 4, 6, 8, 10, 12, 14, 16, 18, 20, 22, 24, 26, 28, 30%, or any amount therebetween, over the length of the coding portion of the oligonucleotide sequence. As described below, a human codon optimized nucleotide sequence, or a nucleotide sequence comprising an increased GC contact (when compared to the wild type nucleotide sequence) exhibits increased expression within a plant, portion of a plant, or a plant cell, when compared to expression of the non-human optimized (or lower GC content) nucleotide sequence.

By an immune response or immunological response, it is meant the response that is elicited following exposure of a subject to a foreign antigen. This response typically involves cognate and non-cognate interactions between the antigen and components of the immune system that ultimately results in activation of the immune components and leading to defense responses, including the production of antibodies against the foreign antigen. Improving the immune response may result in higher neutralizing antibody titers (HAI and MN) and may include increasing avidity. Changes in an immune response within a subject following administration of the modified HA having reduced or no binding to SA as described herein, may be determined, for example, using hemagglutination inhibition (HAI, see example 3.5), microneutralization (MN, see Example 3.5) and/or avidity (see Example 3.5) assays, and comparing the levels obtained in the subject (the first subject) against those obtained in a second subject that was administered a parent HA, under similar conditions. For example, an improved immune response may be indicated by an increase in HAI titers, MN titers, and/or avidity, in the first subject when compared with the HAI titers, MN titers, and/or avidity in the second subject.

Therefore the immune or immunological response may be a cellular immunological response, a humoral immunological response, or both a cellular immunological response and a humoral immunological response.

A cellular or cell-mediated response is an immune response that does not involve antibodies, but rather the involves the activation of phagocytes, antigen-specific cytotoxic T-lymphocytes, and the release of various cytokines in response to an antigen. A humoral immune response is mediated by antibody molecules that are secreted by plasma cells.

Cognate interactions that drive the B cell or humoral response involve recognition of the conformational or linear epitopes of the antigen by naïve B cells via complementarity loops of the germline B cell receptor. Cognate interactions that drive the T lymphocyte or cellular response include recognition of peptides presented by MHC molecules on the surface of antigen-presenting cells. At a molecular level, cognate interactions may include interactions between the B and T cell receptors and their antigens/epitope. At a larger scale, complex interactions between whole T and B cells that are responding to the same antigen may also considered to be ‘cognate’. Cognate interactions may be determined using any method known in the art, for example but not limited to assaying HAI titers, MN titers, avidity. Epitope-antibody interactions may be determined using any suitable method known in the art, for example but not limited to, ELISA and Western blot analysis.

Non-cognate interactions of a potential antigen with immune cells can take many forms. As used herein, binding of an antigen, for example HA, with any glycoprotein expressed on the surface of an immune cell via sialic acid (SA) residues may be considered a non-cognate interaction. Therefore, non-cognate interaction as used herewith includes the interaction or binding to sialic acid. Accordingly, a reduction in non-cognate interaction or binding, includes the reduction in interaction or binding to SA residues. Non-cognate interactions may be determined, for example, by assaying hemagglutination or using surface plasmon resonance (SPR), as described herein.

By “target” it is meant a cell, a cell receptor, a protein on the surface of a cell, a cell surface protein, an antibody, or fragment of an antibody, that is capable of interacting with an antigen. In one example the target may be a protein on the surface of a cell or a cell surface protein.

For example, the suprastructure as described in the current disclosure may comprise a modified influenza hemagglutinin (HA) with one or more than one alteration that reduces interaction of the modified HA to sialic acid (SA) of a target, while maintaining cognate interaction, with the target. For the example, the target may be a protein on the surface of a cell. Accordingly, the suprastructure may comprise modified influenza hemagglutinin (HA) with one or more than one alteration that reduces interaction of the modified HA to sialic acid (SA) of a protein on the surface of a cell, while maintaining cognate interaction with the cell. The cell may be for example be a B cell.

B cells may interact with an antigen via receptor signals through CDR driven antigen complementarity (cognate interaction), or via (non-cognate) interactions provided by, for example, antigen affinity to SA, glycans on HA interacting with glycan receptors on the surface of immune cells or other non-cognate interactions between HA and a cell, for example interactions with any cell receptor comprising SA, for example, a B cell surface protein or a T cell receptor surface protein. Naïve B cells may recognize the conformation of the antigen by the complementarity loops of a germline B cell receptor and interact with the antigen. An antibody, or a fragment of an antibody comprising a complimentary paratope, may bind an antigen and be considered a target. A recombinant cell expressing an antibody comprising a corresponding paratope may also bind an antigen and may also be considered a target.

By avidity it is meant a measure of the overall stability of the antibody-antigen complex, or the strength with which an antibody binds an antigen. Avidity is governed by the intrinsic affinity of the antibody for an epitope, the valency of the antibody and antigen, and the geometric arrangement or conformation of the interacting components. Maturation of the humoral immune response in a subject may be indicated by an increase in antibody avidity over time. Avidity may be determined using competitive inhibition assays over a range of concentration of free antigen, or by eluting the antibody from the antigen using a dissociating agent that disrupts hydrophobic bonds, for example thiocyanate or urea.

In one aspect, the current disclosure provides suprastructure comprising modified influenza hemagglutinin (HA). The suprastructure may be for example a virus-like particle (VLP). For example the VLP may be an influenza HA-VLP, wherein the VLP comprises or consists of modified influenza HA protein. For example, the modified influenza HA may be a type A influenza such for example an HA from H1, H3, H5 or H7 or the HA may be from a type B influenza such for example an HA from the B Yamagata or B Victoria lineage. The modified HA may comprise one or more than one alteration. For example the HA may be:

i) a modified H1 HA, wherein the one or more than one alteration is selected from Y91F; wherein the numbering of the alteration corresponds to the position of reference sequence with SEQ ID NO: 203 (H1 A/California/7/09; “H1/California”);

ii) a modified H3 HA, wherein the one or more than one alteration is selected from Y98F, S136D; Y98F, S136N; Y98F, S137N; Y98F, D190G; Y98F, D190G; Y98F, R222W; Y98F, S228N; Y98F, S228Q; S136D; S136N; D190K; S228N; and S228Q; wherein the numbering of the alteration corresponds to position of reference sequence with SEQ ID NO: 204 (H3 A/Kansas/14/17; “H3/Kansas”);

iii) a modified H5 HA, wherein the one or more than one alteration is selected from Y91F; wherein the numbering of the alteration corresponds to position of reference sequence with SEQ ID NO: 205 (H5 A/Indonesia/5/05; “H5/Indonesia”);

iv) a modified H7 HA, wherein the one or more than one alteration is selected from Y88F; wherein the numbering of the alteration corresponds to position of reference sequence with SEQ ID NO: 206 (H7 A/Shanghai/2/12; “H7/Shanghai”);

v) a modified B HA wherein the one or more than one alteration is selected from S140A; S142A; G138A; L203A; D195G; and L203W; wherein the numbering of the alteration corresponds to position of reference sequence with SEQ ID NO: 207 (B/Phuket/3073/2013: “B/Phuket”);

vi) a modified B HA wherein the one or more than one alteration is selected from S140A; S142A; G138A; L202A; D194G; and L202W; wherein the numbering of the alteration corresponds to position of reference sequence with SEQ ID NO: 208 (B/Maryland/15/16; “B Maryland”);

vii) a modified B HA wherein the one or more than one alteration is selected from S140A; S142A; G138A; L201A; D193G; and L201W; wherein the numbering of the alteration corresponds to position of reference sequence with SEQ ID NO: 209 (B/Victoria/705/2018; “B/Victoria”); or

viii) a combination thereof.

The modified influenza HA proteins comprising one or more than one alteration as disclosed herewith that have been found to result in HA with improved characteristics as compared to the wildtype HA or unmodified HA proteins. Examples of improved characteristics of the modified HA protein include:
reduction of non-cognate interaction with sialic acid (SA) of a target, while maintaining cognate interaction, with the target;
reduction of non-cognate interaction with sialic acid (SA) of a protein on the surface of a cell, while maintaining cognate interaction, with the cell, such for example a B cell;
modulation and/or increase of an immunological response in an animal or a subject in response to an antigen challenge, when compared to an immunological response, wherein the HA does not comprise the one or more than one alteration;
increased HA protein yield when expressed in plant cells as compared to the wildtype or unmodified HA of the same strain or subtype of influenza that does not comprise the one or more than one alteration;
decreased hemagglutination titer of the modified HA protein when compared to the wildtype or unmodified HA protein.

For example, the modified HA may be a modified H1 HA comprising an alteration from Y91F, wherein the modified H1 may exhibit i) non-cognate interaction of the modified HA to sialic acid (SA) of a target for example a protein on the surface of a cell, while maintaining cognate interaction, with the target for example a cell such as a B cell and/or ii) wherein the modified HA exhibits decreased hemagglutination titer when compared to a wildtype or unmodified (parent) HA and/or iii) wherein the modified H1 HA may modulate and/or increase an immunological response in an animal or a subject in response to an antigen challenge, when compared to an immunological response, wherein the HA does not comprise the one or more than one alteration.

Furthermore, the modified HA may be a modified H3 comprising alterations selected from Y98F, S136D; Y98F, S136N; Y98F, S137N; Y98F, D190G; Y98F, D190K; Y98F, R222W; Y98F, S228N; and Y98F, S228Q; S136D; S136N; D190K; S228N; and S228Q, wherein the modified H3 may exhibit i) non-cognate interaction of the modified HA to sialic acid (SA) of a target for example a protein on the surface of a cell, while maintaining cognate interaction, with the target for example a cell such as a B cell and/or ii) wherein the modified HA exhibits decreased hemagglutination titer when compared to a wildtype or unmodified (parent) HA and/or iii) wherein the modified H3 HA may modulate and/or increase an immunological response in an animal or a subject in response to an antigen challenge, when compared to an immunological response, wherein the HA does not comprise the one or more than one alteration.

The modified HA may be a modified H7 HA comprising an alteration from Y88F, wherein the modified H7 exhibit i) non-cognate interaction of the modified HA to sialic acid (SA) of a target for example a protein on the surface of a cell, while maintaining cognate interaction, with the target for example a cell such as a B cell and/or ii) wherein the modified HA exhibits decreased hemagglutination titer when compared to a wildtype or unmodified (parent) HA and/or iii) wherein the modified H7 HA may modulate and/or increase an immunological response in an animal or a subject in response to an antigen challenge, when compared to an immunological response, wherein the HA does not comprise the one or more than one alteration.

In another embodiment the modified HA may be a modified H5 HA comprising an alteration from Y91F, wherein the modified H5 HA exhibit i) non-cognate interaction of the modified HA to sialic acid (SA) of a target for example a protein on the surface of a cell, while maintaining cognate interaction, with the target for example a cell such as a B cell and/or ii) wherein the modified HA exhibits decreased hemagglutination titer when compared to a wildtype or unmodified (parent) HA and/or iii) wherein the modified H5 HA may modulate and/or increase an immunological response in an animal or a subject in response to an antigen challenge, when compared to an immunological response, wherein the HA does not comprise the one or more than one alteration.

In a further embodiment, the modified HA may be a modified B HA comprising alterations selected from S140A; S142A; G138A; L203A; D195G; and L203W, wherein the modified B HA may exhibit i) non-cognate interaction of the modified HA to sialic acid (SA) of a target for example a protein on the surface of a cell, while maintaining cognate interaction, with the target for example a cell such as a B cell and/or ii) modulation and/or increase of immunological response in an animal or a subject in response to an antigen challenge, when compared to an immunological response, wherein the HA does not comprise the one or more than one alteration.

### Influenza HA

The term “influenza virus subtype” as used herein refers to influenza A and influenza B virus variants. Influenza virus subtypes and hemagglutinin (HA) from such virus subtypes may be referred to by their H number, such as, for example but not limited to, “HA of the H1 subtype”, “H1 HA”, or “H1 influenza”. The term “subtype” includes all individual “strains” within each subtype, which usually result from mutations and may show different pathogenic profiles. Such strains may also be referred to as various “isolates” of a viral subtype. Accordingly, as used herein, the terms “strains” and “isolates” may be used interchangeably.

Influenza results in agglutination of red blood cells (RBCs or erythrocytes) through multivalent binding of influenza HA to SA on the cell-surface. Many influenza strains can be serologically typed using reference anti-sera that prevents non-specific hemagglutination (ie: hemagglutination inhibition assay). Antibodies specific for particular influenza strains may bind to the virus and, thus, prevent such agglutination. Assays determining strain types based on such inhibition are typically known as hemagglutinin inhibition assays (HI assays or HAI assays) and are standard and well-known methods in the art to characterize influenza strains.

Hemagglutinin proteins from different virus strains also show significant sequence similarity at both the nucleic acid and amino acid levels. This level of similarity varies when strains of different subtypes are compared, with some strains displaying higher levels of similarity than others. This variation is sufficient to establish discrete subtypes and the evolutionary lineage of the different strains, but the DNA and amino acid sequences of different strains may be aligned using conventional bioinformatics techniques (Air, Proc. Natl. Acad. Sci. USA, 1981, 78:7643; Suzuki and Nei, Mol. Biol. Evol. 2002, 19:501).

An HA protein for use as described herein (i.e. to prepare a modified influenza HA protein that exhibits the property of having reduced, non-detectable, or no non-cognate interaction with SA, for example, reduced, non-detectable or no SA binding) may be derived from a type A influenza, a subtype of type A influenza HA selected from the group of H1, H2, H3, H4, H5, H6, H7, H8, H9, H10, H11, H12, H13, H14, H15, H16, H17 and H18, a type B influenza, a subtype of type B influenza, or a type C influenza. The HA may be from a type A influenza, selected from the group H1, H2, H3, H5, H6, H7, H9 and a type B influenza (for example Yamagata or Victoria lineage). Fragments of the HAs listed above may also be considered an HA protein of interest for use as described herein provided that when modified, the modified HA fragment exhibits reduced, non-detectable, or no non-cognate interaction with SA and that the modified HA fragment elicits an immune response. Furthermore, domains from an HA type or subtype listed above may be combined to produce chimeric HA's (see for example WO2009/076778 which is incorporated herein by reference).

Based on sequence similarities, influenza virus subtypes can further be classified by reference to their phylogenetic group. Phylogenetic analysis (Fouchier et al., J Virol. 2005 March; 79(5):2814-22) has demonstrated a subdivision of HAs that falls into two main groups (Air, Proc. Natl. Acad. Sci. USA, 1981, 78:7643): the H1, H2, H5 and H9 subtypes in phylogenetic group 1, and the H3, H4 and H7 subtypes in phylogenetic group 2.

Non limiting examples of subtypes comprising HA proteins that may be used as described herein (for example to prepare a modified influenza HA protein that may exhibit a modulated or increased immunological response in a subject and/or may exhibit the property of having reduced, non-detectable, or no non-cognate interaction with SA) include A/New Caledonia/20/99 (H1N1), A/California/07/09-H1N1 (A/Cal09-H1), A/California/04/2009 (H1N1), A/PuertoRico/8/34 (H1N1), A/Brisbane/59/2007 (H1N1), A/Brisbane/02/2018 (H1N1)pdm09-like virus, A/Solomon Islands 3/2006 (H1N1), A/Idaho/7/18 (H1N1), H1 A/Hawaii/70/19, A/Hawaii/70/2019 (H1N1)pdm09-like virus, A/chicken/New York/1995, A/Singapore/1/57 (H2N2), A/herring gull/DE/677/88 (H2N8), A/Brisbane 10/2007 (H3N2), A/Wisconsin/67/2005 (H3N2), A/Switzerland/9715293/2013-H3N2 (A/Swi-H3), A/Victoria/361/2011 (H3N2), A/Perth/16/2009 (H3N2), A/Kansas/14/17 (H3N2), A/Kansas/14/2017 (H3N2)-like virus, A/Minnesota/41/19 (H3N2), A/Hong Kong/45/2019 (H3N2)-like virus, A/shoveler/Iran/G54/03, A/Anhui/1/2005 (H5N1), A/Vietnam/1194/2004 (H5N1), A/Indonesia/5/2005 (H5N1), A/Vietnam/1194/2004 (H5N1), A/Egypt/N04915/14 (H5N1), A/Teal/HongKong/W312/97 (H6N1), A/Equine/Prague/56 (H7N7), H7 A/Hangzhou/1/13 (H7N9), A/Anhui/1/2013 (H7N9), A/Shanghai/2/2013 (H7N9), A/HongKong/1073/99 (H9N2), A/Texas/32/2003, A/mallard/MN/33/00, A/duck/Shanghai/1/2000, A/northern pintail/TX/828189/02, A/Turkey/Ontario/6118/68(H8N4), A/chicken/Germany/N/1949(H10N7), A/duck/England/56(H11N6), A/duck/Alberta/60/76(H12N5), A/Gull/Maryland/704/77(H13N6), A/Mallard/Gurjev/263/82, A/duck/Australia/341/83 (H15N8), A/black-headed gull/Sweden/5/99(H16N3), B/Brisbane/60/2008, B/Malaysia/2506/2004, B/Florida/4/2006, B/Phuket/3073/2013 (B/; Yamagata lineage), B/Phuket/3073/2013-like virus (B/Yamagata/16/88 lineage), B/Phuket/3073/2013 (B/Yamagata lineage)-like virus, B/Massachusetts/2/12, B/Wisconsin/1/2010, B/Lee/40, C/Johannesburg/66, B/Singapore/INFKK-16-0569/16 (Yamagata lineage), B/Maryland/15/16 (Victoria lineage), B/Victoria/705/18 (Victoria lineage), B/Washington/12/19 (Victoria lineage), B/Washington/02/2019 (B/Victoria lineage)-like virus, B/Darwin/8/19 (Victoria lineage), B/Darwin/20/19 (Victoria lineage), B/Colorado/06/2017-like virus (B/Victoria/2/87 lineage).

The HA protein for use as described herein (for example to prepare a modified influenza HA protein that may exhibit a modulated or increased immunological response in a subject and/or may exhibit the property of having reduced, non-detectable, or no non-cognate interaction with SA) may be an of influenza A subtype H1, H2, H3, H5, H6, H7, H8, H9, H10, H11, H12, H15, or H16 or the influenza may be an influenza B. For example, the H1 protein may be derived from the A/New Caledonia/20/99 (H1N1), A/PuertoRico/8/34 (H1N1), A/Brisbane/59/2007 (H1N1), A/Brisbane/02/2018 (H1N1)pdm09-like virus, A/Solomon Islands 3/2006 (H1N1), A/Idaho/7/18 (H1N1), H1 A/Hawaii/70/19, /Hawaii/70/2019 (H1N1)pdm09-like virus, A/California/04/2009 (H1N1) or A/California/07/2009 (H1N1) strain. In a further aspect of the invention, the H2 protein may be from the A/Singapore/1/57 (H2N2) strain. The H3 protein may be from the A/Brisbane 10/2007 (H3N2), A/Wisconsin/67/2005 (H3N2), A/Switzerland/9715293/2013-H3N2 (A/Swi-H3), A/Victoria/361/2011 (H3N2), A/Texas/50/2012 (H3N2), A/Kansas/14/17 (H3N2), A/Kansas/14/2017 (H3N2)-like virus, A/Hawaii/22/2012 (H3N2), A/New York/39/2012 (H3N2), A/Perth/16/2009 (H3N2) strain, A/Hong Kong/45/2019 (H3N2) like virus, or A/Minnesota/41/19 (H3N2). The H5 protein may be from the A/Anhui/1/2005 (H5N1), A/Vietnam/1194/2004 (H5N1), A/Vietnam/1194/2004 (H5N1), A/Egypt/N04915/14 (H5N1), or A/Indonesia/5/2005 strain. In an aspect of the invention, the H6 protein may be from the A/Teal/HongKong/W312/97 (H6N1) strain. The H7 protein may be from the A/Equine/Prague/56 (H7N7) strain, or H7 A/Hangzhou/1/2013, A/Anhui/1/2013 (H7N9), or A/Shanghai/2/2013 (H7N9) strain. The H8, H9, H10, H11, H12, H15, or H16 protein may be from the A/Turkey/Ontario/6118/68(H8N4), A/HongKong/1073/99 (H9N2) strain, A/chicken/Germany/N/1949(H10N7), A/duck/England/56(H11N6), A/duck/Alberta/60/76(H12N5), A/duck/Australia/341/83 (H15N8), A/black-headed gull/Sweden/5/99(H16N3). The HA protein for use as described herein may be derived from an influenza virus may be a type B virus, including B/Malaysia/2506/2004, B/Florida/4/2006, B/Brisbane/60/08, B/Massachusetts/2/2012-like virus (Yamagata lineage), or B/Wisconsin/1/2010 (Yamagata lineage), B/Phuket/3073/2013-like virus (B/Yamagata/16/88 lineage), B/Phuket/3073/2013 (B/Yamagata lineage)-like virus, B/Lee/40, B/Singapore/INFKK-16-0569/16 (Yamagata lineage), B/Maryland/15/16 (Victoria lineage), B/Victoria/705/18 (Victoria lineage), B/Washington/12/19 (Victoria lineage), B/Washington/02/2019 (B/Victoria lineage)-like virus, B/Darwin/8/19 (Victoria lineage), B/Darwin/20/19 (Victoria lineage), B/Colorado/06/2017-like virus (B/Victoria/2/87 lineage). Non-limiting examples of amino acid sequences of the HA proteins from H1, H2, H3, H5, H6, H7, H9 or B subtypes include sequences as described in WO 2009/009876, WO 2009/076778, WO 2010/003225, PCT/CA2019/050891, PCT/CA2019/050892, PCT/CA2019/050893 (which are incorporated herein by reference).

HA proteins (parent HAs), that may be modified as described herein to reduce or eliminate non-cognate interaction with SA, for example having reduced or no SA binding, may include wild type HA proteins, including new HA proteins that emerge over time due to natural modifications of the HA amino acid sequence, or non-native HA proteins, that may be produced as a result of altering the HA proteins (e.g. chimeric HA proteins, or HA proteins that have been altered to achieve a desirable property, for example, increasing expression within a host). Similarly, modified HA proteins as described herein to reduce or eliminate SA binding, may be derived from wild type HA proteins, novel HA proteins that emerge over time due to natural modifications of the HA amino acid sequence, non-modified HA proteins, non-native HA proteins for example, chimeric HA proteins, or HA proteins that have been altered to achieve a desirable property, for example, increasing expression of HA or VLPs within a host.

By “parent HA” it is meant that the HA protein from which the modified HA protein may be derived. The parent HA does not comprise a modification that reduces or eliminates non-cognate interactions with SA, for example reduced or no SA binding. Preferably, the parent HA protein exhibits antigenic properties similar to that of a corresponding native or wild-type influenza strain, including binding to SA on host cells. The parent HA may comprise a wild type or native HA, however, the parent HA may comprise an altered amino acid sequence, provided the alteration in the sequence is functionally separate from the modification that reduces or eliminates non-cognate interactions with SA, or reduces or eliminates SA binding. Preferably, the parent HA exhibits similar cognate interactions as those observed with a corresponding native or wild type HA, and comprises a conformation that elicits a similar immune response as that are observed with a corresponding native or wild type HA, when the non-modified HA is introduced into a subject. A parent HA may also be referred to as a non-modified HA.

The HA for use as described herein (i.e. a modified influenza HA protein that exhibits the property of having reduced, non-detectable, or no non-cognate interactions with SA) may also be derived from a parent HA that is non-native and comprises one or more than one amino acid sequence alterations that results in increased expression within a host, for example deletion of the proteolytic loop region of the HA molecule as described in WO2014/153674 (which is incorporated herein by reference), or comprising other substitutions or alterations as described in WO2020/00099, WO2020/000100, WO2020/000101 (each of which is incorporated herein by reference). The HA for use as described herein may also be derived from a non-native (parent) HA comprising one or more than one amino acid sequence alterations that results in an altered glycosylation pattern of the expressed HA protein, for example as described in WO2010/006452, WO2-14/071039, and WO2018/058256 (each of which is incorporated herein by reference).

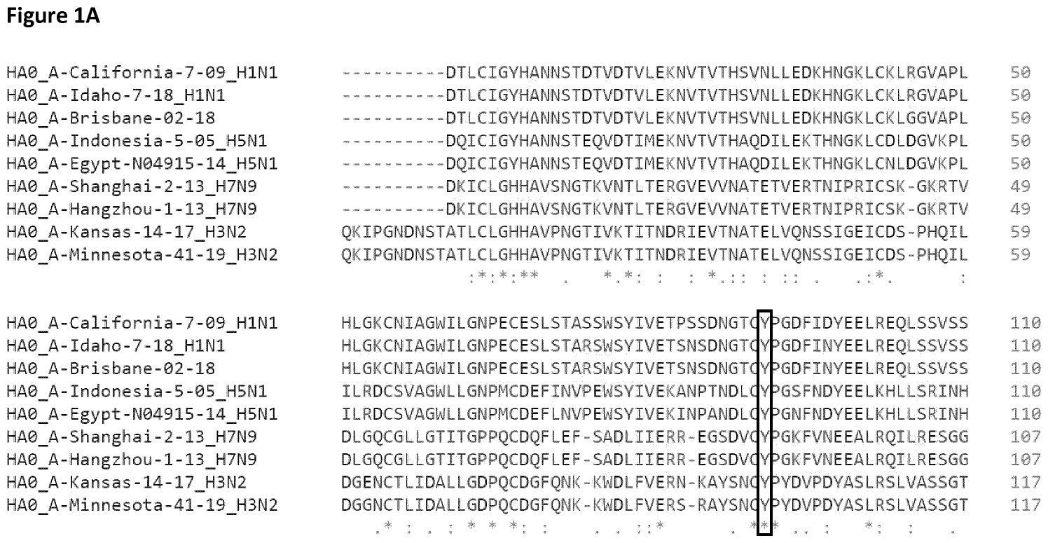

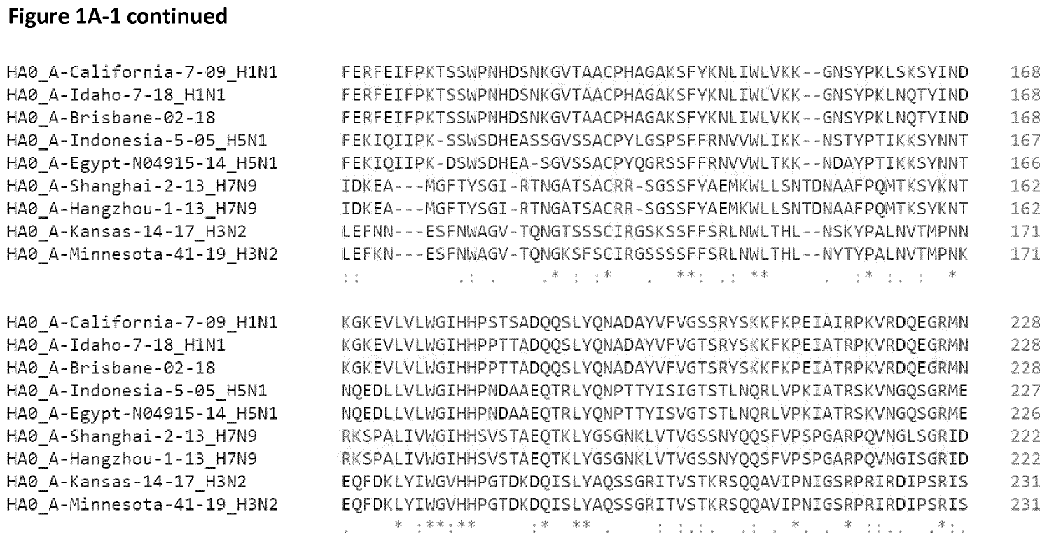

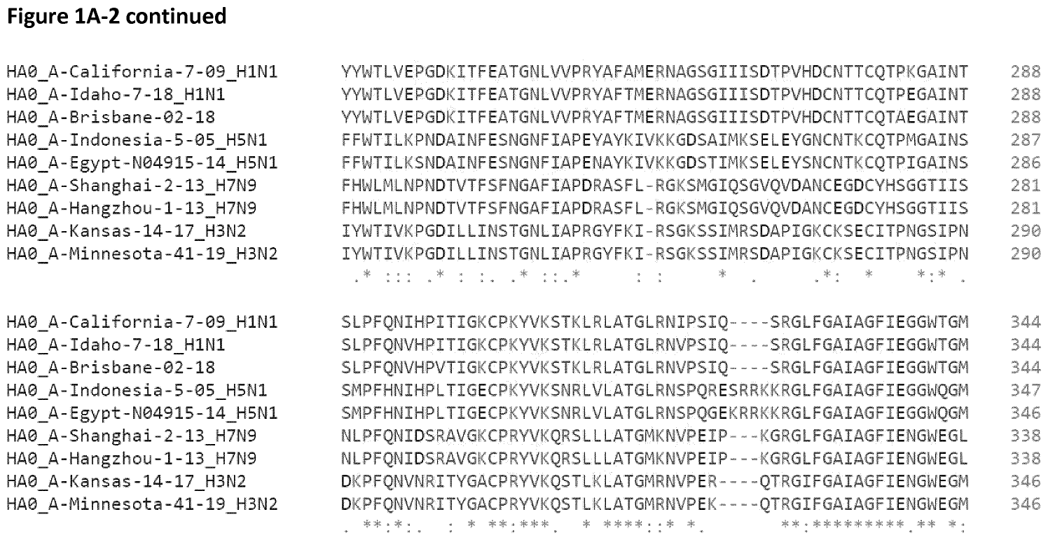

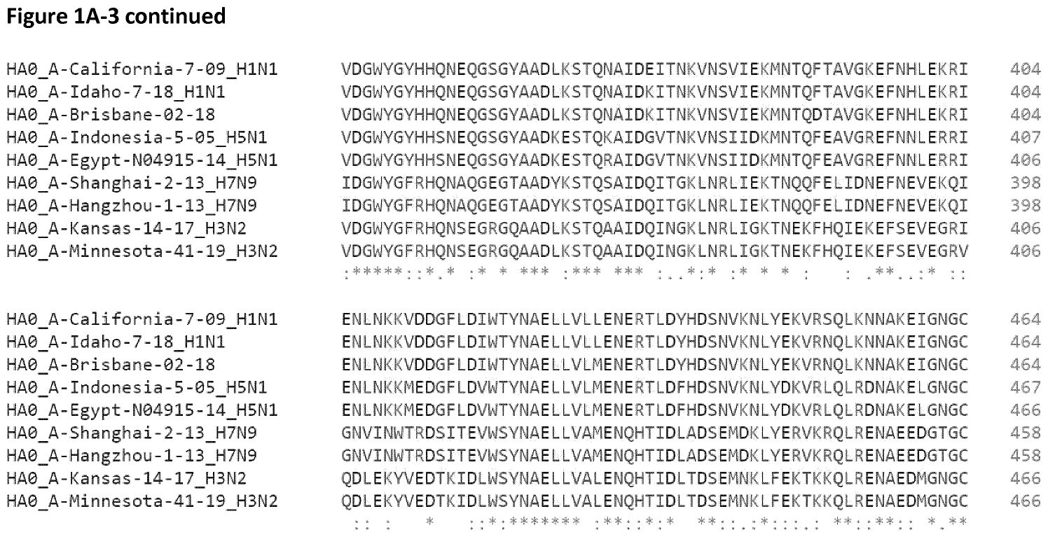

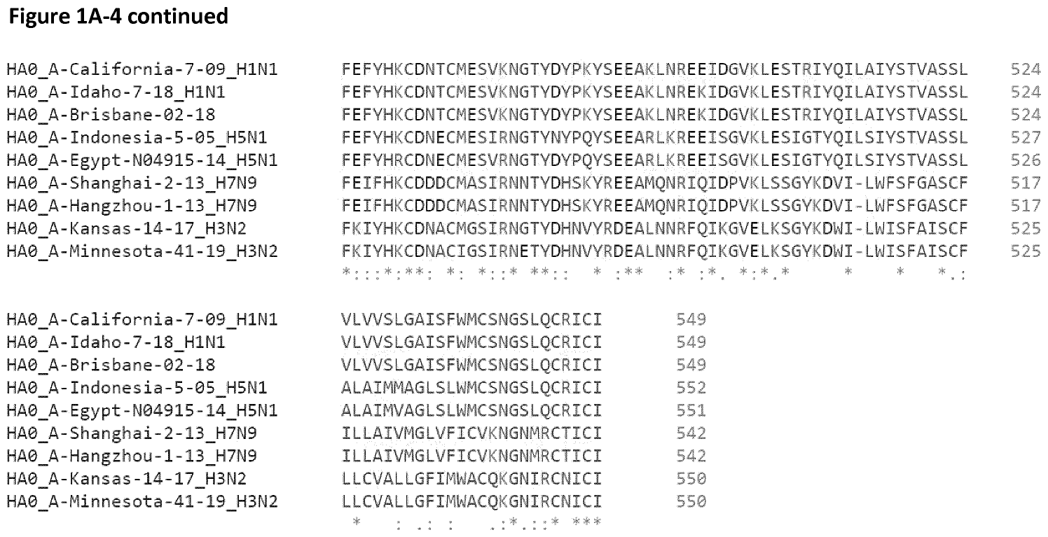

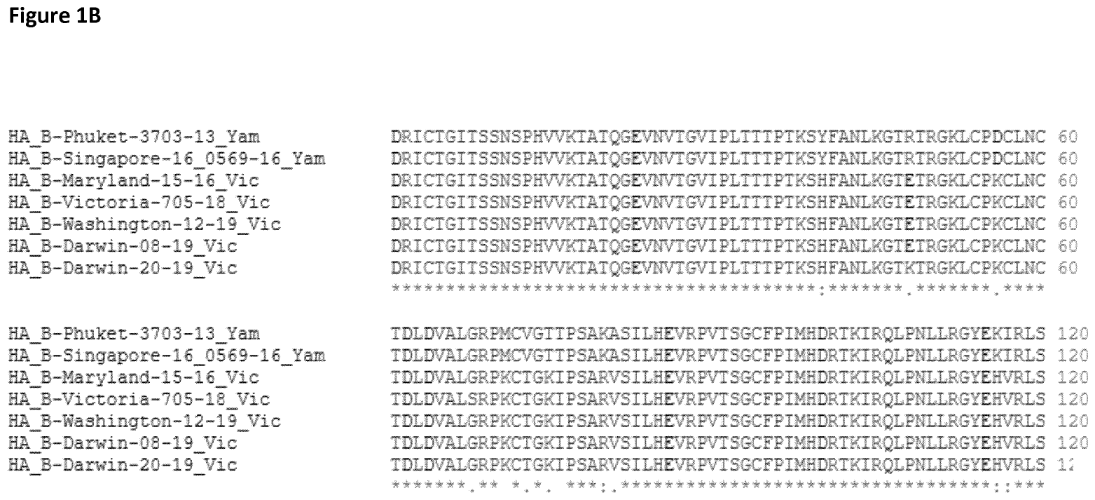

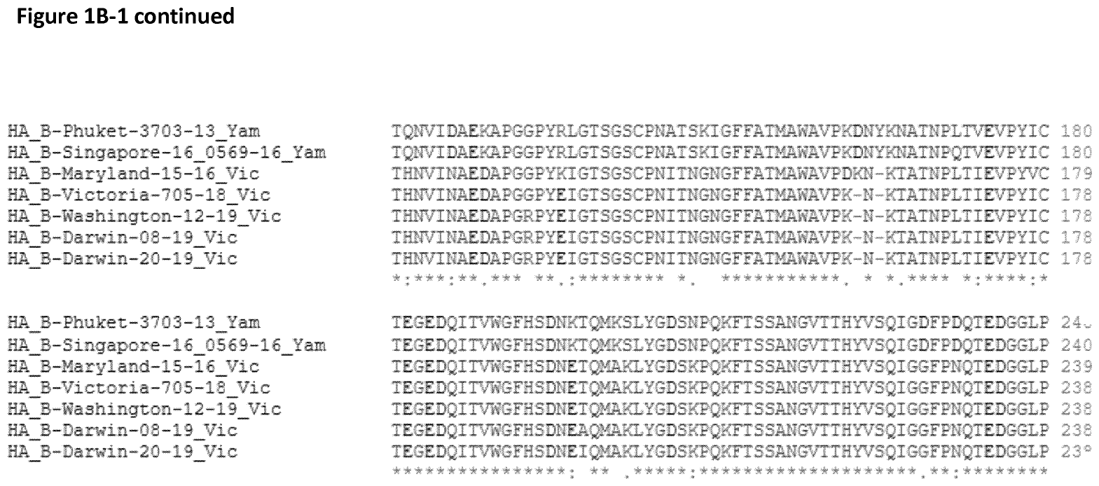

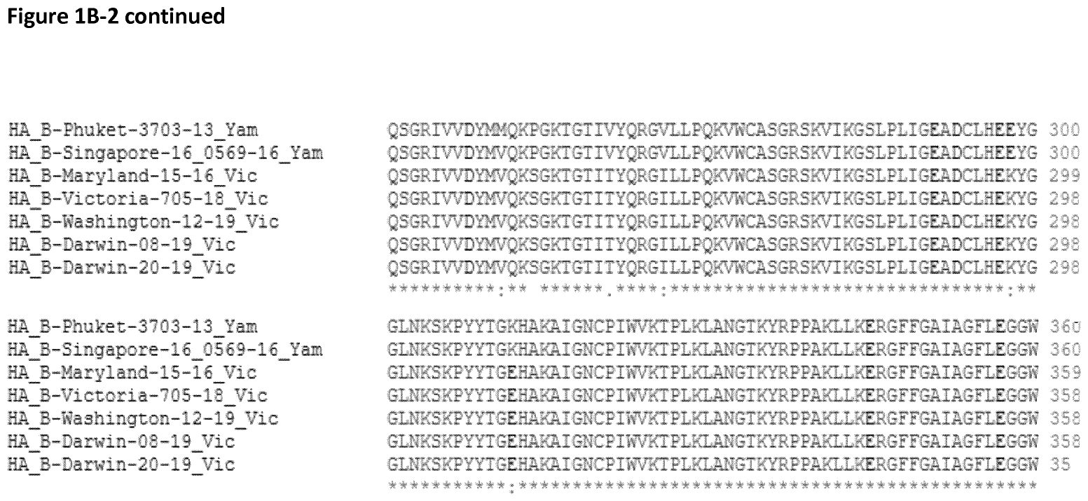

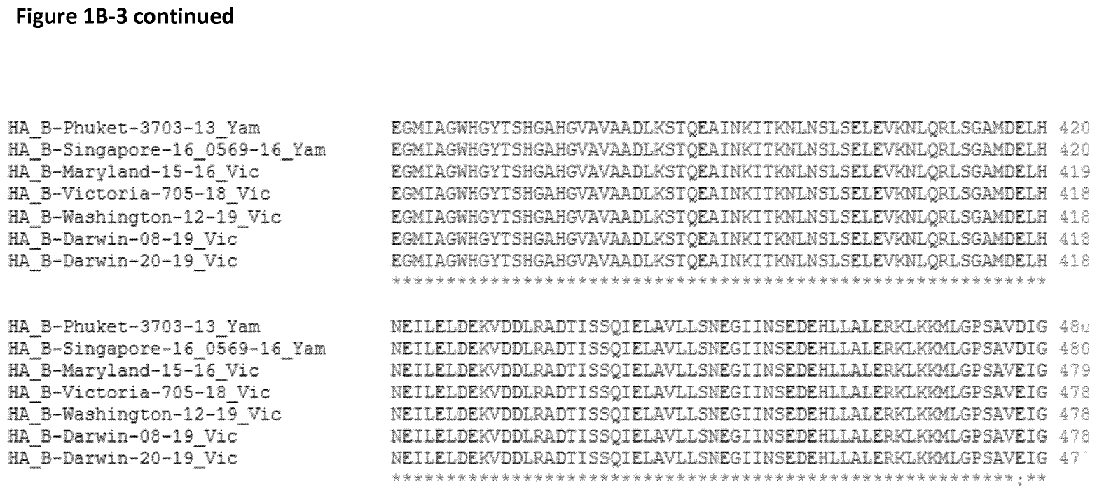

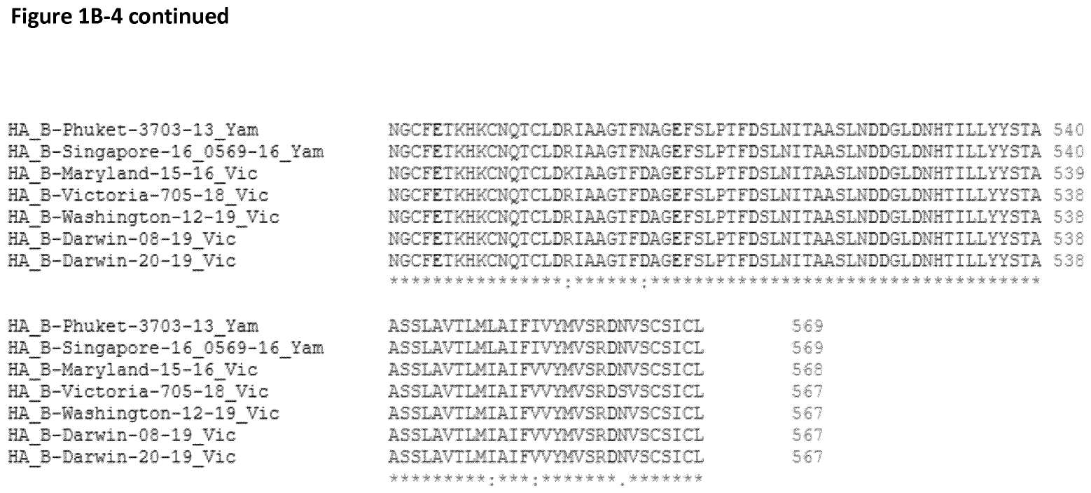

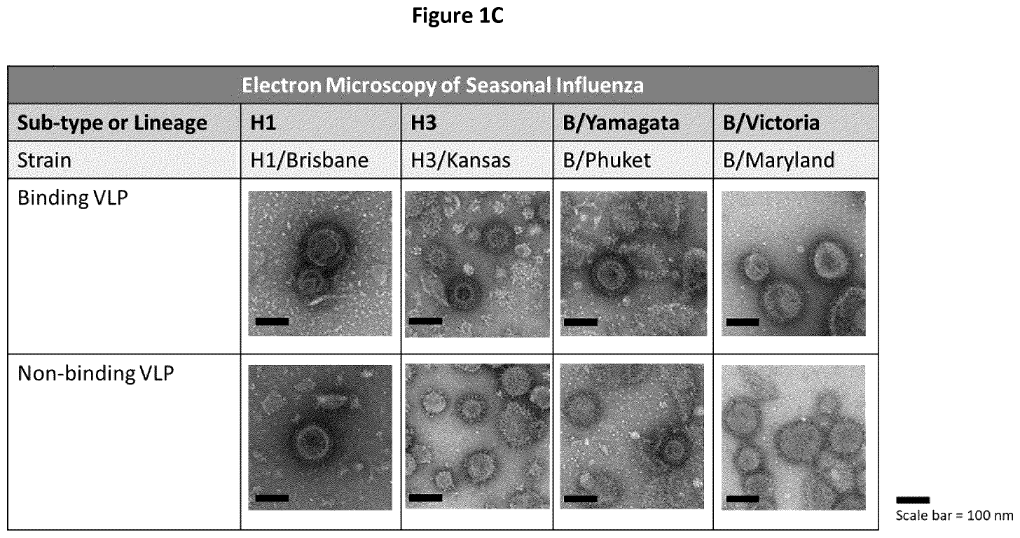

The modified HA that exhibits the property of having reduced, non-detectable, or no non-cognate interaction with SA, for example reduced or no SA binding, may also be derived from a parent HA that is a chimeric HA, wherein a native transmembrane domain of the HA is replaced with a heterologous transmembrane domain. The transmembrane domain of HA proteins is highly conserved (see for example FIG. 1C of WO2010/148511; which is incorporated herein by reference). The heterologous transmembrane domain may be obtained from any HA transmembrane domain, for example but not limited to the transmembrane domain from H1 California, B/Florida/4/2006 (GenBank Accession No. ACA33493.1), B/Malaysia/2506/2004 (GenBank Accession No. ABU99194.1), H1/Bri (GenBank Accession No. ADE28750.1), H1 A/Solomon Islands/3/2006 (GenBank Accession No. ABU99109.1), H1/NC (GenBank Accession No. AAP34324.1), H2 A/Singapore/1/1957 (GenBank Accession No. AAA64366.1), H3 A/Brisbane/10/2007 (GenBank Accession No. ACI26318.1), H3 A/Wisconsin/67/2005 (GenBank Accession No. ABO37599.1), H5 A/Anhui/1/2005 (GenBank Accession No. ABD28180.1), H5 A/Vietnam/1194/2004 (GenBank Accession No. ACR48874.1), or H5-Indo (GenBank Accession No. ABW06108.1). The transmembrane domain may also be defined by the following consensus amino acid sequence:

(SEQ ID NO: 110)
iLXiYystvAiSslXlXXmlagXsXwmcs

Other chimeric, parent, HAs may also be used as described herein, for example a chimeric HA comprising in series, an ectodomain from a virus trimeric surface protein or fragment thereof, fused to an influenza transmembrane domain and cytoplasmic tail as described in WO2012/083445 (which is incorporated herein by reference).

Therefore, the parent HA protein that may be modified as described herein to produce a modified HA exhibiting reduce or eliminate non-cognate interaction with SA, for example reduced or no SA binding, may have from about 80 to about 100%, or any amount therebetween, amino acid sequence identity, from about 90-100% or any amount therebetween, amino acid sequence identity, or from about 95-100% or any amount therebetween, amino acid sequence identity, to a wild type, or non-modified HA protein obtained from an influenza strain including those influenza strains listed herein, provided that the parent HA protein induces immunity to influenza in a subject, when the parent HA protein is administered to a subject. For example, the parent HA protein that may be modified as described herein to reduce or eliminate SA binding, may have from 80, 82, 84, 86, 88, 90, 92, 94, 96, 98, 100%, or any amount therebetween, amino acid sequence identity (sequence similarity; percent identity; percent similarity) with a wild type or non-modified HA protein obtained from any influenza strain including those influenza strains listed herein, provided that the parent HA protein induces immunity to influenza in a subject, when the HA protein is administered to the subject.

For example, it is provided a modified influenza hemagglutinin (HA) protein comprising an amino acid sequence having from about 70% to about 100%, or any amount therebetween, for example 80, 82, 84, 86, 88, 90, 91, 92, 93, 94, 95, 96, 97, 98, 99, 100% or any amount therebetween, sequence identity or sequence similarity with a sequence of the sequences of SEQ ID NO: 203 (exemplary H1 sequence), SEQ ID NO: 204 (exemplary H3 sequence), SEQ ID NO: 205 (exemplary H5 sequence), SEQ ID NO: 206 (exemplary H7 sequence), SEQ ID NO: 207 (exemplary B sequence), SEQ ID NO: 208 (exemplary B sequence), and SEQ ID NO: 209 (exemplary B sequence), provided that the influenza HA protein comprises at least one substitution or alteration as described herewith and is able to form VLPs, reduce non-cognate interaction with a protein on the surface of the cell, induces an immune response when administered to a subject, or a combination thereof.

It is further provided that the modified influenza hemagglutinin (HA) protein may comprise an amino acid sequence having from about 70% to about 100%, or any amount therebetween, sequence identity or sequence similarity or any amount therebetween, for example 80, 82, 84, 86, 88, 90, 91, 92, 93, 94, 95, 96, 97, 98, 99, 100% or any amount therebetween sequence identity or sequence similarity, with amino acids 25 to 573 [H1] of SEQ ID NO:2, SEQ ID NO:12, SEQ ID NO: 101, SEQ ID NO:105, SEQ ID NO:195, or SEQ ID NO:197; with amino acids 25 to 574 [H3] of SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:112, SEQ ID NO:114, SEQ ID NO:116, SEQ ID NO:118, SEQ ID NO:120, or SEQ ID NO: 122; with amino acids 25 to 576 [H5] of SEQ ID NO:199 or SEQ ID NO:202; with amino acids 1 to 551 [H5 A/Egypt/N04915/14] of SEQ ID NO:108; with amino acids 25 to 566 [H7] of SEQ ID NO:21 or SEQ ID NO:26; with amino acids 1 to 542 [H7 A/Hangzhou/1/13] of SEQ ID NO: 109; with amino acids 25 to 576 [B] of SEQ ID NO:28, SEQ ID NO:33, SEQ ID NO:37, SEQ ID NO:41, SEQ ID NO:45, SEQ ID NO:49, SEQ ID NO:53, SEQ ID NO:124, SEQ ID NO:126, SEQ ID NO:128, SEQ ID NO:130, SEQ ID NO:132, SEQ ID NO:134, or SEQ ID NO:136; with amino acids 25 to 575 [B] of SEQ ID NO:138, SEQ ID NO:141, SEQ ID NO:143, SEQ ID NO: 145, SEQ ID NO:147, SEQ ID NO:149, or SEQ ID NO:151; with amino acids 25 to 574 [B] of SEQ ID NO:153, SEQ ID NO:155, SEQ ID NO:157, SEQ ID NO:159, SEQ ID NO:161, SEQ ID NO:163, SEQ ID NO:165, SEQ ID NO:181, SEQ ID NO:183, SEQ ID NO: 185, SEQ ID NO:187, SEQ ID NO:189, SEQ ID NO: 191, or SEQ ID NO:193; with amino acids 1 to 569 [B] of SEQ ID NO:14; with amino acids 1 to 568 [B] of SEQ ID NO:15; or with amino acids 1 to 567 [B] of SEQ ID NO:16, SEQ ID NO:17, SEQ ID NO:18, or SEQ ID NO:19, provided that the modified influenza HA protein comprises at least one substitution or alteration as described herewith and is able to form VLPs, reduce non-cognate interaction with a protein on the surface of a cell, induces an immune response when administered to a subject, or a combination thereof.

It is further provided that the modified influenza hemagglutinin (HA) protein may comprise an amino acid sequence having from about 70% to about 100%, or any amount therebetween, sequence identity or sequence similarity or any amount therebetween, for example 80, 82, 84, 86, 88, 90, 91, 92, 93, 94, 95, 96, 97, 98, 99, 100% or any amount therebetween, sequence identity or sequence similarity with amino acids of SEQ ID NO:2, SEQ ID NO:12, SEQ ID NO:101, SEQ ID NO: 105, SEQ ID NO:195, SEQ ID NO:197; SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:112, SEQ ID NO:114, SEQ ID NO:116, SEQ ID NO:118, SEQ ID NO:120, SEQ ID NO:122, SEQ ID NO:199 or SEQ ID NO:202, SEQ ID NO:108, SEQ ID NO:21 SEQ ID NO:26; SEQ ID NO:109; SEQ ID NO:28, SEQ ID NO:33, SEQ ID NO:37, SEQ ID NO:41, SEQ ID NO:45, SEQ ID NO:49, SEQ ID NO:53, SEQ ID NO:124, SEQ ID NO:126, SEQ ID NO:128, SEQ ID NO:130, SEQ ID NO:132, SEQ ID NO:134, or SEQ ID NO:136; SEQ ID NO:138, SEQ ID NO:141, SEQ ID NO:143, SEQ ID NO:145, SEQ ID NO:147, SEQ ID NO:149, or SEQ ID NO:151 SEQ ID NO:153, SEQ ID NO:155, SEQ ID NO:157, SEQ ID NO:159, SEQ ID NO:161, SEQ ID NO:163, SEQ ID NO:165, SEQ ID NO:181, SEQ ID NO:183, SEQ ID NO:185, SEQ ID NO:187, SEQ ID NO:189, SEQ ID NO:191, SEQ ID NO:193; SEQ ID NO: 14; SEQ ID NO:15; SEQ ID NO:16, SEQ ID NO:17, SEQ ID NO:18, or SEQ ID NO:19, provided that the modified influenza HA protein comprises at least one substitution or alteration as described herewith and is able to form VLPs, reduce non-cognate interaction with a protein on the surface of a cell, induces an immune response when administered to a subject, or a combination thereof.

Hemagglutinin proteins are known to aggregate to form dimers, trimers, multimeric complexes, or larger structures, for example HA rosettes, protein complexes comprising a plurality of HA proteins, multimeric HA complexes comprising a plurality of HA proteins, metaprotein HA complexes comprising a plurality of HA proteins, nanoparticles comprising a plurality of HA proteins, or VLPs comprising HA. Such aggregates of HA proteins are collectively referred to as “suprastructures”. Unless specified otherwise, the terms “multimeric complex”, “VLPs”, “nanoparticles”, and “metaproteins” may be used interchangeably, and they are examples of suprastructures comprising HA. Any form and number of HA proteins, from dimers, trimers, rosettes, multimeric complexes, metaprotein complexes, nanoparticles, VLPs, or other suprastructures comprising HA may be used to prepare immunogenic compositions and used as described herein.

The terms “percent similarity”, “sequence similarity”, “percent identity”, or “sequence identity”, when referring to a particular sequence, are used for example as set forth in the University of Wisconsin GCG software program, or by manual alignment and visual inspection (see, e.g., Current Protocols in Molecular Biology, Ausubel et al., eds. 1995 supplement). Methods of alignment of sequences for comparison are well-known in the art. Optimal alignment of sequences for comparison can be conducted, using for example the algorithm of Smith & Waterman, (1981, Adv. Appl. Math. 2:482), by the alignment algorithm of Needleman & Wunsch, (1970, J. Mol. Biol. 48:443), by the search for similarity method of Pearson & Lipman, (1988, Proc. Natl. Acad. Sci. USA 85:2444), by computerized implementations of these algorithms (for example: GAP, BESTFIT, FASTA, and TFASTA in the Wisconsin Genetics Software Package, Genetics Computer Group (GCG), 575 Science Dr., Madison, Wis.).

An example of an algorithm suitable for determining percent sequence identity and sequence similarity are the BLAST and BLAST 2.0 algorithms, which are described in Altschul et al., (1977, Nuc. Acids Res. 25:3389-3402) and Altschul et al., (1990, J. Mol. Biol. 215:403-410), respectively. BLAST and BLAST 2.0 are used, with the parameters described herein, to determine percent sequence identity for the nucleic acids and amino acids of the invention. For example, the BLASTN program (for nucleotide sequences) may use as defaults a wordlength (W) of 11, an expectation (E) of 10, M=5, N=−4 and a comparison of both strands. For amino acid sequences, the BLASTP program may use as defaults a word length of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands. Software for performing BLAST analyses is publicly available through the National Center for Biotechnology Information (see URL: ncbi.nlm.nih.gov/).

### Modified HA Protein

A nucleotide sequence (or nucleic acid) of interest encodes a modified influenza HA protein (also termed modified HA protein, modified HA, modified influenza HA), as described herein, if the modified HA protein exhibits the property of having reduced, non-detectable, or no non-cognate interaction with SA, for example having reduced, non-detectable, or no SA binding. Likewise, a protein of interest, as described herein, is a modified influenza HA protein if the protein of interest exhibits the property of having reduced, non-detectable, or no non-cognate interaction with SA, for example having reduced, non-detectable, or no SA binding. Preferably, the modified HA comprises a conformation that elicits an improved immune response when compared with the immune response observed using the corresponding parent HA, and the modification that results in reduced or non-detectable non-cognate interaction with SA does not alter cognate interactions of the modified HA protein with a target (for example, with targets mediated by the B cell receptor), when compared with the parent HA protein and the same target(s). The modification that results in reduced or non-detectable non-cognate interaction with SA does not alter recognition of the modified HA by antibodies or antigen-specific immune cells (i.e. B cells and T cells), for example, peripheral blood mononuclear cells (PBMC) or B cells expressing antibody against HA following vaccination with HA, or other cells, for example a transfected cell expressing a membrane bound IgM-HA. The modification that reduces non-cognate interactions between the HA and SA may involve substituting, deleting or adding one or more than one amino acid residue in the receptor binding site of HA, or altering the glycosylation pattern at or near the receptor binding site of HA, thereby sterically hindering non-cognate interactions between the HA and SA.

Amino acids that may be substituted in a HA of interest to reduce or eliminate SA binding may be determined by sequence alignment of a reference HA amino acid sequence with the HA of interest, and identifying the position of the corresponding amino acid(s) (see FIG. 1A for amino acid alignment of H1, H3, H5, H7 HAs, and FIG. 1B for alignment of B HAs). As one of skill would understand, HAs obtained from different strains may not comprise the same number of amino acids and the relative position of an amino acid location within a reference HA sequence may not be the same as that of the HA of interest. Non limiting examples of amino acid residues of HAs that may be substituted in order to obtain an HA with reduced, non-detectable, or no non-cognate interaction with SA are provided in Table 1.

TABLE 1
amino acid residues that may be substituted to
produce a modified influenza hemagglutinin (HA)

| HA | Parent strain amino acid # | Relative to reference amino |
|---|---|---|
| strain | (parent strain) | acid # (reference strain) |
| A/H1 | 91 (H1) | 98 (H3); 88 (H7) |
| A/H3 | 98 (H3) | 91 (H1); 88 (H7) |
| A/H5 | 91 (H5) | 98 (H3); 88 (H7) |
| A/H7 | 88 (H7) | 91 (H1); 98 (H3) |
| B | 138 (B/Phuket, B/Maryland, | 138 (B/Phuket, B/Maryland, |
| | B/Victoria) | B/Victoria) |
| B | 140 (B/Phuket, B/Maryland, | 140 (B/Phuket, B/Maryland, |
| | B/Victoria) | B/Victoria) |
| B | 142 (B/Phuket, B/Maryland, | 142 (B/Phuket, B/Maryland, |
| | B/Victoria) | B/Victoria) |
| B | 195 (B/Phuket) | 194 (B/Maryland) |
| | | 193 (B/Victoria) |
| B | 194 (B/Maryland) | 193 (B/Victoria) |
| | | 195 (B/Phuket) |
| B | 193 (B/Victoria) | 194 (B/Maryland) |
| | | 195 (B/Phuket) |
| B | 203 (B/Phuket) | 202 (B/Maryland) |
| | | 201 (B/Victoria) |
| | 202 (B/Maryland) | 203 (B/Phuket) |
| | | 201 (B/Victoria) |
| | 201 (B/Victoria) | 202 (B/Maryland) |
| | | 203 (B/Phuket) |

Amino acid residue numbers correspond to representative HA sequences for each strain with the following sequences: H1 (SEQ ID NO: 203), H3 (SEQ ID NO: 204), H5 (SEQ ID NO: 205), H7 (SEQ ID NO: 206) B/Phuket (SEQ ID NO: 207), B/Maryland (SEQ ID NO: 208), B/Victoria (SEQ ID NO: 209).

As shown above, residues 194 and 202 in reference strain with SEQ ID NO: 208 (B/Maryland) and residues 193 and 201 in references strain with SEQ ID NO 209 (B/Victoria) correspond to residues 195 and 203 in reference strain of SEQ ID NO: 207 (B/Phuket).

The property of non-cognate interaction with SA, SA binding (or SA binding affinity), between a wild type (or non-modified) HA and the modified HA, with a blood cell, a transfected cell expressing membrane bound IgM HA, an antibody, a peptide comprising SA, or binding to a target comprising a terminal α-2,3 linked (avian) or α-2,6 linked (human) SA, and cognate interactions between the wild type (or non-modified) HA and the modified HA and a blood cell, or an antibody, may be determined using one or more assays that are known in the art. Non limiting examples of assays or combinations of assays that may be used are described in Hendin H., et. al. (Hendin H., et. al., 2017, Vaccine 35:2592-2599; which is incorporated herein by reference), Whittle J., et. al. (Whittle J., et. al., 2014, J. Virol. 88:4047-4057; which is incorporated herein by reference), Lingwood, D., et. al., (Lingwood, D., et. al., 2012 Nature 489:566-570 (which is incorporated herein by reference), Villar, R., et. al., (Villar, R., et. al., 2016, Scientific Reports (Nature) 6:36298), and may include the use of flow cytometry (see Example 3.7), using wild type (or non-modified) HA, and modified HA with reduced, non-detectable, or no non-cognate interaction with SA, to probe control and transfected cells expressing membrane bound HA. Surface plasmon resonance (SPR) analysis (see example 3.3), and/or hemagglutination assays (Example 3.1), microscopy or imaging (to determine HA-SA binding), coupled with Western blot analysis (to determine HA yield) and/or ELISA, may also be used to derive the amount of HA-SA interaction, and HA-epitope recognition (an example of cognate interaction), that a candidate HA protein exhibits.

By a modified HA having “reduced, non-detectable or no non-cognate interaction with SA”, or “reduced, non-detectable, or no binding to SA” it is meant that the non-cognate interaction, for example binding, of the modified HA to SA is reduced, reduced to undetectable levels, or eliminated, when compared to the non-cognate interaction, for example binding, of a corresponding parent HA that does not comprise the modification that results in reduced, undetectable, or no non-cognate interaction with SA. The parent HA may include for example, a wild type influenza HA, an HA comprising a sequence that is altered, but the alteration is not associated with non-cognate interaction with SA, for example binding with HA (i.e. a non-modified HA), a suprastructure comprising the parent HA, for example, a VLP. A modified HA having reduced, undetectable, or no non-cognate interaction with SA may exhibit from about 60 to about 100%, or any amount therebetween, binding with SA, when compared to the binding of the corresponding parent HA that does not comprise the modification that alters SA binding, with SA. This may also be restated as the modified HA comprising from about 0 to about 40%, or any amount therebetween, of the binding affinity with SA, when compared to the binding affinity of the corresponding parent HA, that does not comprise the modification, with SA.

For example, an alteration that reduces binding of the modified HA to SA may reduce binding of the modified HA from about 70 to about 100%, or any amount therebetween, from about 80 to about 100%, or any amount therebetween, or from about 90 to about 100%, or any amount therebetween, when compared to the binding of the corresponding parent HA to SA. For example the alteration may reduce the binding of the modified HA to SA by about 60, 62, 64, 66, 68, 70, 72, 74, 76, 78, 80, 82, 84, 86, 88, 90, 92, 94, 96, 98 or 100%, or any amount therebetween, when compared to binding of the corresponding parent HA to SA. Alternatively, the alteration that reduces binding of the modified HA to SA may exhibit from about 0 to about 30%, or any amount therebetween, of the binding affinity of a corresponding parent HA to SA, or from about 0 to about 20%, or any amount therebetween, of the binding affinity of a corresponding wild type (or non-modified) HA to SA, or from 0-10%, or any amount therebetween, of the binding affinity of the corresponding parent HA. For example, from about 0, 2, 4, 6, 8, 10, 112, 14, 16, 8, 20, 22, 24, 26, 28 or about 30%, or any amount therebetween, of the binding affinity of a corresponding parent HA to SA.

A modified HA cognitively interacts with a target, when from about 80 to 100%, or any amount therebetween of the modified HA associates with a target, such as a blood cell for example, a B cell, or other target, while also exhibiting the property of reduced, or non-detectable, binding to SA. Furthermore, a modified HA exhibits cognate interaction with a target if about 85 to about 100%, or any amount therebetween of the modified HA associates with the target, from about 90-100%, or any amount therebetween of the modified HA associates with the target, from about 95-100%, or any amount therebetween of the modified HA associates with the target, or from about 80, 82, 84, 86, 88, 90, 92, 94, 96, 98 or 100%, or any amount therebetween of the modified HA associates with the target, while also exhibiting reduced, or non-detectable, SA binding. Cognate interaction between a modified HA or a parent HA and a target can be determined, for example, by determining the avidity between the modified HA or parent HA and the target.

The modified influenza HA sequence, nucleic acid, or protein may be derived from a corresponding wild type, non-modified, or altered HA sequence, nucleic acid or protein, from any influenza strain, for example, an influenza strain obtained from the group of H1, H2, H3, H4, H5, H6, H7, H8, H9, H10, H11, H12, H13, H14, H15, H16, H17 and H18, or influenza from a type B strain.

Modified influenza HA proteins that result in reduced, non-detectable, or no non-cognate interaction with SA, and methods of producing modified influenza HA proteins in a suitable host, for example but not limited to a plant, are described herein.

The modified influenza HA proteins disclosed herein, that result in reduced or no non-cognate interaction with SA, have been found to result in improved HA characteristics, for example, use of the modified HA protein, suprastructure or VLP comprising the modified HA protein, as an influenza vaccine that exhibits increased immunogenicity and efficacy when compared to the immunogenicity and efficacy of an influenza vaccine comprising the corresponding parent (non-modified, or wild type) influenza HA, suprastructure or VLP comprising the parent HA protein. The alteration in the modified HA reduces binding of the modified HA to SA may be a result of a substitution, a deletion or an insertion of one or more amino acid within the HA sequence, or it may be a result of a chemical modification of the HA protein, for example by altering the glycosylation pattern of HA, or by removing one or more than one glycosylation site of HA.

Modified influenza HA proteins, suprastructures comprising modified HAs, nanoparticles comprising HAs, suprastructures or VLPs comprising the modified proteins, and methods of producing modified influenza HA proteins, suprastructures or VLPs, in a suitable host, for example but not limited to plants, are also described herein.

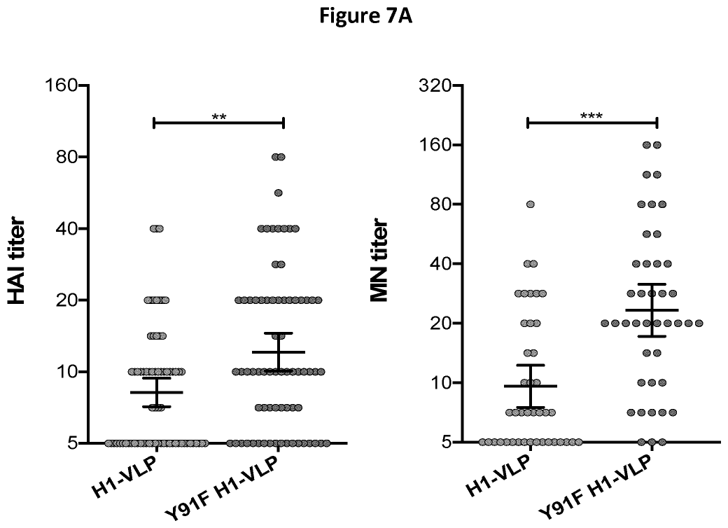

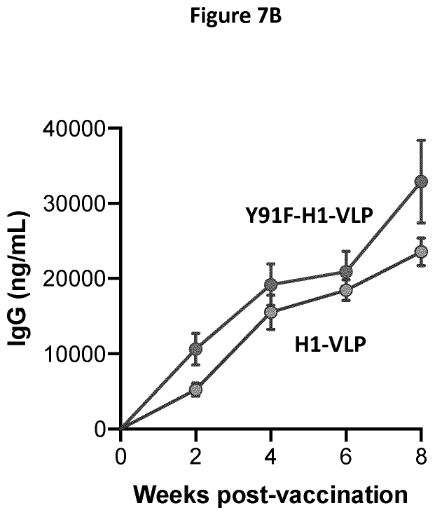

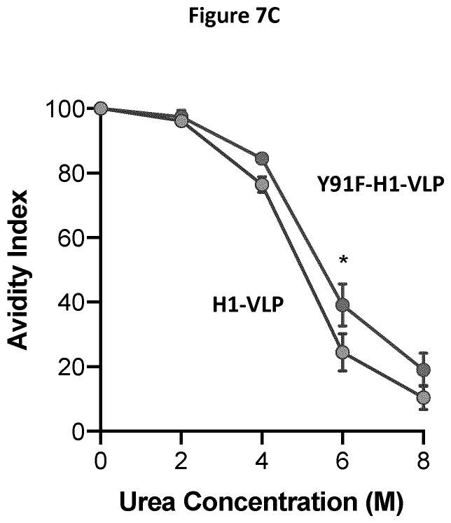

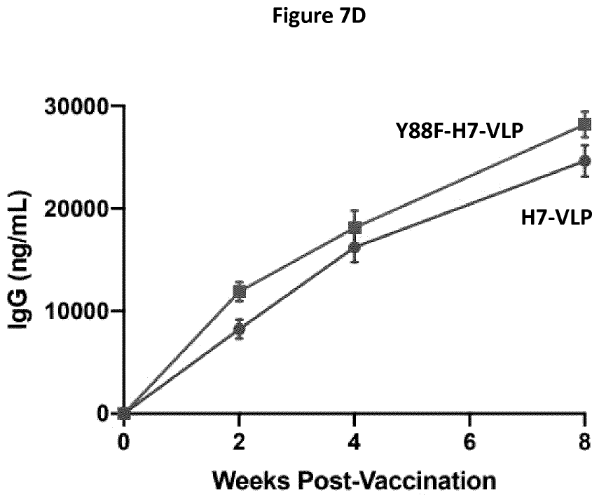

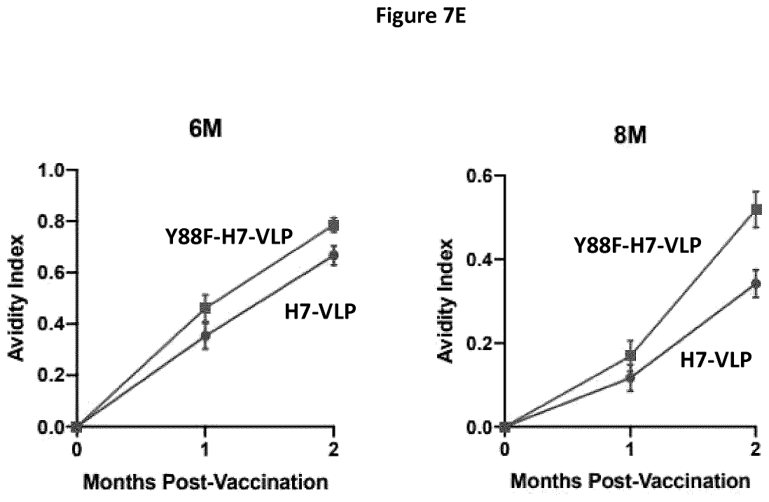

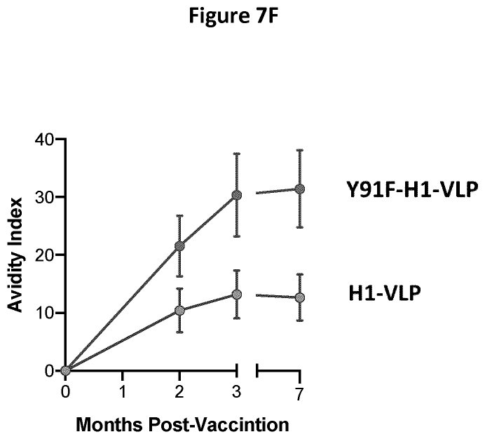

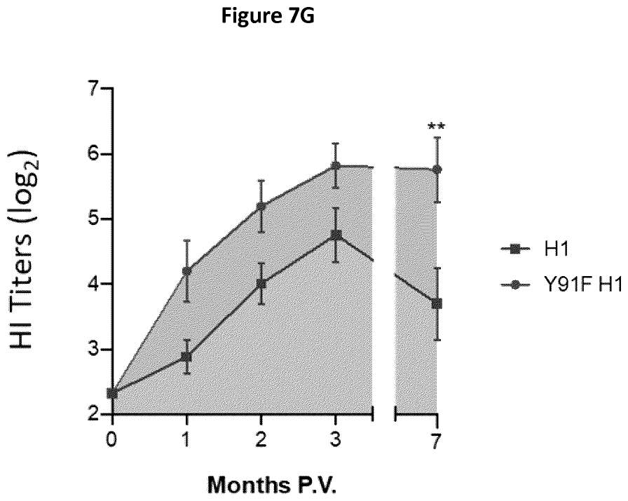

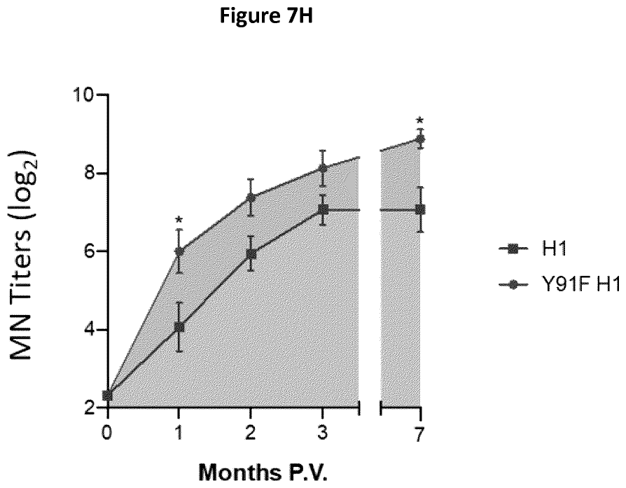

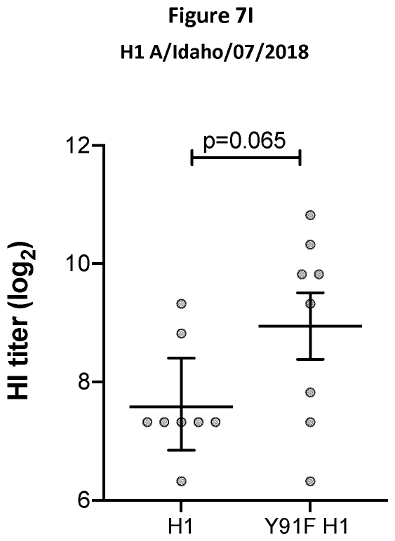

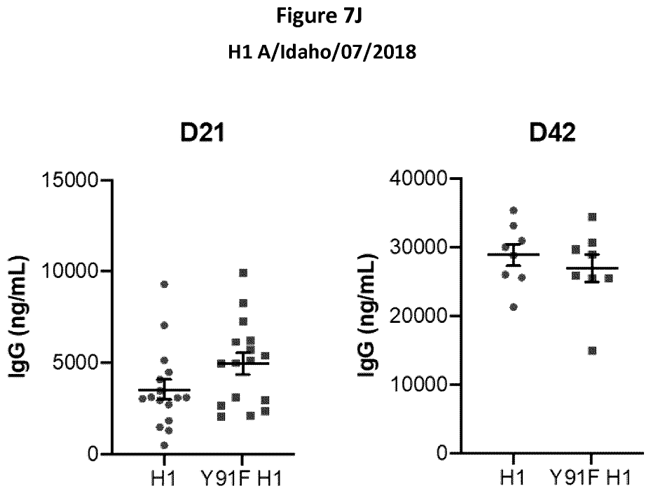

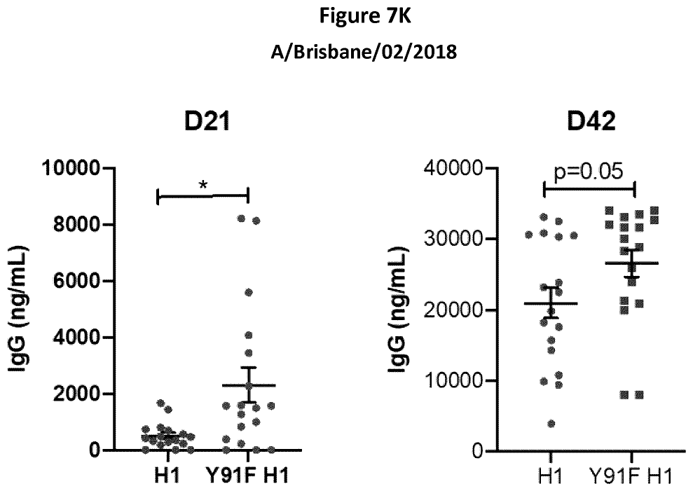

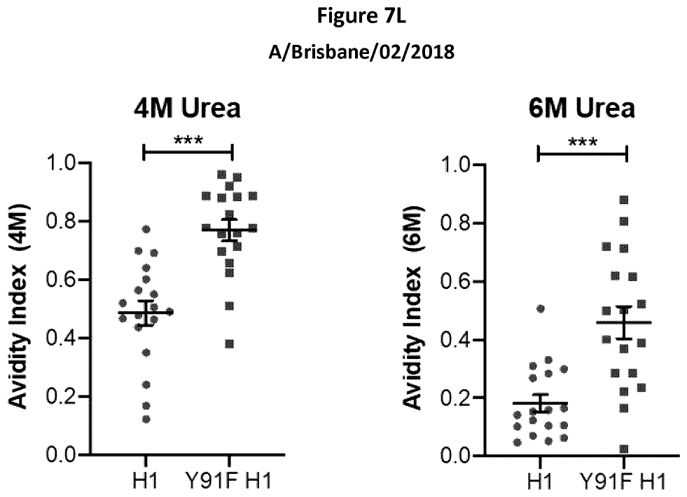

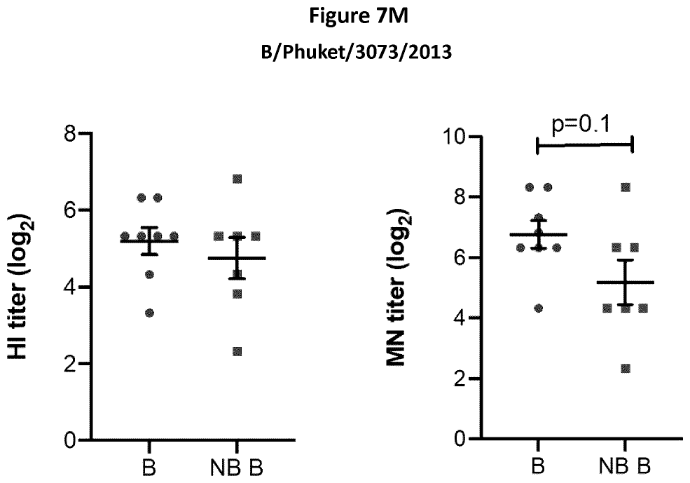

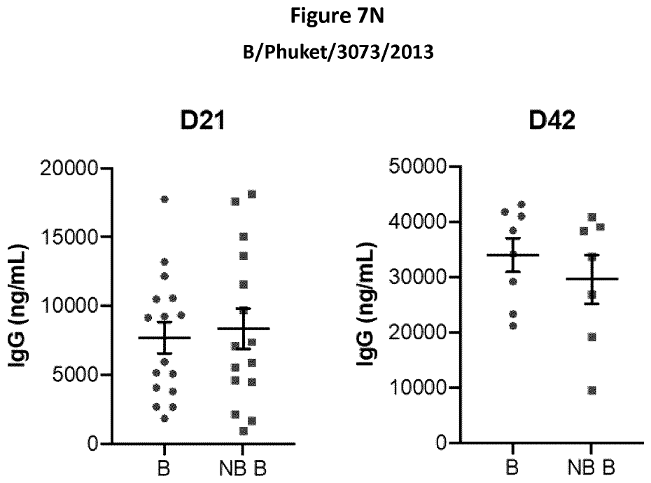

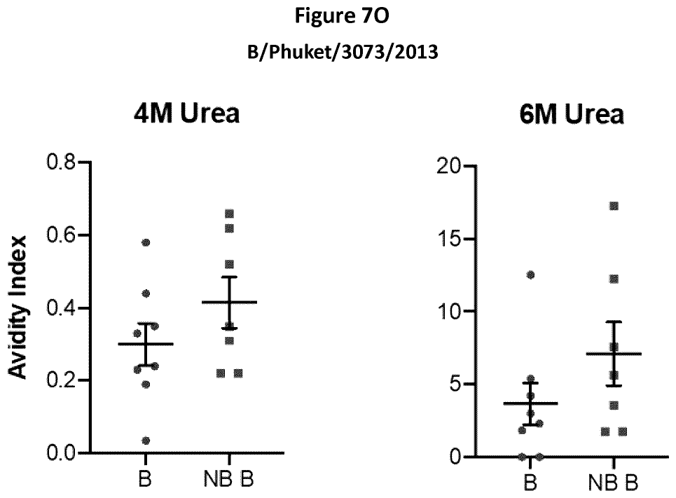

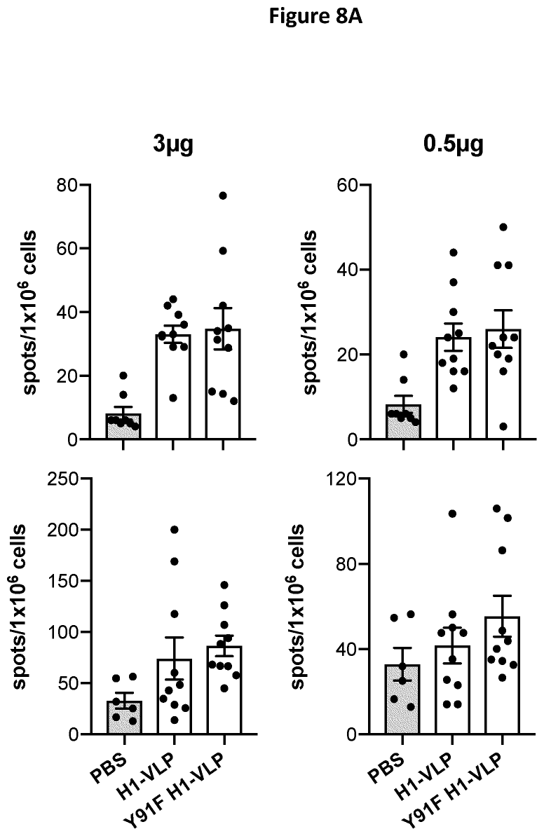

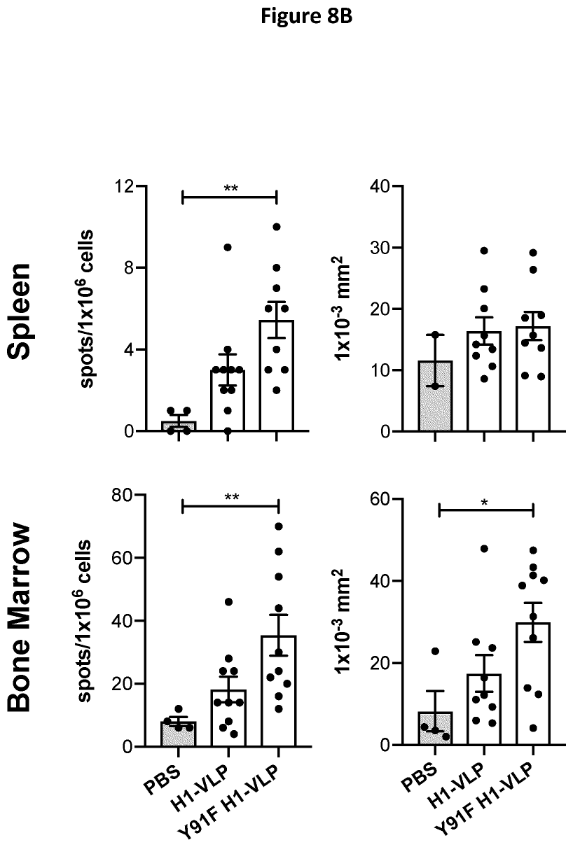

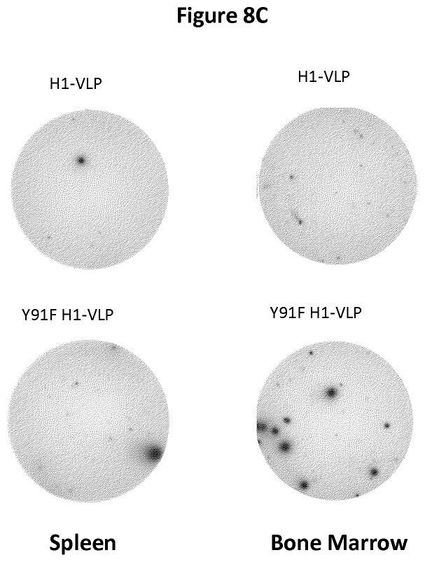

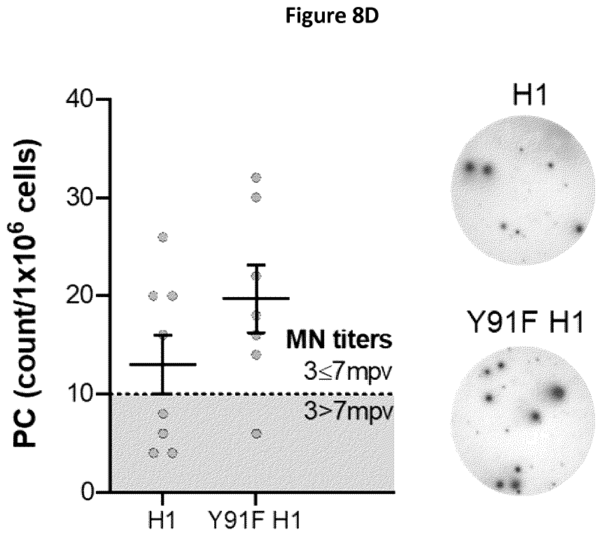

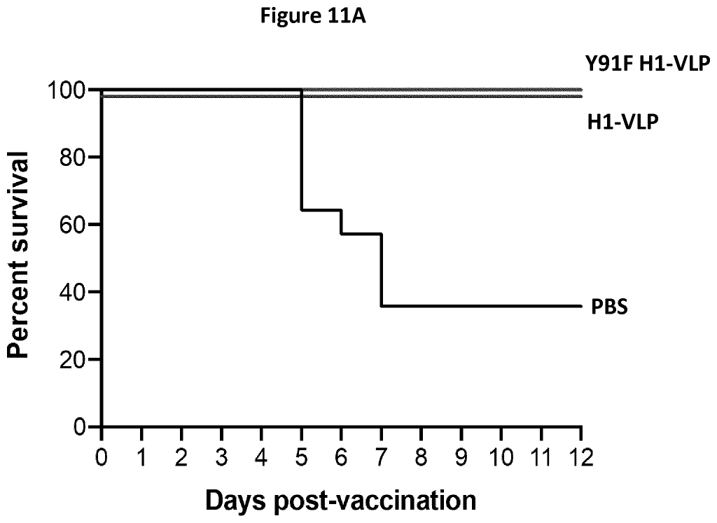

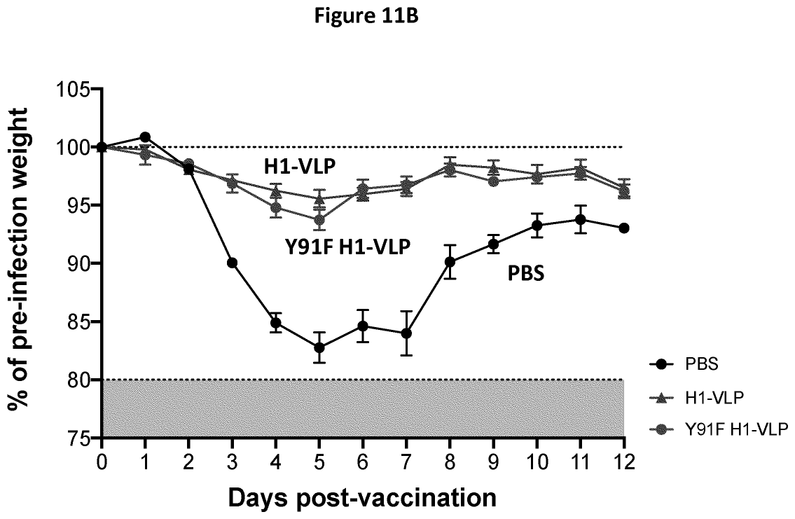

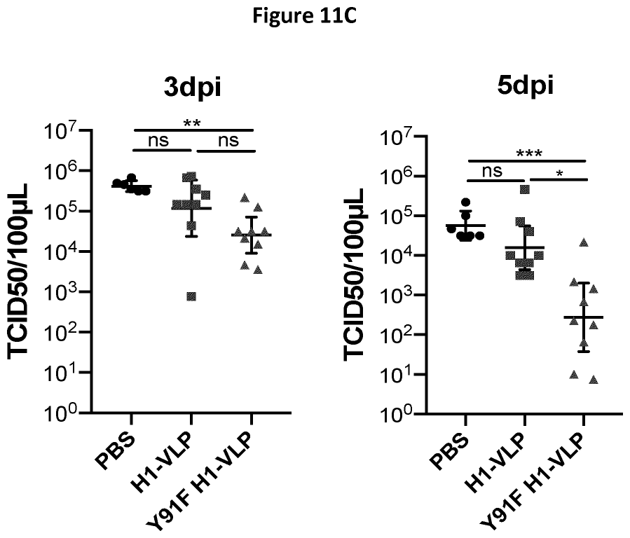

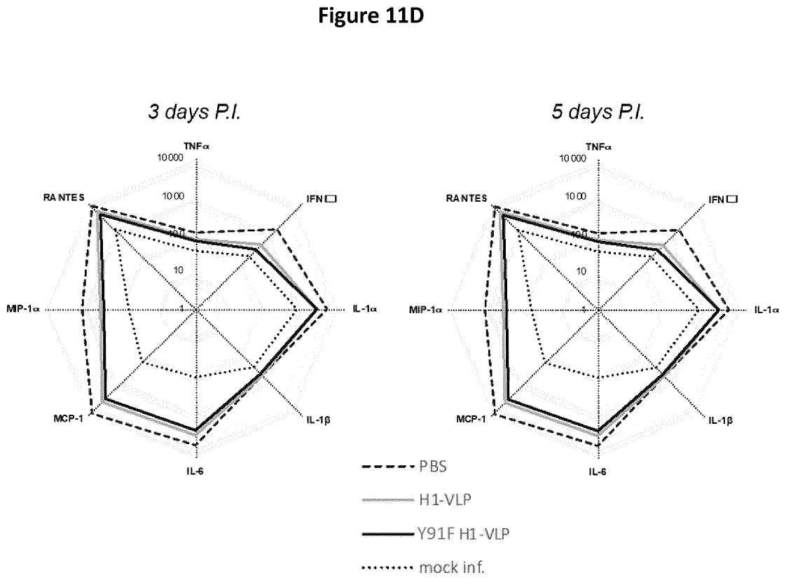

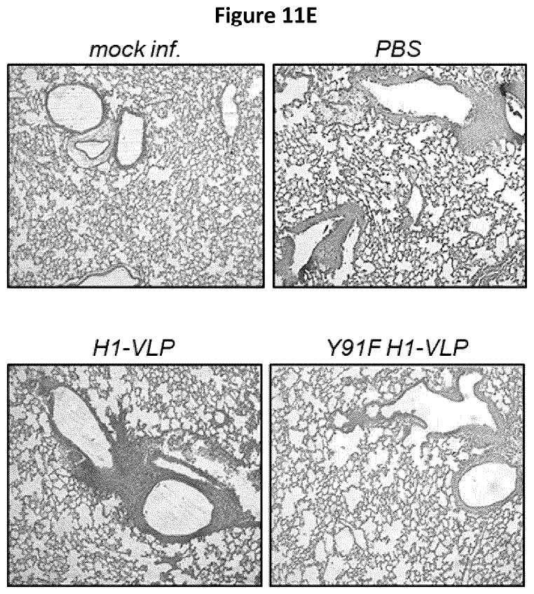

Suprastructures comprising modified HAs, nanoparticles comprising modified HAs, or VLPs comprising modified HA with reduced, non-detectable, or no non-cognate interaction with SA, for example reduced or no SA binding, exhibit improved characteristics when compared to the corresponding suprastructure, nanoparticle, or VLP comprising wildtype HA protein (or unmodified HA protein that exhibits wild type SA binding). For example, use of modified HA protein, suprastructure comprising modified HA, nanoparticle comprising modified HA, or VLP comprising the modified HA protein, as an influenza vaccine exhibited increased immunogenicity and efficacy when compared to the immunogenicity and efficacy of an influenza vaccine comprising the corresponding parent influenza HA, or VLP comprising the parent HA protein. For example, comparison of a binding parent (wild type/non-modified) H1-VLP to a modified (non-binding) H1-VLP (Y91F-H1 HA) in mice demonstrated that the VLP comprising the modified H1 HA elicited higher neutralizing antibody titers (HAI and MN; see FIG. 7A, Example 4.2), higher IgG titers and avidity (FIGS. 7C and 7F; Example 4.2), and an increase in long-lived antibody secreting cells (ASC) in the bone marrow (FIGS. 8A-8C; Example 4.2). There was improved lymphatic germinal center activation following vaccination using a VLP comprising modified HA (Y91F-HA) and viral clearance from the lungs after challenge was significantly enhanced in the animals that received the modified H1-VLP (2 log reduction in lung viral loads; FIG. 11C; Example 4.2). Mice that had received the modified H1-VLP exhibited reduced inflammatory cytokine levels in the lungs including IFN-γ (FIG. 11D). Furthermore, following vaccination using the modified H1 HA, an increase in avidity was observed over a seven-month period compared to the corresponding wild type HA (FIG. 7F) and an increase in HAI titers was observed when sera were collected on a monthly basis to measure HI titers (FIG. 7G) and MN titers (FIG. 7H).

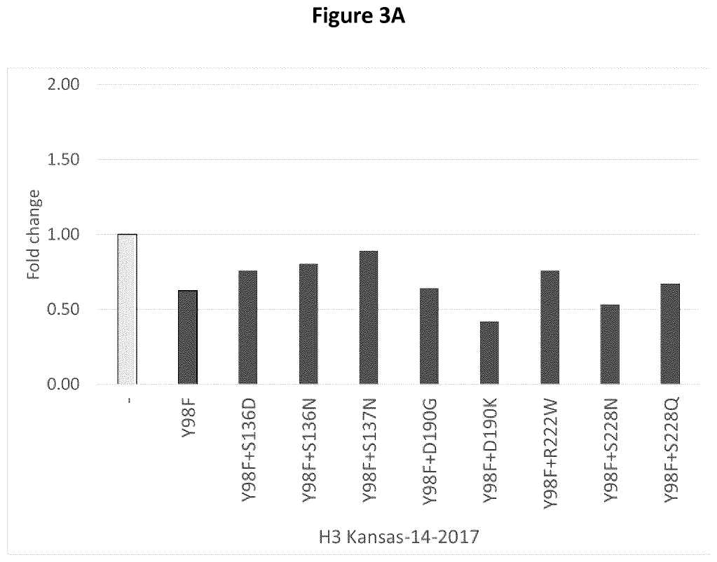

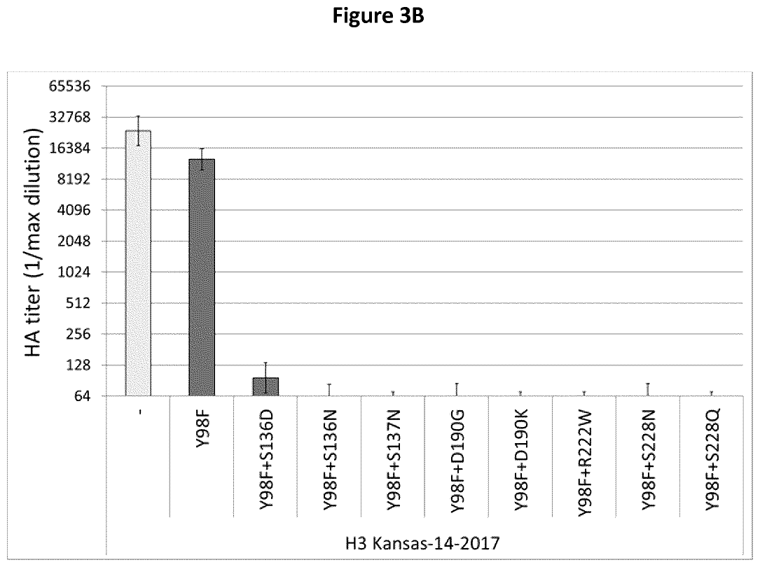

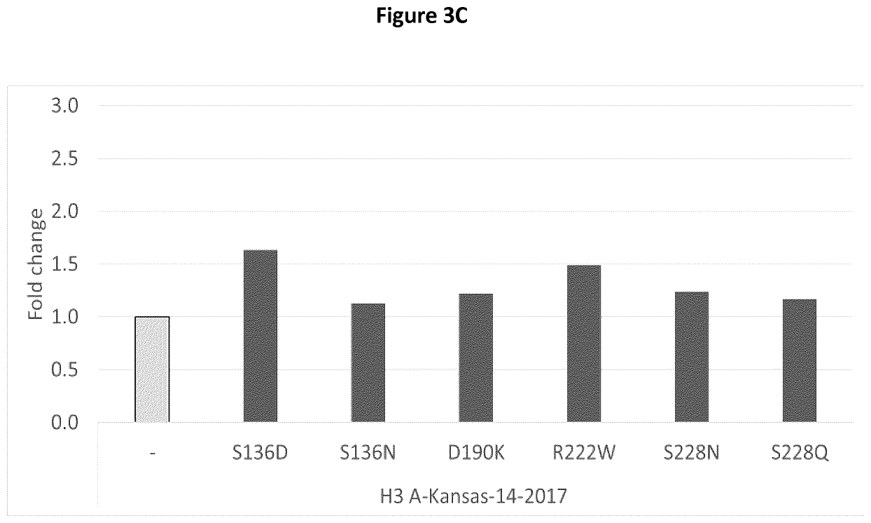

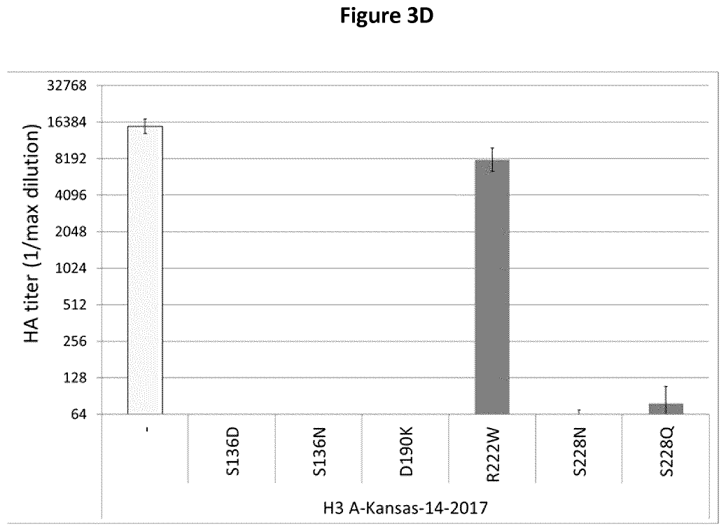

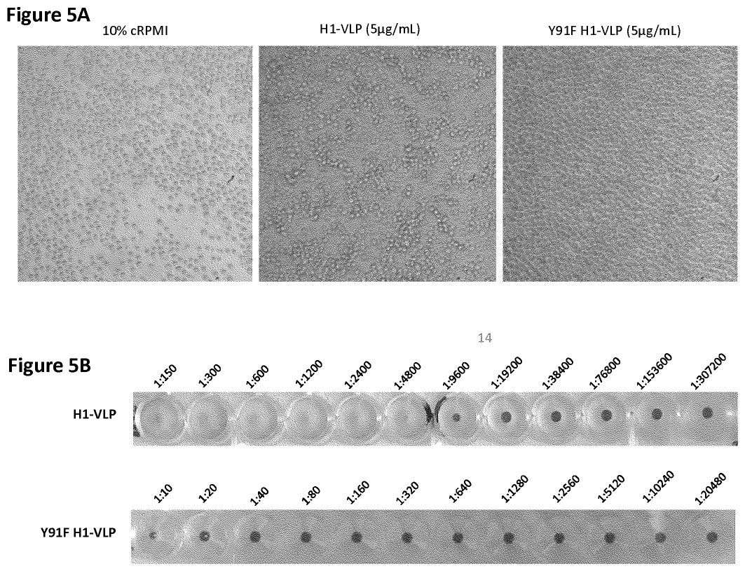

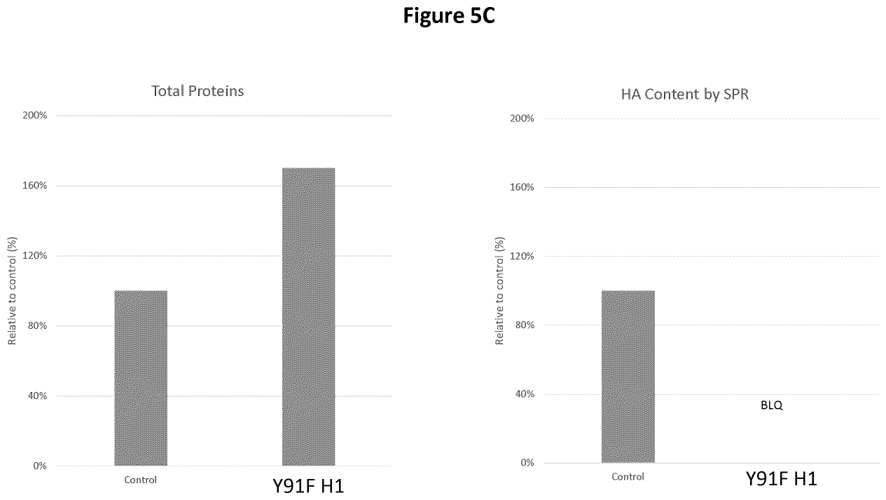

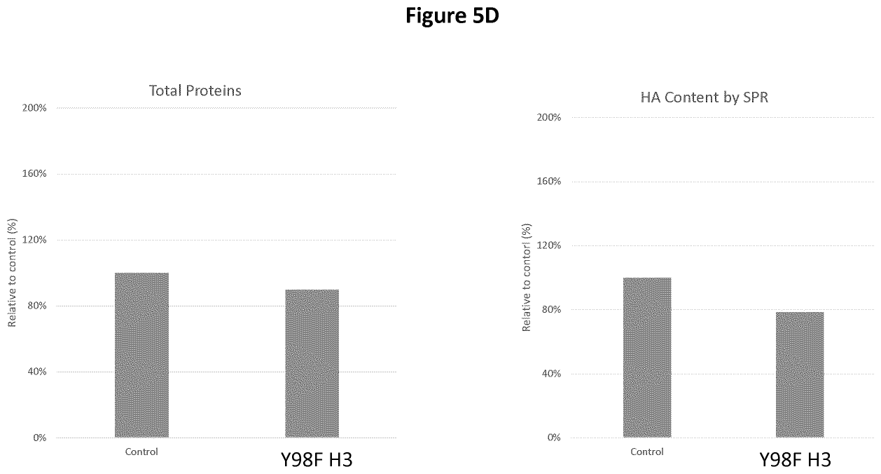

The mutation Y98F is reported to prevent the binding of H3 A/Aichi to SA (Bradley et al., 2011, J. Virol 85:12387-12398). However, the Y98F mutation does not prevent the binding of H3 A/Kansas to SA as significant hemagglutination occurred (FIG. 3B) and H3-SA binding (determined using SPR, FIG. 5D) were observed. As shown in FIG. 3B, additional modifications to H3 HA result in a significant reduction, or non-detectable levels, of hemagglutination. Examples of modifications to H3 HA that reduce H3 HA binding to SA, include Y98F in combination with any of S136D, S136N, S137N, D190G, D190K, R222W, S228N, S228Q.

Vaccination with Y88F H7-VLP resulted in an increase in IgG compared to parent H7-VLP-vaccinated mice, up to 8 weeks post vaccination (FIG. 7D). Additionally, an increase in avidity of Y88F H7 HA was observed over a 2 month period post vaccination, when compared to the parent H7-VLP (FIG. 7E, Example 4.2).

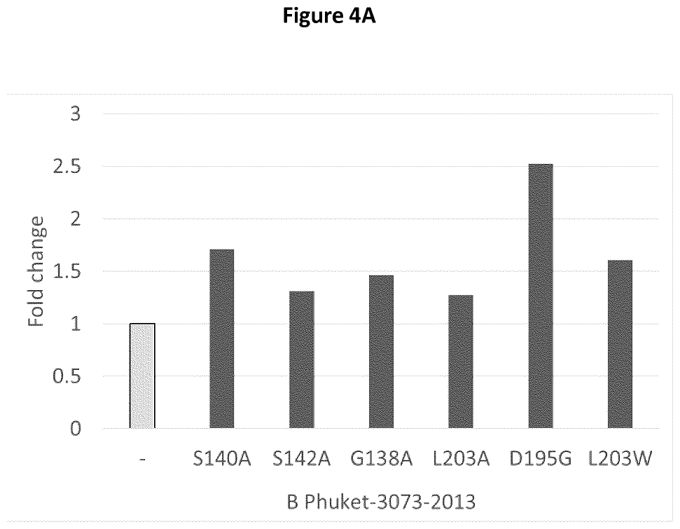

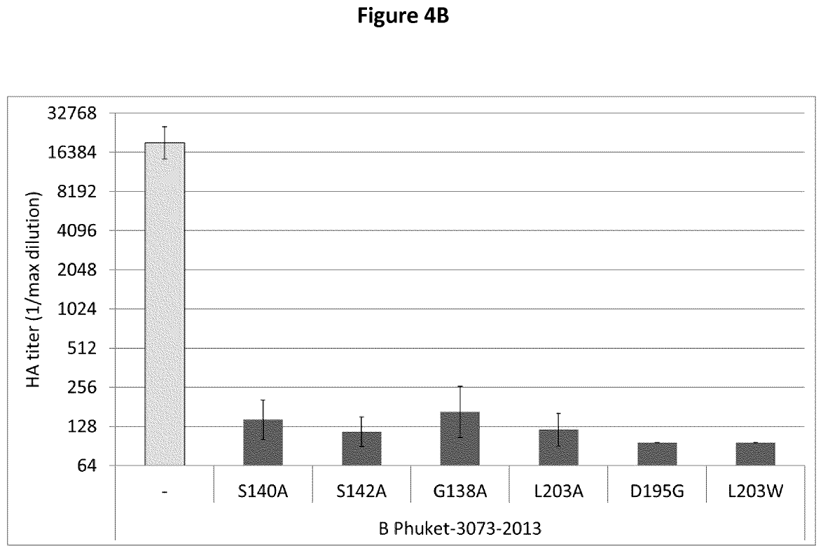

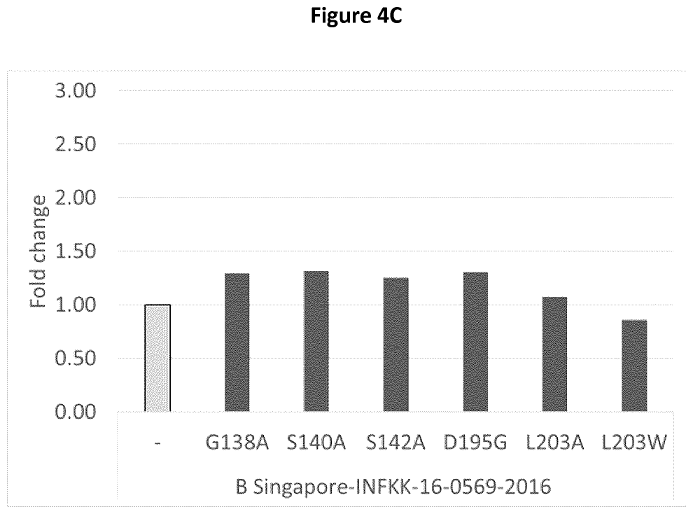

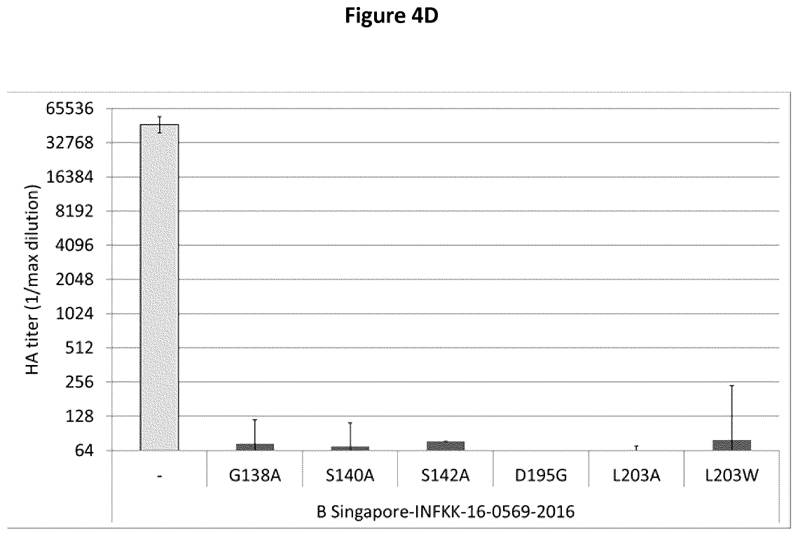

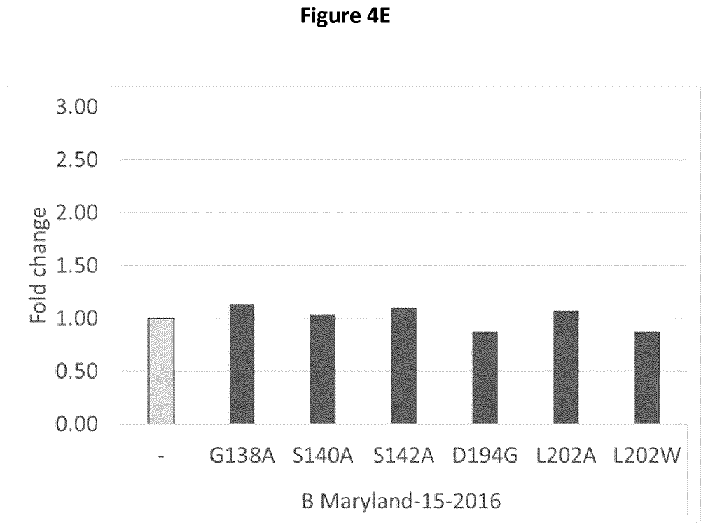

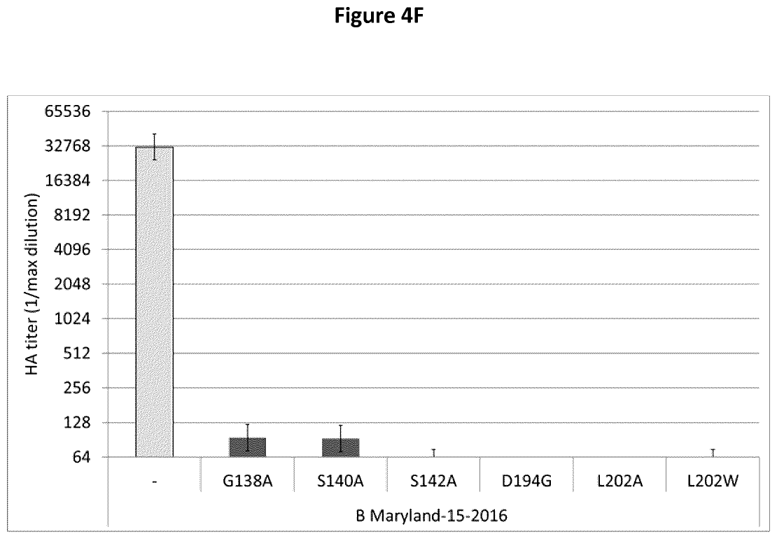

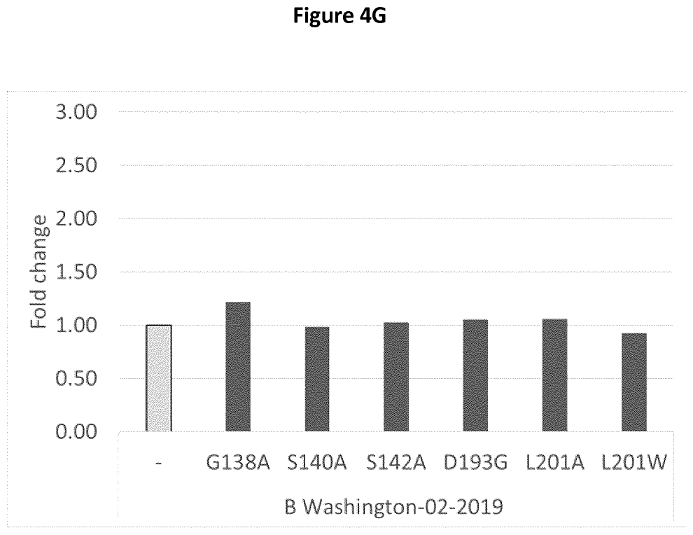

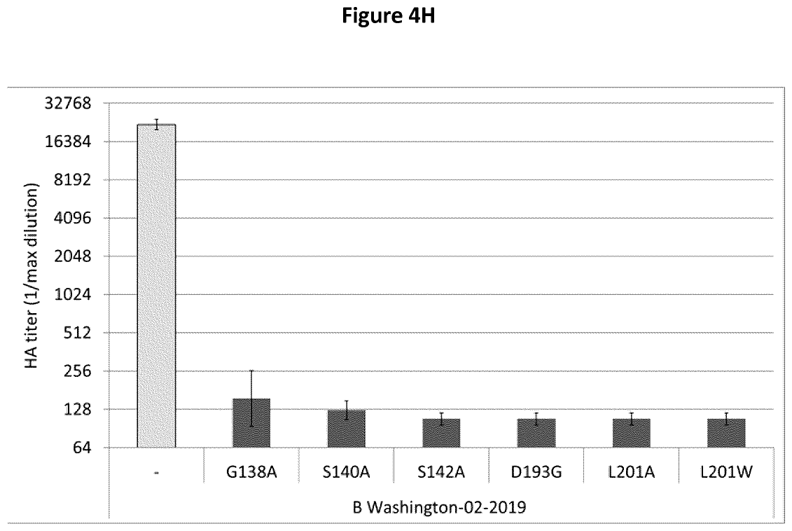

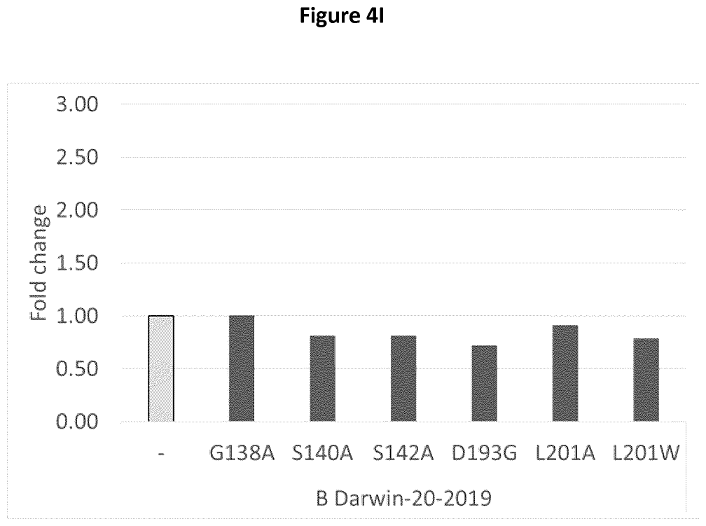

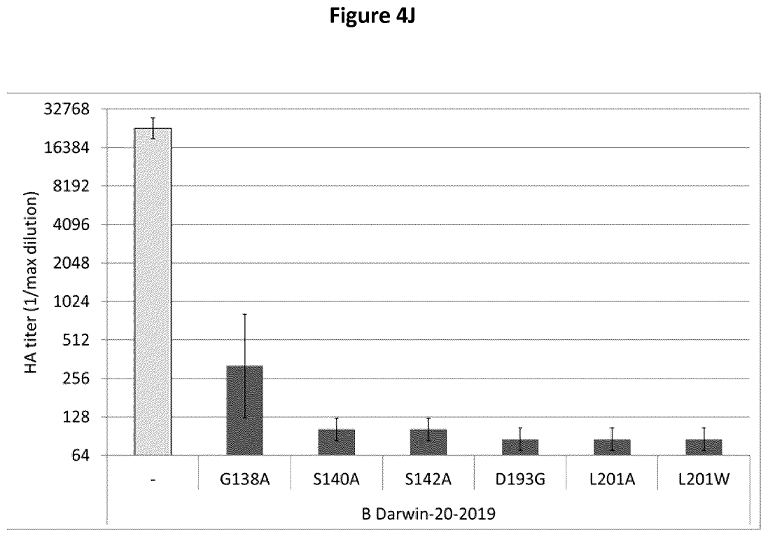

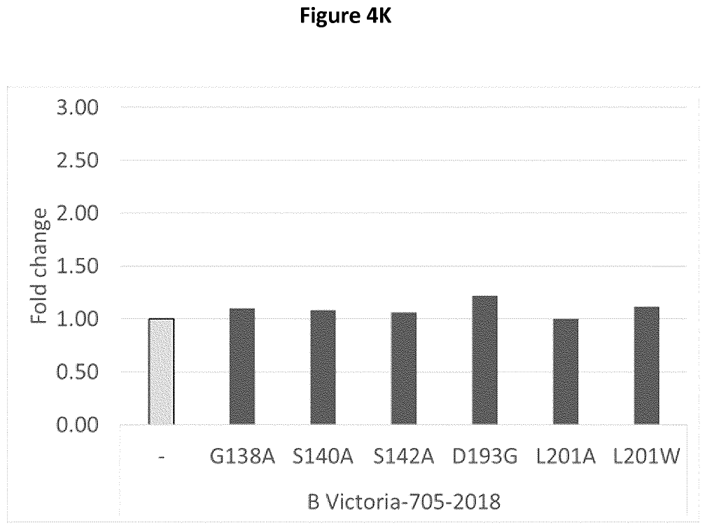

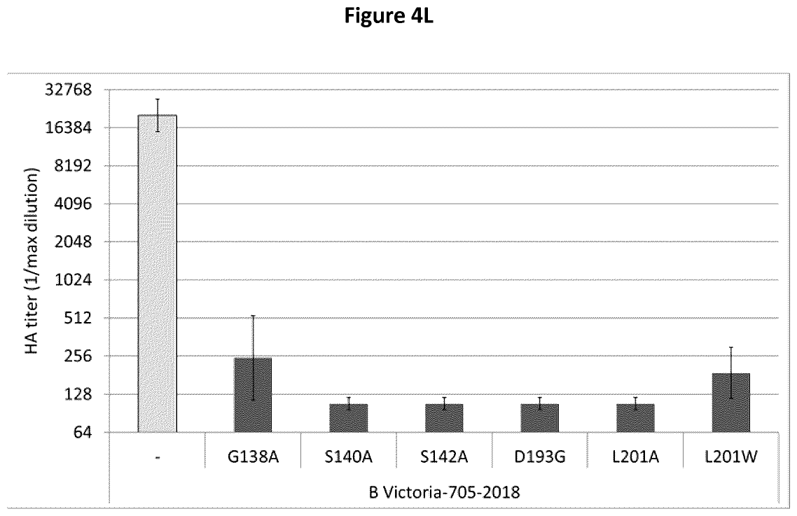

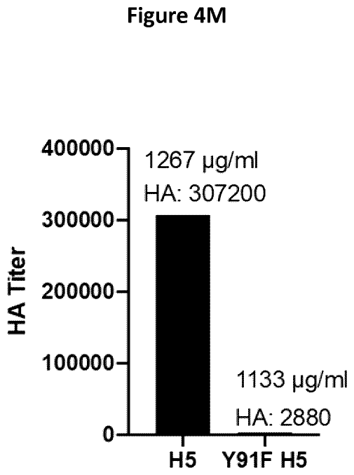

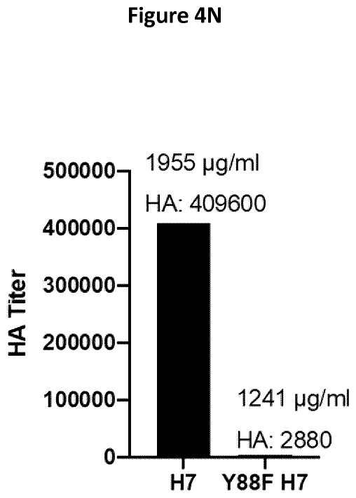

Furthermore, modified B-HA comprising a substitution selected from the group: S140A, S142A, G138A, D195G, L203W and L203A was observed to reduce binding between B HA and SA as these modified B HAs resulted in a significant reduction of HA titer (FIGS. 4B, 4D, 4F, 4H, 4J, 4L) when compared with the HA titer of the parent B HA. In addition, modified B-HA comprising a substitution selected from the group: S140A, S142A, G138A, D195G, L202A and L203W resulted in near equal or greater VLP yield (FIGS. 4C, 4E, 4G, 4I, 4K). Modified B-HA comprising a substitution selected from the group: S140A, S142A, G138A, D195G, L203W and L203A also resulted in decreased hemagglutination activity (FIG. 4D).

The modified HA protein as described herein comprises one or more than one alteration, mutation, modification, or substitution in its amino acid sequence at any one or more amino acid that correspond with amino acids of the parent HA from which the modified HA is derived. By “correspond to an amino acid” or “corresponding to an amino acid”, it is meant that an amino acid corresponds to an amino acid in a sequence alignment with an influenza reference strain, or reference amino acid sequence, as described below (see for example Table 1). Two or more nucleotide sequences, or corresponding polypeptide sequences of HA may be aligned to determine a “consensus” or “consensus sequence” of a subtype HA sequence as is known in the art.

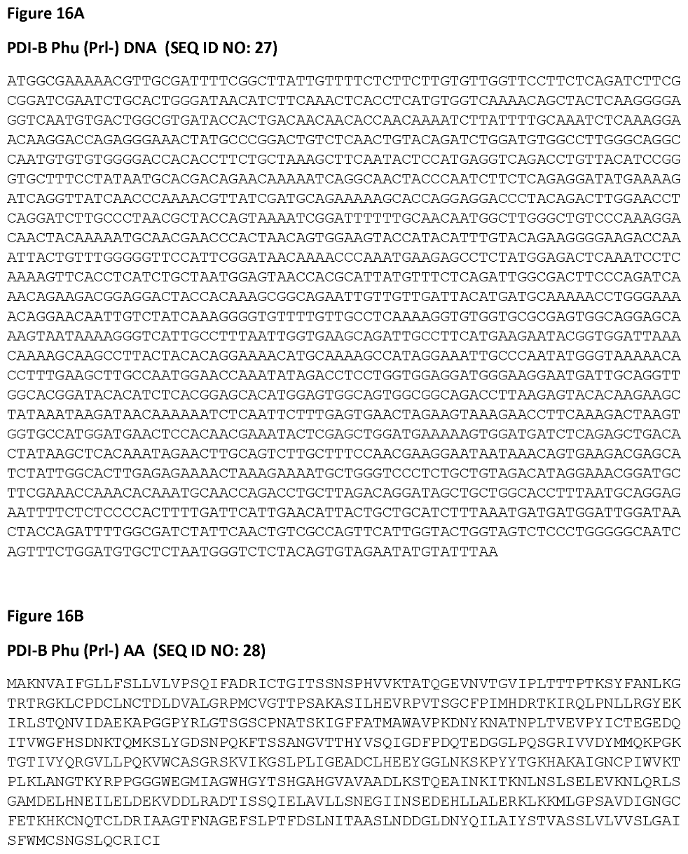

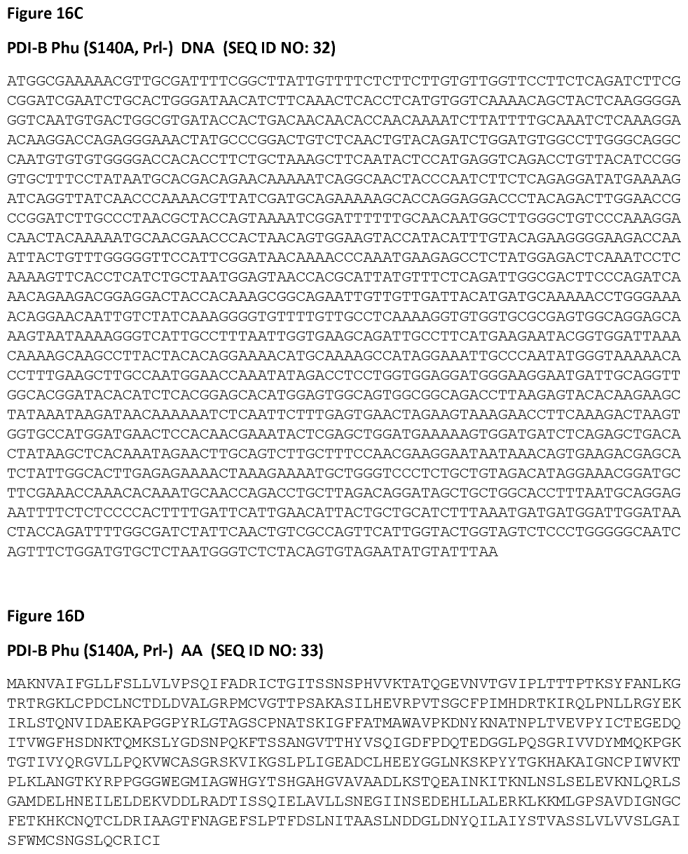

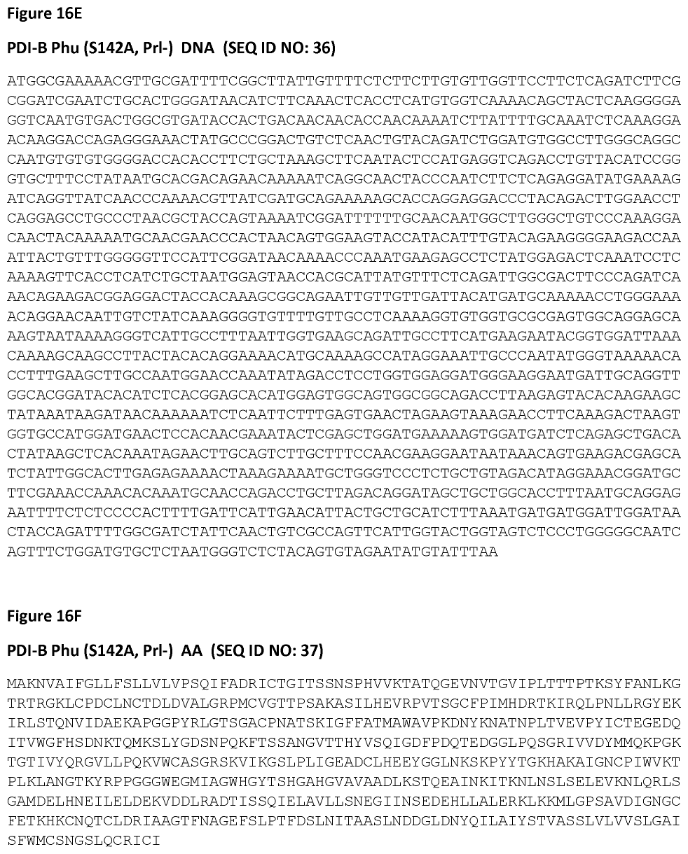

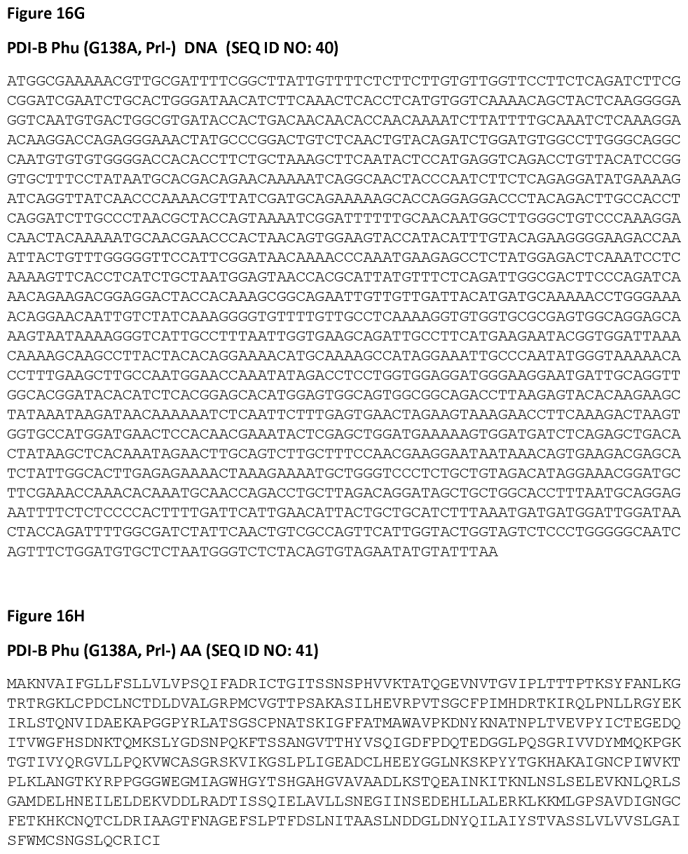

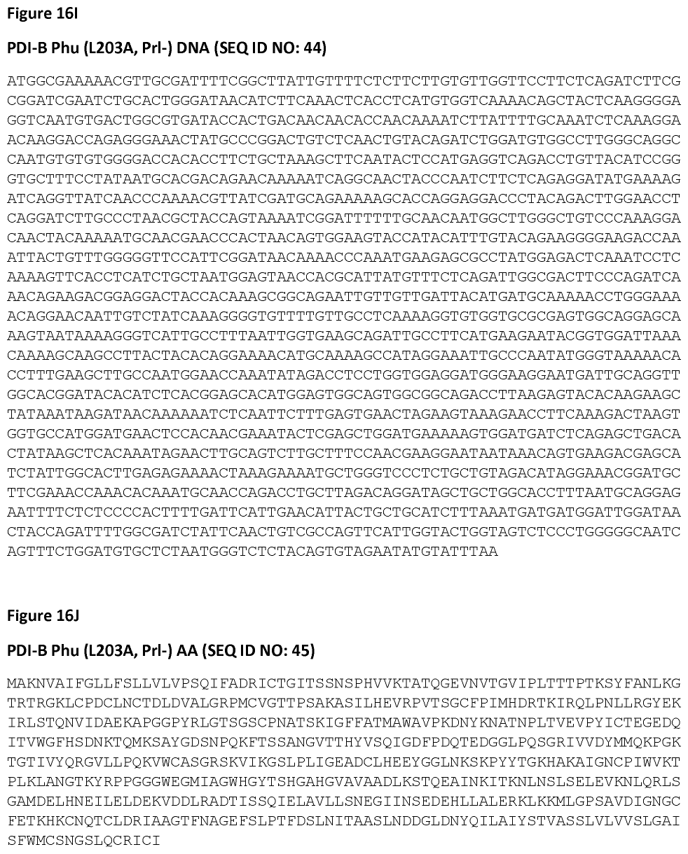

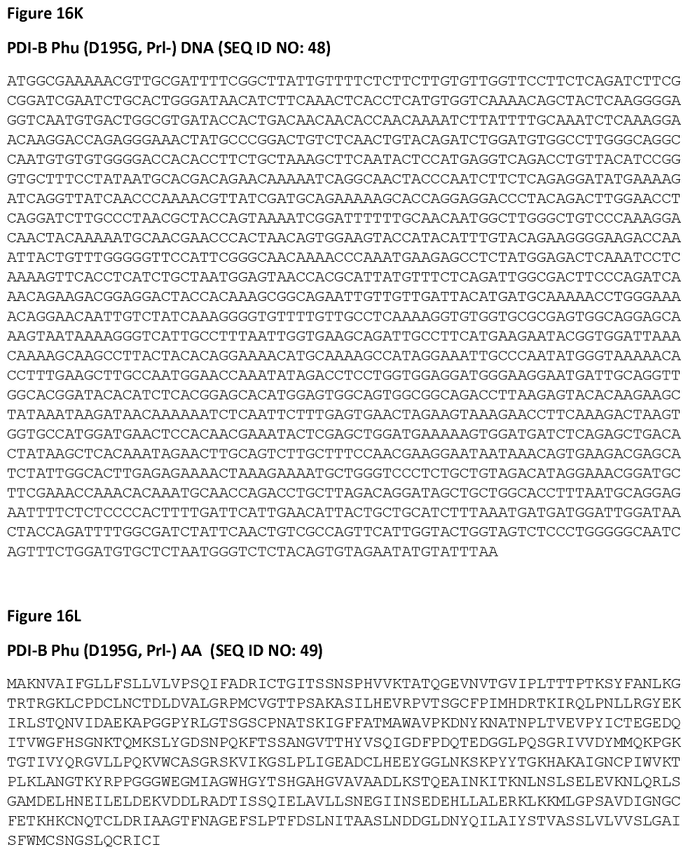

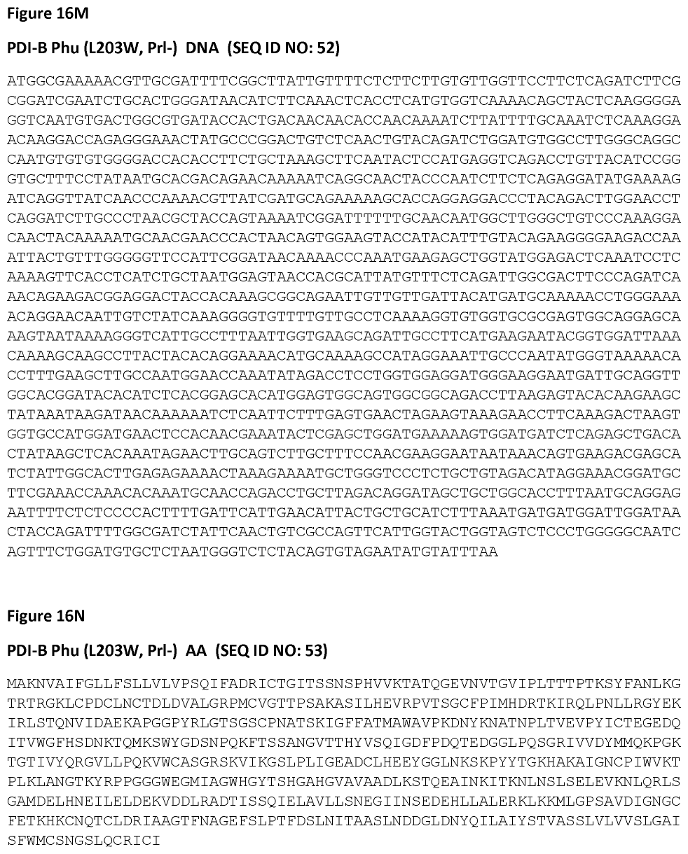

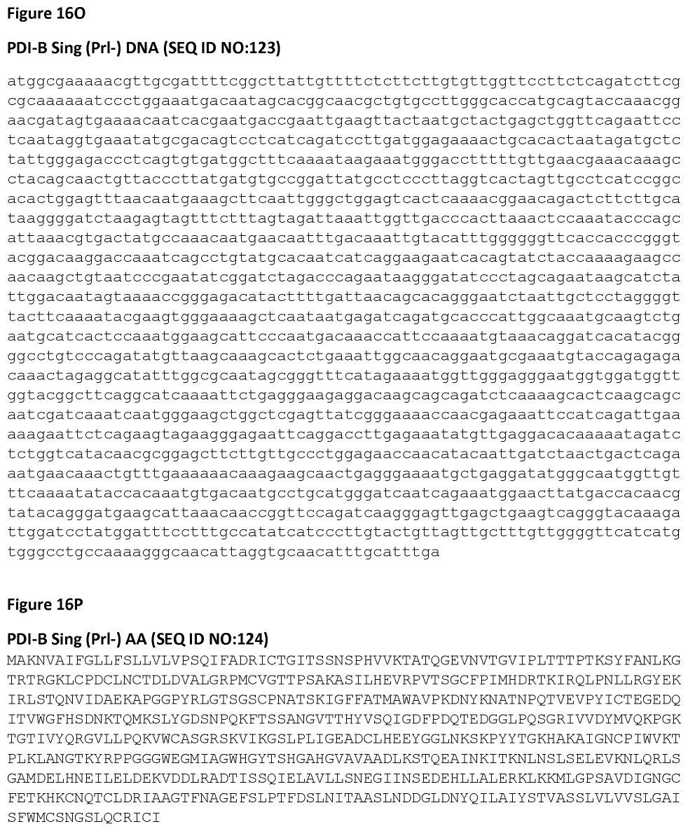

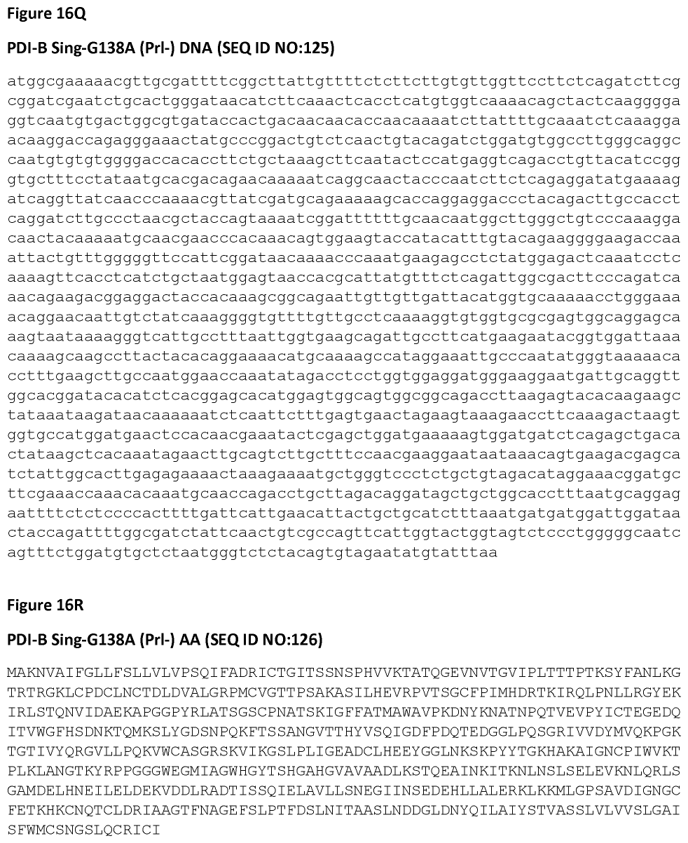

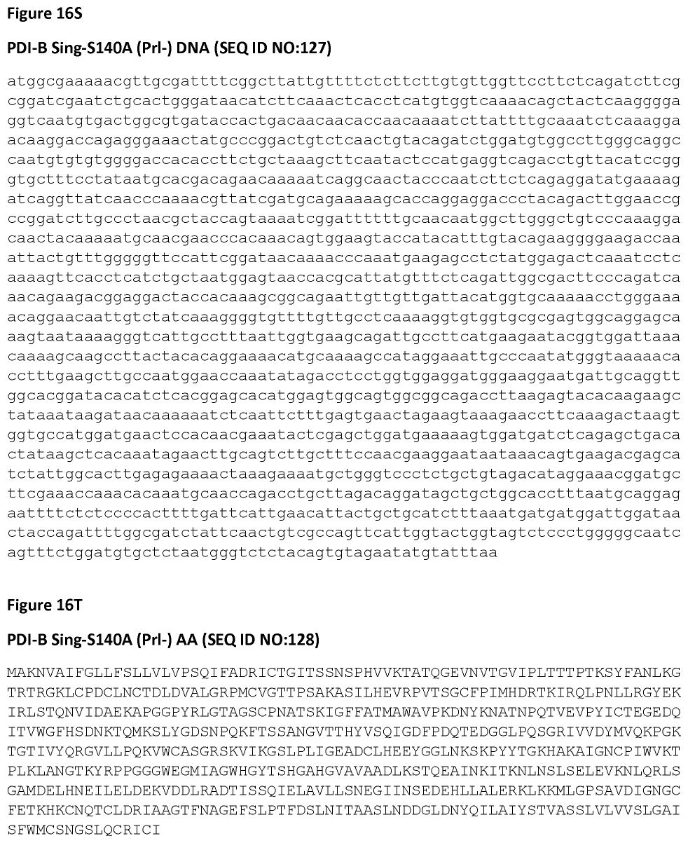

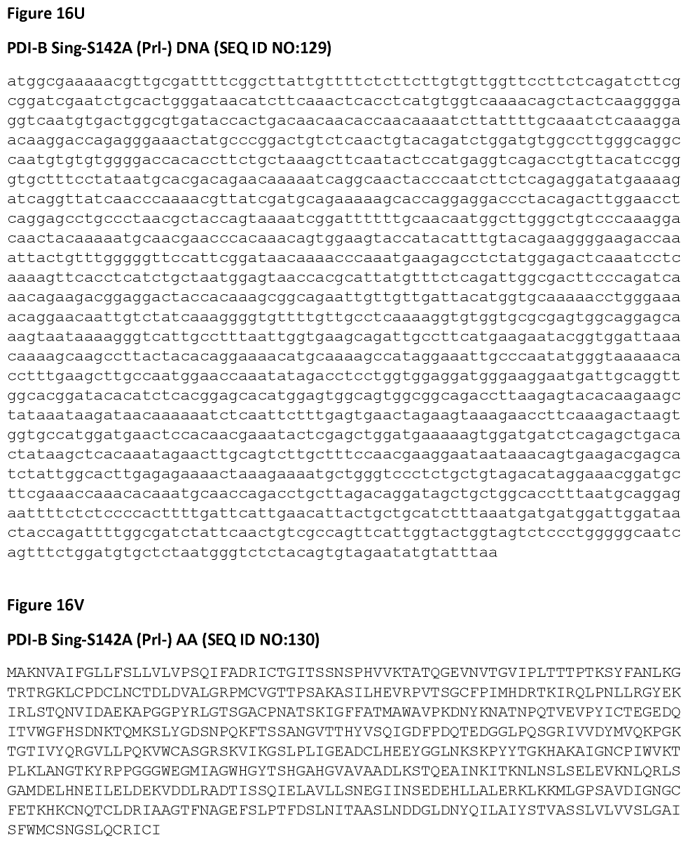

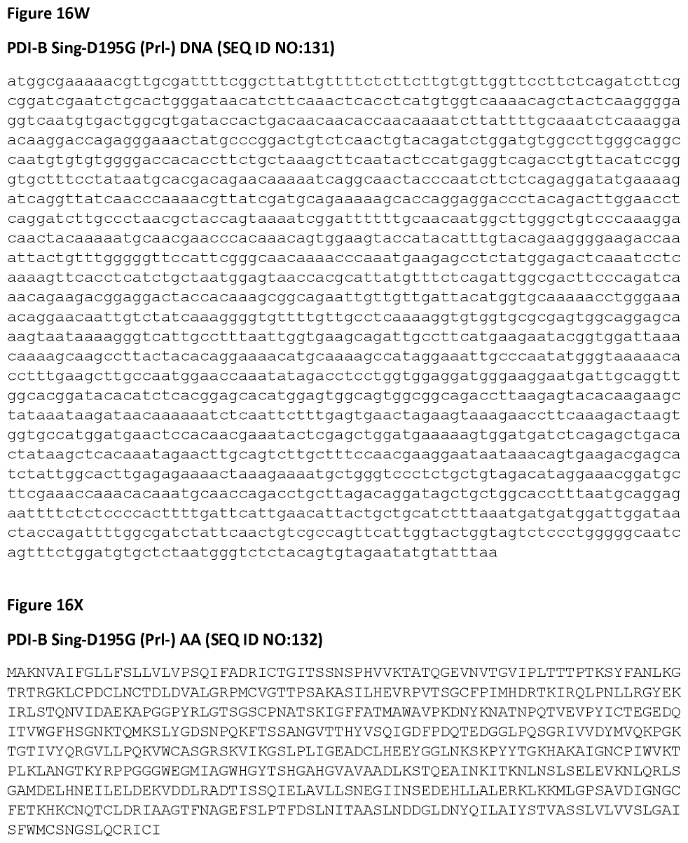

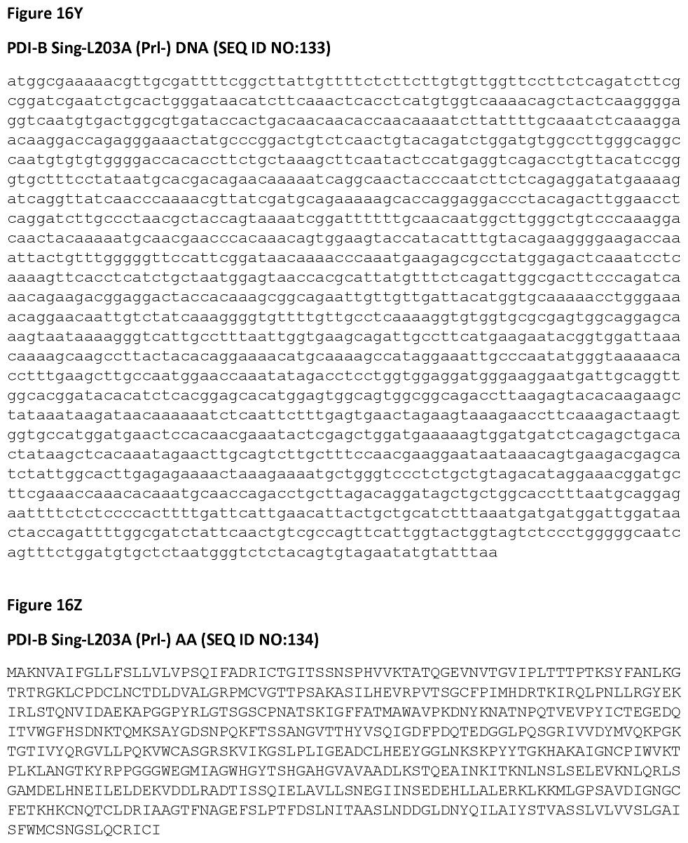

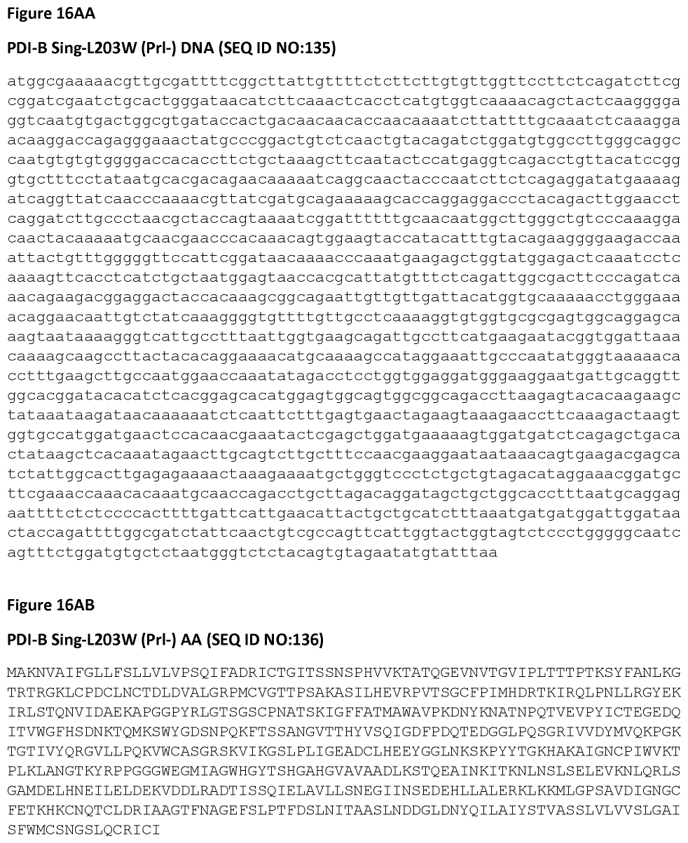

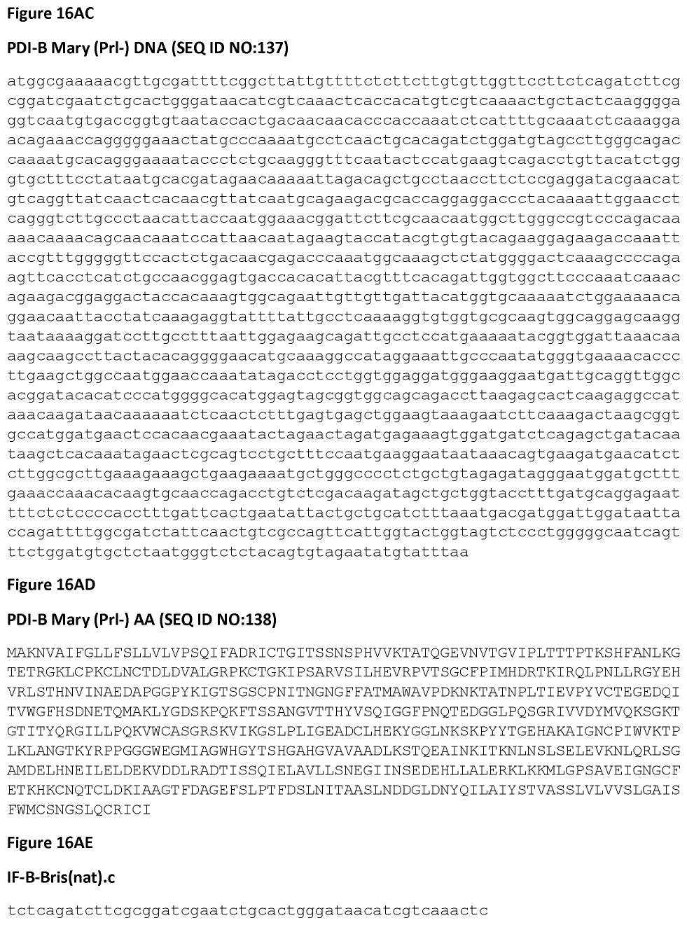

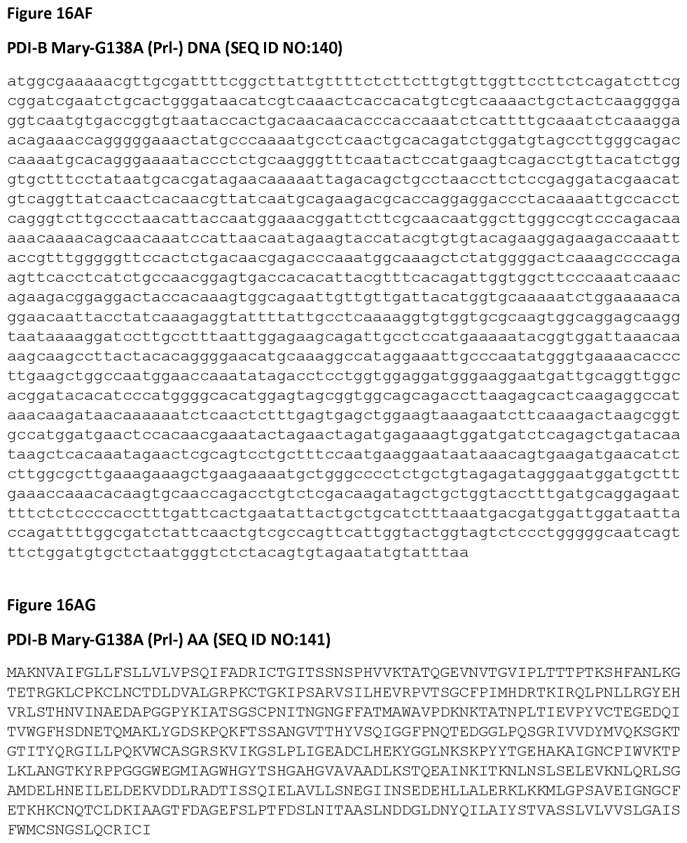

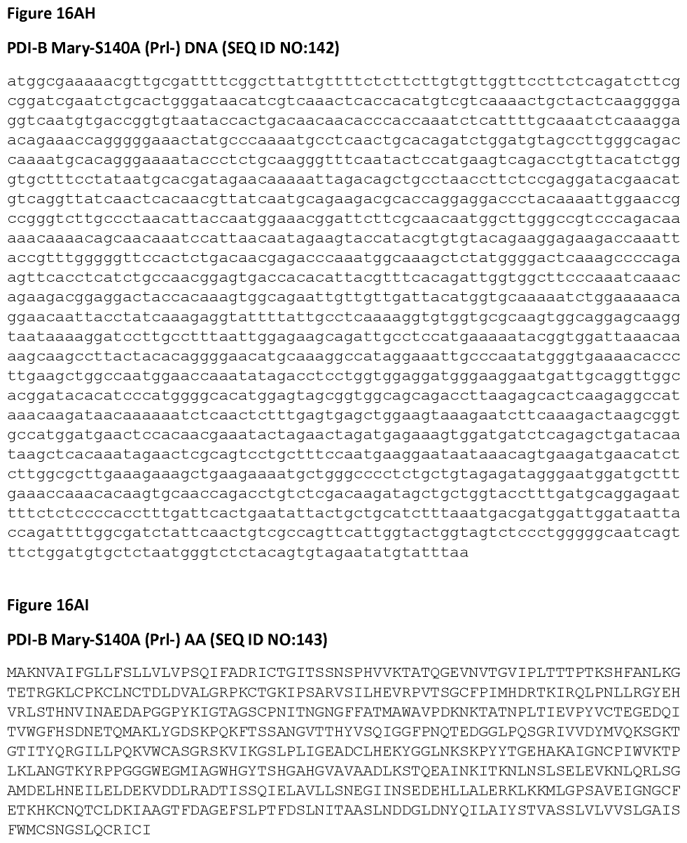

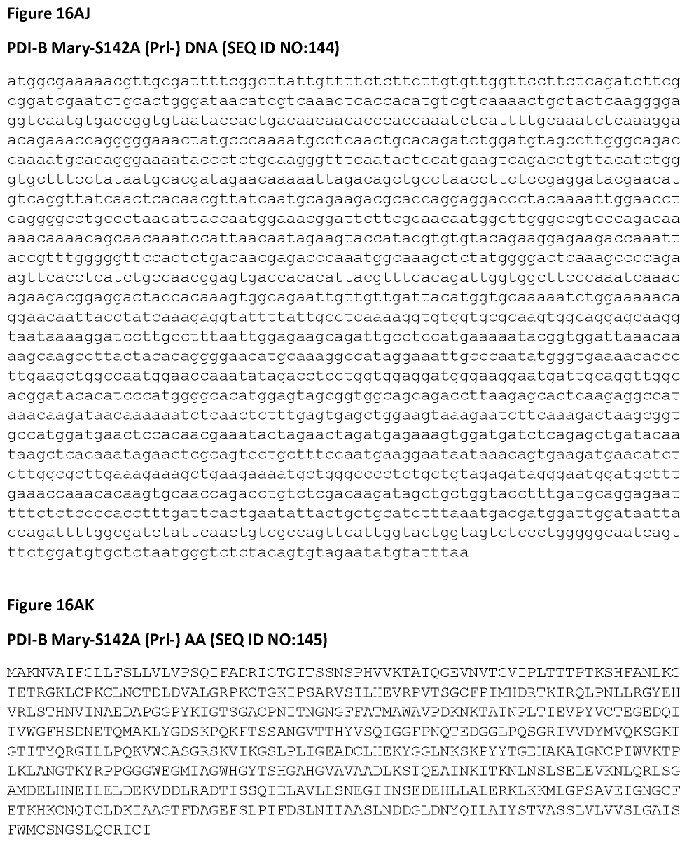

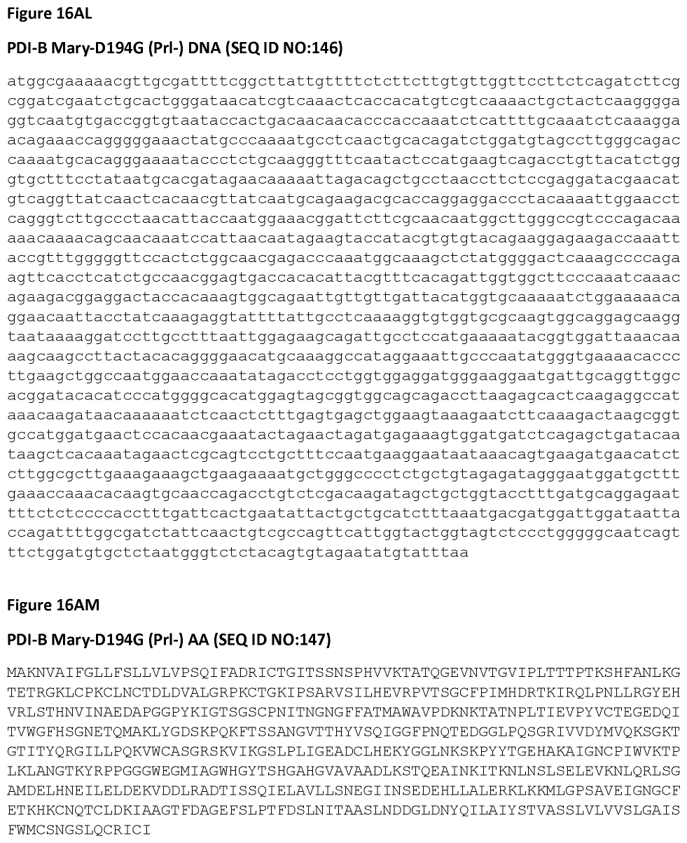

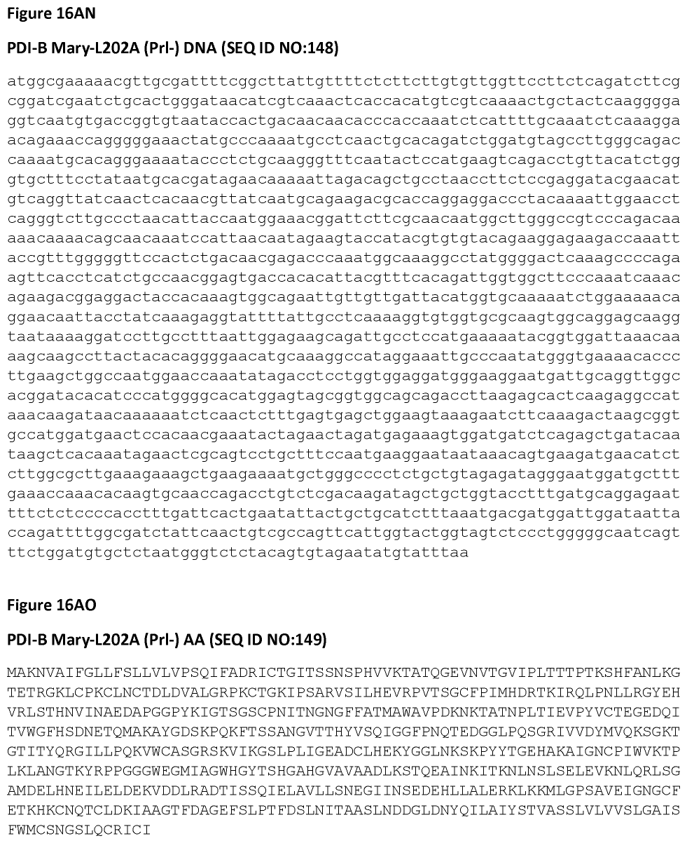

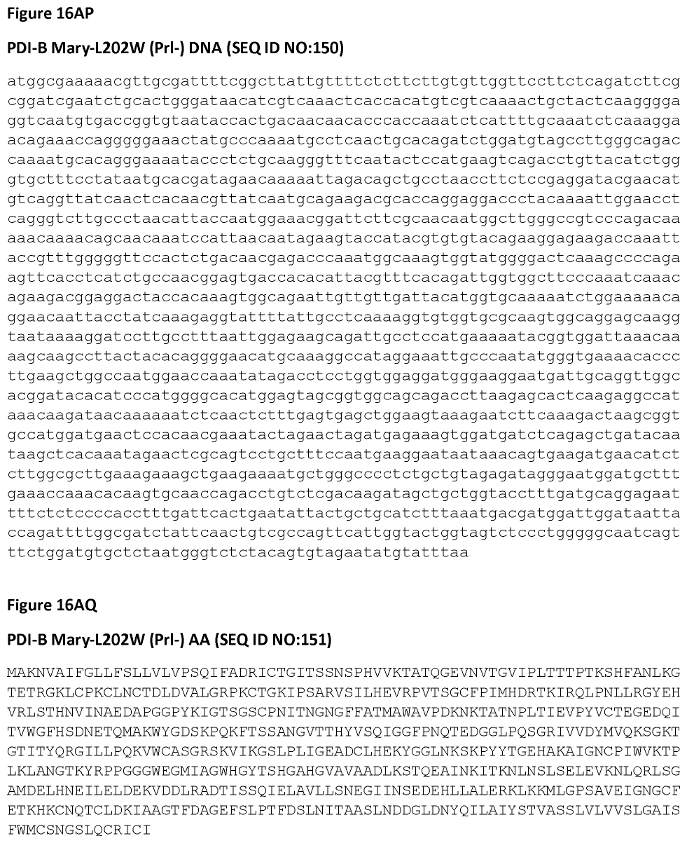

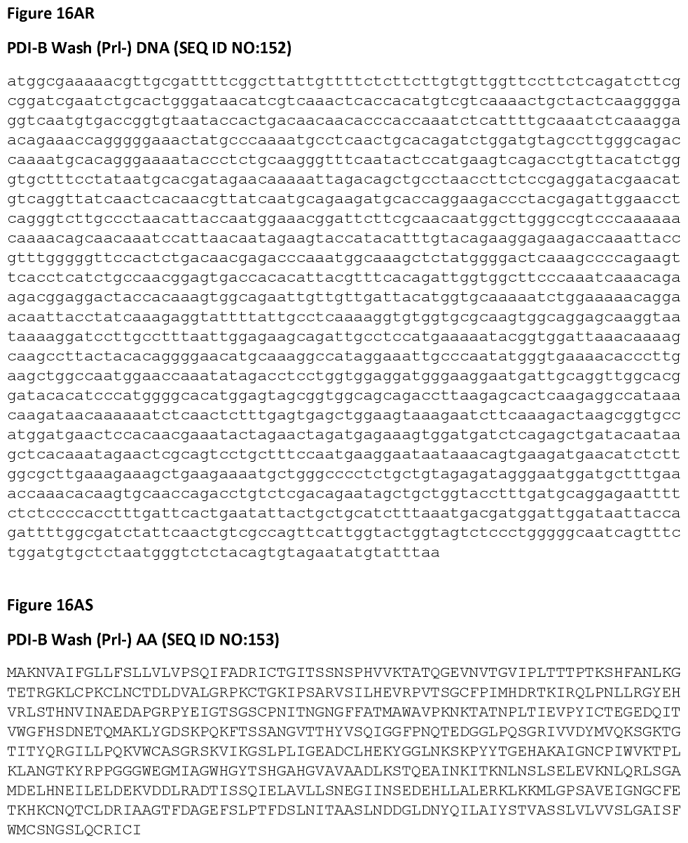

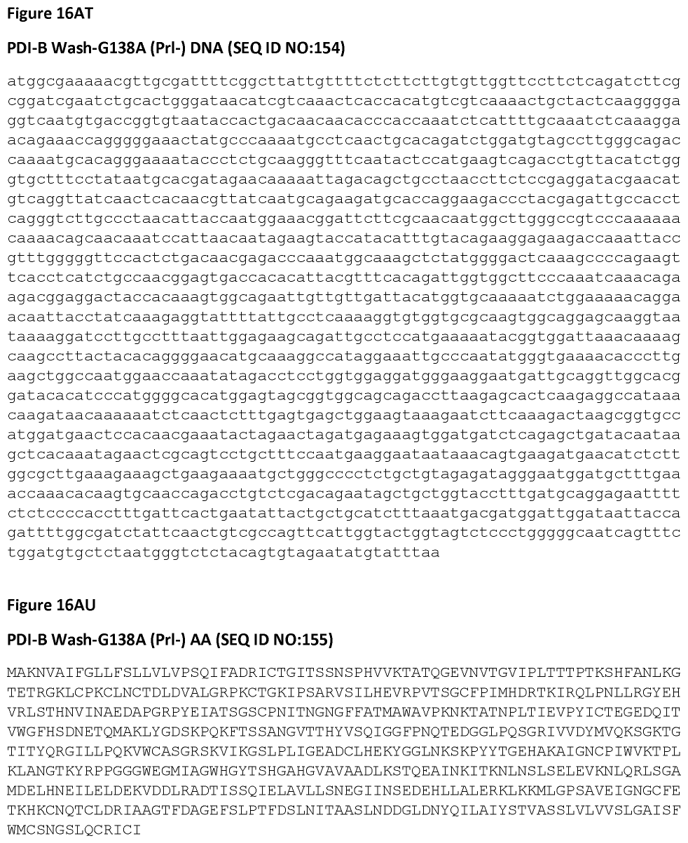

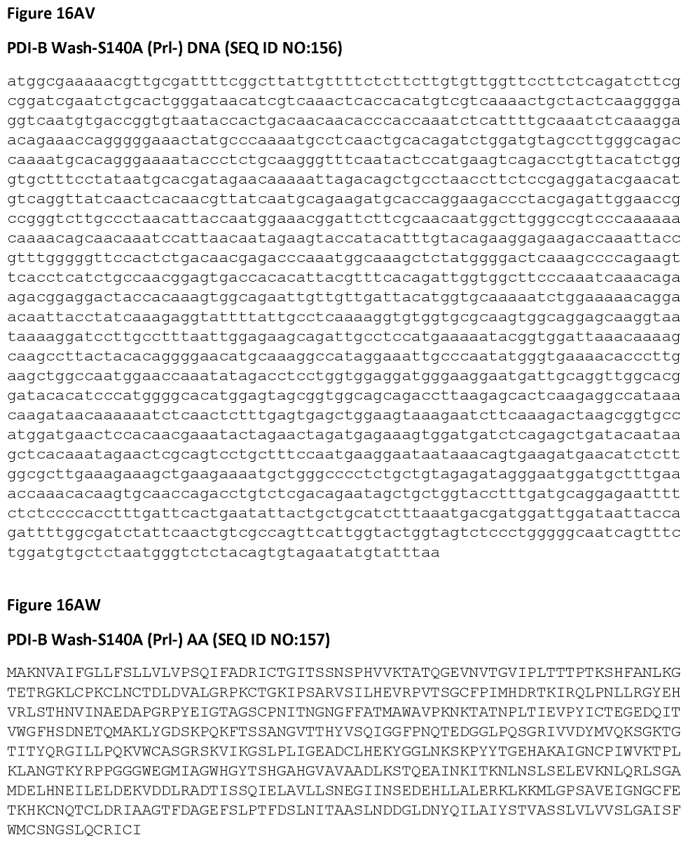

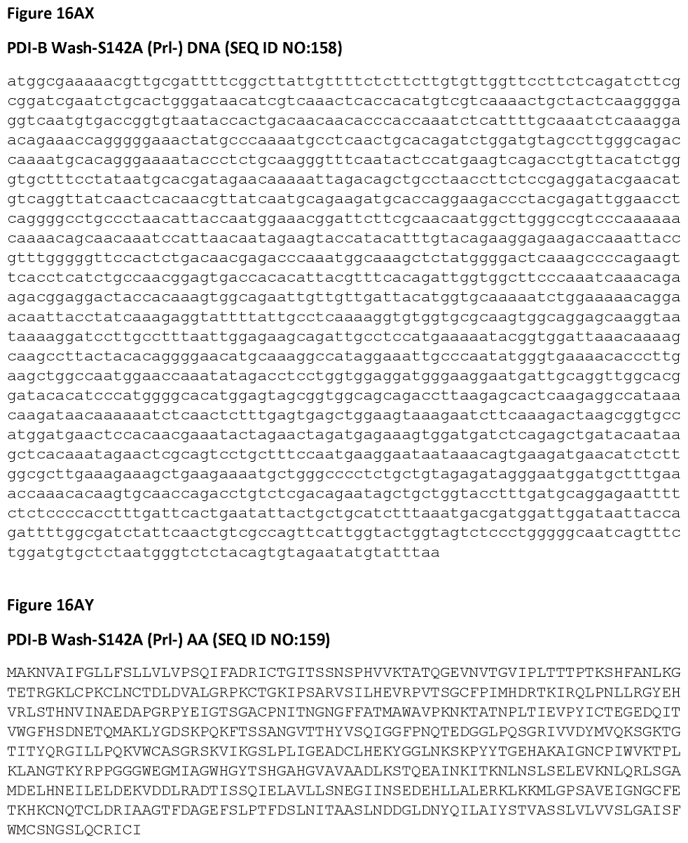

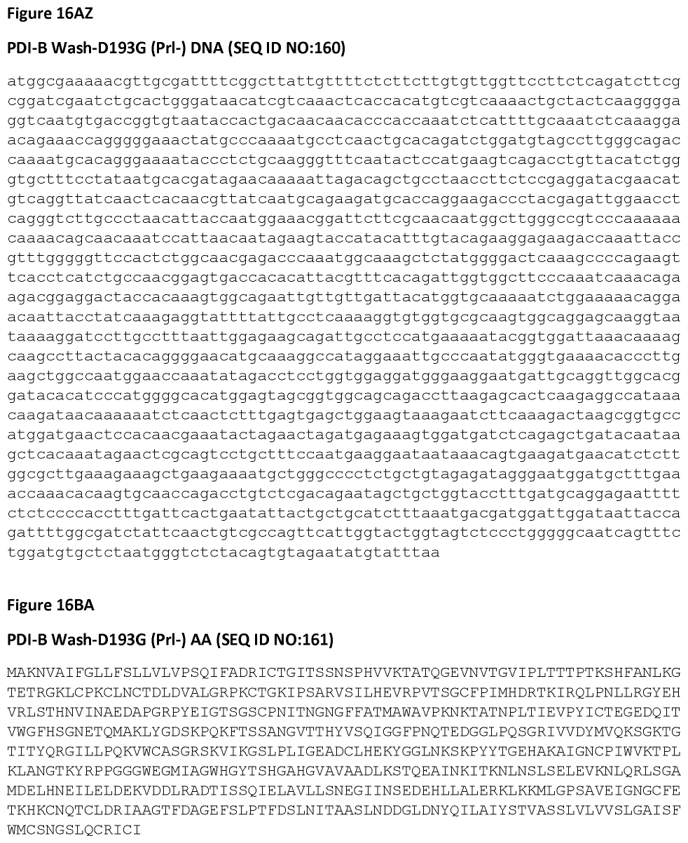

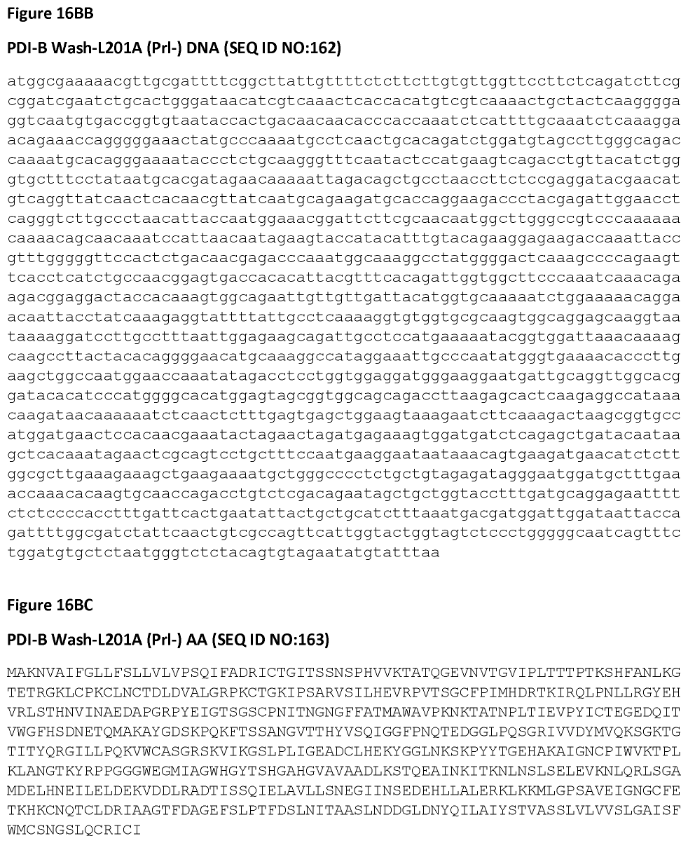

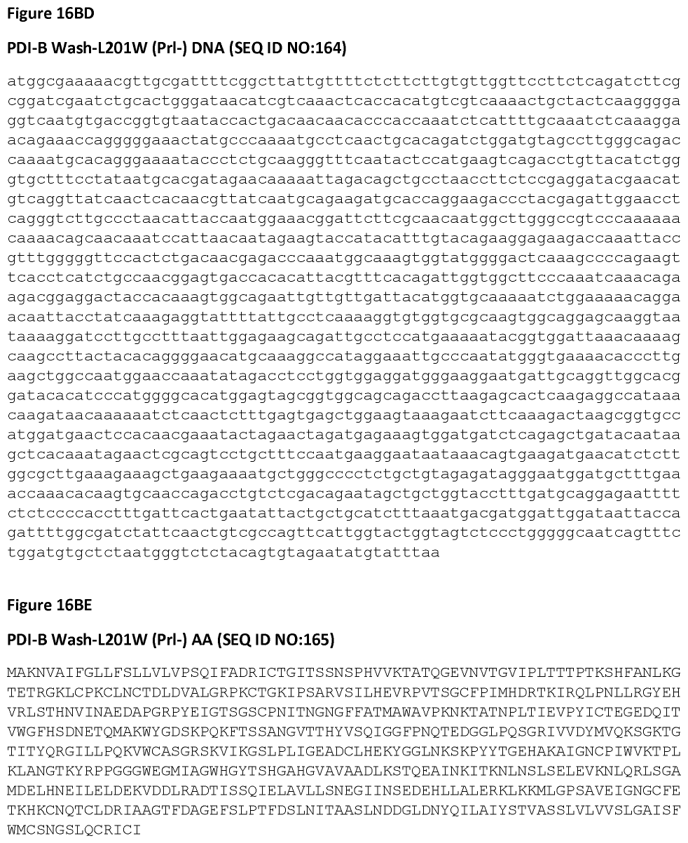

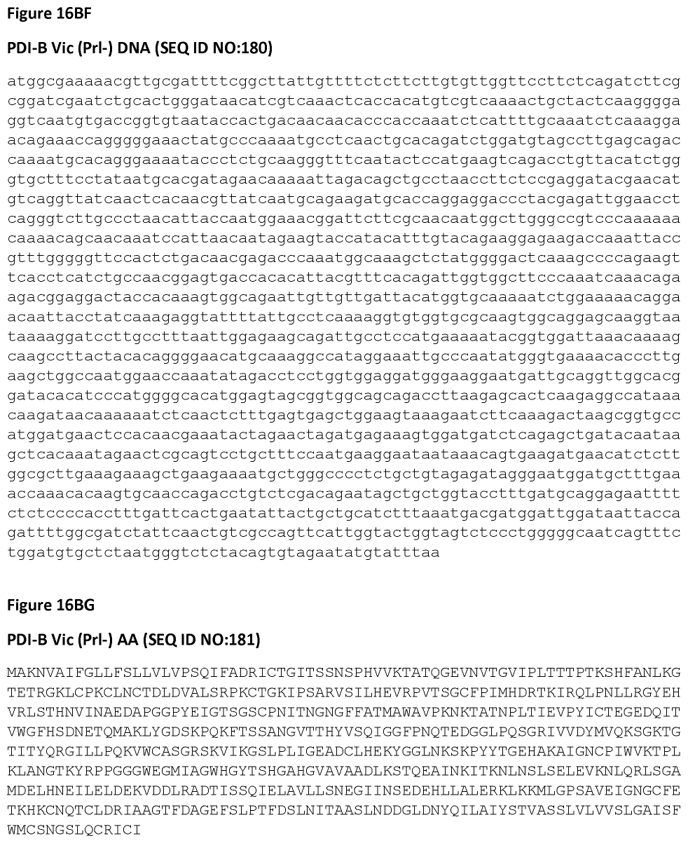

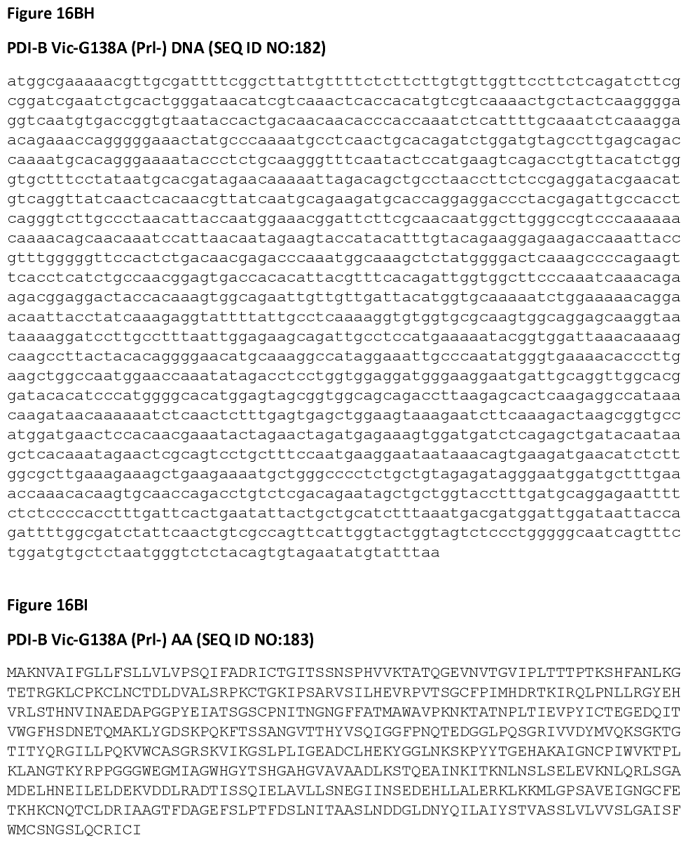

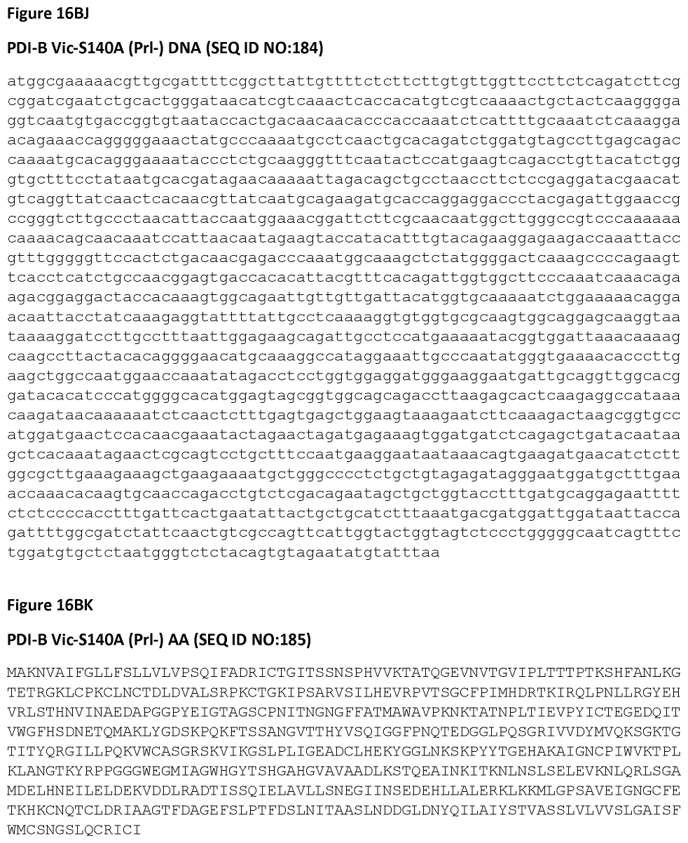

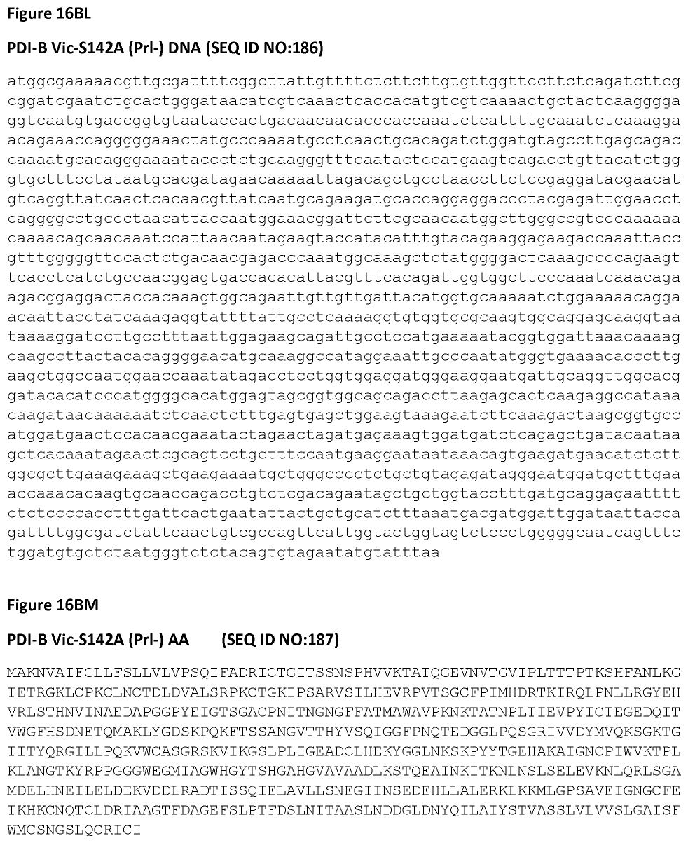

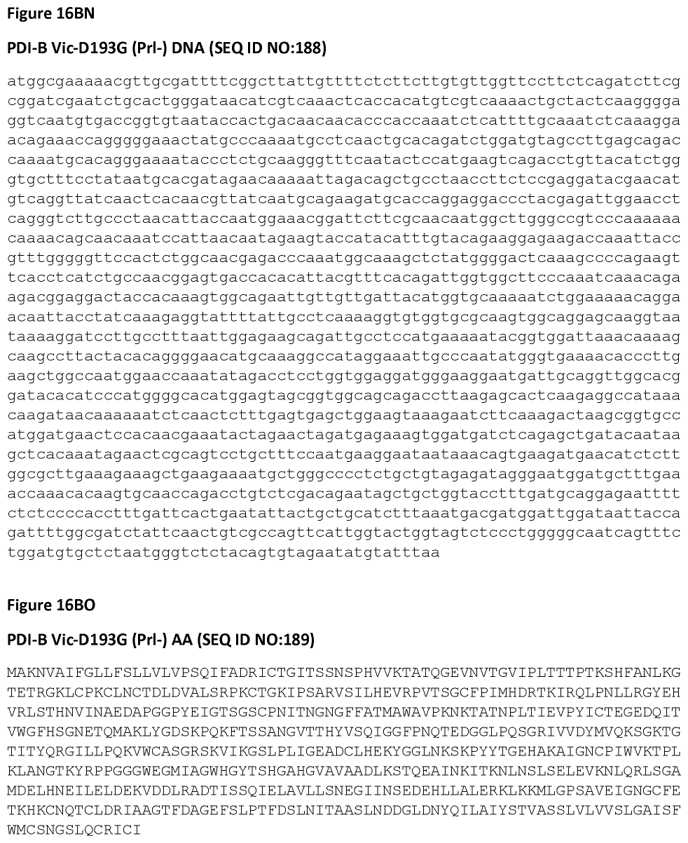

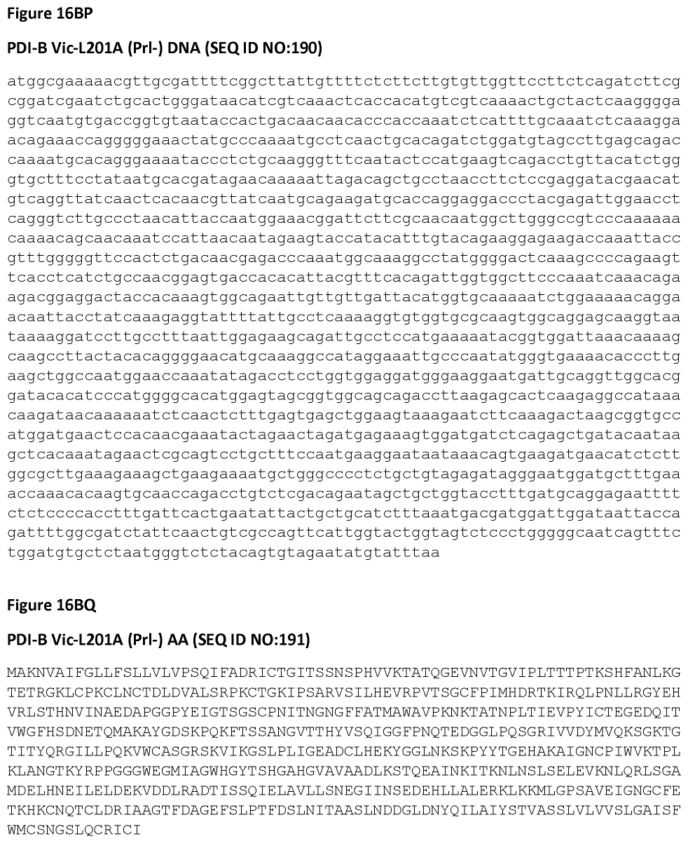

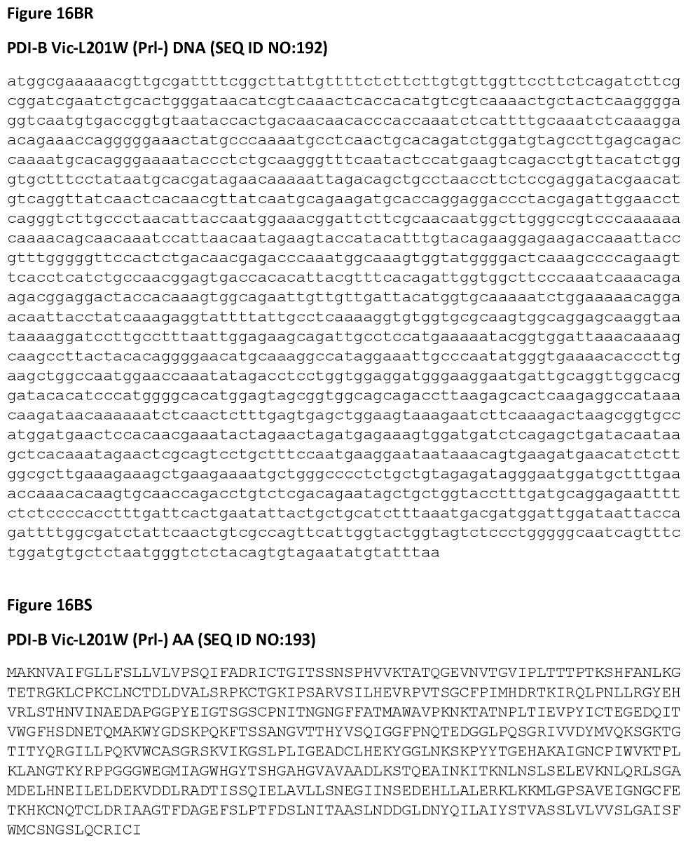

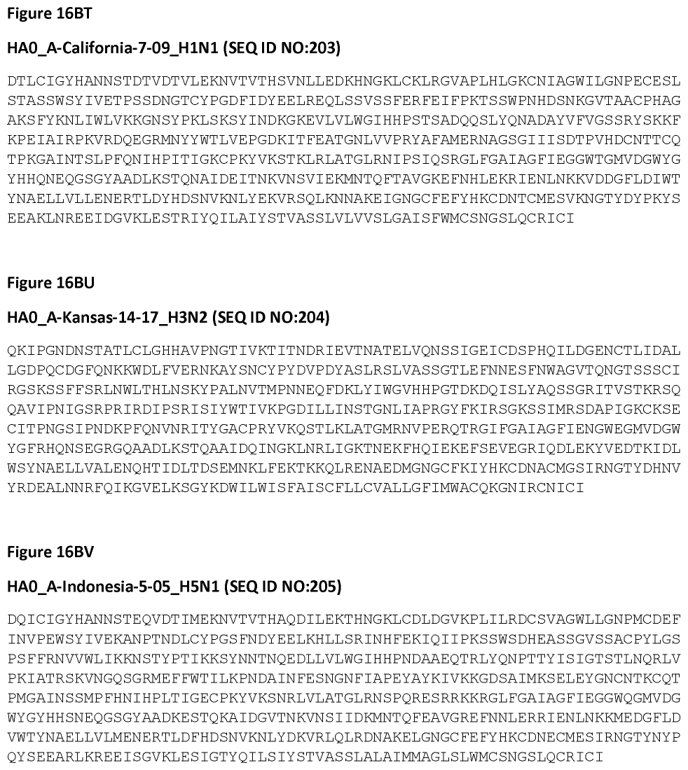

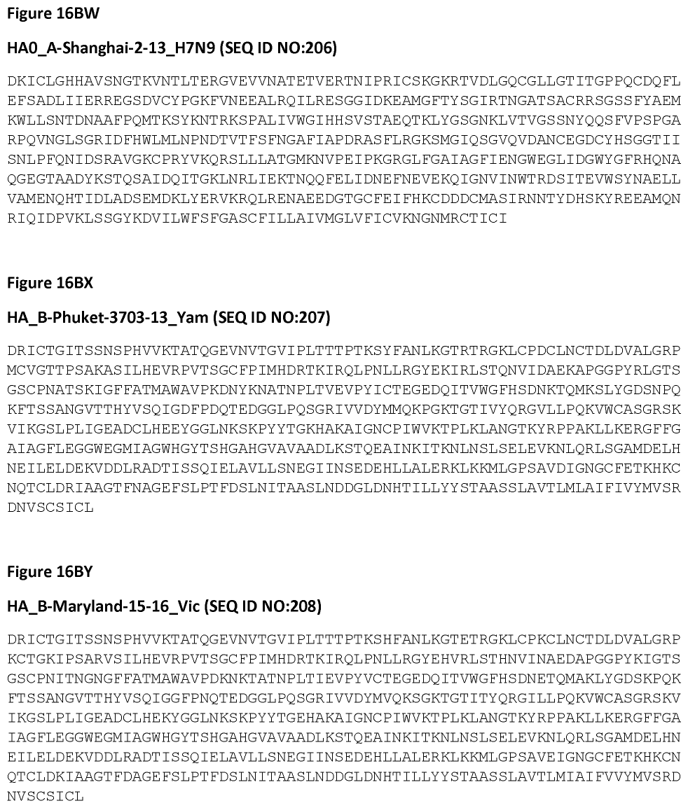

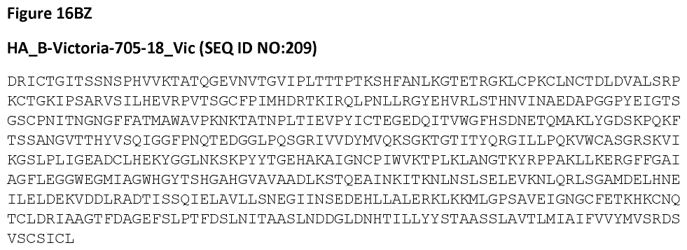

The amino acid residue number or residue position of HA is in accordance with the numbering of the HA of an influenza reference strain. For example the HA from the following reference strains may be used:
H1 A/California/07/2009 (SEQ ID NO:203, see FIG. 16BT),
H3 A/Kansas/14/2017 (SEQ ID NO:204, see FIG. 16BU);
H5 A/Indonesia/05/2005 (SEQ ID NO:205, see FIG. 16BV);
H7 A/Shanghai/2/2013 (SEQ ID NO:206, see FIG. 16BW);
B B/Phuket/3073/2013 (SEQ ID NO:207, see FIG. 16BX);
B B/Maryland/15/2016 (SEQ ID NO:208, see FIG. 16BY);
B B/Victoria/705/2018 (SEQ ID NO:209, see FIG. 16BZ).

The corresponding amino acid positions may be determined by aligning the sequences of the HA (for example H1, H3, H5, H7 or B HA) with the sequence of HA of their respective reference strain.

The amino acid residue number or residue position of HA is in accordance with the numbering of the HA of an influenza reference strain, or reference sequence. The reference sequence may be the wild type HA from which the modified HA is derived, or the reference sequence may be another defined reference sequence. For example, the HA reference sequence may be a wild type or non-modified (parent) H1 HA sequence (for example SEQ ID NO: 203), H3 HA sequence (for example SEQ ID NO: 204), H5 HA sequence (for example SEQ ID NO: 205), H7 HA sequence (for example SEQ ID NO: 206), or B HA sequence (for example SEQ ID NO: 207, SEQ ID NO: 208, or SEQ ID NO: 209; also see FIG. 1A, 1B and Table 1). The corresponding amino acid positions may be determined by aligning the sequences of the HA of interest with the reference sequence (or the sequence from which the modified HA sequence is derived; the parent HA sequence) as shown for example in FIG. 1A and Table 1. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

The term “residue” refers to an amino acid, and this term may be used interchangeably with the term “amino acid” and “amino acid residue”.

As used herein, the term “conserved substitution” or “conservative substitution” refers to the presence of an amino acid residue in the sequence of the HA protein that is different from, but it is in the same class of amino acid as the described substitution. For example, a nonpolar amino acid may be used to replace a nonpolar amino acid, an aromatic amino acid to replace an aromatic amino acid, a polar-uncharged amino acid to replace a polar-uncharged amino acid, and/or a charged amino acid to replace a charged amino acid). In addition, conservative substitutions can encompass an amino acid having an interfacial hydropathy value of the same sign and generally of similar magnitude as the amino acid that is replacing the corresponding wild type amino acid. As used herein, the term:
“nonpolar amino acid” refers to glycine (G, Gly), alanine (A, Ala), valine (V, Val), leucine (L, Leu), isoleucine (I, Ile), and proline (P, Pro);
“aromatic residue” (or aromatic amino acid) refers to phenylalanine (F, Phe), tyrosine (Y, Tyr), and tryptophan (W, Trp);
“polar uncharged amino acid” refers to serine (S, Ser), threonine (T, Thr), cysteine (C, Cys), methionine (M, Met), asparagine (N, Asn) and glutamine (Q, Gln);
“charged amino acid” refers to the negatively charged amino acids aspartic acid (D, Asp) and glutamic acid (E, Glu), as well as the positively charged amino acids lysine (K, Lys), arginine (R, Arg), and histidine (H, His).
amino acids with hydrophobic side chain (aliphatic) refers to Alanine (A, Ala), Isoleucine (I, Ile), Leucine (L, Leu), Methionine (M, Met) and Valine (V, Val);
amino acids with hydrophobic side chain (aromatic) refers to Phenylalanine (F, Phe), Tryptophan (W, Trp), Tyrosine (Y, Tyr);
amino acids with polar neutral side chain refers to Asparagine (N, Asn), Cysteine (C, Cys), Glutamine (Q, Gln), Serine (S, Ser) and Threonine (T, Thr);
amino acids with electrically charged side chains (acidic) refers to Aspartic acid (D, Asp), Glutamic acid (E, Glu);
amino acids with electrically charged side chains (basic) refers to Arginine (R, Arg); Histidine (H, His); Lysine (K, Lys), Glycine G, Gly) and Proline (P, Pro).

Conservative amino acid substitutions are likely to have a similar effect on the activity of the resultant modified HA protein as the original substitution or modification. Further information about conservative substitutions can be found, for example, in Ben Bassat et al. (J. Bacteriol, 169:751-757, 1987), O'Regan et al. (Gene, 77:237-251, 1989), Sahin-Toth et al. (Protein ScL, 3:240-247, 1994), Hochuli et al (Bio/Technology, 6:1321-1325, 1988).

The Blosum matrices are commonly used for determining the relatedness of polypeptide sequences (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity. The following table shows examples of conservative amino acid substitutions: Table 2.

TABLE 2
Exemplary conservative amino acid substitutions.

| | Very Highly - | Highly Conserved | |
|---|---|---|---|
| Original | Conserved | Substitutions (from the | Conserved Substitutions |
| Residue | Substitutions | Blosum90 Matrix) | (from the Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, Lys, Met, Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

When referring to modifications, mutants or variants, the wild type amino acid residue (also referred to as simply ‘amino acid’) is followed by the residue number and the new or substituted amino acid. For example, which is not to be considered limiting, substitution of tyrosine (Y, Tyr) for phenylalanine (F, Phe) in residue or amino acid at position 98, is denominated Y98F.

Examples of modifications that may be used as described herein to produce a modified HA that exhibits the property of having reduced, non-detectable, or no non-cognate interaction with SA, for example, reduced, non-detectable or no SA binding, while maintaining cognate interaction of the modified HA protein with a target, and/or a modified HA that modulates and/or increases an immunological response in an animal or a subject in response to an antigen challenge, for example, targets mediated by the B cell receptor, include:
an H1-HA comprising a Y91F substitution. The amino acid substitution at position 91 may be determined by sequence alignment with the H1 reference sequence H1 A/California/7/09 (SEQ ID NO:203). An alternate amino acid substitution at position 91 with an aromatic side chain may include Tryptophan (W, Trp; Y91W);
an H3-HA comprising a Y98F substitution in combination with a substitution selected from the group of S136D, S136N, S137N, D190G, D190K, R222W, S228N, S228Q determined by sequence alignment with the reference sequence H3 A/Kansas/14/17 (SEQ ID NO:204). Alternate amino acid substitutions at position 98 may include an aromatic side chain, Tryptophan (W, Trp; Y98W); alternate substitutions at positions 136, 137 and 228 may include polar uncharged amino acids, for example: Asparagine (N, Asn; S136N; S137N), Cysteine (C, Cys; S136C; S137C; S228C), Glutamine (Q, Gln; S136Q; S137Q), and Threonine (T, Thr; S136T; S137T; S228T); alternate substitutions at position 190 may include electrically charged side chains, for example glutamic acid (E; Glu; D190E); (R, Arg; D190R); Histidine (H, His: D190H); and Proline (P, Pro; D190P); alternate substitutions at position 222 may include Histidine (H, His; R222H); Lysine (K, Lys; R222K), Glycine G, Gly; R222G) and Proline (P, Pro; R222P);
an H3-HA comprising a substitution selected from the group of S136D, S136N, D190K, R222W, S228N or S228Q determined by sequence alignment with the reference sequence H3 A/Kansas/14/17 (SEQ ID NO:204). Alternate substitutions at positions 136 and 228 may include polar uncharged amino acids, for example: Asparagine (N, Asn; S136N), Cysteine (C, Cys; S136C; S228C), Glutamine (Q, Gln; S136Q), and Threonine (T, Thr; S136T; S228T); alternate substitutions at position 190 may include electrically charged side chains, for example glutamic acid (E; Glu; D190E); (R, Arg; D190R); Histidine (H, His: D190H); and Proline (P, Pro; D190P); alternate substitutions at position 222 may include Histidine (H, His; R222H); Lysine (K, Lys; R222K), Glycine G, Gly; R222G) and Proline (P, Pro; R222P);
an H5-HA comprising a Y91F substitution. The amino acid substitution at position 91 may be determined by sequence alignment with the reference sequence H5 A/Indonesia/5/05 (SEQ ID NO:205). An alternate amino acid substitution at position 91 with an aromatic side chain may include Tryptophan (W, Trp; Y91W);
an H7-HA comprising a Y88F substitution. The amino acid substitution at position 88 may be determined by sequence alignment with the reference sequence H7 A/Shanghai/2/12 (SEQ ID NO:206). An alternate amino acid substitution at position 88 with an aromatic side chain may include Tryptophan (W, Trp; Y88W);
a B-HA comprising a substitution selected from the group: S140A, S142A, G138A, D195G, L203W and L203A determined with reference to the B/Phuket/3073/2013 (SEQ ID NO:207). Alternate amino acid substitution at positions 140 and 142 may include polar uncharged amino acids, for example: Asparagine (N, Asn; S140N; S142N), Cysteine (C, Cys; S140C; S142C), Glutamine (Q, Gln; S140Q; S142Q), and Threonine (T, Thr; S140T; S142T); alternate amino acid substitution at position 138 may include other nonpolar amino acids, for example, valine (V, Val; G138V), leucine (L, Leu; G138L), isoleucine (I, Ile; G138I), and proline (P, Pro; G138P); alternate amino acid substitution at position 195 may include the charged amino acid glutamic acid (E, Glu; D195E); alternate amino acid substitution at position 203 may include nonpolar amino acids, for example glycine (G, Gly; L203G), valine (V, Val; L203V), isoleucine (I, Ile; L203I), and proline (P, Pro; L203P).
a B-HA comprising a substitution selected from the group: S140A, S142A, G138A, D194G, L202W and L202A determined with reference to the B/Maryland/15/2016 (SEQ ID NO:208). Alternate amino acid substitution at positions 140 and 142 may include polar uncharged amino acids, for example: Asparagine (N, Asn; S140N; S142N), Cysteine (C, Cys; S140C; S142C), Glutamine (Q, Gln; S140Q; S142Q), and Threonine (T, Thr; S140T; S142T); alternate amino acid substitution at position 138 may include other nonpolar amino acids, for example, valine (V, Val; G138V), leucine (L, Leu; G138L), isoleucine (I, Ile; G138I), and proline (P, Pro; G138P); alternate amino acid substitution at position 194 may include the charged amino acid glutamic acid (E, Glu; D194E); alternate amino acid substitution at position 202 may include nonpolar amino acids, for example glycine (G, Gly; L202G), valine (V, Val; L202V), isoleucine (I, Ile; L202I), and proline (P, Pro; L202P).
a B-HA comprising a substitution selected from the group: S140A, S142A, G138A, D193G, L201W and L201A determined with reference to the B/Victoria/705/2018 (SEQ ID NO:209). Alternate amino acid substitution at positions 140 and 142 may include polar uncharged amino acids, for example: Asparagine (N, Asn; S140N; S142N), Cysteine (C, Cys; S140C; S142C), Glutamine (Q, Gln; S140Q; S142Q), and Threonine (T, Thr; S140T; S142T); alternate amino acid substitution at position 138 may include other nonpolar amino acids, for example, valine (V, Val; G138V), leucine (L, Leu; G138L), isoleucine (I, Ile; G138I), and proline (P, Pro; G138P); alternate amino acid substitution at position 193 may include the charged amino acid glutamic acid (E, Glu; D194E); alternate amino acid substitution at position 201 may include nonpolar amino acids, for example glycine (G, Gly; L201G), valine (V, Val; L201V), isoleucine (I, Ile; L201I), and proline (P, Pro; L201P).

A nucleic acid encoding the modified HA with reduced, non-detectable, or no non-cognate interaction with SA as described herein is also provided. Furthermore, hosts that comprise the nucleic acid are also described. Suitable hosts are described below, and may include, but are not limited to, a eukaryotic host, cultured eukaryotic cells, an avian host, an insect host, or a plant host. For example, a plant, portion of a plant, plant matter, plant extract, plant cell, may comprise the nucleic acid encoding the modified influenza HA with reduced, non-detectable, or no non-cognate interaction with SA.

Also provided is a method to produce a modified HA with reduced, non-detectable, or no non-cognate interaction with SA, a suprastructure comprising the modified HA, a nanoparticle comprising the modified HA, or a VLP (or suprastructure) comprising the modified HA, by expressing the nucleic acid encoding the modified HA with reduced, non-detectable, or no non-cognate interaction with SA within a suitable host, for example, but not limited to a eukaryotic host, cultured eukaryotic cells, an avian host, an insect host, or a plant host. The method may involve introducing the nucleic acid encoding the modified HA with reduced, non-detectable, or no non-cognate interaction with SA into the plant and growing the plant under conditions that result in the expression of the nucleic acid and production of the modified HA, the suprastructure comprising the modified HA, a nanoparticle comprising the modified HA, or the VLP comprising the modified HA, or a combination thereof, and harvesting the plant. Alternatively, the method may involve growing a plant that already comprises the nucleic acid encoding the modified HA with reduced, non-detectable, or no non-cognate interaction with SA under conditions that result in the expression of the nucleic acid and production of the modified HA, the suprastructure comprising the modified HA, the nanoparticle comprising the modified HA, or the VLP comprising the modified HA, or a combination thereof, and harvesting the plant. The modified HA, the suprastructure comprising the modified HA, the nanoparticle comprising the modified HA, or the VLP comprising modified HA may be purified as described herein or by using purification protocols known to one of skill in the art.

### VLPs

Described herein are VLPs comprising a modified influenza HA with reduced, non-detectable, or no non-cognate interaction with SA. Also described is the use of these VLPs as an influenza vaccine that exhibits increased immunogenicity and efficacy when compared to the immunogenicity and efficacy of an influenza vaccine comprising VLPs comprising the corresponding wild type (or non-modified) influenza HA. As described above, a VLP may be considered an example of a nanoparticle or a suprastructure comprising HA or a modified HA, and unless otherwise stated, these terms may be used interchangeably.

The term “virus like particle” (VLP), or “virus-like particles” or “VLPs” refers to structures that self-assemble and comprise structural proteins such as influenza HA protein. VLPs are generally morphologically and antigenically similar to virions produced in an infection but lack genetic information sufficient to replicate and thus are non-infectious. The VLP may comprise an HA0, HA1 or HA2 peptide. In some examples, VLPs may comprise a single protein species, or more than one protein species. For VLPs comprising more than one protein species, the protein species may be from the same species of virus, or may comprise a protein from a different species, genus, subfamily or family of virus (as designated by the ICTV nomenclature). As described herein, the one or more of the protein species comprising a VLP may be modified from the naturally occurring sequence. VLPs may be produced in suitable host cells including plant and insect host cells. Following extraction from the host cell and upon isolation and further purification under suitable conditions, VLPs may be purified as intact structures.

In plants, influenza VLPs bud from the plasma membrane therefore the lipid composition of the VLPs reflects their origin. The plant-derived lipids may be in the form of a lipid bilayer and may further comprise an envelope surrounding the VLP. The plant derived lipids may comprise lipid components of the plasma membrane of the plant where the VLP is produced, including, but not limited to, phosphatidylcholine (PC), phosphatidylethanolamine (PE), glycosphingolipids, phytosterols or a combination thereof. A plant-derived lipid may alternately be referred to as a ‘plant lipid’. Examples of phytosterols are known in the art, and include, for example, stigmasterol, sitosterol, 24-methylcholesterol and cholesterol. Therefore, a VLP as described herein may be complexed with a plant-derived lipid bilayer. The phytosterols present in an influenza VLP complexed with a lipid bilayer, such as a plasma-membrane derived envelope may provide for an advantageous vaccine composition. Without wishing to be bound by theory, plant-made VLPs complexed with a lipid bilayer, such as a plasma-membrane derived envelope, may induce a stronger immune reaction than VLPs made in other expression systems, and may be similar to the immune reaction induced by live or attenuated whole virus vaccines. Furthermore, the conformation of the VLP may be advantageous for the presentation of the antigen and enhance the adjuvant effect of VLP when complexed with a plant derived lipid layer.

PC and PE, as well as glycosphingolipids can bind to CD1 molecules expressed by mammalian immune cells such as antigen-presenting cells (APCs) like dendritic cells and macrophages and other cells including B and T lymphocytes in the thymus and liver (Tsuji M., 2006). CD1 molecules are structurally similar to major histocompatibility complex (MHC) molecules of class I and their role is to present glycolipid antigens to NKT cells (Natural Killer T cells). Upon activation, NKT cells activate innate immune cells such as NK cells and dendritic cells, and also activate adaptive immune cells like the antibody-producing B cells and T-cells.

The VLP produced within a plant may comprise HA that comprises plant-specific N-glycans. Therefore, a VLP comprising HA having plant specific N-glycans is also described.

Modification of N-glycan in plants is known (see for example WO2008/151440; WO2010/006452; WO2014/071039; WO/2018058256, each of which is incorporated herein by reference) and HA having modified N-glycans may be produced. HA comprising a modified glycosylation pattern, for example with reduced or non-detectable levels of fucosylated, xylosylated, or both, fucosylated and xylosylated, N-glycans may be obtained, or HA having a modified glycosylation pattern may be obtained, wherein the protein lacks fucosylation, xylosylation, or both, when compared to a wild-type plant expressing HA. Without wishing to be bound by theory, the presence of plant N-glycans on HA may stimulate the immune response by promoting the binding of HA by antigen presenting cells. Therefore, the present invention also includes VLP's comprising HA having modified N-glycans.

VLPs may be assessed for structure and size by, for example, hemagglutination assay, electron microscopy, gradient density centrifugation, by size exclusion chromatography, ion exchange chromatography, affinity chromatography, or other size determining assay as would be known to one of skill in the art. For example, which is not to be considered limiting, total soluble proteins may be extracted from plant tissue by enzymatic digestion, for example as described in WO2011/035422, WO2011/035423, WO2012/126123 (each of which is incorporated herein by reference), homogenizing (Polytron) samples of fresh or frozen-crushed plant material in extraction buffer, and insoluble material removed by centrifugation or depth filtration. Precipitation with PEG, salt, or pH, may also be used. The soluble protein may be passed through a size exclusion column, an ion exchange column, or an affinity column. Following chromatography, fractions may be further analyzed by PAGE, Western, or immunoblot to determine the protein complement of the fraction. The relative abundance of the modified HA may also be determined using a hemagglutination assay.

### Hosts

The modified influenza HA as described herein, the VLP comprising the modified HA, or both the modified HA and the VLP comprising the modified HA as described herein, may be produced within any suitable host, for example, but not limited to a eukaryotic host, a eukaryotic cell, a mammalian host, a mammalian cell, an avian host, an avian cell, an insect host, an insect cell, a baculovirus cell, or a plant host, a plant or a portion of a plant, a plant cell. For example the host may be an animal or non-human host. For example, a plant may be used to produce a modified influenza HA with reduced, non-detectable, or no non-cognate interaction with SA, a VLP comprising the modified HA, or both the modified influenza HA with reduced, non-detectable, or no non-cognate interaction with SA and a VLP comprising the modified HA. Therefore, also described are plants that comprise a VLP comprising a modified influenza HA with reduced, non-detectable, or no non-cognate interaction with SA. Furthermore, plants that that comprise the modified influenza HA with reduced, non-detectable, or no non-cognate interaction with SA are also described.

Plants may include, but are not limited to, herbaceous plants. Furthermore plants may include, but are not limited to, agricultural crops including for example canola, Brassica spp., maize, Nicotiana spp., (tobacco) for example, Nicotiana benthamiana, Nicotiana rustica, Nicotiana, tabacum, Nicotiana alata, Arabidopsis thaliana, alfalfa (Medicago spp., for example, Medicago trunculata), potato, sweet potato (Ipomoea batatus), ginseng, pea, oat, rice, soybean, wheat, barley, sunflower, cotton, corn, rye (Secale cereale), sorghum (Sorghum bicolor, Sorghum vulgare), safflower (Carthamus tinctorius), lettuce and cabbage.

### Compositions

Also described herein is a composition comprising one or more than one modified influenza HA with reduced, non-detectable, or no non-cognate interaction with SA, or one or more than one VLP comprising one or more than one modified influenza HA with reduced, non-detectable, or no non-cognate interaction with SA, and a pharmaceutically acceptable carrier, adjuvant, vehicle, or excipient. The composition comprising the modified influenza HA, or VLP comprising the modified HA protein, may be used as a vaccine for use in administering to a subject in order to induce an immune response. Therefore, the present disclosure provides a vaccine comprising the composition comprising one or more than one modified influenza HA with reduced, non-detectable, or no non-cognate interaction with SA, or one or more than one VLP comprising one or more than one modified influenza HA with reduced, non-detectable, or no non-cognate interaction with SA.

The composition may comprise a mixture of VLPs provided that at least one of the VLPs within the composition comprises modified HA protein as described herein. For example, each HA including one or more than one modified HA, from each of the one or more than one influenza subtypes may be expressed and the corresponding VLPs purified. Virus like particles obtained from two or more than two influenza strains (for example, two, three, four, five, six, seven, eight, nine, 10 or more strains or subtypes) may be combined as desired to produce a mixture of VLPs, provided that one or more than one VLP in the mixture of VLPs comprises a modified HA as described herein. The VLPs may be combined or produced in a desired ratio, for example about equivalent ratios, or may be combined in such a manner that one subtype or strain comprises the majority of the VLPs in the composition.

Selection of the combination of HAs may be determined by the intended use of the vaccine prepared from the VLP. For example a vaccine for use in inoculating birds may comprise any combination of HA subtypes, while VLPs useful for inoculating humans may comprise subtypes one or more than one of subtypes H1, H2, H3, H5, H7, H9, H10, N1, N2, N3 and N7. However, other HA subtype combinations may be prepared depending upon the use of the inoculum. For example, the choice of combination of strains and subtypes may also depend on the geographical area of the subjects likely to be exposed to influenza, proximity of animal species to a human population to be immunized (e.g. species of waterfowl, agricultural animals such as swine, etc) and the strains they carry, are exposed to or are likely to be exposed to, predictions of antigenic drift within subtypes or strains, or combinations of these factors. Examples of combinations used in past years are available (see URL: who.int/csr/disease/influenza/vaccine recommendations1/en).

Therefore, a composition is provided that comprise a VLP comprising a modified HA as described herein, or that comprises a mixture of VLPs, each VLP comprising a different HA subtype or strain, provided that one of the HA's is a modified HA as described herein.

The composition comprising a VLP comprising a modified HA, or a composition comprising a mixture of VLPs as described above, may be use for inducing immunity to influenza virus infection in an animal or subject. For example, an effective dose of a vaccine comprising the composition may be administered to an animal or subject. The vaccine may be administered orally, intradermally, intranasally, intramuscularly, intraperitoneally, intravenously, or subcutaneously. For example, which is not to be considered limiting, the subject may be selected from the group comprising humans, primates, horses, pigs, birds, water fowl, migratory birds, quail, duck, geese, poultry, chicken, swine, sheep, equine, horse, camel, canine, dogs, feline, cats, tiger, leopard, civet, mink, stone marten, ferrets, house pets, livestock, rabbits, guinea pigs or other rodents, mice, rats, seal, fish, whales and the like.

Therefore, the present disclosure also provides a method of inducing immunity to influenza virus infection in an animal or subject in need thereof, comprising administering the VLP comprising the modified influenza HA with reduced, non-detectable, or no non-cognate interaction with SA to the animal or subject. As described below, the use of the modified influenza HA with reduced, non-detectable, or no non-cognate interaction with SA elicits an improved immune response when compared with the immune response obtained following vaccination of the subject using the corresponding wild type or non-modified HA that does not comprise a modification that reduces SA binding.

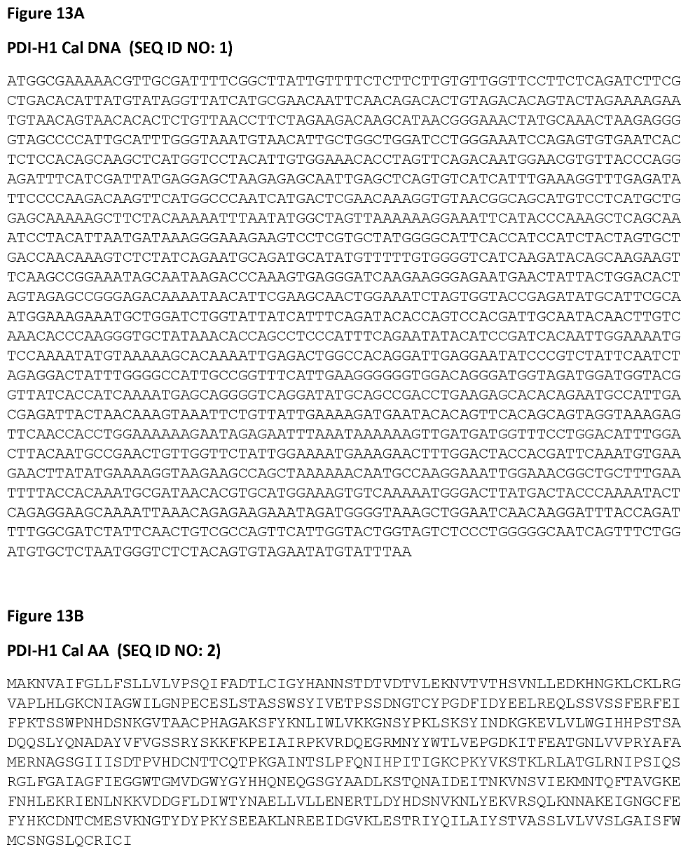

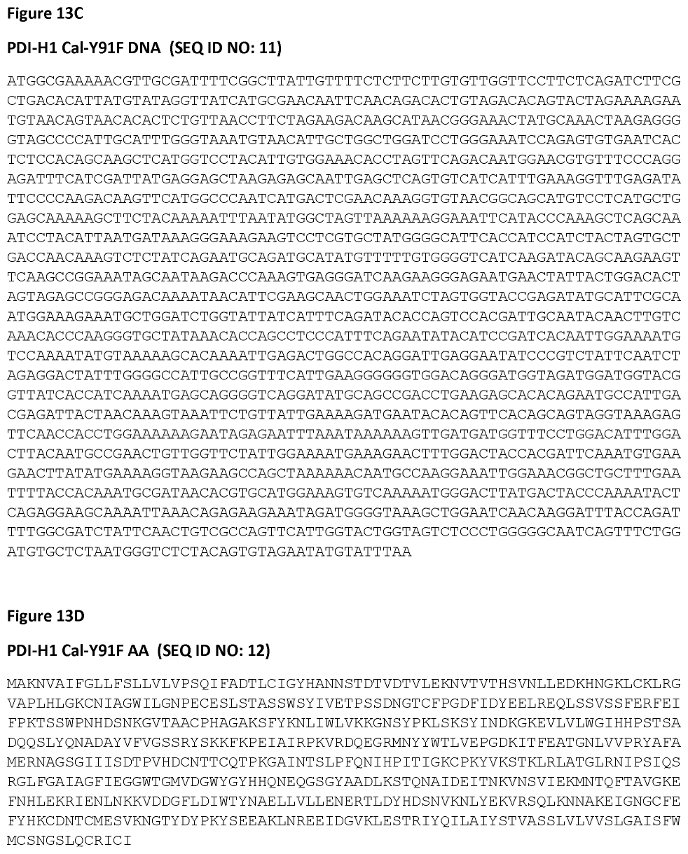

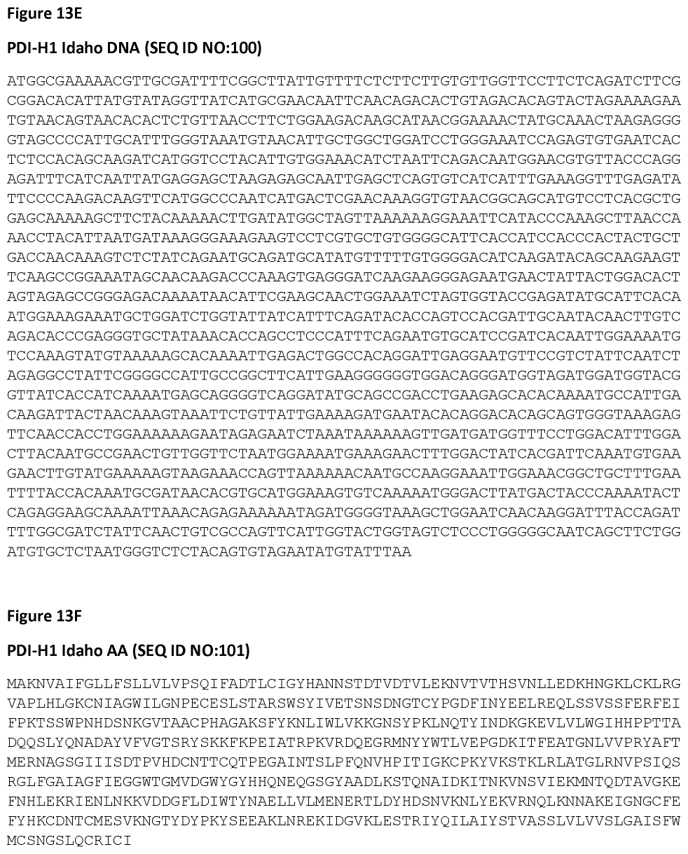

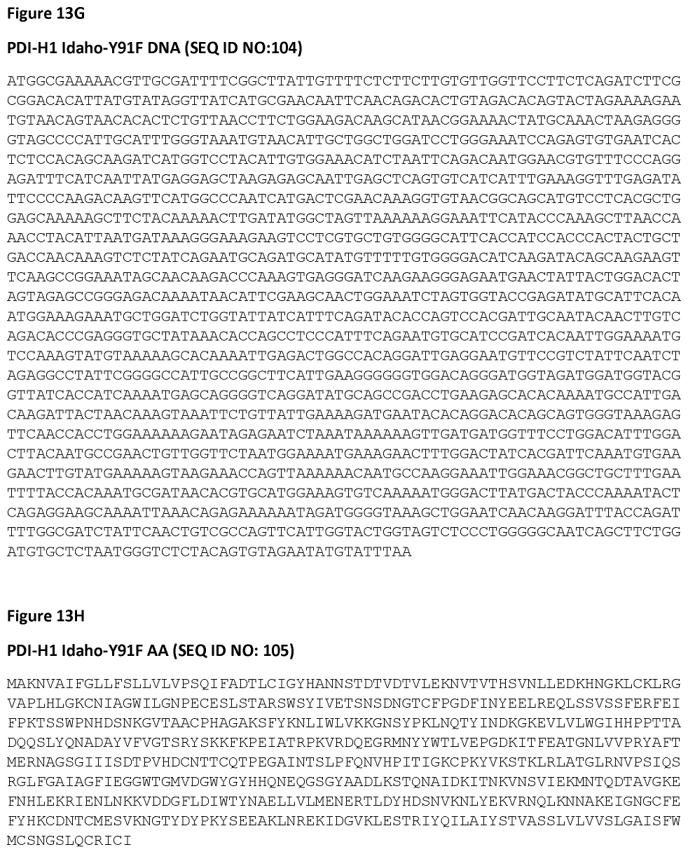

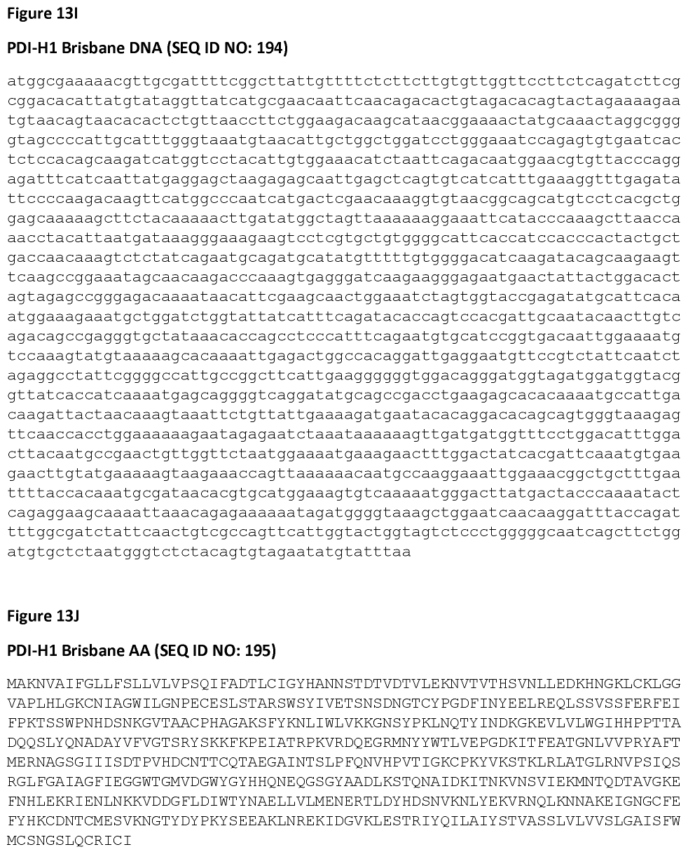

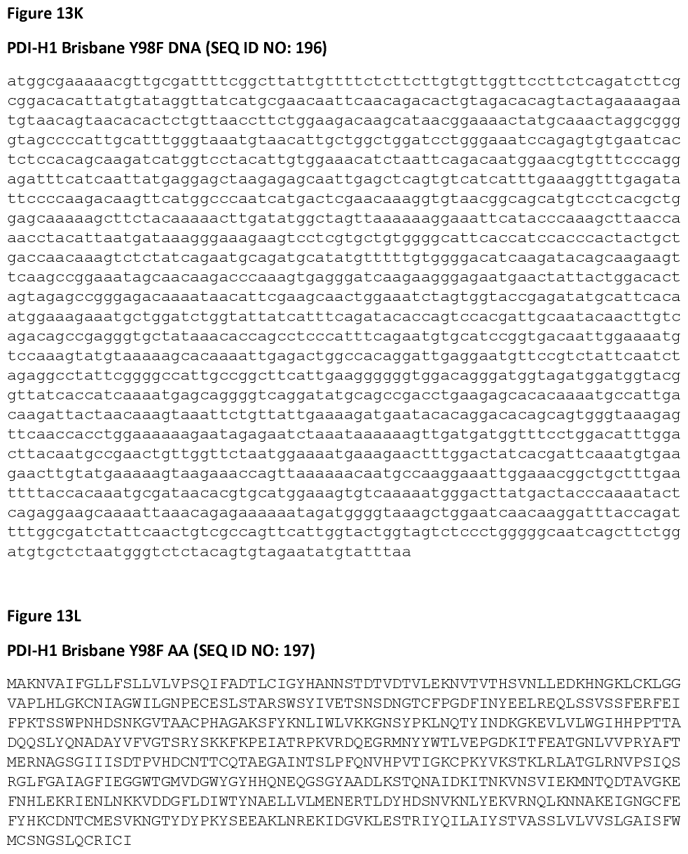

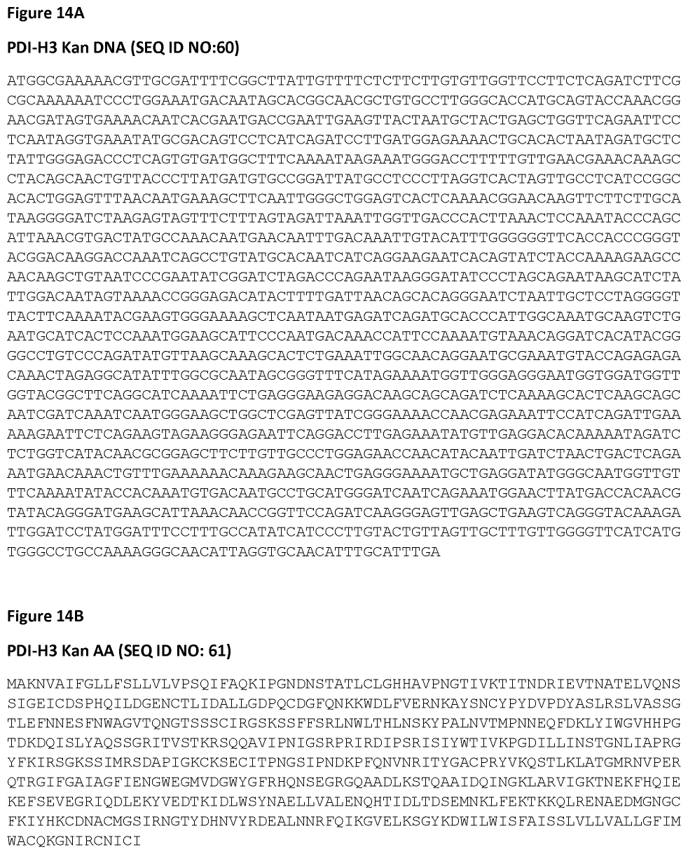

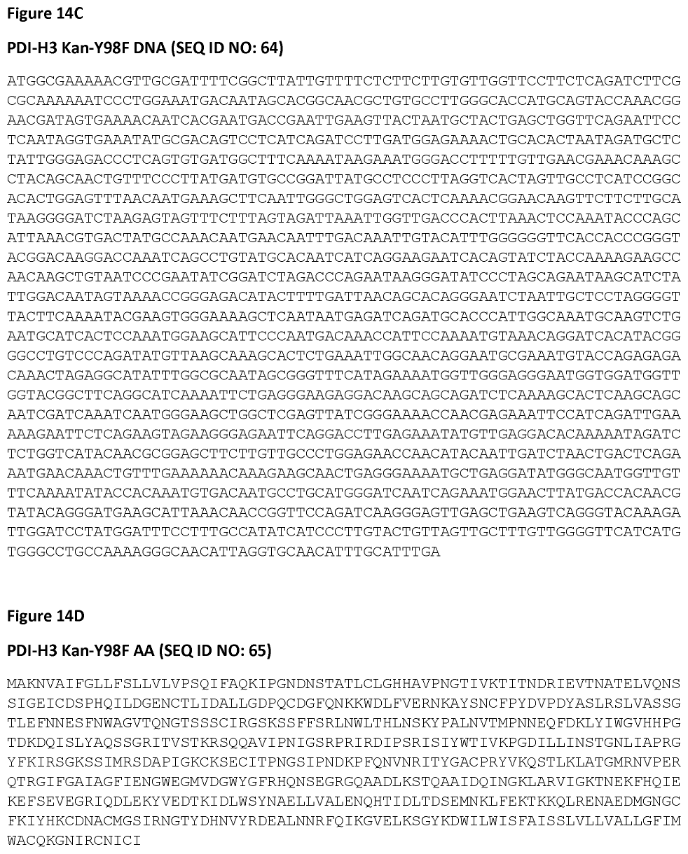

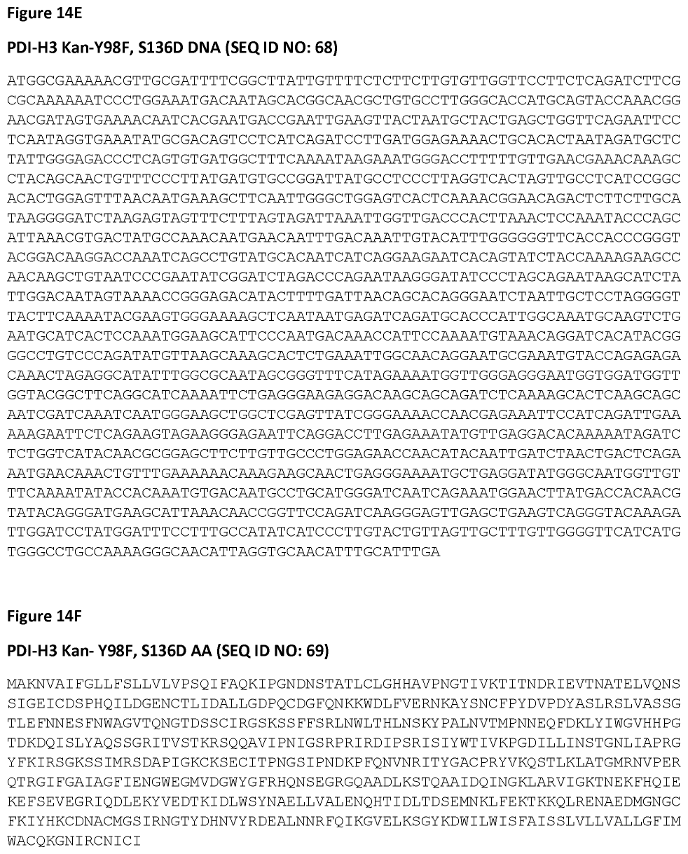

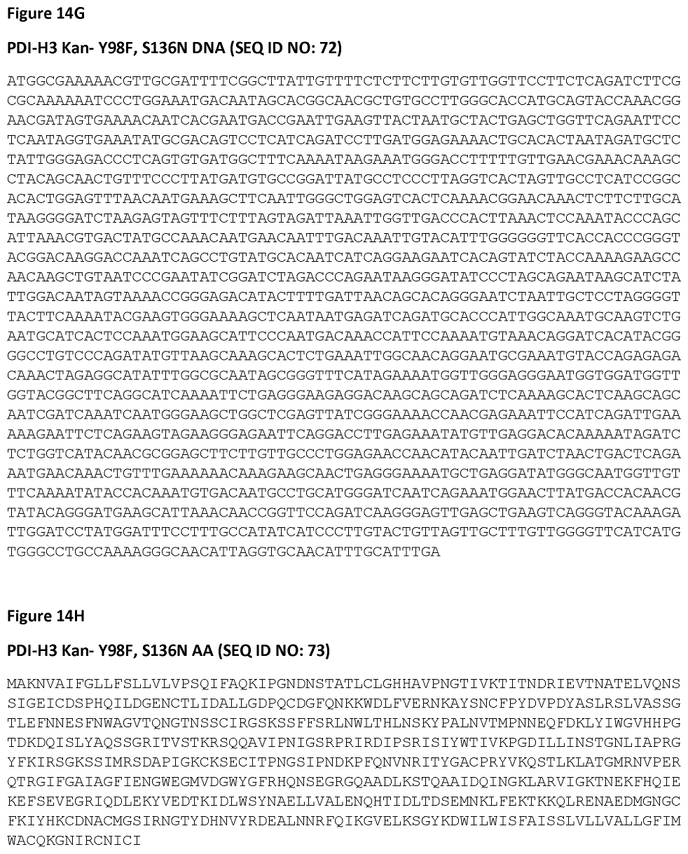

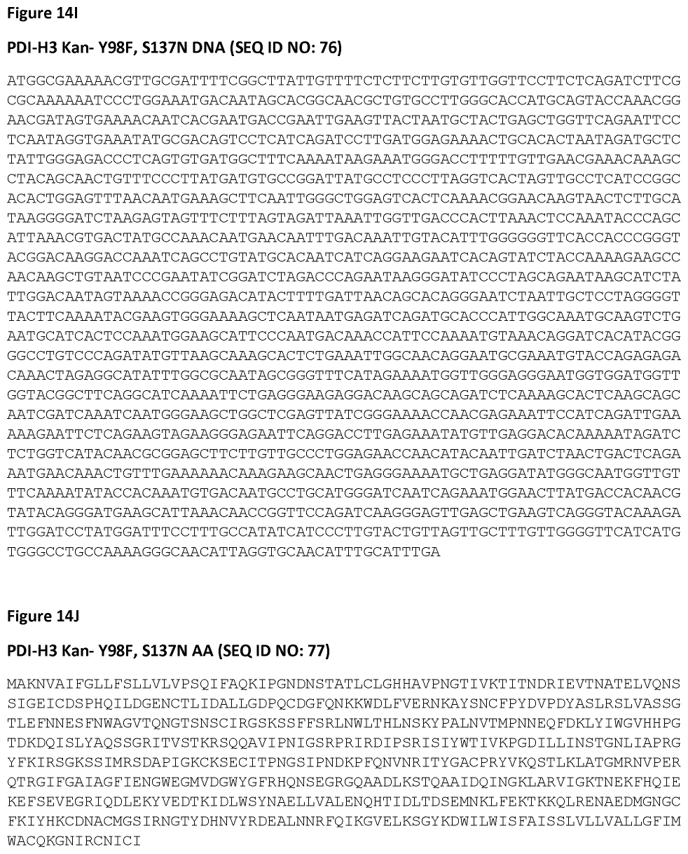

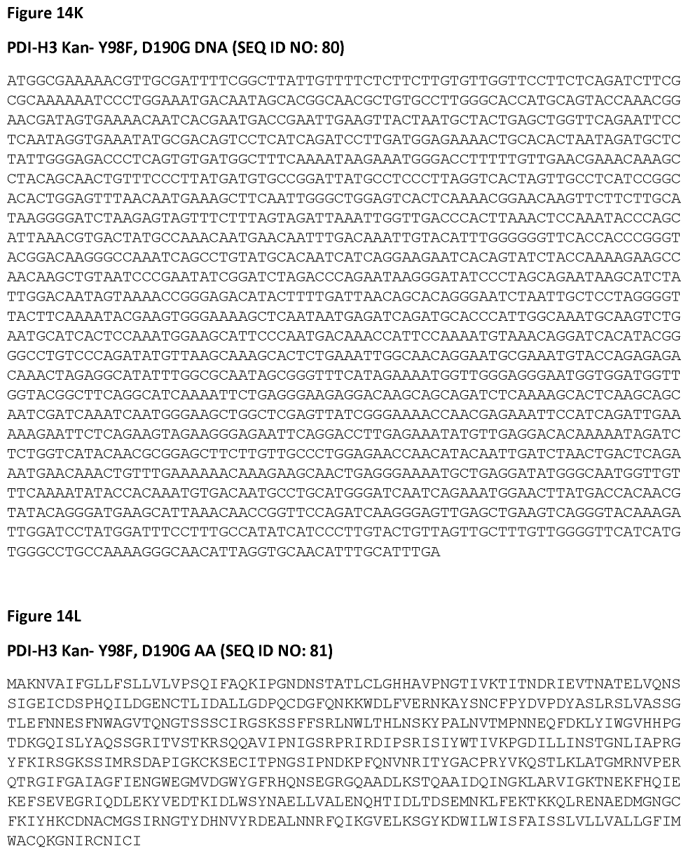

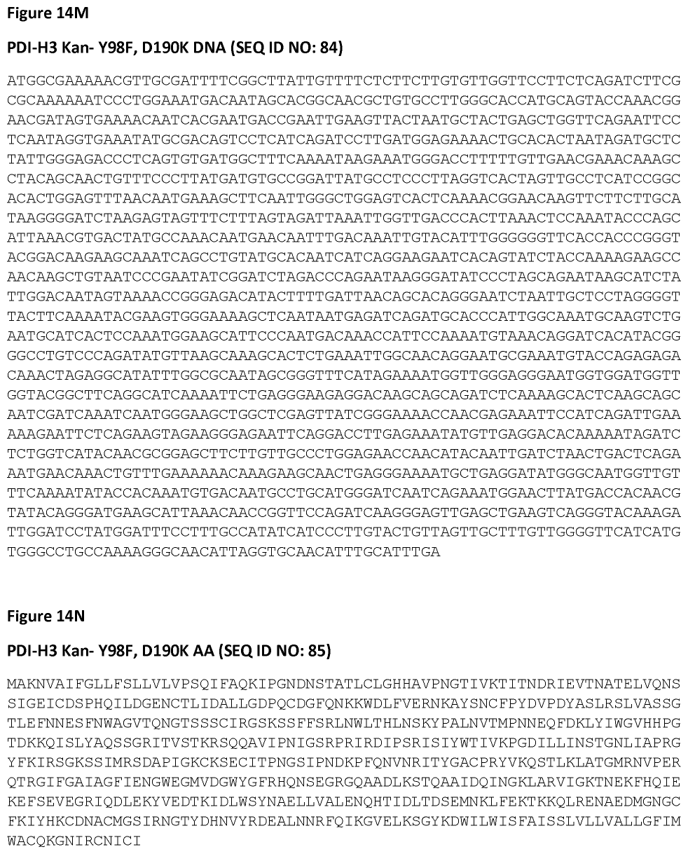

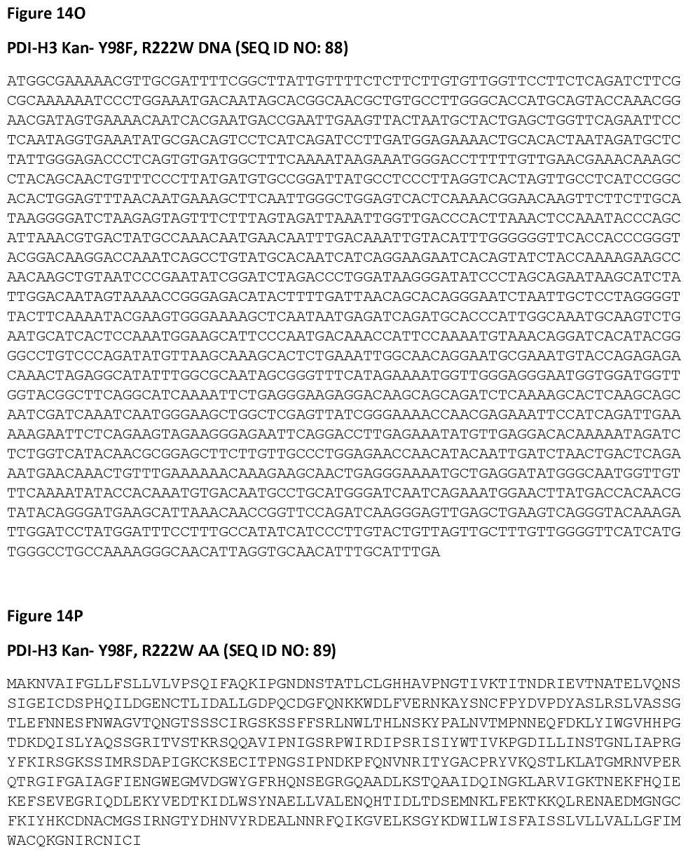

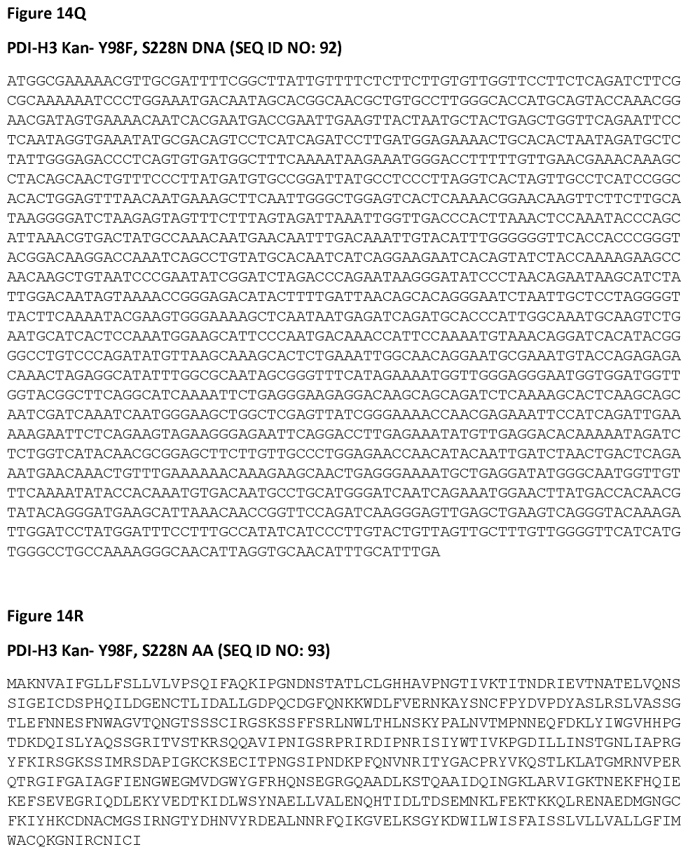

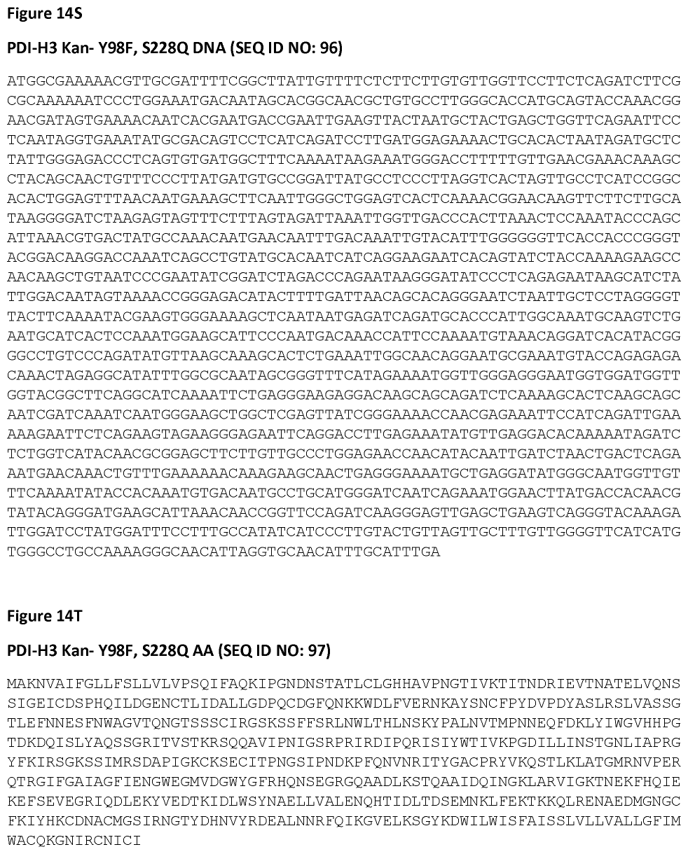

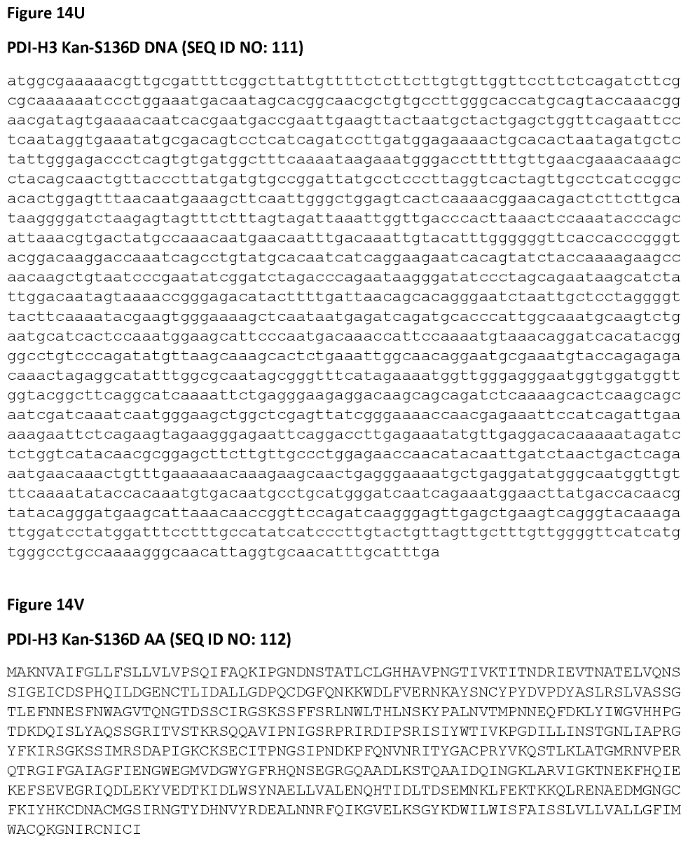

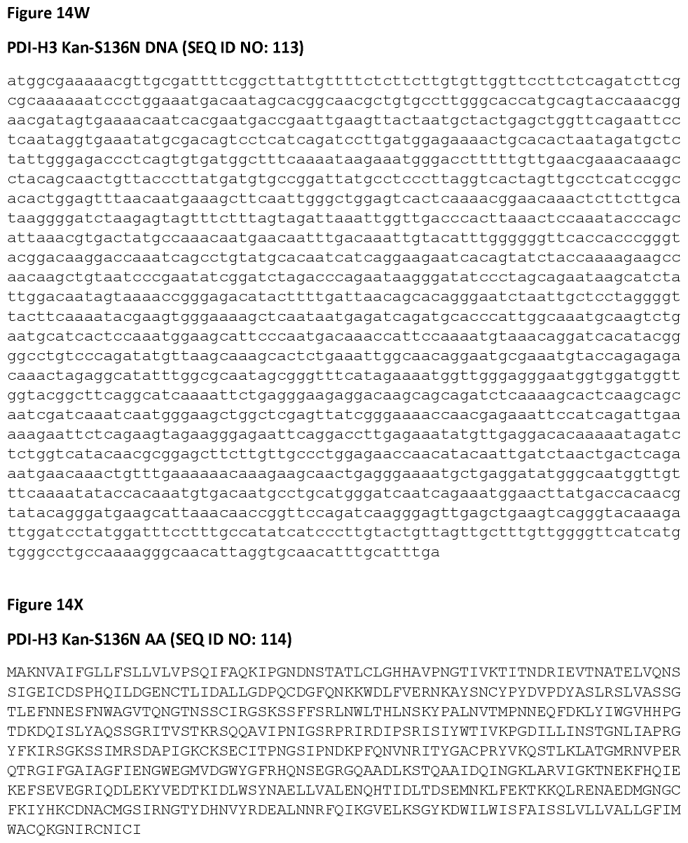

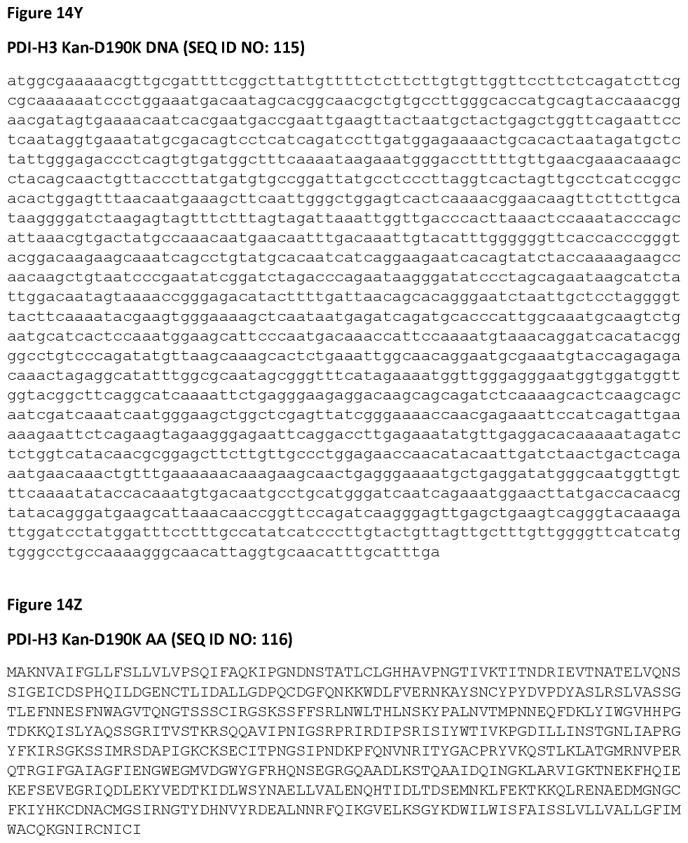

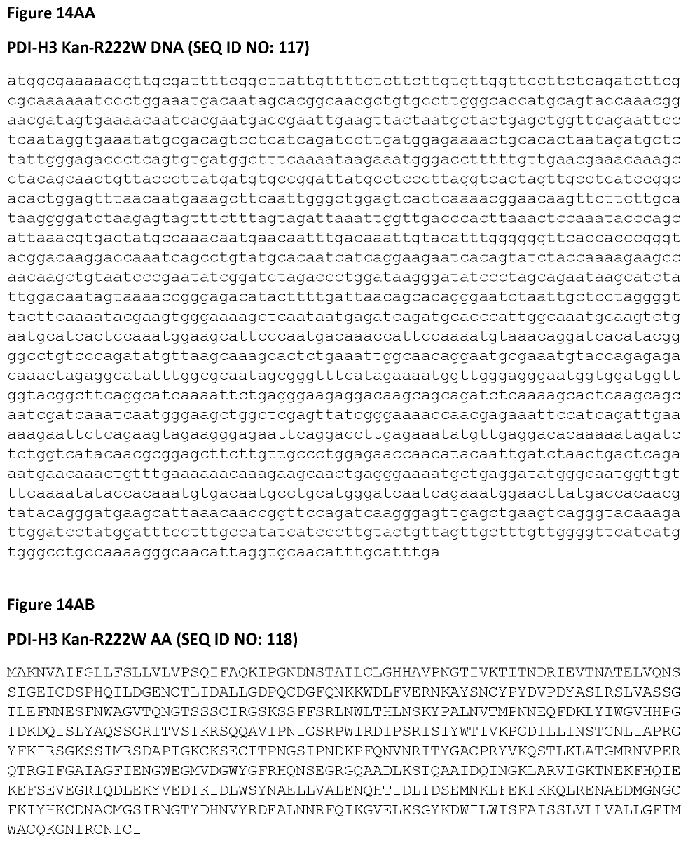

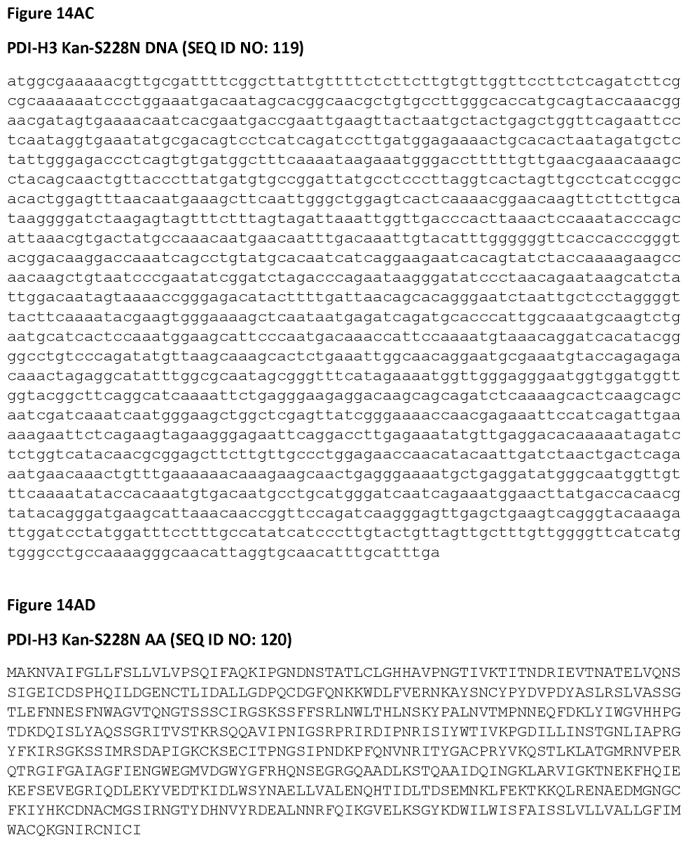

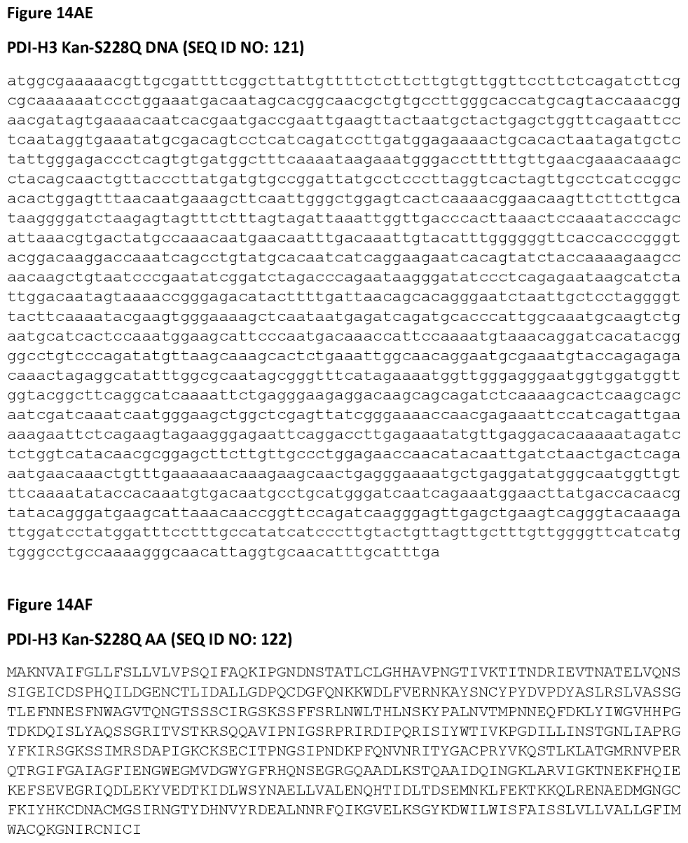

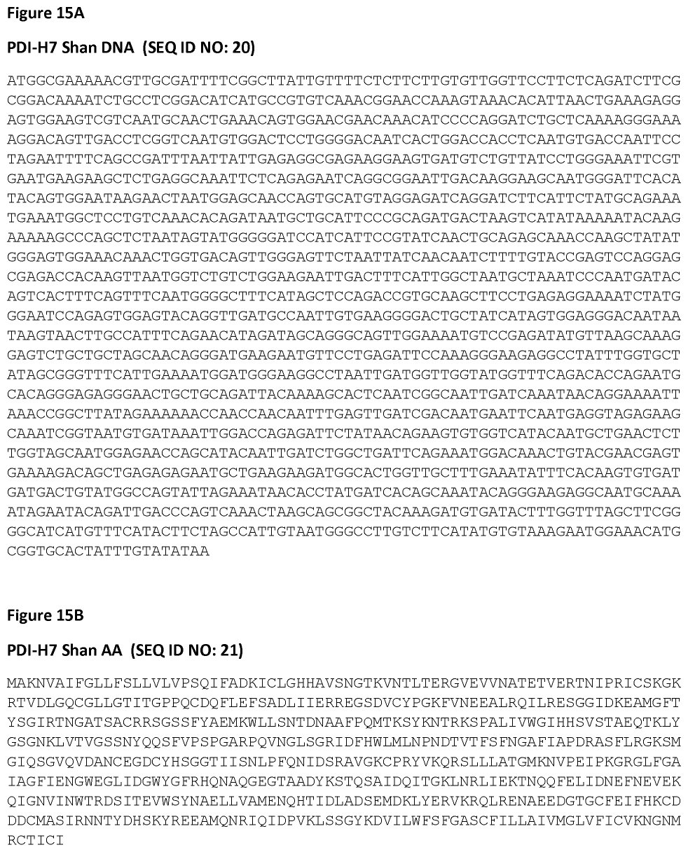

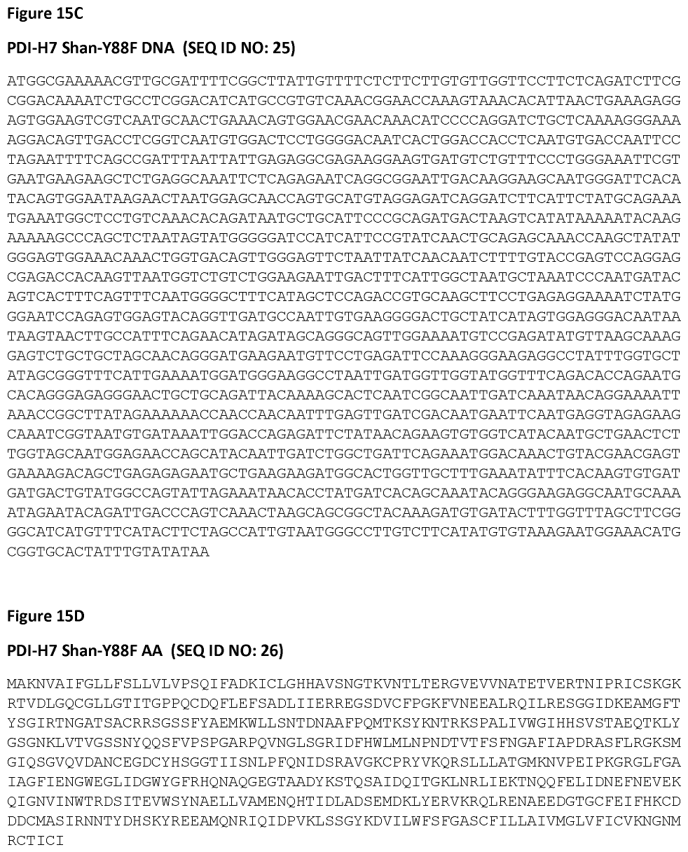

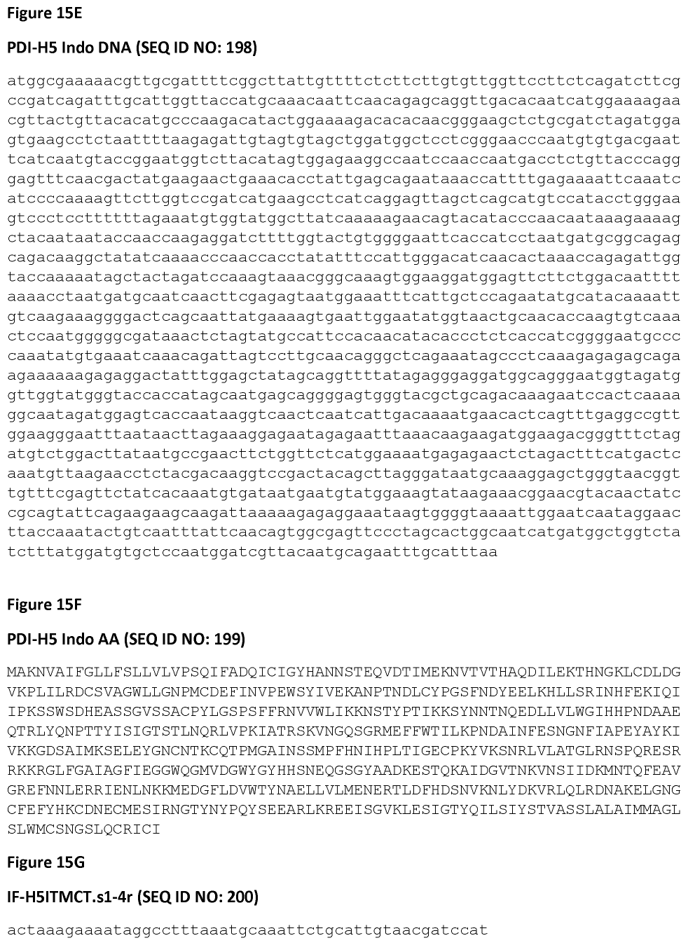

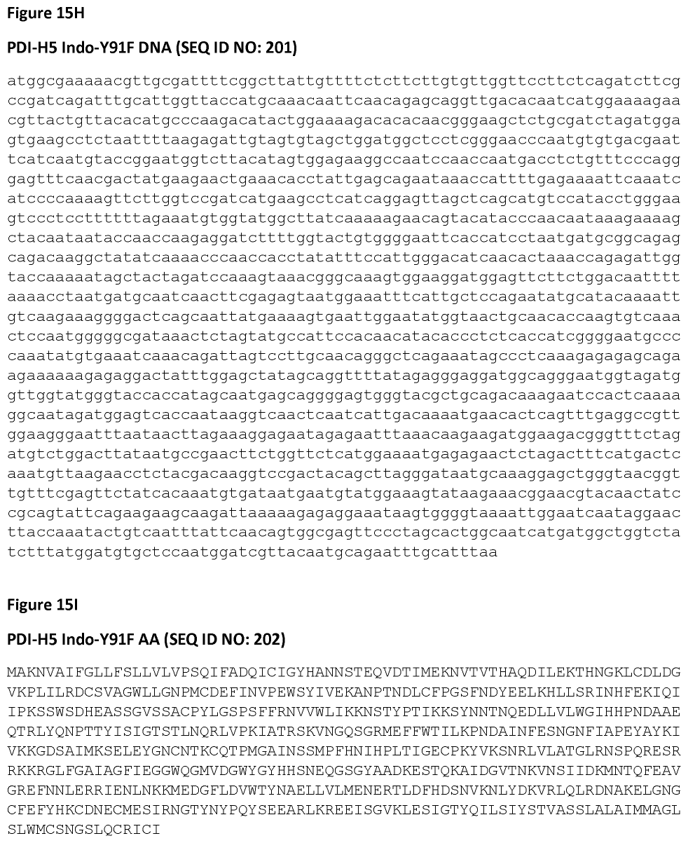

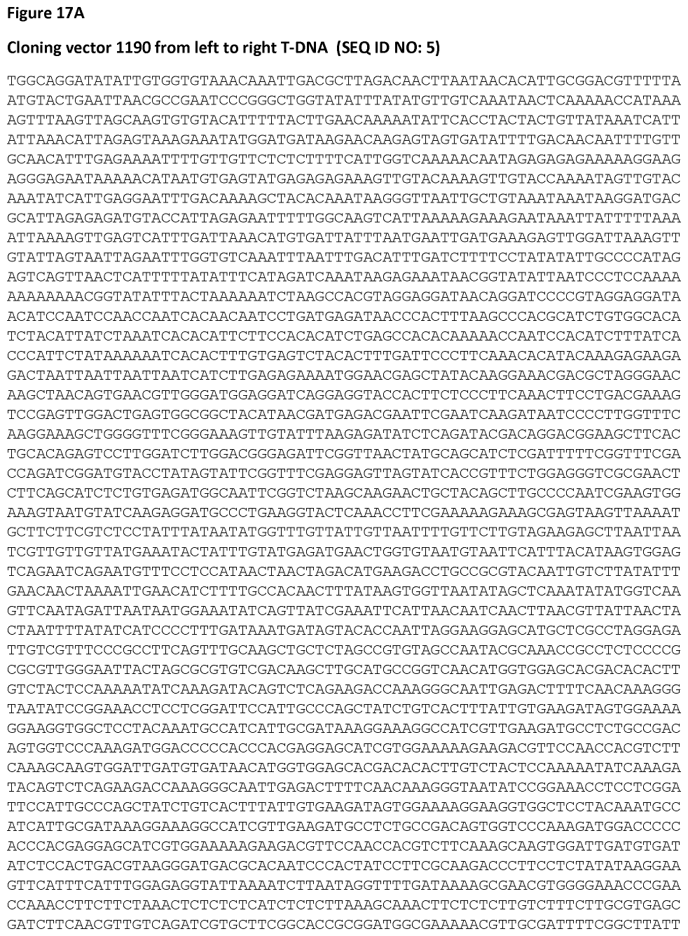

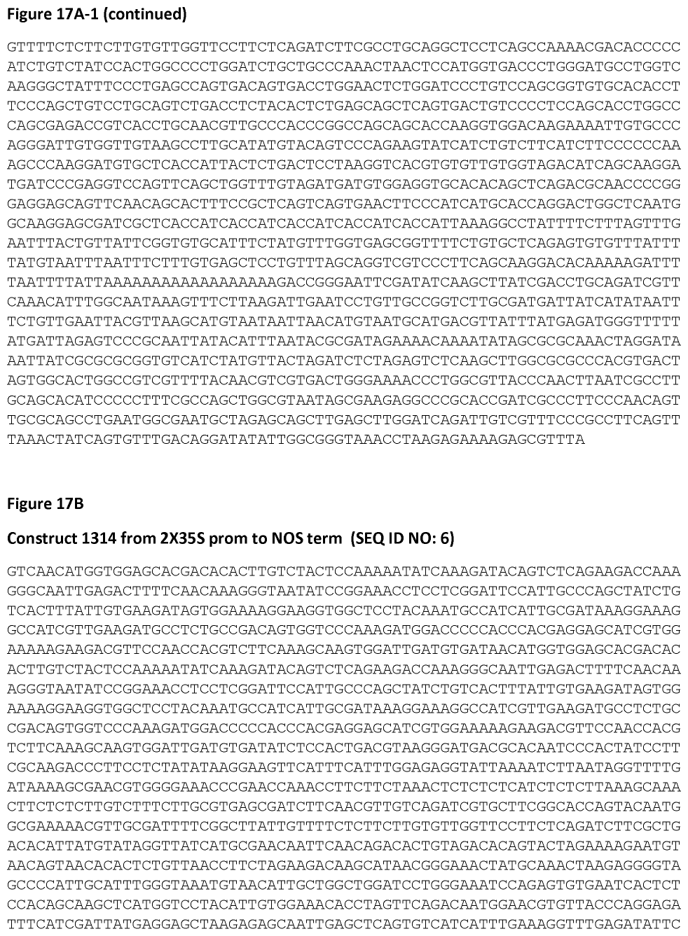

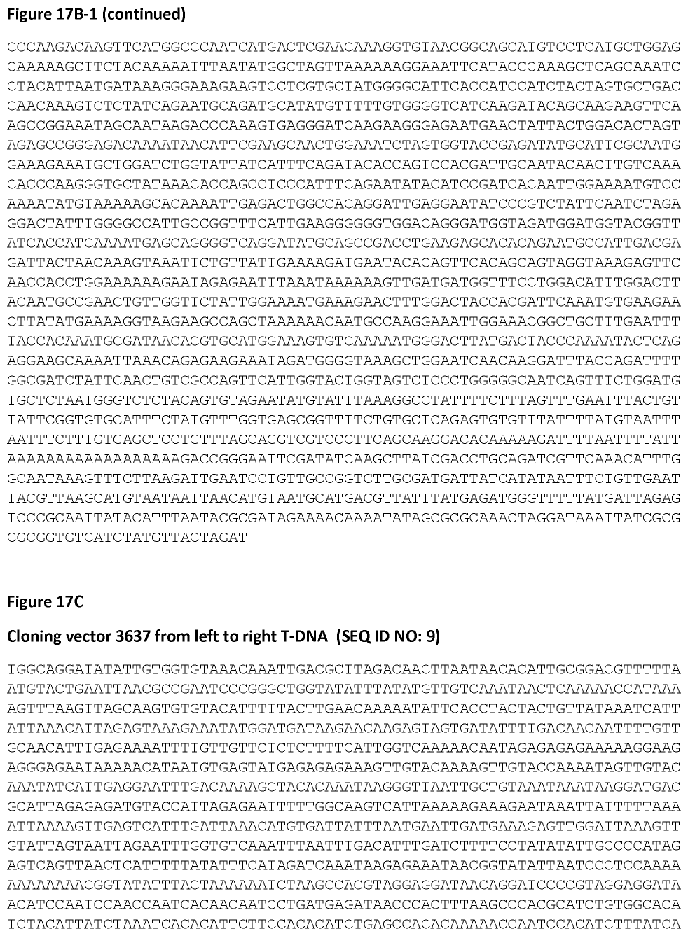

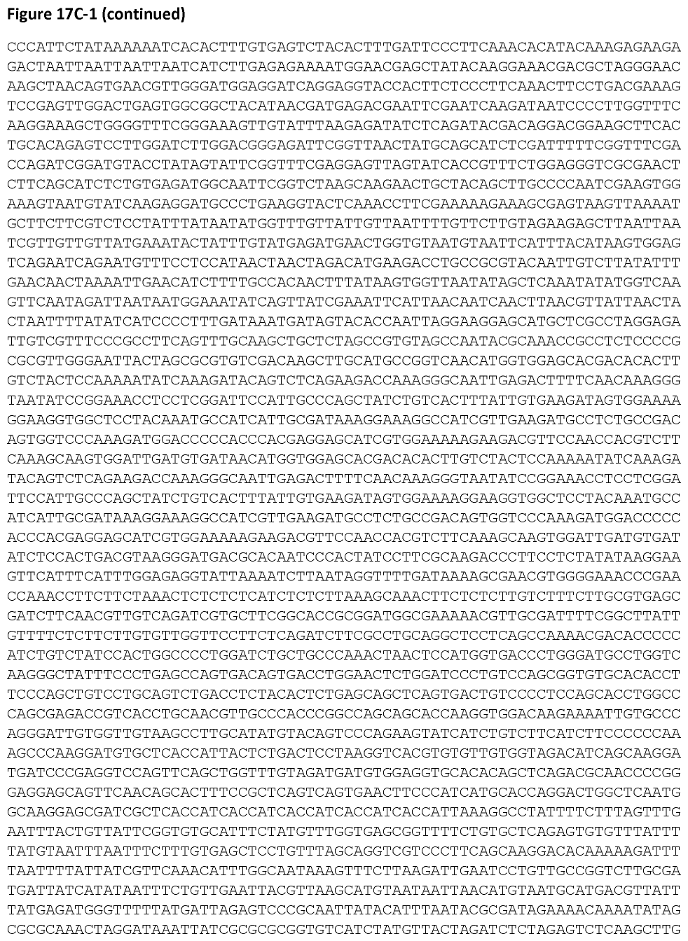

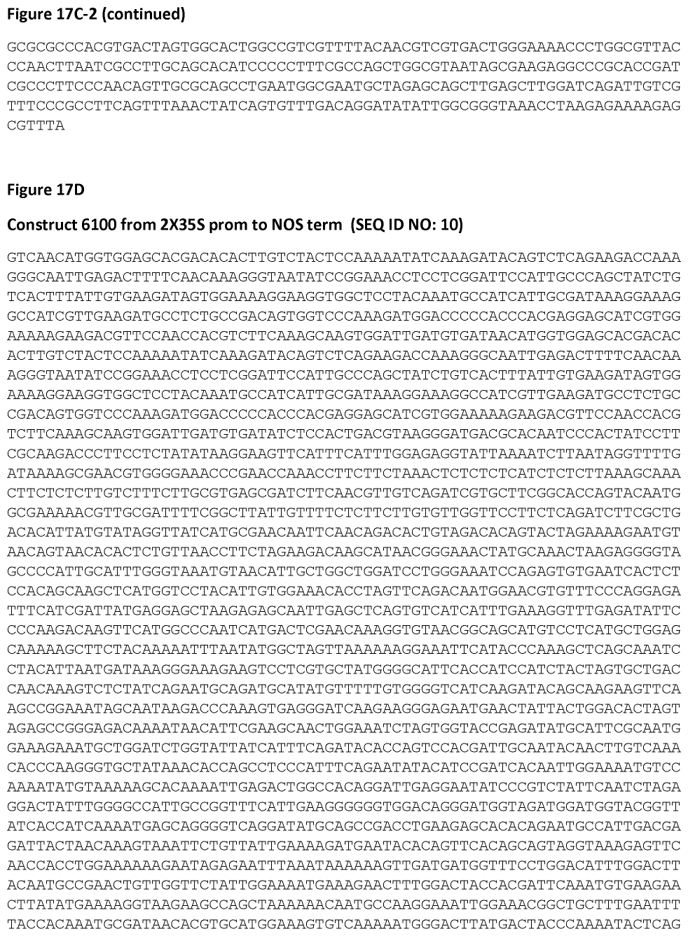

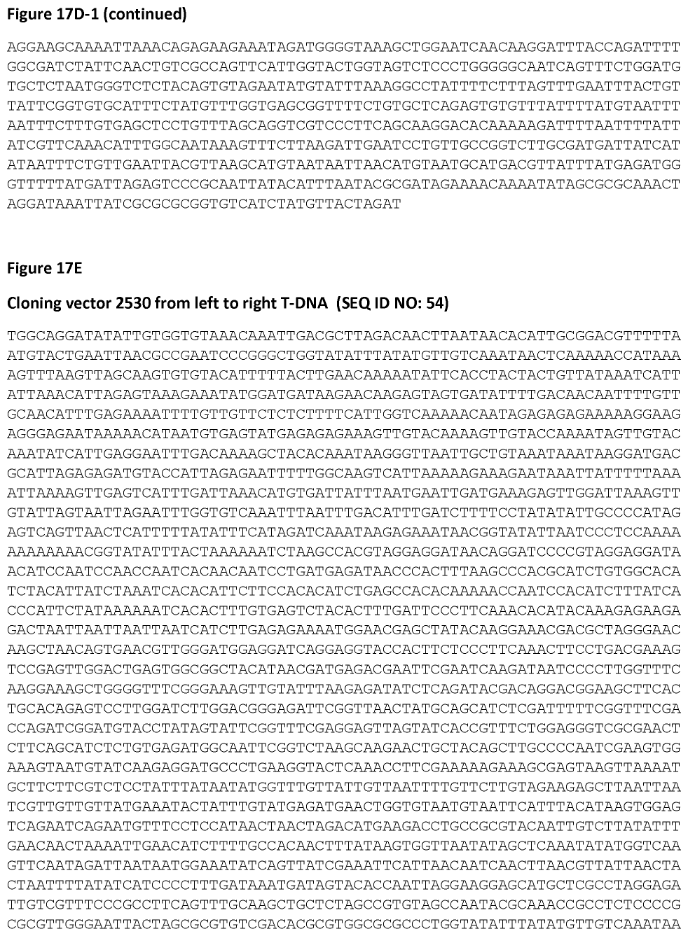

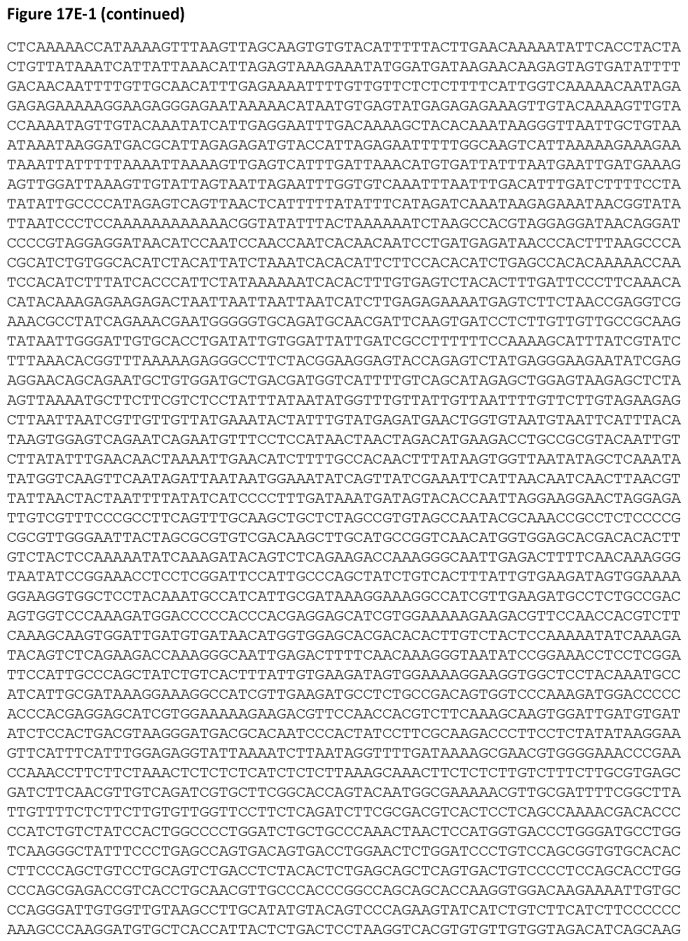

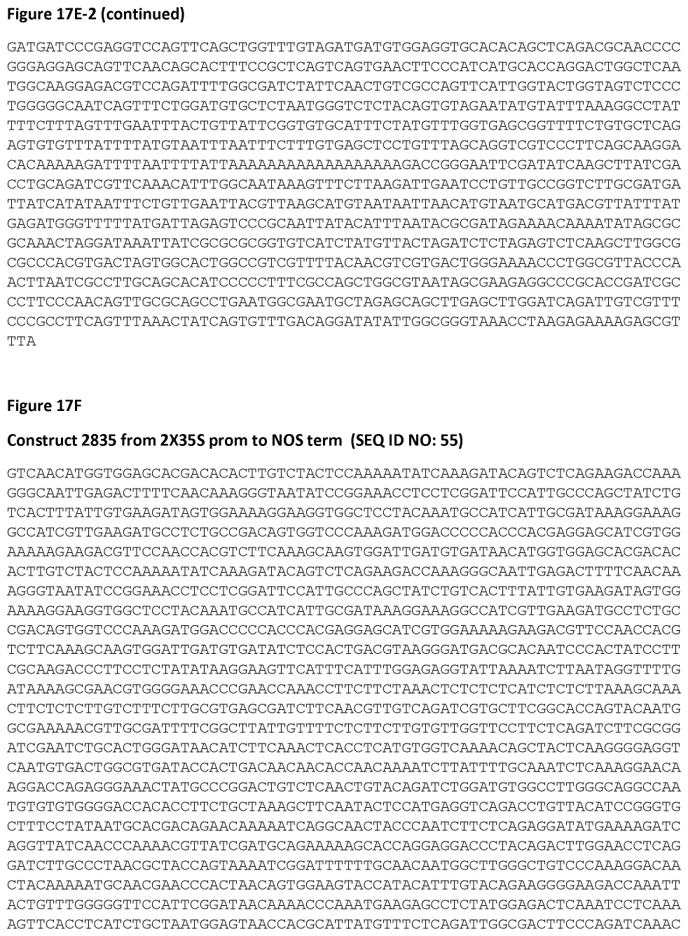

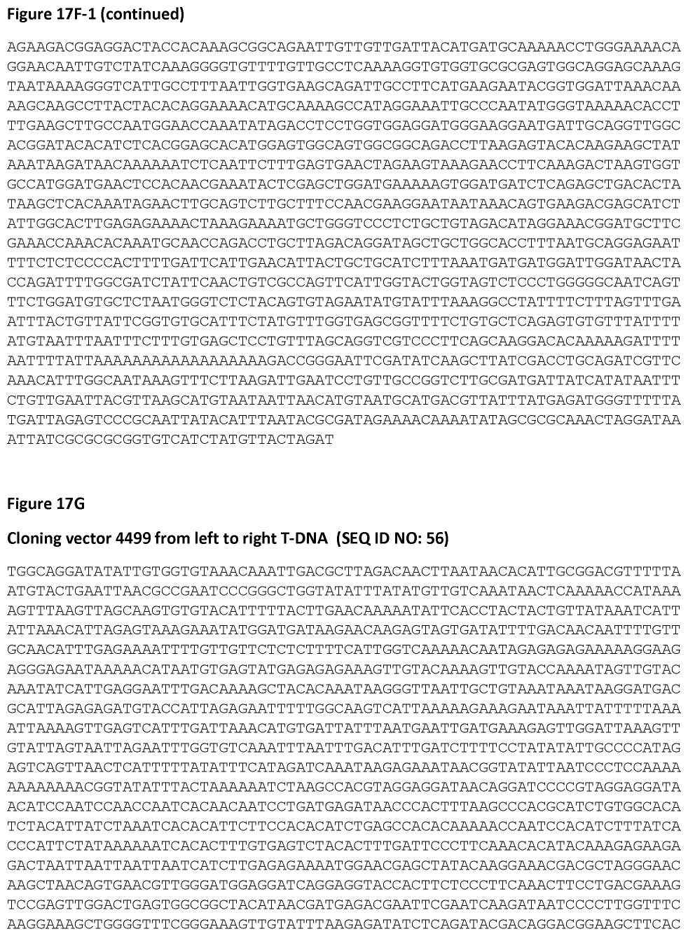

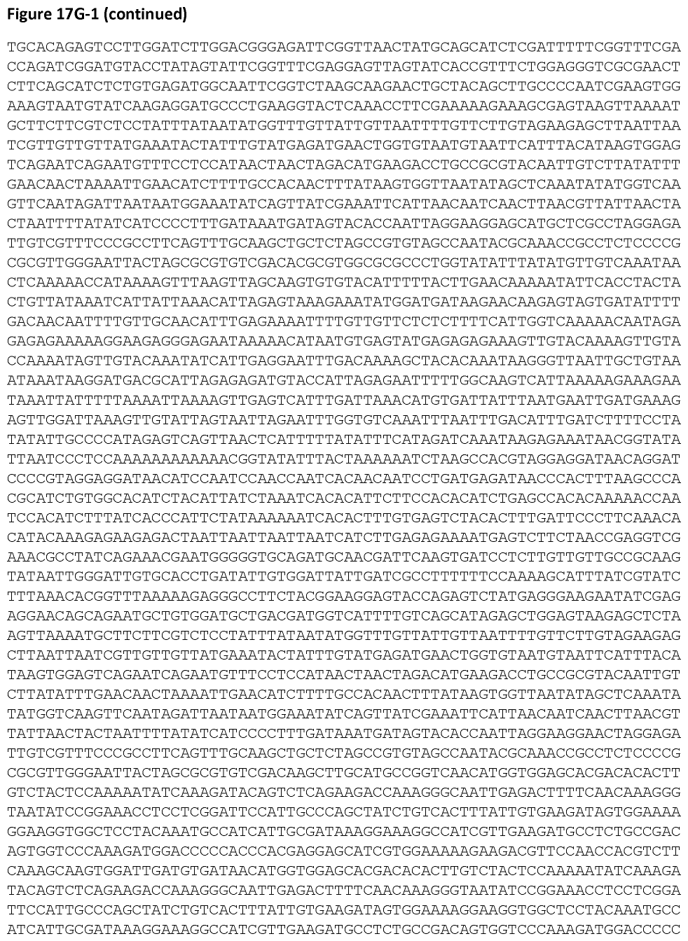

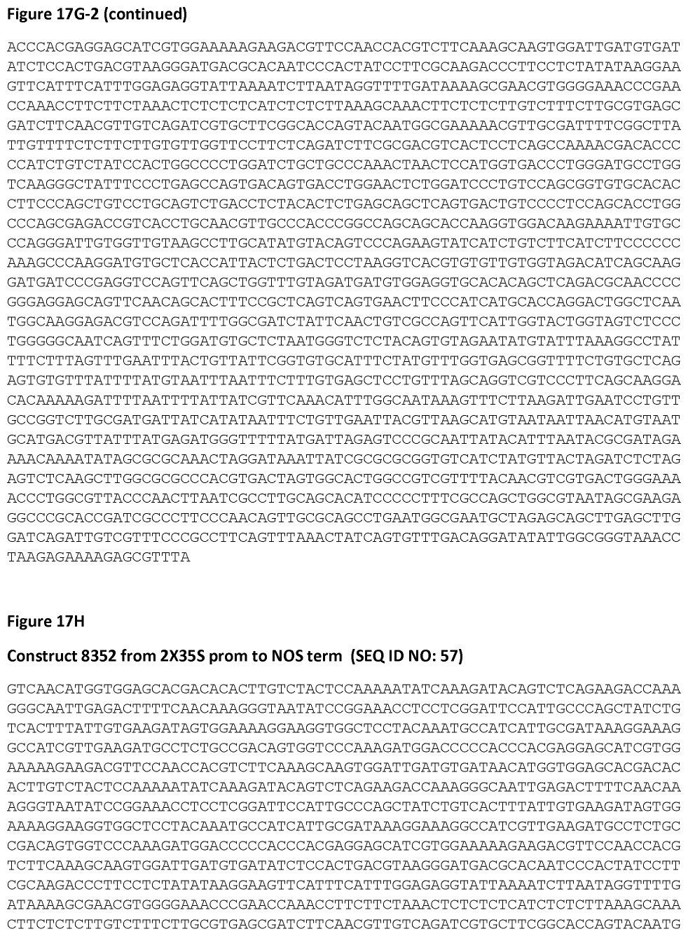

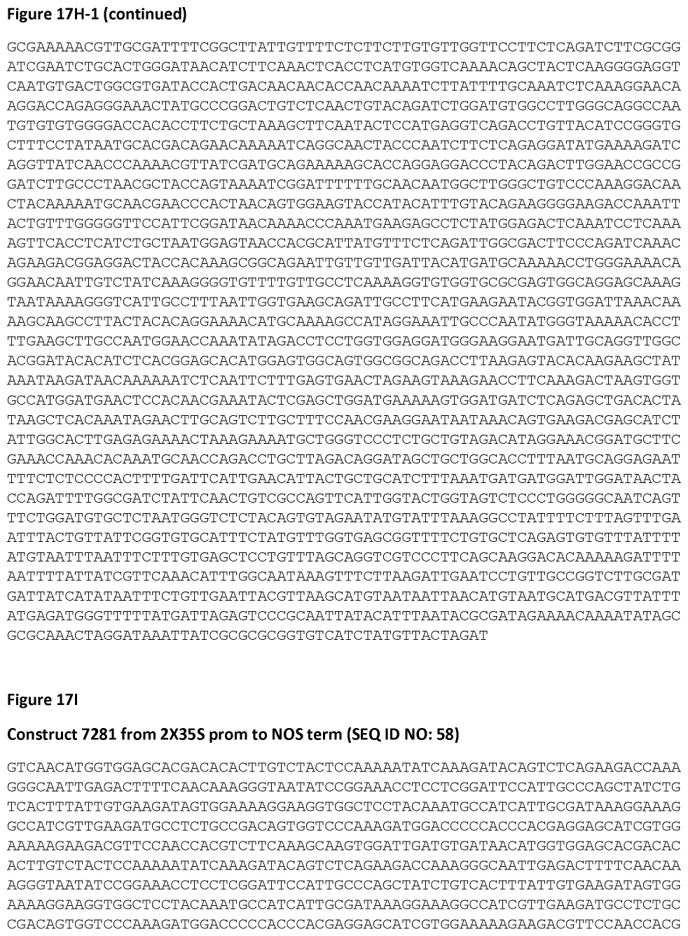

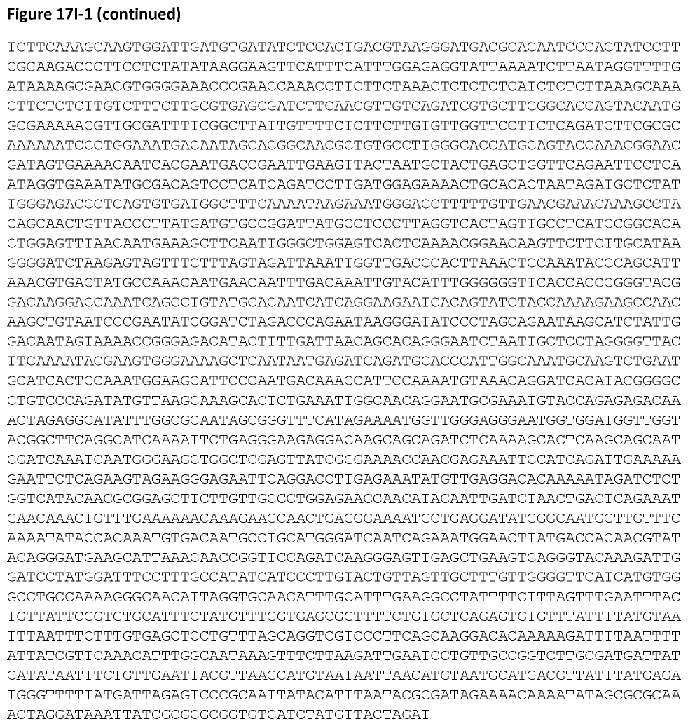

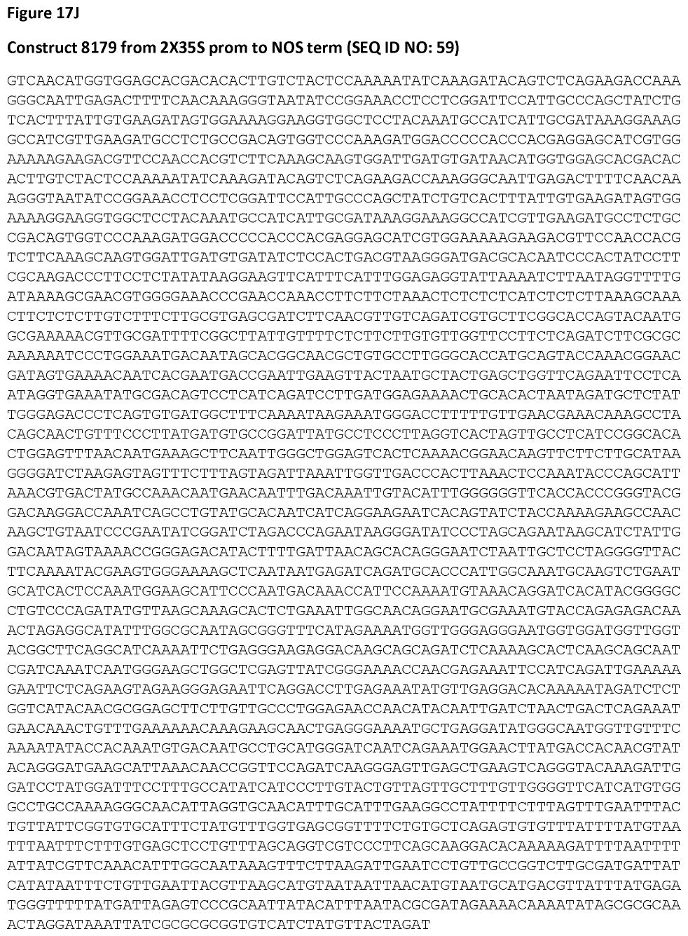

TABLE 3
Summary of sequences

| SEQ ID NO: | Name | FIG./Table |
|---|---|---|
| SEQ ID NO: 1 | PDI-H1 Cal/7/09 DNA | FIG. 13A |
| SEQ ID NO: 2 | PDI-H1 Cal/7/09 AA | FIG. 13B |
| SEQ ID NO: 3 | IF-CPMV(fl5′UTR)_SpPDI.c | Tab. 4 |
| SEQ ID NO: 4 | IF-H1cTMCT.S1-4r | Tab. 4 |
| SEQ ID NO: 5 | Cloning vector 1190 from left to right T-DNA | FIG. 17A |
| SEQ ID NO: 6 | Construct 1314 from 2X35S prom to NOS term | FIG. 17B |
| SEQ ID NO: 7 | H1_Cal(Y91F).r | Tab. 4 |
| SEQ ID NO: 8 | H1_Cal(Y91F).c | Tab. 4 |
| SEQ ID NO: 9 | Cloning vector 3637 from left to right T-DNA | FIG. 17C |
| SEQ ID NO: 10 | Construct 6100 from 2X35S prom to NOS term | FIG. 17D |
| SEQ ID NO: 11 | PDI-H1 Cal-Y91F DNA | 1 FIG. 8C |
| SEQ ID NO: 12 | PDI-H1 Cal-Y91F AA | FIG. 13D |
| SEQ ID NO: 13 | A/Minnesota/41/19 (H3N2) | FIG. 1A |
| SEQ ID NO: 14 | B/Singapore/INFKK-16-0569/16 (Yamagata) | FIG. 1B |
| SEQ ID NO: 15 | B/Maryland/15/16 (Victoria) | FIG. 1B |
| SEQ ID NO: 16 | B/Victoria/705/18 (Victoria) | FIG. 1B |
| SEQ ID NO: 17 | B/Washington/12/19 (Victoria) | FIG. 1B |
| SEQ ID NO: 18 | B/Darwin/8/19 (Victoria) | FIG. 1B |
| SEQ ID NO: 19 | B/Darwin/20/19 (Victoria) | FIG. 1B |
| SEQ ID NO: 20 | PDI-H7 Shan DNA | FIG. 15A |
| SEQ ID NO: 21 | PDI-H7 Shan AA | FIG. 15B |
| SEQ ID NO: 22 | IF-H7Shang.r | Tab. 4 |
| SEQ ID NO: 23 | H7Shang(Y88F).c | Tab. 4 |
| SEQ ID NO: 24 | H7Shang(Y88F).r | Tab. 4 |
| SEQ ID NO: 25 | PDI-H7 Shan-Y88F DNA | FIG. 15C |
| SEQ ID NO: 26 | PDI-H7 Shan-Y88F AA | FIG. 15D |
| SEQ ID NO: 27 | PDI-B Phu/3073/2013 DNA | FIG. 16A |
| SEQ ID NO: 28 | PDI-B Phu/3073/2013 AA | FIG. 16B |
| SEQ ID NO: 29 | IF.HBPhu3073.c | Tab. 4 |
| SEQ ID NO: 30 | B_Phuket(S140A).c | Tab. 4 |
| SEQ ID NO: 31 | B_Phuket(S140A).r | Tab. 4 |
| SEQ ID NO: 32 | PDI-B Phu-S140A/3073/2013 (S140A) DNA | FIG. 16C |
| SEQ ID NO: 33 | PDI-B Phu-S140A/3073/2013 (S140A) AA | FIG. 16D |
| SEQ ID NO: 34 | B_Phuket(S142A).c | Tab. 4 |
| SEQ ID NO: 35 | B_Phuket(S142A).r | Tab. 4 |
| SEQ ID NO: 36 | PDI-B Phu-S142A DNA | FIG. 16E |
| SEQ ID NO: 37 | PDI-B Phu-S142A AA | FIG. 16F |
| SEQ ID NO: 38 | B_Phuket(G138A).c | Tab. 4 |
| SEQ ID NO: 39 | B_Phuket(G138A).r | Tab. 4 |
| SEQ ID NO: 40 | PDI-B Phu-G138A DNA | FIG. 16G |
| SEQ ID NO: 41 | PDI-B Phu-G138A AA | FIG. 16H |
| SEQ ID NO: 42 | B_Phuket(L203A).c | Tab. 4 |
| SEQ ID NO: 43 | B_Phuket(L203A).r | Tab. 4 |
| SEQ ID NO: 44 | PDI-B Phu-L203A DNA | FIG. 16I |
| SEQ ID NO: 45 | PDI-B Phu-L203A AA | FIG. 16J |
| SEQ ID NO: 46 | B_Phuket(D195G).c | Tab. 4 |
| SEQ ID NO: 47 | B_Phuket(D195G).r | Tab. 4 |
| SEQ ID NO: 48 | PDI-B Phu-D195G DNA | FIG. 16K |
| SEQ ID NO: 49 | PDI-B Phu-D195G AA | FIG. 16L |
| SEQ ID NO: 50 | B_Phuket(L203W).c | Tab. 4 |
| SEQ ID NO: 51 | B_Phuket(L203W).r | Tab. 4 |
| SEQ ID NO: 52 | PDI-B Phu-L203W DNA | FIG. 16M |
| SEQ ID NO: 53 | PDI-B Phu-L203W AA | FIG. 16N |
| SEQ ID NO: 54 | Cloning vector 2530 from left to right T-DNA | FIG. 17E |
| SEQ ID NO: 55 | Construct 2835 from 2X35S prom to NOS term | FIG. 17F |
| SEQ ID NO: 56 | Cloning vector 4499 from left to right T-DNA | FIG. 17G |
| SEQ ID NO: 57 | Construct 8352 from 2X35S prom to NOS term | FIG. 17H |
| SEQ ID NO: 58 | Construct 7281 from 2X35S prom to NOS term | FIG. 17I |
| SEQ ID NO: 59 | Construct 8179 from 2X35S prom to NOS term | FIG. 17J |
| SEQ ID NO: 60 | PDI-H3 Kan DNA | FIG. 14A |
| SEQ ID NO: 61 | PDI-H3 Kan AA | FIG. 14B |
| SEQ ID NO: 62 | IF-H3NewJer.c | Tab. 4 |
| SEQ ID NO: 63 | IF-H3_Swi_13.r | Tab. 4 |
| SEQ ID NO: 64 | PDI-H3 Kan-Y98F DNA | FIG. 14C |
| SEQ ID NO: 65 | PDI-H3 Kan-Y98F AA | FIG. 14D |
| SEQ ID NO: 66 | H3_Kansas(Y98F).c | Tab. 4 |
| SEQ ID NO: 67 | H3_Kansas(Y98F).r | Tab. 4 |
| SEQ ID NO: 68 | PDI-H3 Kan-Y98F + S136D DNA | FIG. 14E |
| SEQ ID NO: 69 | PDI-H3 Kan-Y98F + S136D AA | FIG. 14F |
| SEQ ID NO: 70 | H3Kansas(S136D).c | Tab. 4 |
| SEQ ID NO: 71 | H3Kansas(S136D).r | Tab. 4 |
| SEQ ID NO: 72 | PDI-H3 Kan-Y98F + S136N DNA | FIG. 14G |
| SEQ ID NO: 73 | PDI-H3 Kan-Y98F + S136N AA | FIG. 14H |
| SEQ ID NO: 74 | H3Kansas(S136N).c | Tab. 4 |
| SEQ ID NO: 75 | H3Kansas(S136N).r | Tab. 4 |
| SEQ ID NO: 76 | PDI-H3 Kan-Y98F + S137N DNA | FIG. 14I |
| SEQ ID NO: 77 | PDI-H3 Kan-Y98F + S137N AA | FIG. 14J |
| SEQ ID NO: 78 | H3Kansas(S137N).c | Tab. 4 |
| SEQ ID NO: 79 | H3Kansas(S137N).r | Tab. 4 |
| SEQ ID NO: 80 | PDI-H3 Kan-Y98F + D190G DNA | FIG. 14K |
| SEQ ID NO: 81 | PDI-H3 Kan-Y98F + D190G AA | FIG. 14L |
| SEQ ID NO: 82 | H3Kansas(D190G).c | Tab. 4 |
| SEQ ID NO: 83 | H3Kansas(D190G).r | Tab. 4 |
| SEQ ID NO: 84 | PDI-H3 Kan-Y98F + D190K DNA | FIG. 14M |
| SEQ ID NO: 85 | PDI-H3 Kan-Y98F + D190K AA | FIG. 14N |
| SEQ ID NO: 86 | H3Kansas(D190K).c | Tab. 4 |
| SEQ ID NO: 87 | H3Kansas(D190K).r | Tab. 4 |
| SEQ ID NO: 88 | PDI-H3 Kan-Y98F + R222W DNA | FIG. 14O |
| SEQ ID NO: 89 | PDI-H3 Kan-Y98F + R222W AA | FIG. 14P |
| SEQ ID NO: 90 | H3Kansas(R222W).c | Tab. 4 |
| SEQ ID NO: 91 | H3Kansas(R222W).r | Tab. 4 |
| SEQ ID NO: 92 | PDI-H3 Kan-Y98F + S228N DNA | FIG. 14Q |
| SEQ ID NO: 93 | PDI-H3 Kan-Y98F + S228N AA | FIG. 14R |
| SEQ ID NO: 94 | H3Kansas(S228N).c | Tab. 4 |
| SEQ ID NO: 95 | H3Kansas(S228N).r | Tab. 4 |
| SEQ ID NO: 96 | PDI-H3 Kan-S228Q DNA | FIG. 14S |
| SEQ ID NO: 97 | PDI-H3 Kan-S228Q AA | FIG. 14T |
| SEQ ID NO: 98 | H3Kansas(S228Q).c | Tab. 4 |
| SEQ ID NO: 99 | H3Kansas(S228Q).r | Tab. 4 |
| SEQ ID NO: 100 | PDI-H1 Idaho DNA | FIG. 13E |
| SEQ ID NO: 101 | PDI-H1 Idaho AA | FIG. 13F |
| SEQ ID NO: 102 | IF-H1_Cal-7-09.c | Tab. 4 |
| SEQ ID NO: 103 | IF-H1cTMCT.s1-4r | Tab. 4 |
| SEQ ID NO: 104 | PDI-H1 Idaho-Y91F DNA | FIG. 13G |
| SEQ ID NO: 105 | PDI-H1 Idaho-Y91F AA | FIG. 13H |
| SEQ ID NO: 106 | H1_Idaho(Y91F).c | Tab. 4 |
| SEQ ID NO: 107 | H1_Idaho(Y91F).r | Tab. 4 |
| SEQ ID NO: 108 | A/Egypt/NO4915/14 (H5N1) | FIG. 1A |
| SEQ ID NO: 109 | A/Hangzhou/1/13 (H7N9) | FIG. 1A |
| SEQ ID NO: 110 | transmembrane domain consensus sequence | — |
| SEQ ID NO: 111 | PDI-H3 Kan-S136D DNA | FIG. 14U |
| SEQ ID NO: 112 | PDI-H3 Kan-S136D AA | FIG. 14V |
| SEQ ID NO: 113 | PDI-H3 Kan-S136N DNA | FIG. 14W |
| SEQ ID NO: 114 | PDI-H3 Kan-S136N AA | FIG. 14X |
| SEQ ID NO: 115 | PDI-H3 Kan-D190K DNA | FIG. 14Y |
| SEQ ID NO: 116 | PDI-H3 Kan-D190K AA | FIG. 14Z |
| SEQ ID NO: 117 | PDI-H3 Kan-R222W DNA | FIG. 14AA |
| SEQ ID NO: 118 | PDI-H3 Kan-R222W AA | FIG. 14AB |
| SEQ ID NO: 119 | PDI-H3 Kan-S228N DNA | FIG. 14AC |
| SEQ ID NO: 120 | PDI-H3 Kan-S228N AA | FIG. 14AD |
| SEQ ID NO: 121 | PDI-H3 Kan-S228Q DNA | FIG. 14AE |
| SEQ ID NO: 122 | PDI-H3 Kan-S228Q AA | FIG. 14AF |
| SEQ ID NO: 123 | PDI-B Sing DNA | FIG. 16O |
| SEQ ID NO: 124 | PDI-B Sing AA | FIG. 16P |
| SEQ ID NO: 125 | PDI-B Sing-G138A DNA | FIG. 16Q |
| SEQ ID NO: 126 | PDI-B Sing-G138A AA | FIG. 16R |
| SEQ ID NO: 127 | PDI-B Sing-S140A DNA | FIG. 16S |
| SEQ ID NO: 128 | PDI-B Sing-S140A AA | FIG. 16T |
| SEQ ID NO: 129 | PDI-B Sing-S142A DNA | FIG. 16U |
| SEQ ID NO: 130 | PDI-B Sing-S142A AA | FIG. 16V |
| SEQ ID NO: 131 | PDI-B Sing-D195G DNA | FIG. 16W |
| SEQ ID NO: 132 | PDI-B Sing-D195G AA | FIG. 16X |
| SEQ ID NO: 133 | PDI-B Sing-L203A DNA | FIG. 16Y |
| SEQ ID NO: 134 | PDI-B Sing-L203A AA | FIG. 16Z |
| SEQ ID NO: 135 | PDI-B Sing-L203W DNA | FIG. 16AA |
| SEQ ID NO: 136 | PDI-B Sing-L203W AA | FIG. 16AB |
| SEQ ID NO: 137 | PDI-B Mary DNA | FIG. 16AC |
| SEQ ID NO: 138 | PDI-B Mary AA | FIG. 16AD |
| SEQ ID NO: 139 | IF-B-Bris(nat).c | FIG. 16AE |
| SEQ ID NO: 140 | PDI-B Mary-G138A DNA | FIG. 16AF |
| SEQ ID NO: 141 | PDI-B Mary-G138A AA | FIG. 16AG |
| SEQ ID NO: 142 | PDI-B Mary-S140A DNA | FIG. 16AH |
| SEQ ID NO: 143 | PDI-B Mary-S140A AA | FIG. 16AI |
| SEQ ID NO: 144 | PDI-B Mary-S142A DNA | FIG. 16AJ |
| SEQ ID NO: 145 | PDI-B Mary-S142A AA | FIG. 16AK |
| SEQ ID NO: 146 | PDI-B Mary-D194G DNA | FIG. 16AL |
| SEQ ID NO: 147 | PDI-B Mary-D194G AA | FIG. 16AM |
| SEQ ID NO: 148 | PDI-B Mary-L202A DNA | FIG. 16AN |
| SEQ ID NO: 149 | PDI-B Mary-L202A AA | FIG. 16AO |
| SEQ ID NO: 150 | PDI-B Mary-L202W DNA | FIG. 16AP |
| SEQ ID NO: 151 | PDI-B Mary-L202W AA | FIG. 16AQ |
| SEQ ID NO: 152 | PDI-B Wash DNA | FIG. 16AR |
| SEQ ID NO: 153 | PDI-B Wash AA | FIG. 16AS |
| SEQ ID NO: 154 | PDI-B Wash-G138A DNA | FIG. 16AT |
| SEQ ID NO: 155 | PDI-B Wash-G138A AA | FIG. 16AU |
| SEQ ID NO: 156 | PDI-B Wash-S140A DNA | FIG. 16AV |
| SEQ ID NO: 157 | PDI-B Wash-S140A AA | FIG. 16AW |
| SEQ ID NO: 158 | PDI-B Wash-S142A DNA | FIG. 16AX |
| SEQ ID NO: 159 | PDI-B Wash-S142A AA | FIG. 16AY |
| SEQ ID NO: 160 | PDI-B Wash-D193G DNA | FIG. 16AZ |
| SEQ ID NO: 161 | PDI-B Wash-D193G AA | FIG. 16BA |
| SEQ ID NO: 162 | PDI-B Wash-L201A DNA | FIG. 16BB |
| SEQ ID NO: 163 | PDI-B Wash-L201A AA | FIG. 16BC |
| SEQ ID NO: 164 | PDI-B Wash-L201W DNA | FIG. 16BD |
| SEQ ID NO: 165 | PDI-B Wash-L201W AA | FIG. 16BE |
| SEQ ID NO: 180 | PDI-B Vic DNA | FIG. 16BF |
| SEQ ID NO: 181 | PDI-B Vic AA | FIG. 16BG |
| SEQ ID NO: 182 | PDI-B Vic-G138A DNA | FIG. 16BH |
| SEQ ID NO: 183 | PDI-B Vic-G138A AA | FIG. 16BI |
| SEQ ID NO: 184 | PDI-B Vic-S140A DNA | FIG. 16BJ |
| SEQ ID NO: 185 | PDI-B Vic-S140A AA | FIG. 16BK |
| SEQ ID NO: 186 | PDI-B Vic-S142A DNA | FIG. 16BL |
| SEQ ID NO: 187 | PDI-B Vic-S142A AA | FIG. 16BM |
| SEQ ID NO: 188 | PDI-B Vic-D193G DNA | FIG. 16BN |
| SEQ ID NO: 189 | PDI-B Vic-D193G AA | FIG. 16BO |
| SEQ ID NO: 190 | PDI-B Vic-L201A DNA | FIG. 16BP |
| SEQ ID NO: 191 | PDI-B Vic-L201A AA | FIG. 16BQ |
| SEQ ID NO: 192 | PDI-B Vic-L201W DNA | FIG. 16BR |
| SEQ ID NO: 193 | PDI-B Vic-L201W AA | FIG. 16BS |
| SEQ ID NO: 194 | PDI-H1 Bris DNA | FIG. 13I |
| SEQ ID NO: 195 | PDI-H1 Bris AA | FIG. 13J |
| SEQ ID NO: 196 | PDI-H1 Bris-Y98F DNA | FIG. 13K |
| SEQ ID NO: 197 | PDI-H1 Bris-Y98F AA | FIG. 13L |
| SEQ ID NO: 198 | PDI-H5 Indo DNA | FIG. 15E |
| SEQ ID NO: 199 | PDI-H5 Indo AA | FIG. 15F |
| SEQ ID NO: 200 | IF-H5ITMCT.s1-4r | FIG. 15G |
| SEQ ID NO: 201 | PDI-H5 Indo-Y91F DNA | FIG. 15H |
| SEQ ID NO: 202 | PDI-H5 Indo-Y91F AA | FIG. 15I |
| SEQ ID NO: 203 | Reference sequence H1 (H1 A/California/07/2009) | FIG. 16BT |
| SEQ ID NO: 204 | Reference sequence H3 (H3 A/Kansas/14/2017) | FIG. 16BU |
| SEQ ID NO: 205 | Reference sequence H5 (A/Indonesia/05/2005) | FIG. 16BV |
| SEQ ID NO: 206 | Reference sequence H7 (H7 A/Shanghai/2/2013) | FIG. 16BW |
| SEQ ID NO: 207 | Reference sequence B (B/Phuket/3073/2013) | FIG. 16BX |
| SEQ ID NO: 208 | Reference sequence B (B/Maryland/15/2016) | FIG. 16BY |
| SEQ ID NO: 209 | Reference sequence B (B/Victoria/705/2018) | FIG. 16BZ |

The present invention will be further illustrated in the following examples.

### Example 1: Constructs

The influenza HA constructs were produced using techniques well known within the art. For example H1 A-California-07-09 HA, H1 A-California-7-09 (Y91F) HA, H3 A-Kansas-14-2017 HA, B-Phuket-3073-2013 HA and B-Phuket-3073-2013(S140A) HA were cloned as described below. Other modified HA were obtained using similar techniques and the HA sequences primers, templates and products are described below. A summary of the wildtype and mutated HA proteins, primers, templates, accepting vectors and products is provided in Tables 4 and 5 below.

### Example 1.1: 2X35S/CPMV 160/PDISP-HA0 H1 A-California-7-09/NOS (Construct Number 1314)

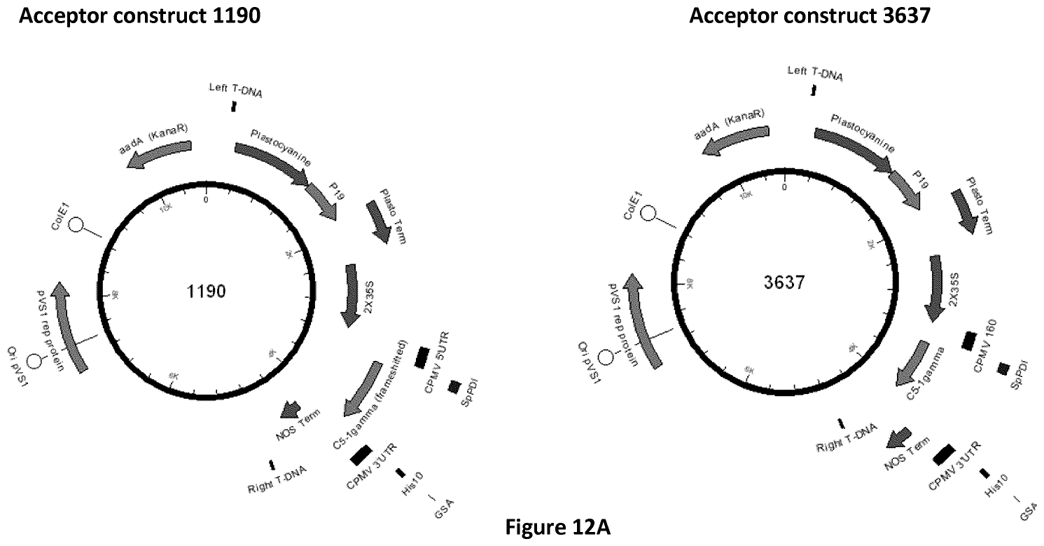

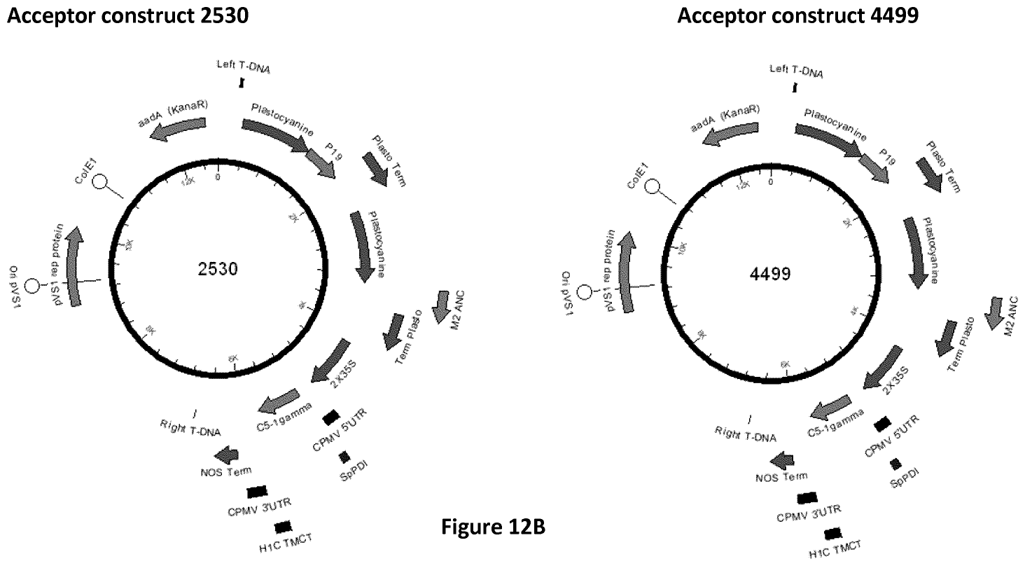

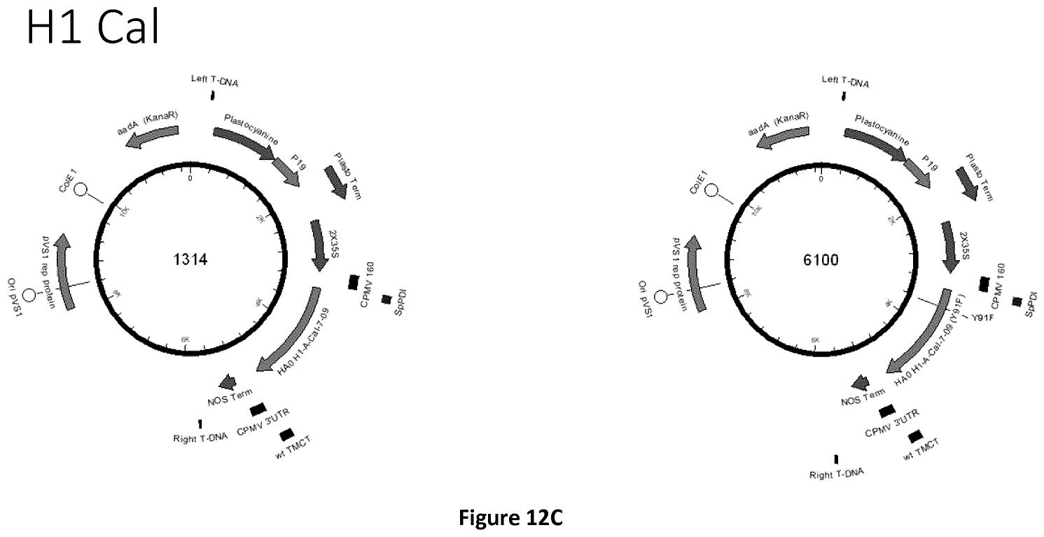

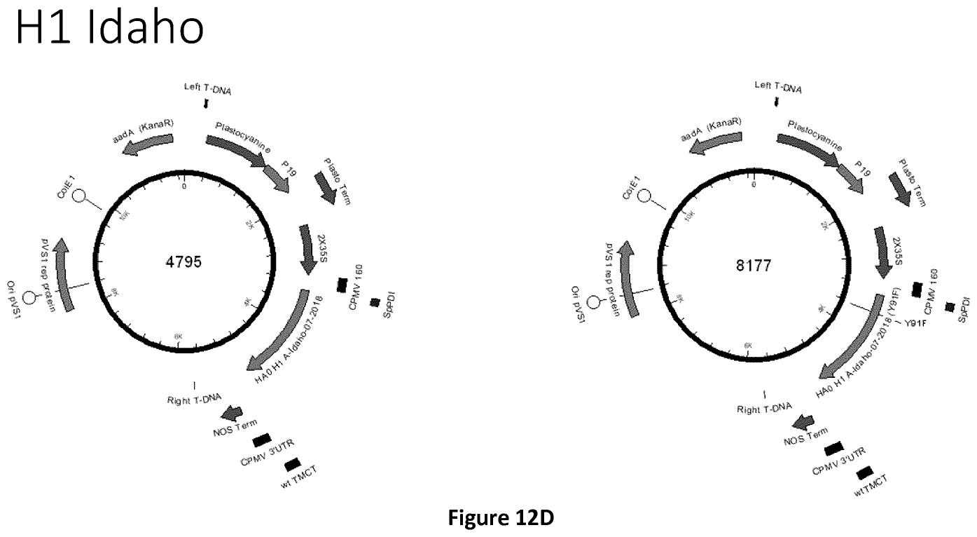

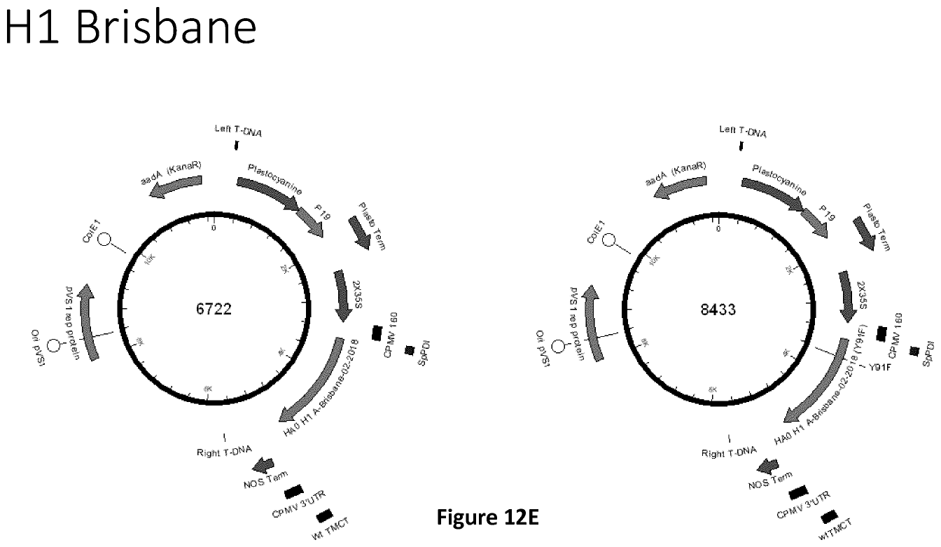

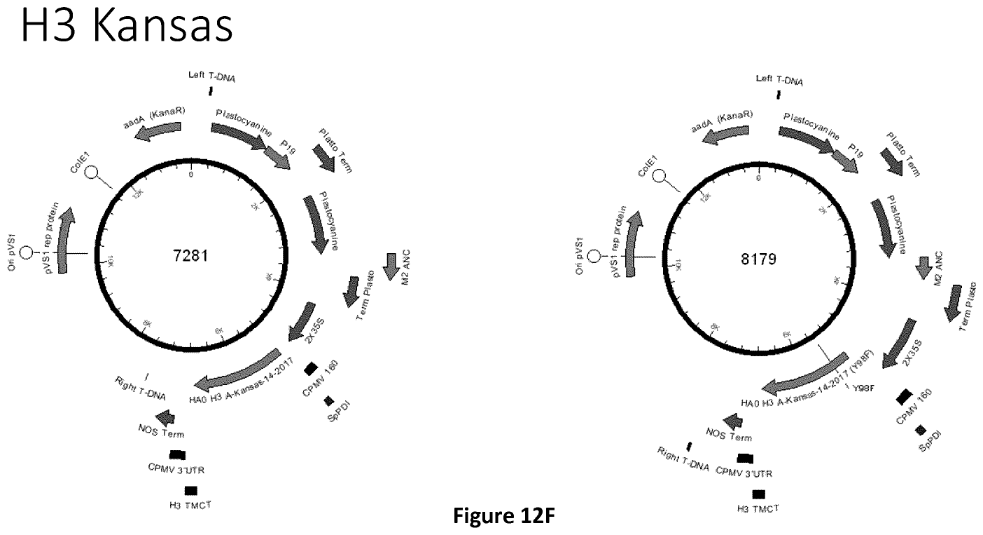

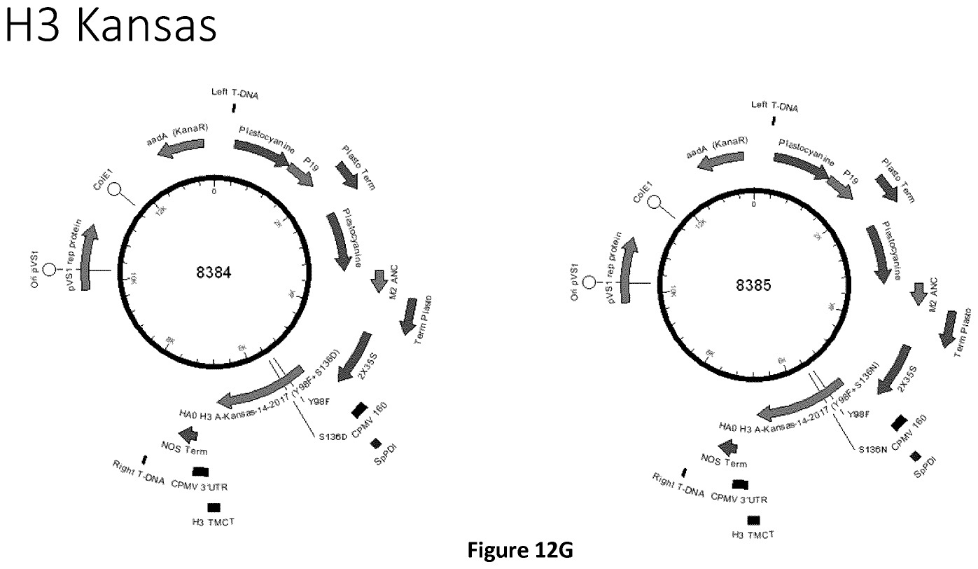

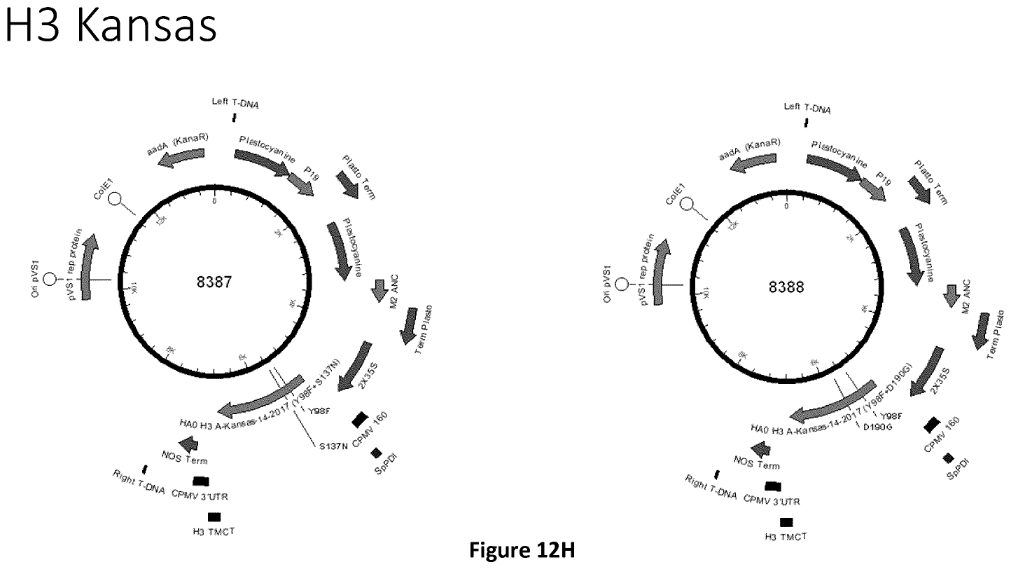

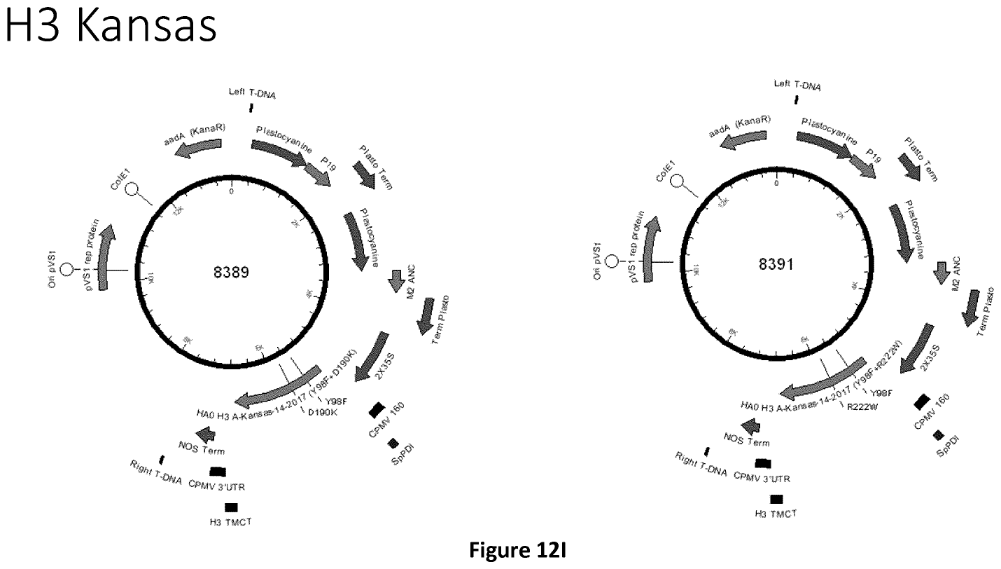

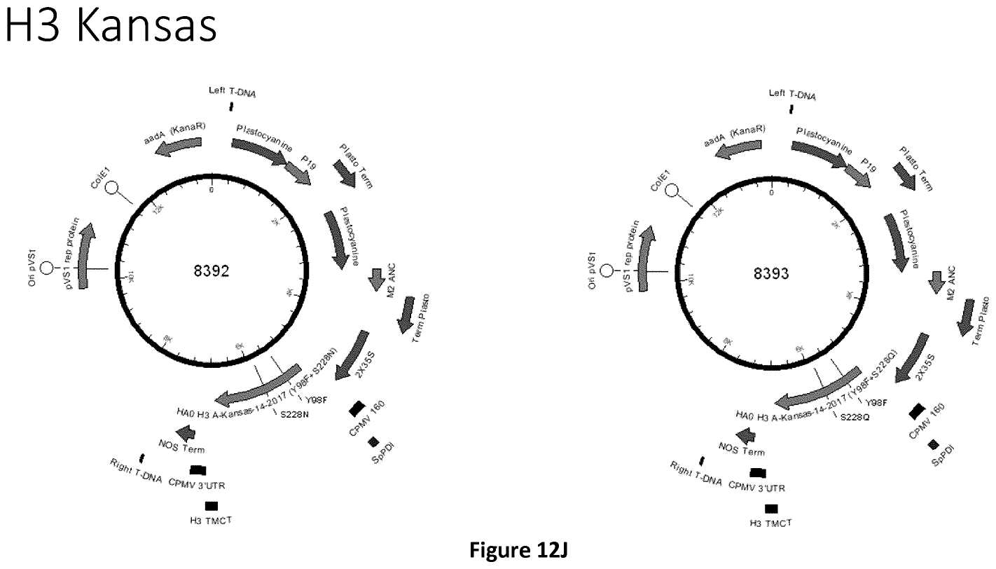

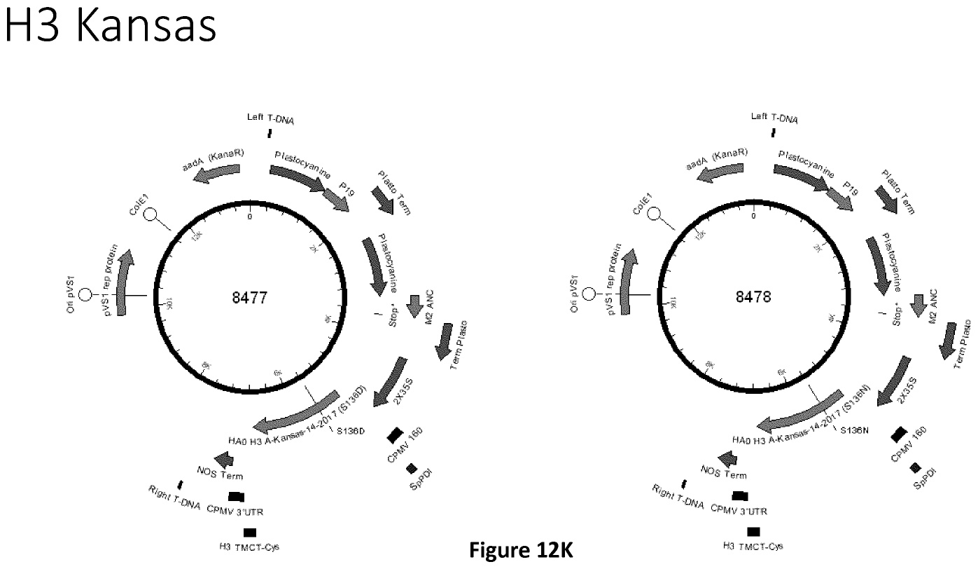

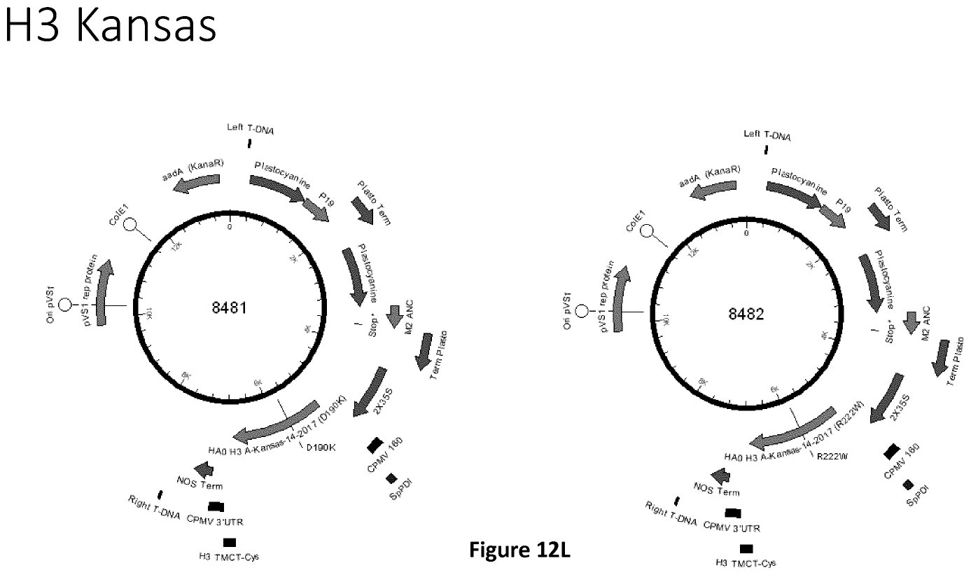

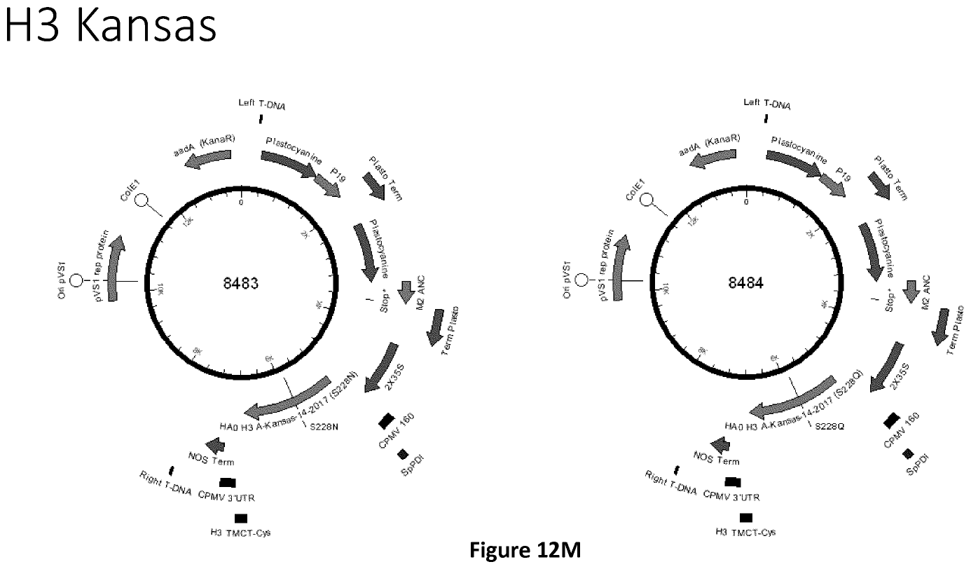

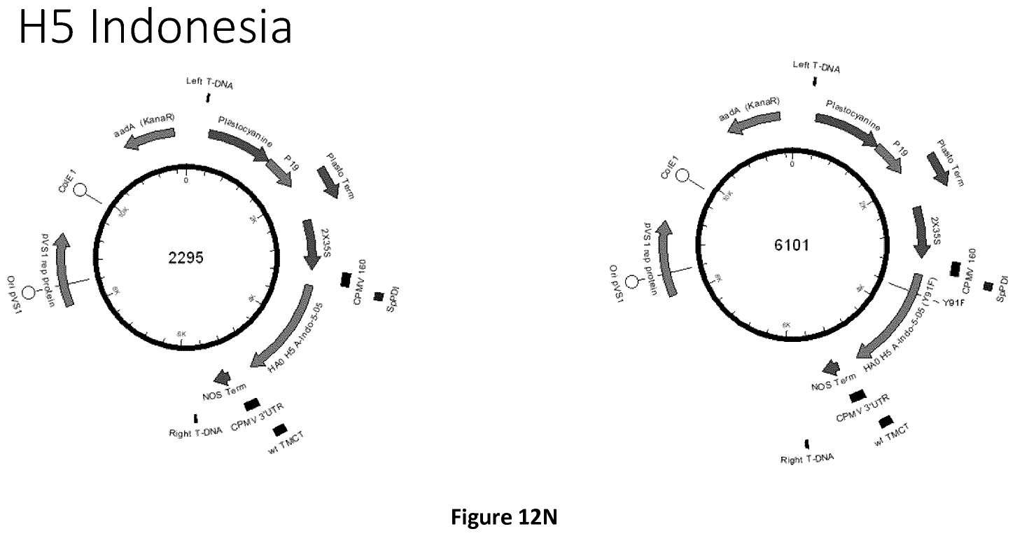

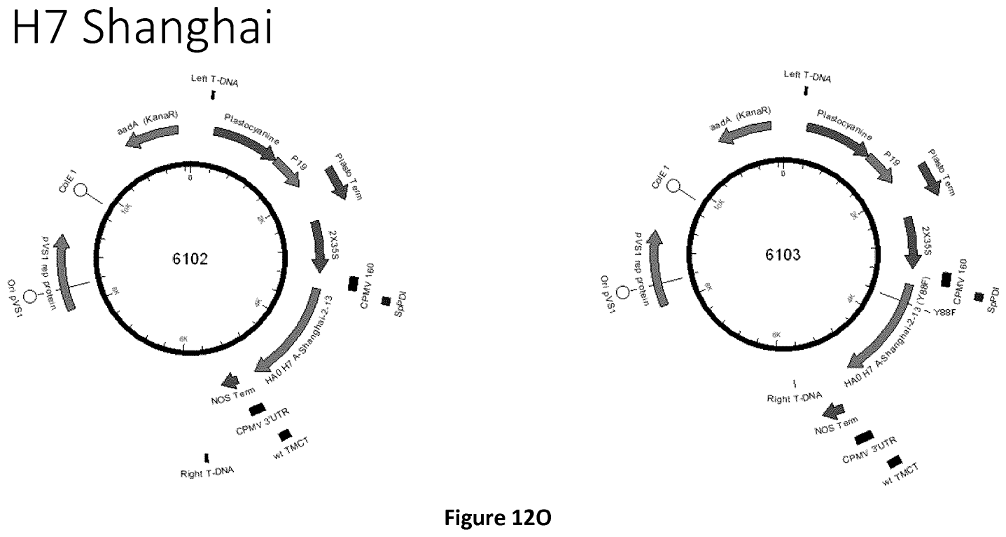

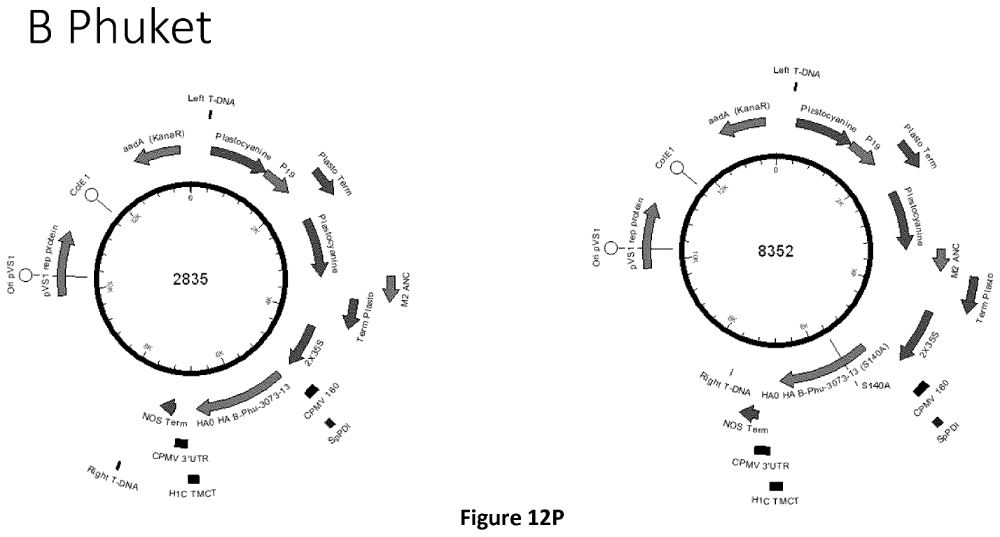

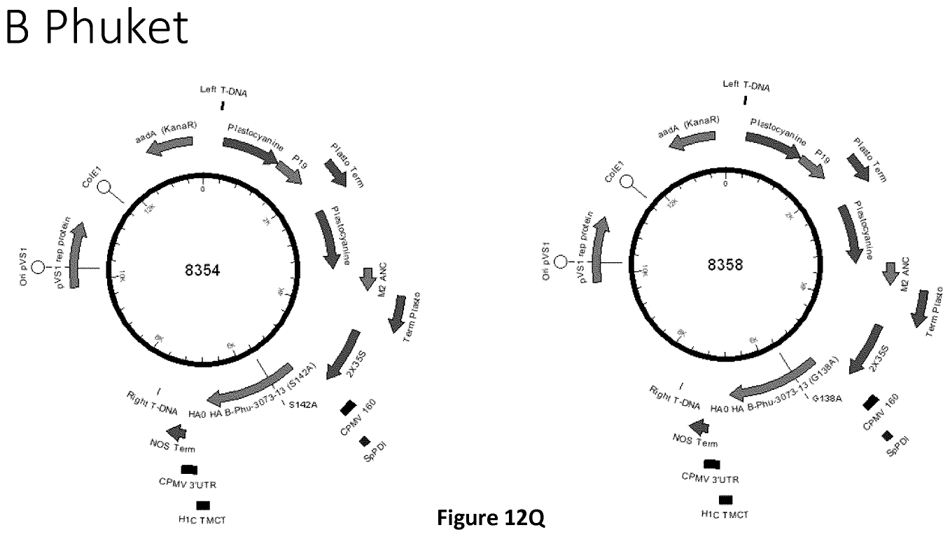

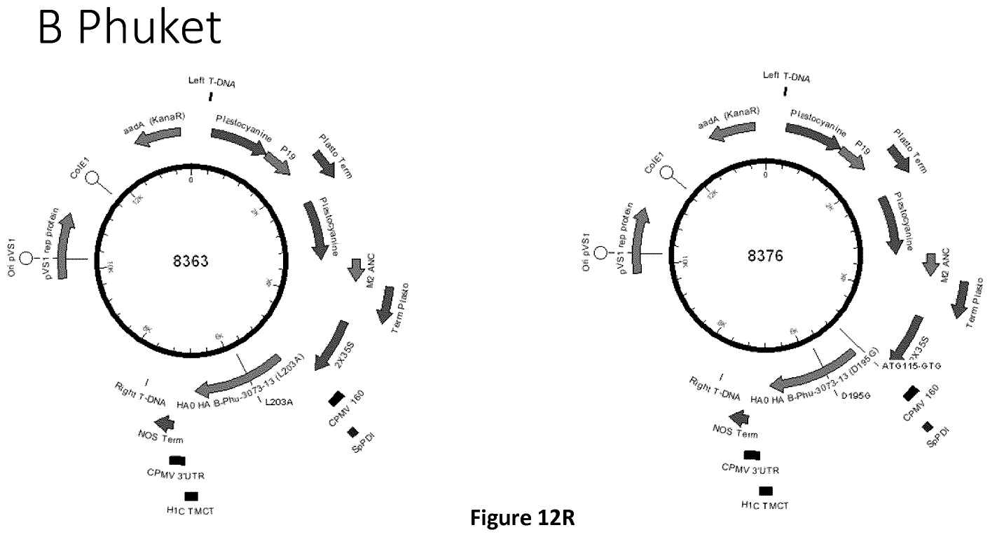

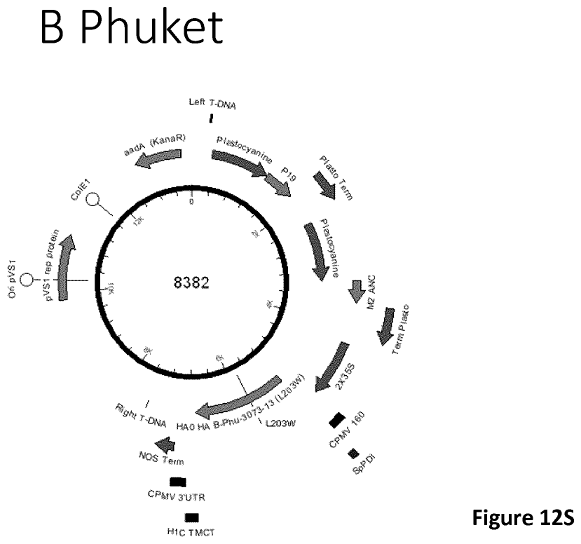

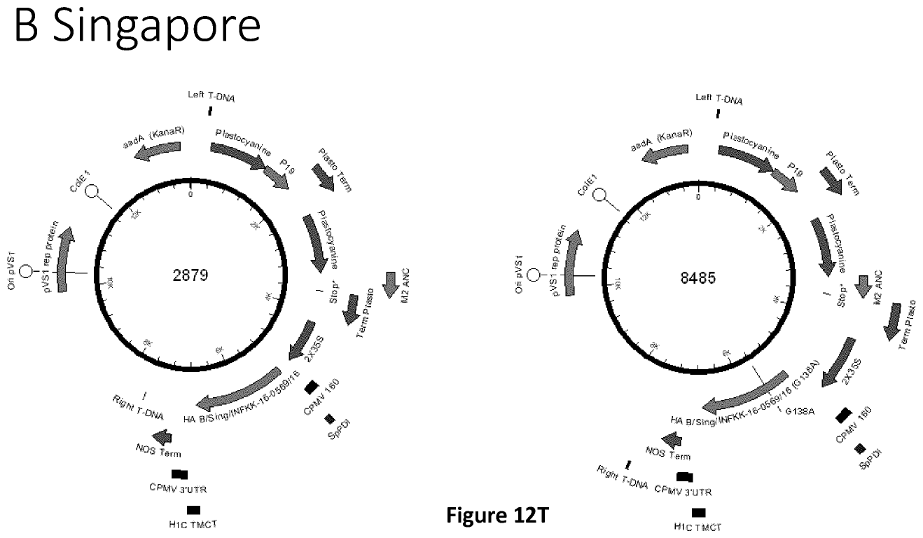

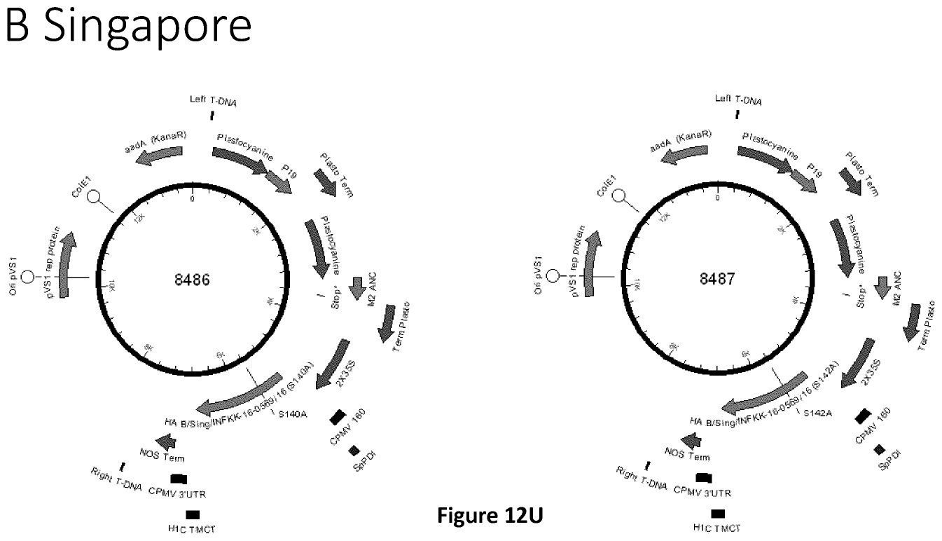

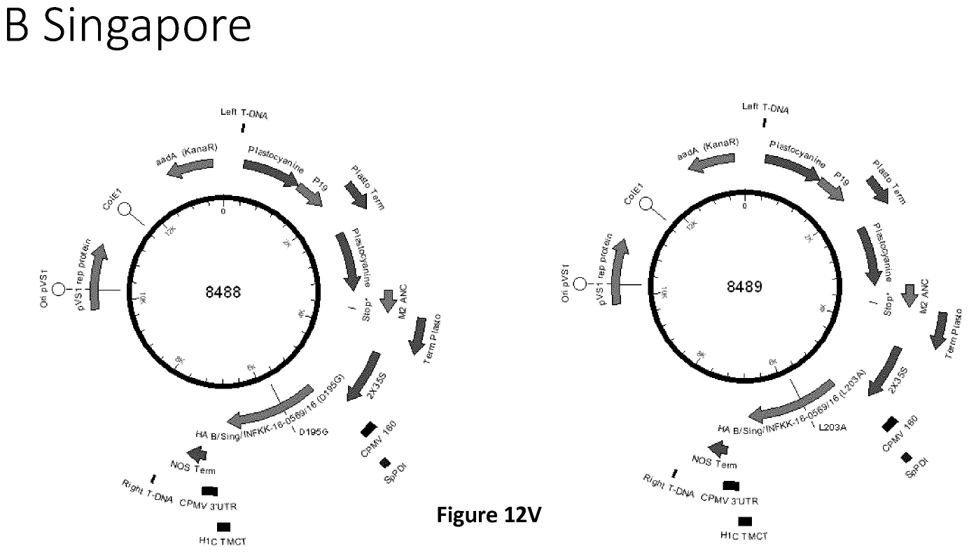

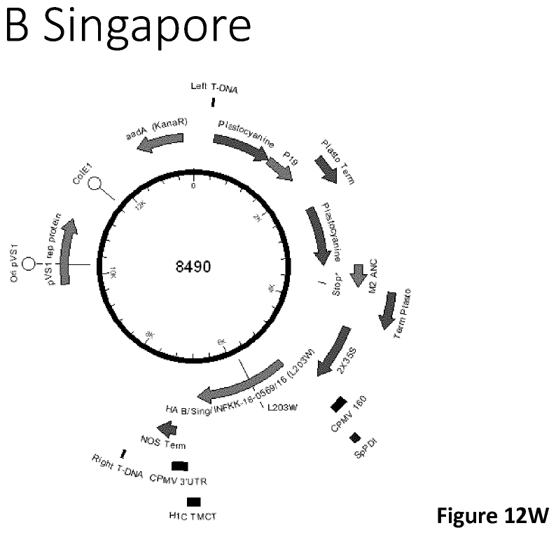

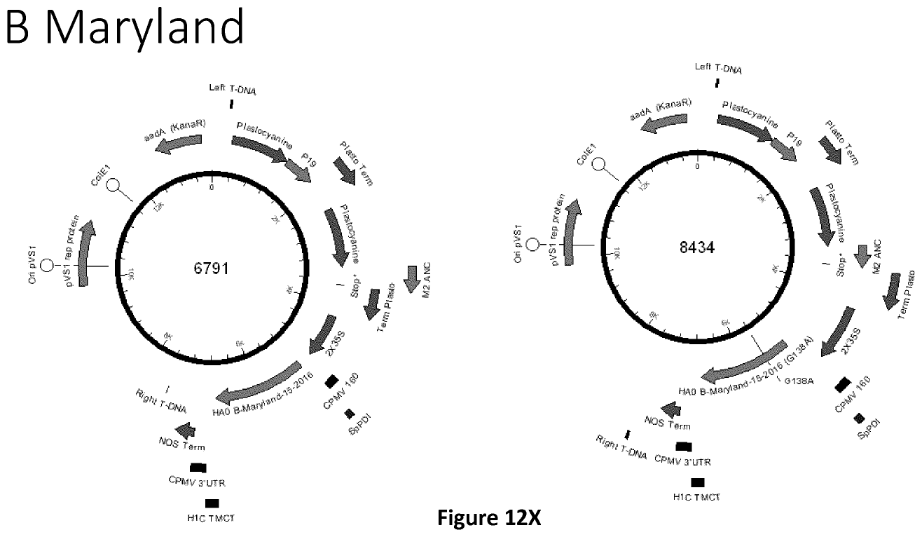

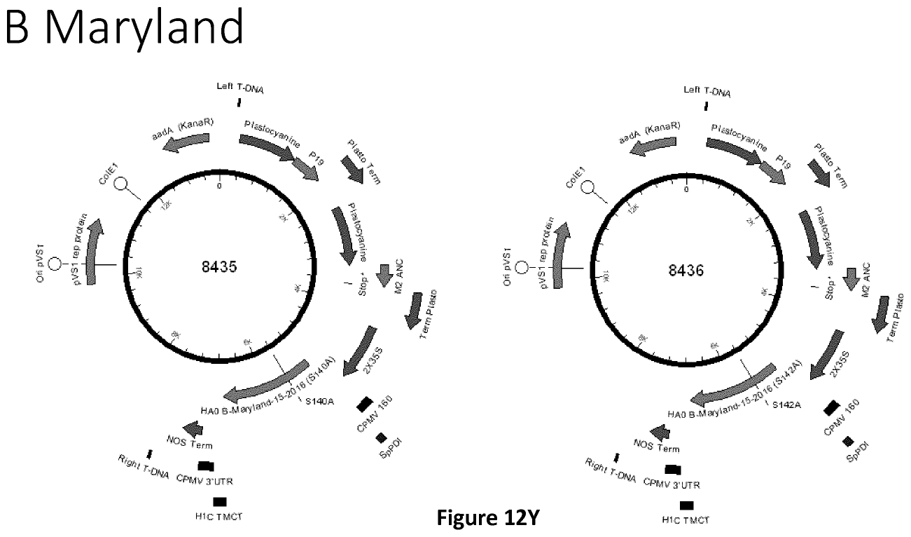

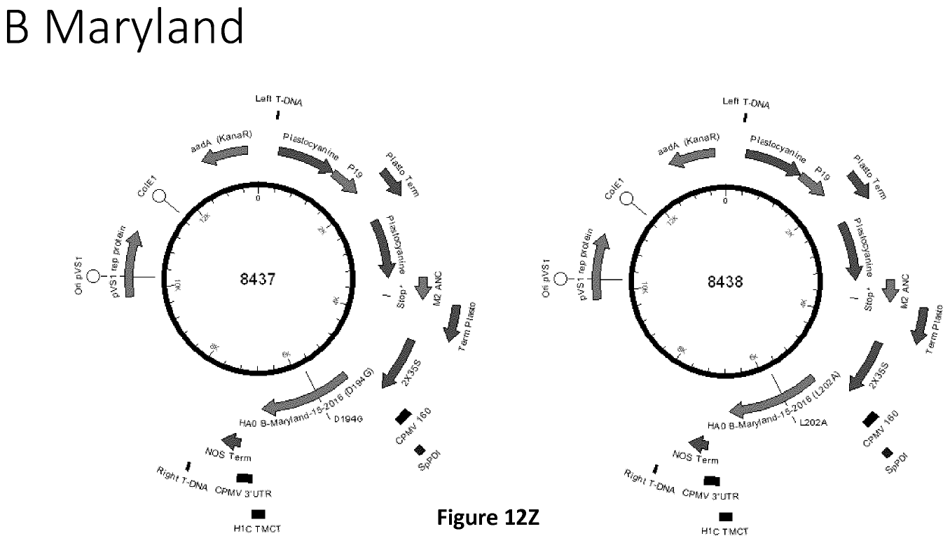

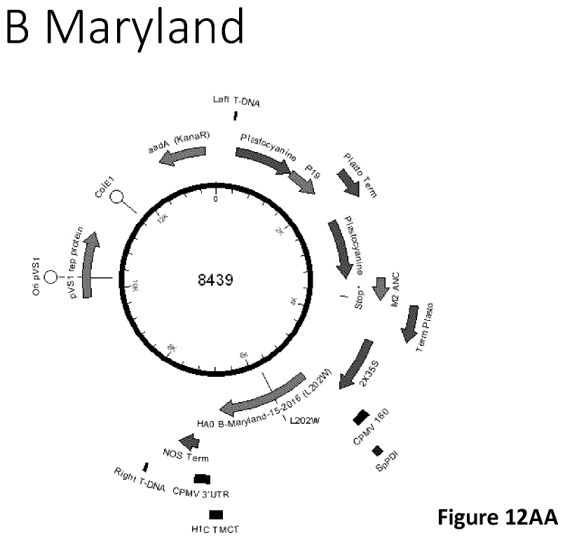

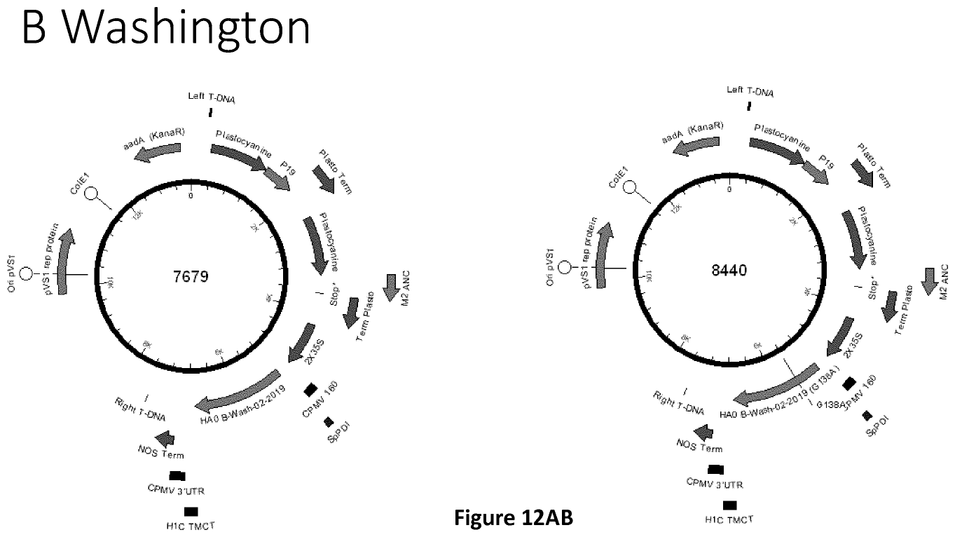

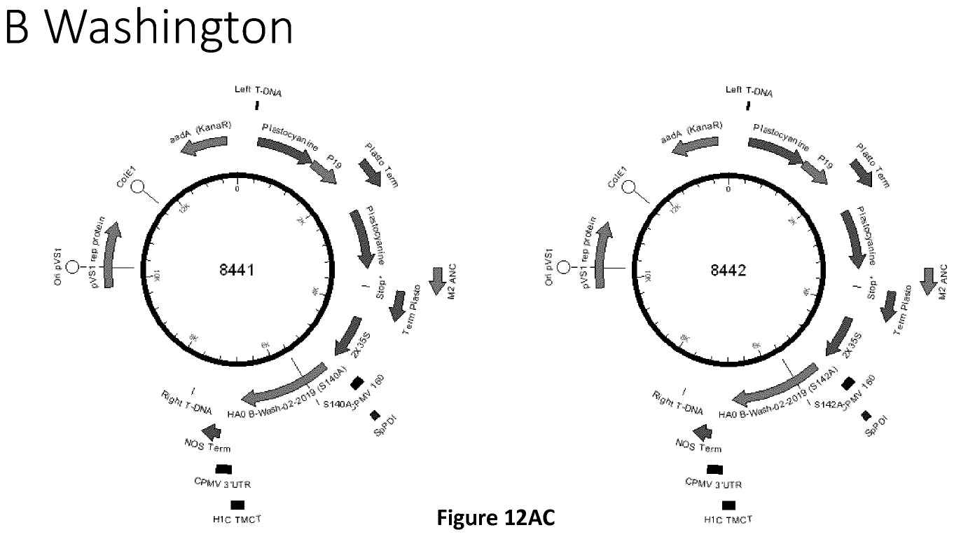

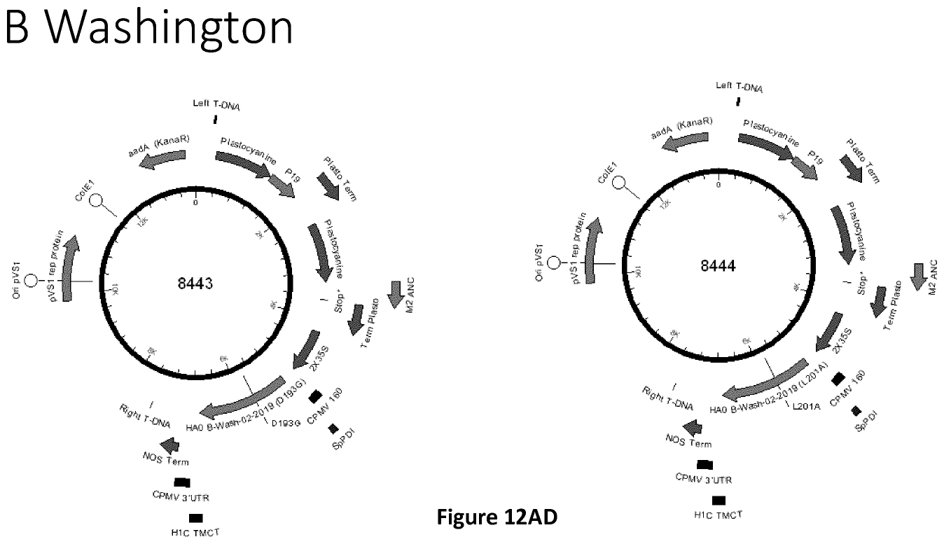

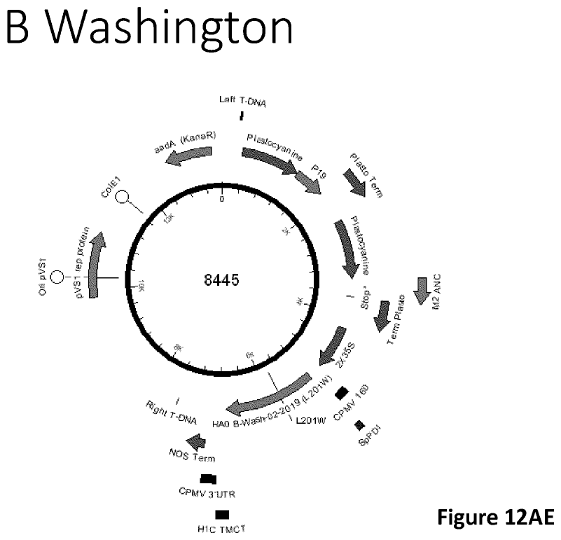

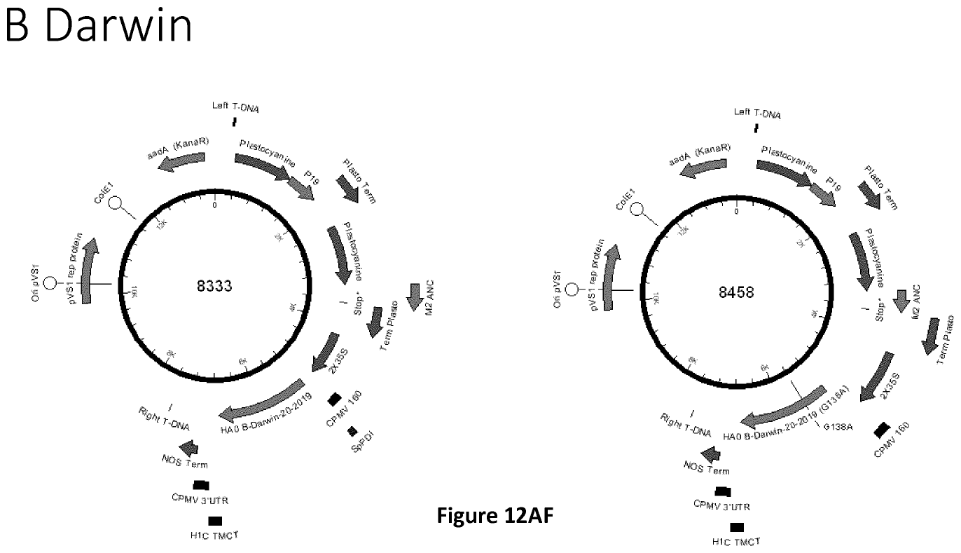

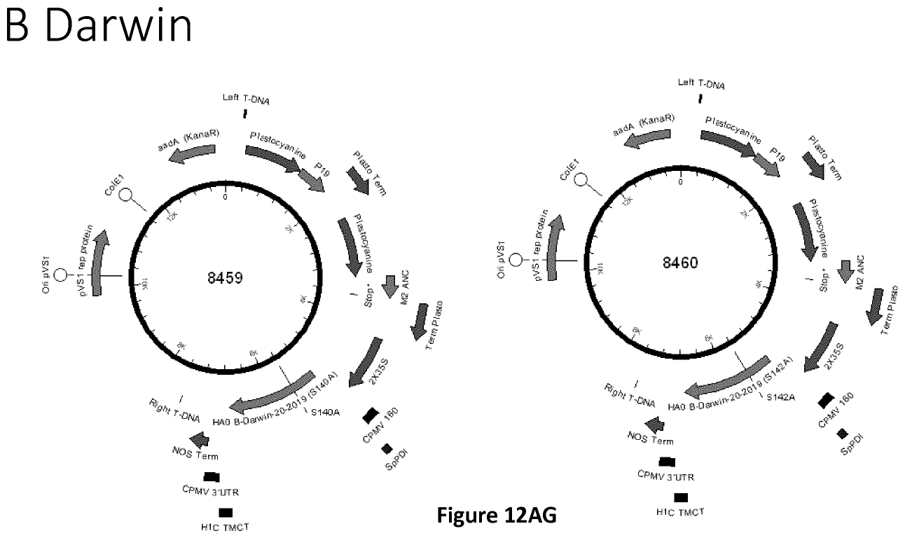

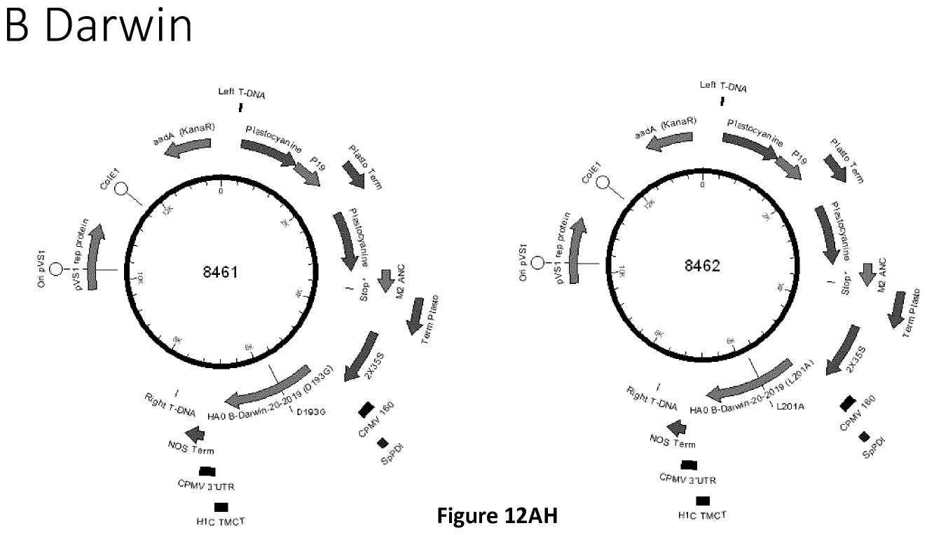

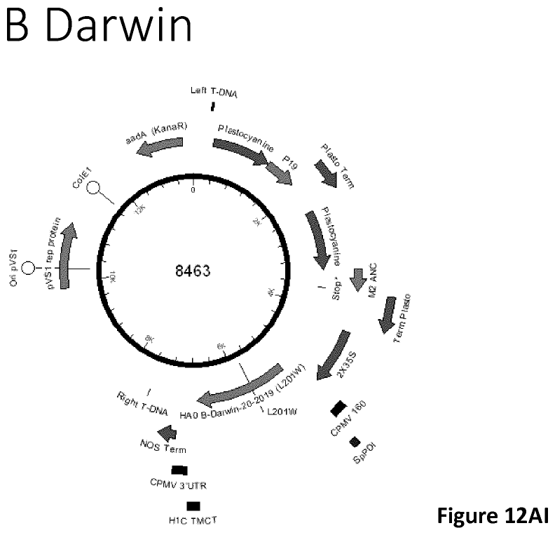

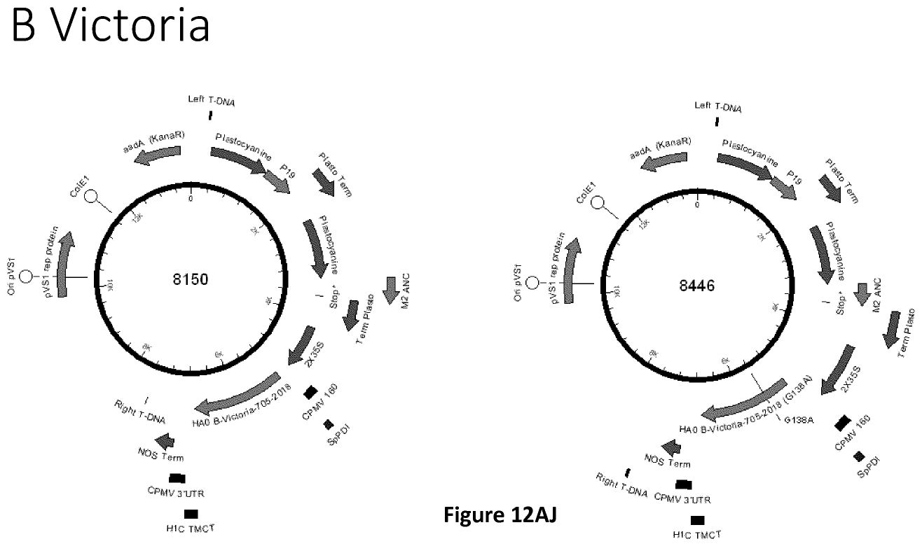

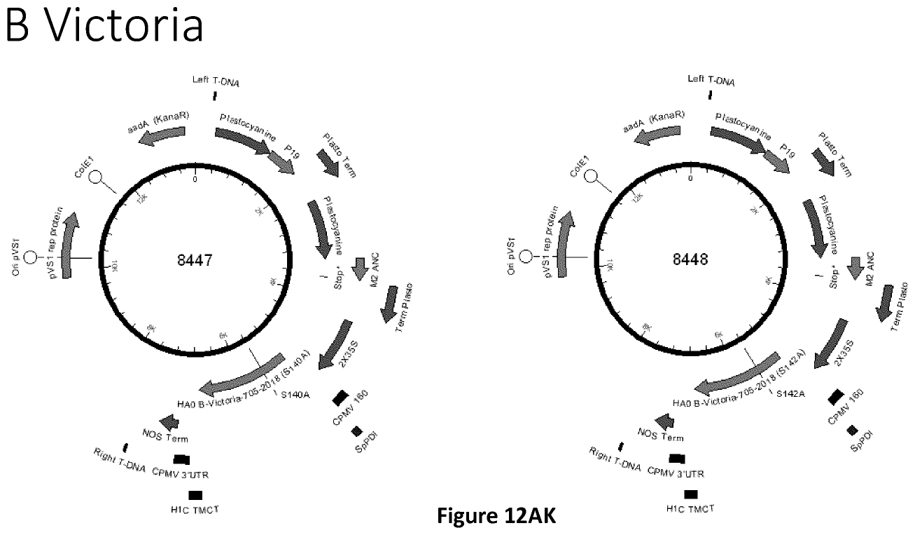

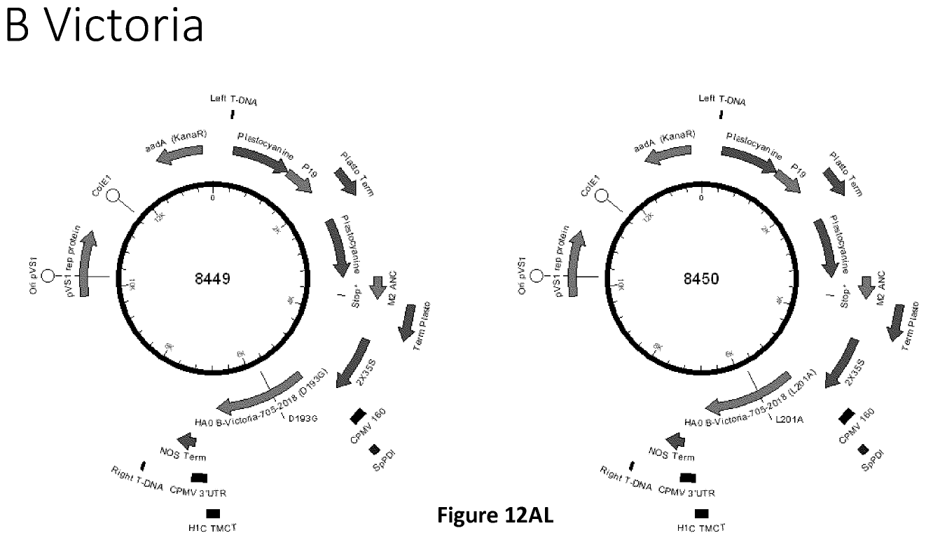

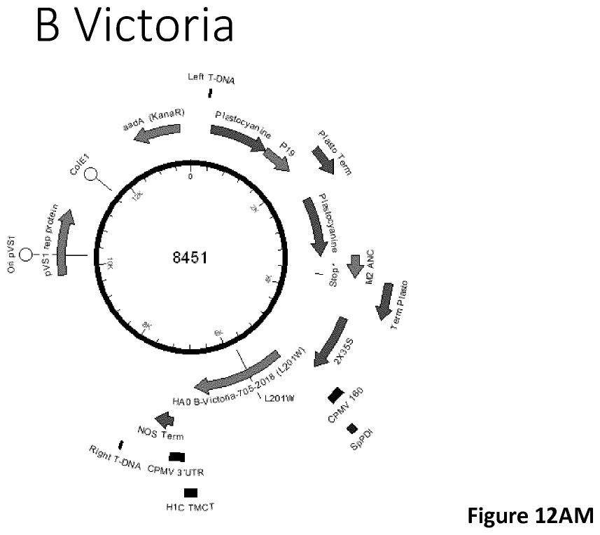

A sequence encoding mature HA0 from influenza HA from A/California/7/09 fused to alfalfa PDI secretion signal peptide (PDISP) was cloned into 2X35S/CPMV 160/NOS expression system using the following PCR-based method. A fragment containing the PDISP-A/California/7/09 coding sequence was amplified using primers IF-CPMV(fl5′UTR)_SpPDI.c (SEQ ID NO:3) and IF-H1cTMCT.S1-4r (SEQ ID NO:4), using PDISP-H1 A/California/7/09 nucleotide sequence (SEQ ID NO:1) as template. The PCR product was cloned in 2X35S/CPMV 160/NOS expression system using In-Fusion cloning system (Clontech, Mountain View, Calif.). Construct number 1190 (FIGS. 17A, 23A) was digested with SacII and StuI restriction enzyme and the linearized plasmid was used for the In-Fusion assembly reaction. Construct number 1190 is an acceptor plasmid intended for “In Fusion” cloning of genes of interest in a 2X35S/CPMV 160/NOS-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO:5. The resulting construct was given number 1314 (SEQ ID NO:6). The amino acid sequence of mature HA0 from influenza HA from A/California/7/09 fused to alfalfa PDI secretion signal peptide (PDISP) is presented in SEQ ID NO:2. A representation of plasmid 1314 is presented in FIGS. 12A, 23B.

### Example 1.2: 2X35S/CPMV 160/PDISP-HA0 H1 A-California-7-09 (Y91F)/NOS (Construct Number 6100)

A sequence encoding mature HA0 from influenza HA from A/California/7/09 (Y91F) fused to alfalfa PDI secretion signal peptide (PDISP) was cloned into 2X35S/CPMV 160/NOS expression system using the following PCR-based method. In a first round of PCR, a fragment containing the PDISP-H1 A/California/7/09 with the mutated Y91F amino acid was amplified using primers IF-CPMV(fl5′UTR)_SpPDI.c (SEQ ID NO:3) and H1_Cal(Y91F).r (SEQ ID NO:7), using PDISP-H1 A/California/7/09 gene sequence (SEQ ID NO: 1) as template. A second fragment containing the Y91F mutation with the remaining of the H1 A/California/7/09 was amplified using H1_Cal(Y91F).c (SEQ ID NO:8) and IF-H1cTMCT.S1-4r (SEQ ID NO:4), using PDISP-H1 A/California/07/09 nucleotide sequence (SEQ ID NO:1) as template. The PCR products from both amplifications were then mixed and used as template for a second round of amplification using IF-CPMV(fl5′UTR)_SpPDI.c (SEQ ID NO:3) and IF-H1cTMCT.S1-4r (SEQ ID NO:4) as primers. The final PCR product was cloned in 2X35S/CPMV 160/NOS expression system using In-Fusion cloning system (Clontech, Mountain View, Calif.). Construct number 3637 (FIGS. 17A, 23C) was digested with SacII and StuI restriction enzyme and the linearized plasmid was used for the In-Fusion assembly reaction. Construct number 3637 is an acceptor plasmid intended for “In Fusion” cloning of genes of interest in a 2X35S/CPMV 160/NOS-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO:9. The resulting construct was given number 6100 (SEQ ID NO:10). The amino acid sequence of mutated PDISP-HA from A/California/07/09 (Y91F) is presented in SEQ ID NO:12. A representation of plasmid 6100 is presented in FIGS. 12A, 23D.

### Example 1.3: 2X35S/CPMV 160/PDISP-HA0 H3 A-Kansas-14-2017/NOS (Construct Number 7281)

A sequence encoding mature HA0 from influenza HA from H3 A/Kansas/14/2017 (N382A+L384V, Cys™) fused to alfalfa PDI secretion signal peptide (PDISP) was cloned into 2X35S/CPMV 160/NOS expression system using the following PCR-based method. A fragment containing the H3 A-Kansas-14-2017 with the mutated amino acids N382A and L384V was amplified using primers IF-H3NewJer.c (SEQ ID NO: 62) and IF-H3_Swi_13.r (SEQ ID NO: 63), using PDISP-H3 A/Kansas/14/2017 (N382A+L384V, Cys™) gene sequence (SEQ ID NO: 60) as template. The final PCR product was cloned in 2X35S/CPMV 160/NOS expression system using In-Fusion cloning system (Clontech, Mountain View, Calif.). Construct number 4499 (FIGS. 17B, 23G) was digested with AatII and StuI restriction enzyme and the linearized plasmid was used for the In-Fusion assembly reaction. Construct number 4499 is an acceptor plasmid intended for “In Fusion” cloning of genes of interest in a 2X35S/CPMV 160/NOS-based expression cassette. It includes the alfalfa PDI secretion signal peptide (PDISP) and incorporates a gene construct for the co-expression of the TBSV P19 suppressor of silencing under the alfalfa Plastocyanin gene promoter and terminator and an influenza M2 ion channel gene under the control of the alfalfa Plastocyanin gene promoter and terminator. The backbone is a pCAMBIA binary plasmid and the sequence from left to right t-DNA borders is presented in SEQ ID NO: 56. The resulting construct was given number 7281 (SEQ ID NO: 58). The amino acid sequence of PDISP-HA from H3 A/Kansas/14/2017 (N382A+L384V, Cys™) is presented in SEQ ID NO: 61. A representation of plasmid 7281 is presented in FIGS. 13A, 23I.

### Example 1.4: 2X35S/CPMV 160/PDISP-HA0 H3 A-Kansas-14-2017/NOS (Construct Number 8179)

A sequence encoding mature HA0 from influenza HA from H3 A/Kansas/14/2017 (Y98F+N382A+L384V, Cys™) fused to alfalfa PDI secretion signal peptide (PDISP) was cloned into 2X35S/CPMV 160/NOS expression system using the following PCR-based method. In a first round of PCR, a fragment containing the H3 A-Kansas-14-2017 with the mutated amino acid Y98F was amplified using primers IF-H3NewJer.c (SEQ ID NO: 62) and H3_Kansas(Y98F).r (SEQ ID NO: 67), using PDISP-H3 A/Kansas/14/2017 (N382A+L384V, Cys™) gene sequence (SEQ ID NO: 60) as template. A second fragment containing the remaining of the H3 A/Kansas/14/2017 (N382A+L384V, Cys™) was amplified using H3_Kansas(Y98F).c (SEQ ID NO: 66) and IF-H3_Swi_13.r (SEQ ID NO: 63), using PDISP-H3 A/Kansas/14/2017 (N382A+L384V, Cys™) gene sequence (SEQ ID NO: 60) as template. The PCR products from both amplifications were then mixed and used as template for a second round of amplification using IF-H3NewJer.c (SEQ ID NO: 62) and IF-H3_Swi_13.r (SEQ ID NO: 63) as primers. The final PCR product was cloned in 2X35S/CPMV 160/NOS expression system using In-Fusion cloning system (Clontech, Mountain View, Calif.). Construct number 4499 (FIGS. 17B, 23G) was digested with AatII and StuI restriction enzyme and the linearized plasmid was used for the In-Fusion assembly reaction. Construct number 4499 is an acceptor plasmid intended for “In Fusion” cloning of genes of interest in a 2X35S/CPMV 160/NOS-based expression cassette. It includes the alfalfa PDI secretion signal peptide (PDISP) and incorporates a gene construct for the co-expression of the TBSV P19 suppressor of silencing under the alfalfa Plastocyanin gene promoter and terminator and an influenza M2 ion channel gene under the control of the alfalfa Plastocyanin gene promoter and terminator. The backbone is a pCAMBIA binary plasmid and the sequence from left to right t-DNA borders is presented in SEQ ID NO: 56. The resulting construct was given number 8179 (SEQ ID NO: 59). The amino acid sequence of PDISP-HA from H3 A/Kansas/14/2017 (Y98F+N382A+L384V, Cys™) is presented in SEQ ID NO: 65. A representation of plasmid 8179 is presented in FIGS. 13A, 23J.

### Example 1.5: 2X35S/CPMV 160/PDISP-HA0 B-Phuket-3073-2013 NOS (Construct Number 2835)

A sequence encoding mature HA0 from influenza HA from B/Phuket/3073/2013 with proteolytic loop removed was fused to the alfalfa PDI secretion signal peptide (PDISP) and cloned into 2X35S/CPMV 160/NOS expression system using the following PCR-based method. A fragment containing the B/Phuket/3073/2013(PrL-) coding sequence was amplified using primers IF.HBPhu3073.c (SEQ ID NO:29) and IF-H1cTMCT.S1-4r (SEQ ID NO:4), using PDISP-B/Phuket/3073/2013(PrL-) nucleotide sequence (SEQ ID NO:27) as template. The PCR product was cloned in 2X35S/CPMV 160/NOS expression system using In-Fusion cloning system (Clontech, Mountain View, Calif.). Construct number 2530 (FIGS. 17B, 23E) was digested with AatII restriction enzyme and the linearized plasmid was used for the In-Fusion assembly reaction. Construct number 2530 is an acceptor plasmid intended for “In Fusion” cloning of genes of interest in a 2X35S/CPMV 160/NOS-based expression cassette. It includes the alfalfa PDI secretion signal peptide (PDISP) and incorporates a gene construct for the co-expression of the TBSV P19 suppressor of silencing under the alfalfa Plastocyanin gene promoter and terminator and an influenza M2 ion channel gene under the control of the alfalfa Plastocyanin gene promoter and terminator. The backbone is a pCAMBIA binary plasmid and the sequence from left to right t-DNA borders is presented in SEQ ID NO:54. The resulting construct was given number 2835 (SEQ ID NO:55). The amino acid sequence of mature HA0 from influenza HA from B/Phuket/3073/2013(PrL-) fused to alfalfa PDI secretion signal peptide (PDISP) is presented in SEQ ID NO:28. A representation of plasmid 2835 is presented in FIGS. 16A, 23F.

### Example 1.6: 2X35S/CPMV 160/PDISP-HA0 B-Phuket-3073-2013(S140A)/NOS (Construct Number 8352)

A sequence encoding mature HA0 from influenza HA from B/Phuket/3073/2013 (PrL-, S140A) fused to alfalfa PDI secretion signal peptide (PDISP) was cloned into 2X35S/CPMV 160/NOS expression system using the following PCR-based method. In a first round of PCR, a fragment containing the PDISP-B/Phuket/3073/2013(PrL-) with the mutated S140A amino acid was amplified using primers IF.HBPhu3073.c (SEQ ID NO:29) and B Phuket(S140A).r (SEQ ID NO:31), using PDISP-B/Phuket/3073/2013(PrL-) gene sequence (SEQ ID NO:27) as template. A second fragment containing the S140A mutation with the remaining of the B/Phuket/3073/2013(PrL-) was amplified using B_Phuket(S140A).c (SEQ ID NO:30) and IF-H1cTMCT.S1-4r (SEQ ID NO:4), using PDISP-B/Phuket/3073/2013(PrL-) gene sequence (SEQ ID NO:27) as template. The PCR products from both amplifications were then mixed and used as template for a second round of amplification using IF.HBPhu3073.c (SEQ ID NO:29) and IF-H1cTMCT.S1-4r (SEQ ID NO:4) as primers. The final PCR product was cloned in 2X35S/CPMV 160/NOS expression system using In-Fusion cloning system (Clontech, Mountain View, Calif.). Construct number 4499 (FIGS. 17B, 23G) was digested with AatII and StuI restriction enzyme and the linearized plasmid was used for the In-Fusion assembly reaction. Construct number 4499 is an acceptor plasmid intended for “In Fusion” cloning of genes of interest in a 2X35S/CPMV 160/NOS-based expression cassette. It also incorporates a gene construct for the co-expression of the TBSV P19 suppressor of silencing under the alfalfa Plastocyanin gene promoter and terminator and an influenza M2 ion channel gene under the control of the alfalfa Plastocyanin gene promoter and terminator. The backbone is a pCAMBIA binary plasmid and the sequence from left to right t-DNA borders is presented in SEQ ID NO:56. The resulting construct was given number 8352 (SEQ ID NO:57). The amino acid sequence of mutated PDISP-HA from B/Phuket/3073/2013 (PrL-, S140A) is presented in SEQ ID NO:33. A representation of plasmid 8352 is presented in FIGS. 16A, 23H.

A summary of the wildtype and mutated HA proteins, primers, templates, accepting vectors and products is provided in Tables 4 and 5 below.

TABLE 4
Primers used to prepare constructs as disclosed herein

| SEQ ID NO: | Identifier | Sequence |
|---|---|---|
| 3 | IF-CPMV(fl5′UTR)_SpPDI.c | TCGTGCTTCGGCACCAGTACAATGGCGAAAAACGTTGCGATTTTCGGCT |
| 4 | IF-H1cTMCT.S1-4r | ACTAAAGAAAATAGGCCTTTAAATACATATTCTACACTGTAGAGAC |
| 7 | H1_Cal(Y91F).r | AAATCTCCTGGGAAACACGTTCCATTGTCTGAACTAGGTGTTTCCACAA |
| 8 | H1_Cal(Y91F).c | AGACAATGGAACGTGTTTCCCAGGAGATTTCATCGATTATGAGGAGCTA |
| 15 | IF-H5ITMCT.s1-4r | ACTAAAGAAAATAGGCCTTTAAATGCAAATTCTGCATTGTAACGATCCAT |
| 16 | H5Indo(Y91F).c | AACCAATGACCTCTGTTTCCCAGGGAGTTTCAACGACTATGAAGAACTGAA |
| 17 | H5Indo(Y91F).r | GAAACTCCCTGGGAAACAGAGGTCATTGGTTGGATTGGCCTTCTCCACTATGTAAGA |
| 22 | IF-H7Shang.r | ACTAAAGAAAATAGGCCTTTATATACAAATAGTGCACCGCATGTTTCCAT |
| 23 | H7Shang(Y88F).c | AGGAAGTGATGTCTGTTTCCCTGGGAAATTCGTGAATGAAGAAGCTCTGA |
| 24 | H7Shang(Y88F).r | ACGAATTTCCCAGGGAAACAGACATCACTTCCTTCTCGCCTCTCAATAAT |
| 29 | IF.HBPhu3073.c | TCTCAGATCTTCGCGGATCGAATCTGCACTGGGATAACATCTTCAAACTCAC |
| 30 | B_Phuket(S140A).c | GACCCTACAGACTTGGAACCGCCGGATCTTGCCCTAACGCTACCAGTAAAATCGGATTT |
| 31 | B_Phuket(S140A).r | CGTTAGGGCAAGATCCGGCGGTTCCAAGTCTGTAGGGTCCTCCTGGTGCTTTTTCTG |
| 34 | B_Phuket(S142A).c | TGGAACCTCAGGAGCCTGCCCTAACGCTACCAGTAAAATCGGATTTTTTGCAACAATG |
| 35 | B_Phuket(S142A).r | TGGTAGCGTTAGGGCAGGCTCCTGAGGTTCCAAGTCTGTAGGGTCCTC |
| 38 | B_Phuket(G138A).c | GACCCTACAGACTTGCCACCTCAGGATCTTGCCCTAACGCTACCAGTAA |
| 39 | B_Phuket(G138A).r | GGCAAGATCCTGAGGTGGCAAGTCTGTAGGGTCCTCCTGGTGCTTTTTCTG |
| 42 | B_Phuket(L203A).c | CCCAAATGAAGAGCGCCTATGGAGACTCAAATCCTCAAAAGTTCACCTC |
| 43 | B_Phuket(L203A).r | GATTTGAGTCTCCATAGGCGCTCTTCATTTGGGTTTTGTTATCCGAAT |
| 46 | B_Phuket(D195G).c | GGGGGTTCCATTCGGGCAACAAAACCCAAATGAAGAGCCTCTATGGAGA |
| 47 | B_Phuket(D195G).r | TCATTTGGGTTTTGTTGCCCGAATGGAACCCCCAAACAGTAATTTGGT |
| 50 | B_Phuket(L203W).c | CCCAAATGAAGAGCTGGTATGGAGACTCAAATCCTCAAAAGTTCACCTC |
| 51 | B_Phuket(L203W).r | GATTTGAGTCTCCATACCAGCTCTTCATTTGGGTTTTGTTATCCGAAT |
| 62 | IF-H3NewJer.c | TCTCAGATCTTCGCGCAAAAAATCCCTGGAAATGACAATAGCACGGCAACGCTGTGC |
| 63 | IF-H3_Swi_13.r | ACTAAAGAAAATAGGCCTTCAAATGCAAATGTTGCACCTAATGTTGCCCTT |
| 66 | H3_Kansas(Y98F).c | CCTACAGCAACTGTTTCCCTTATGATGTGCCGGATTATGCCTCCCTTA |
| 67 | H3_Kansas(Y98F).r | CCGGCACATCATAAGGGAAACAGTTGCTGTAGGCTTTGTTTCGTTCAACA |
| 70 | H3Kansas(S136D).c | AAACGGAACAGACTCTTCTTGCATAAGGGGATCTAAGAGTAGTTTCTT |
| 71 | H3Kansas(S136D).r | CAAGAAGAGTCTGTTCCGTTTTGAGTGACTCCAGCCCAATTGAAGCTTTC |
| 74 | H3Kansas(S136N).c | AAACGGAACAAACTCTTCTTGCATAAGGGGATCTAAGAGTAGTTTCTT |
| 75 | H3Kansas(S136N).r | CAAGAAGAGTTTGTTCCGTTTTGAGTGACTCCAGCCCAATTGAAGCTTTC |
| 78 | H3Kansas(S137N).c | CGGAACAAGTAACTCTTGCATAAGGGGATCTAAGAGTAGTTTCTTTAGTAG |
| 79 | H3Kansas(S137N).r | ATGCAAGAGTTACTTGTTCCGTTTTGAGTGACTCCAGCCCAATTGAAGCTTTCAT |
| 82 | H3Kansas(D190G).c | TACGGACAAGGGCCAAATCAGCCTGTATGCACAATCATCAGGAAGAATC |
| 83 | H3Kansas(D190G).r | CTGATTTGGCCCTTGTCCGTACCCGGGTGGTGAACCCCCCAAATGTAC |
| 86 | H3Kansas(D190K).c | TACGGACAAGAAGCAAATCAGCCTGTATGCACAATCATCAGGAAGAATC |
| 87 | H3Kansas(D190K).r | CTGATTTGCTTCTTGTCCGTACCCGGGTGGTGAACCCCCCAAATGTAC |
| 90 | H3Kansas(R222W).c | ATCTAGACCCTGGATAAGGGATATCCCTAGCAGAATAAGCATCTATTGGA |
| 91 | H3Kansas(R222W).r | TCCCTTATCCAGGGTCTAGATCCGATATTCGGGATTACAGCTTGTTGGC |
| 94 | H3Kansas(S228N).c | GGATATCCCTAACAGAATAAGCATCTATTGGACAATAGTAAAACCGGGAGA |
| 95 | H3Kansas(S228N).r | CTTATTCTGTTAGGGATATCCCTTATTCTGGGTCTAGATCCGATATTCGGG |
| 98 | H3Kansas(S228Q).c | GGATATCCCTCAGAGAATAAGCATCTATTGGACAATAGTAAAACCGGGAGACATA |
| 99 | H3Kansas(S228Q).r | CTTATTCTCTGAGGGATATCCCTTATTCTGGGTCTAGATCCGATATTCGGG |
| 102 | IF-H1_Cal-7-09.c | TCTCAGATCTTCGCGGACACATTATGTATAGGTTATCATGCGAACAAT |
| 103 | IF-H1cTMCT.s1-4r | ACTAAAGAAAATAGGCCTTTAAATACATATTCTACACTGTAGAGAC |
| 106 | H1_Idaho(Y91F).c | ACAATGGAACGTGTTTCCCAGGAGATTTCATCAATTATGAGGAGCTAA |
| 107 | H1_Idaho(Y91F).r | TGATGAAATCTCCTGGGAAACACGTTCCATTGTCTGAATTAGATGTTT |
| 139 | IF-B-Bris(nat).c | tctcagatcttcgcggatcgaatctgcactgggataacatcgtcaaactc |
| 200 | IF-H5ITMCT.s1-4r | actaaagaaaataggcctttaaatgcaaattctgcattgtaacgatccat |

TABLE 5
Primers, templates, acceptor plasmids used to prepare constructs as disclosed herein

| | | P1* | P2** | P3*** | P4**** | PCR1# | NA## | Protein~ | |
|---|---|---|---|---|---|---|---|---|---|
| Nucleic acid of interest | Const. # | SEQ ID NO: | | | | | | | Acceptor plasmid |
| H1 A/California/7/2009 | 1314 | 3 | 4 | — | — | 1 | 1 | 2 | 1190 (SacII-StuI) |
| H1 A/California/7/2009 (Y91F) | 6100 | 3 | 7 | 8 | 4 | 1 | 11 | 12 | 3637 (SacII-StuI) |
| H5 A/Indonesia/5/2005 | 2295 | 3 | 15 | — | — | 13 | 13 | 14 | 1190 (SacII-StuI) |
| H1 A/Idaho/07/2018 | 4795 | 3 | 103 | — | — | 100 | 100 | 101 | 3637 (SacII-StuI) |
| H1 A/Idaho/07/2018 (Y91F) | 8177 | 3 | 107 | 106 | 103 | 100 | 104 | 105 | 3637 (SacII-StuI) |
| H3 A/Kansas/14/2017 | 7281 | 62 | 63 | — | — | 60 | 60 | 61 | 4499 (AatII-StuI) |
| (N382A + L384V) | | | | | | | | | |
| H3 A/Kansas/14/2017 | 8179 | 62 | 67 | 66 | 63 | 60 | 64 | 65 | 4499 (AatII-StuI) |
| (Y98F + N382A + L384V) | | | | | | | | | |
| H3 A/Kansas/14/2017 | 8384 | 62 | 70 | 71 | 63 | 64 | 68 | 69 | 4499 (AatII-StuI) |
| (Y98F + S136D + N382A + L384V) | | | | | | | | | |
| H3 A/Kansas/14/2017 | 8385 | 62 | 75 | 74 | 63 | 64 | 72 | 73 | 4499 (AatII-StuI) |
| (Y98F + S136N + N382A + L384V) | | | | | | | | | |
| H3 A/Kansas/14/2017 | 8387 | 62 | 79 | 78 | 63 | 64 | 76 | 77 | 4499 (AatII-StuI) |
| (Y98F + S137N + N382A + L384V) | | | | | | | | | |
| H3 A/Kansas/14/2017 | 8388 | 62 | 83 | 82 | 63 | 64 | 80 | 81 | 4499 (AatII-StuI) |
| (Y98F + D190G + N382A + L384V) | | | | | | | | | |
| H3 A/Kansas/14/2017 | 8389 | 62 | 87 | 86 | 63 | 64 | 84 | 85 | 4499 (AatII-StuI) |
| (Y98F + D190K + N382A + L384V) | | | | | | | | | |
| H3 A/Kansas/14/2017 | 8391 | 62 | 91 | 90 | 63 | 64 | 88 | 89 | 4499 (AatII-StuI) |
| (Y98F + R222W + N382A + L384V) | | | | | | | | | |
| H3 A/Kansas/14/2017 | 8392 | 62 | 95 | 94 | 63 | 64 | 92 | 93 | 4499 (AatII-StuI) |
| (Y98F + S228N + N382A + L384V) | | | | | | | | | |
| H3 A/Kansas/14/2017 | 8393 | 62 | 99 | 98 | 63 | 64 | 96 | 97 | 4499 (AatII-StuI) |
| (Y98F + S228Q + N382A + L384V) | | | | | | | | | |
| H5 A/Indonesia/5/2005 (Y91F) | 6101 | 3 | 17 | 16 | 15 | 13 | 18 | 19 | 3637 (SacII-StuI) |
| H7 A/Shanghai/2/2013 | 6102 | 3 | 22 | — | — | 20 | 20 | 21 | 3637 (SacII-StuI) |
| H7 A/Shanghai/2/2013 (Y88F) | 6103 | 3 | 24 | 23 | 22 | 20 | 25 | 26 | 3637 (SacII-StuI) |
| B/Phuket/3073/2013 | 2835 | 29 | 4 | — | — | 27 | 27 | 28 | 2530 (AatII) |
| B/Phuket/3073/2013 (S140A, PrL−) | 8352 | 29 | 31 | 30 | 4 | 27 | 32 | 33 | 4499 (AatII-StuI) |
| B/Phuket/3073/2013 (S142A, PrL−) | 8354 | 29 | 35 | 34 | 4 | 27 | 36 | 37 | 4499 (AatII-StuI) |
| B/Phuket/3073/2013 (G138A, PrL−) | 8358 | 29 | 39 | 38 | 4 | 27 | 40 | 41 | 4499 (AatII-StuI) |
| B/Phuket/3073/2013 (L203A, PrL−) | 8363 | 29 | 43 | 42 | 4 | 27 | 44 | 45 | 4499 (AatII-StuI) |
| B/Phuket/3073/2013 (D195G, PrL−) | 8376 | 29 | 47 | 46 | 4 | 27 | 48 | 49 | 4499 (AatII-StuI) |
| B/Phuket/3073/2013 (L203W, PrL−) | 8382 | 29 | 51 | 50 | 4 | 27 | 52 | 53 | 4499 (AatII-StuI) |
| H3 A/Kansas/14/2017 (S136D) | 8477 | 62 | 71 | 70 | 63 | 60 | 111 | 112 | 4499 (AatII-StuI) |
| H3 A/Kansas/14/2017 (S136N) | 8478 | 62 | 75 | 74 | 63 | 60 | 113 | 114 | 4499 (AatII-StuI) |
| H3 A/Kansas/14/2017 (D190K) | 8481 | 62 | 87 | 86 | 63 | 60 | 115 | 116 | 4499 (AatII-StuI) |
| H3 A/Kansas/14/2017 (R222W) | 8482 | 62 | 91 | 90 | 63 | 60 | 117 | 118 | 4499 (AatII-StuI) |
| H3 A/Kansas/14/2017 (S228N) | 8483 | 62 | 95 | 94 | 63 | 60 | 119 | 120 | 4499 (AatII-StuI) |
| H3 A/Kansas/14/2017 (S228Q) | 8484 | 62 | 99 | 98 | 63 | 60 | 121 | 122 | 4499 (AatII-StuI) |
| B/Singapore/INFKK-16-0569/2016 | 2879 | 29 | 103 | — | — | 123 | 123 | 124 | 4499 (AatII-StuI) |
| B/Singapore/INFKK-16-0569/2016 | 8485 | 29 | 103 | — | — | 125 | 125 | 126 | 4499 (AatII-StuI) |
| (G138A) | | | | | | | | | |
| B/Singapore/INFKK-16-0569/2016 | 8486 | 29 | 103 | — | — | 127 | 127 | 128 | 4499 (AatII-StuI) |
| (S140A) | | | | | | | | | |
| B/Singapore/INFKK-16-0569/2016 | 8487 | 29 | 103 | — | — | 129 | 129 | 130 | 4499 (AatII-StuI) |
| (S142A) | | | | | | | | | |
| B/Singapore/INFKK-16-0569/2016 | 8488 | 29 | 103 | — | — | 131 | 131 | 132 | 4499 (AatII-StuI) |
| (D195G) | | | | | | | | | |
| B/Singapore/INFKK-16-0569/2016 | 8489 | 29 | 103 | — | — | 133 | 133 | 134 | 4499 (AatII-StuI) |
| (L203A) | | | | | | | | | |
| B/Singapore/INFKK-16-0569/2016 | 8490 | 29 | 103 | — | — | 135 | 135 | 136 | 4499 (AatII-StuI) |
| (L203W) | | | | | | | | | |
| B/Maryland/15/2016 | 6791 | 139 | 103 | — | — | 137 | 137 | 138 | 4499 (AatII-StuI) |
| B/Maryland/15/2016 (G138A) | 8434 | 139 | 103 | — | — | 140 | 140 | 141 | 4499 (AatII-StuI) |
| B/Maryland/15/2016 (S140A) | 8435 | 139 | 103 | — | — | 142 | 142 | 143 | 4499 (AatII-StuI) |
| B/Maryland/15/2016 (S142A) | 8436 | 139 | 103 | — | — | 144 | 144 | 145 | 4499 (AatII-StuI) |
| B/Maryland/15/2016 (D194G) | 8437 | 139 | 103 | — | — | 146 | 146 | 147 | 4499 (AatII-StuI) |
| B/Maryland/15/2016 (L202A) | 8438 | 139 | 103 | — | — | 148 | 148 | 149 | 4499 (AatII-StuI) |
| B/Maryland/15/2016 (L202W) | 8439 | 139 | 103 | — | — | 150 | 150 | 151 | 4499 (AatII-StuI) |
| B/Washington/02/2019 | 7679 | 139 | 103 | — | — | 152 | 152 | 153 | 4499 (AatII-StuI) |
| B/Washington/02/2019 (G138A) | 8440 | 139 | 103 | — | — | 154 | 154 | 155 | 4499 (AatII-StuI) |
| B/Washington/02/2019 (S140A) | 8441 | 139 | 103 | — | — | 156 | 156 | 157 | 4499 (AatII-StuI) |
| B/Washington/02/2019 (S142A) | 8442 | 139 | 103 | — | — | 158 | 158 | 159 | 4499 (AatII-StuI) |
| B/Washington/02/2019 (D193G) | 8443 | 139 | 103 | — | — | 160 | 160 | 161 | 4499 (AatII-StuI) |
| B/Washington/02/2019 (L201A) | 8444 | 139 | 103 | — | — | 162 | 162 | 163 | 4499 (AatII-StuI) |
| B/Washington/02/2019 (L201W) | 8445 | 139 | 103 | — | — | 164 | 164 | 165 | 4499 (AatII-StuI) |
| B/Darwin/20/2019 | 8333 | 139 | 103 | — | — | 166 | 166 | 167 | 4499 (AatII-StuI) |
| B/Darwin/20/2019 (G138A) | 8458 | 139 | 103 | — | — | 168 | 168 | 169 | 4499 (AatII-StuI) |
| B/Darwin/20/2019 (S140A) | 8459 | 139 | 103 | — | — | 170 | 170 | 171 | 4499 (AatII-StuI) |
| B/Darwin/20/2019 (S142A) | 8460 | 139 | 103 | — | — | 172 | 172 | 173 | 4499 (AatII-StuI) |
| B/Darwin/20/2019 (D193G) | 8461 | 139 | 103 | — | — | 174 | 174 | 175 | 4499 (AatII-StuI) |
| B/Darwin/20/2019 (L201A) | 8462 | 139 | 103 | — | — | 176 | 176 | 177 | 4499 (AatII-StuI) |
| B/Darwin/20/2019 (L201W) | 8463 | 139 | 103 | — | — | 178 | 178 | 179 | 4499 (AatII-StuI) |
| B/Victoria/705/2018 | 8150 | 139 | 103 | — | — | 180 | 180 | 181 | 4499 (AatII-StuI) |
| B/Victoria/705/2018 (G138A) | 8446 | 139 | 103 | — | — | 182 | 182 | 183 | 4499 (AatII-StuI) |
| B/Victoria/705/2018 (S140A) | 8447 | 139 | 103 | — | — | 184 | 184 | 185 | 4499 (AatII-StuI) |
| B/Victoria/705/2018 (S142A) | 8448 | 139 | 103 | — | — | 186 | 186 | 187 | 4499 (AatII-StuI) |
| B/Victoria/705/2018 (D193G) | 8449 | 139 | 103 | — | — | 188 | 188 | 189 | 4499 (AatII-StuI) |
| B/Victoria/705/2018 (L201A) | 8450 | 139 | 103 | — | — | 190 | 190 | 191 | 4499 (AatII-StuI) |
| B/Victoria/705/2018 (L201W) | 8451 | 139 | 103 | — | — | 192 | 192 | 193 | 4499 (AatII-StuI) |
| H1 A/Brisbane/02/2018 | 6722 | 3 | 103 | — | — | 194 | 194 | 195 | 3637 (SacII-StuI) |
| H1 A/Brisbane/02/2018 (Y91F) | 8433 | 3 | 103 | — | — | 196 | 196 | 197 | 3637 (SacII-StuI) |
| H5 A/Indonesia/5/05 | 2295 | 3 | 200 | — | — | 198 | 198 | 199 | 1190 (SacII-StuI) |
| H5 A/Indonesia/5/05 (Y91F) | 6101 | 3 | 200 | — | — | 201 | 201 | 202 | 3637 (SacII-StuI) |

*Primer 1 (forward primer of fragment 1),
**Primer 2 (reverse primer of fragment 1),
***Primer 3 (forward primer of fragment 2 if needed),
****Primer 4 (reverse primer of fragment 2 if needed)
#Templates for first PCR
##Resulting nucleic acid
~Resulting protein

### Example 2: Plant-Derived VLPs Comprising Parent HA and Modified HA

Virus-like particles bearing parent or modified HA were produced and purified as previously described (WO2020/000099, which is incorporated herein by reference). Briefly, N. benthamiana plants (41-44 days old) were vacuum infiltrated in batches with an Agrobacterium inoculum carrying either parent HA or modified HA expression cassettes. Six days after infiltration, the aerial parts of the plants were harvested and stored at −80° C. until purification. Frozen plant leaves were homogenized in one volume of buffer [50 mM Tris, 150 mM NaCl: 0.04% (w/v) Na₂S₂O₅, pH 8.0]/kg biomass. The homogenate was pressed through a 400 μm nylon filter and the fluid was retained. Filtrates were clarified by centrifugation 5000×g and filtration (1.2 μm glass fiber, 3M Zeta Plus, 0.45-0.42m filter) and then concentrated by centrifugation (75000×g, 20 min). VLPs were further concentrated and purified by ultracentrifugation over an iodixanol density gradient (120000×g, 2h). VLP-rich fractions were pooled and dialyzed against 50 mM NaPO₄, 65 mM NaCl, 0.01% Tween 80 (pH 6.0). This clarified extract was captured on a Poros HS column (Thermo Scientific) equilibrated in 50 mM NaPO₄, 1M NaCl, 0.005% Tween 80. After washing with 25 mM Tris, 0.005% Tween 80 (pH 8.0), the VLPs were eluted with 50 mM NaPO₄, 700 mM NaCl, 0.005% Tween 80 (pH 6.0). Purified VLPs were dialyzed against formulation buffer (100 mM NaKPO₄, 150 mM NaCl, 0.01% Tween 80 (pH 7.4)) and passed through a 0.22 μm filter for sterilization.

The composition of the VLP preparations was determined by gel electrophoresis followed by Coomassie staining and western blotting. Both VLP preparations are primarily composed of the uncleaved form of HA (HA0). Purity was determined by densitometry analysis of stained gels and was used to calculate the total HA content [total protein (BCA) x % purity]. The purity of preparations was approx. 95%.

VLPs comprising non-modified or modified HA were visualized for particle formation and morphology by electron microscopy. Exemplary electron micrograph images for VLPs comprising either non-modified or modified HA from H1/Brisbane, H3/Kansas, B/Phuket

and B/Maryland are shown in FIG. 1C. No differences were observed between VLPs comprising either non-modified or modified HA. The production of VLPs was also confirmed for H1/California, H1/Idaho, B/Singapore and B/Washington (data not shown).

### H1 HA

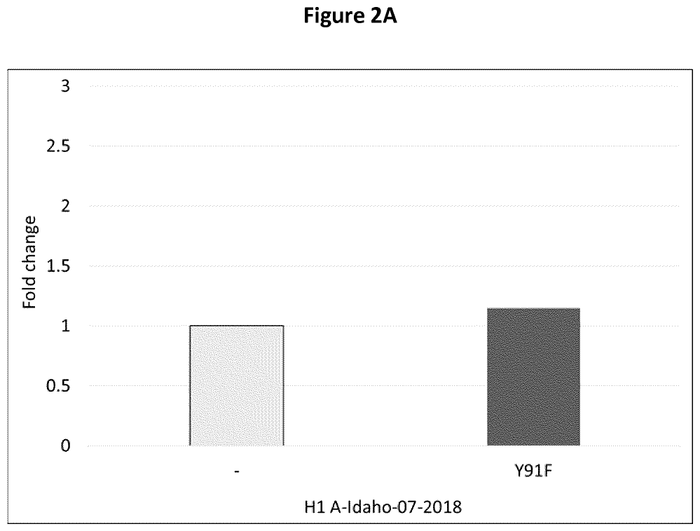

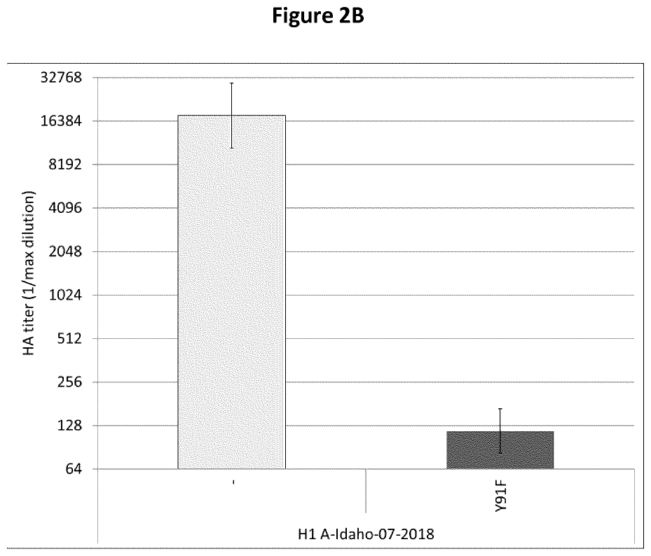

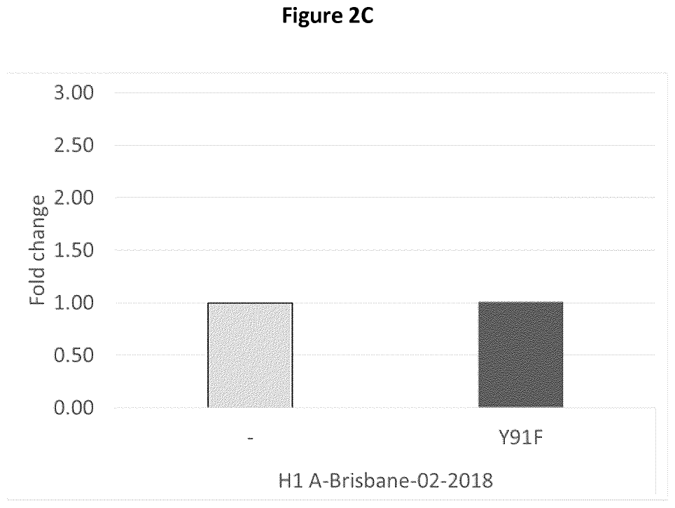

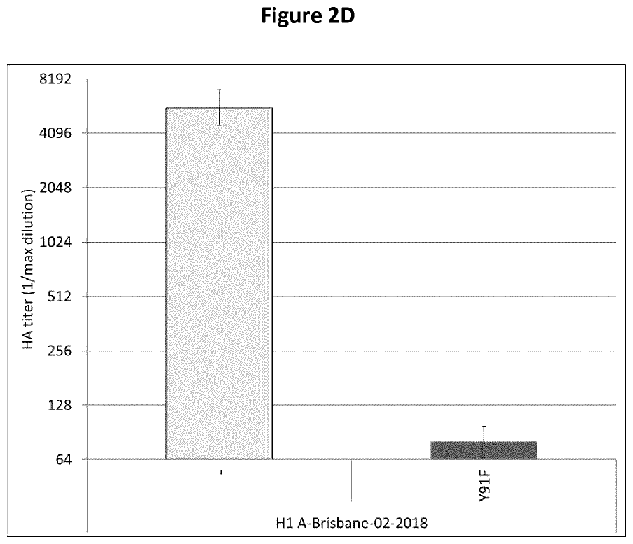

The yield of VLP comprising modified HAs produced in a plant was similar or greater than the yield of the corresponding parent or non-modified HA for VLPs comprising modified H1 A/Idaho/07/2018 (H1 Idaho Y91F; FIG. 2A). However, the modified H1-HA exhibited a significant reduction in hemagglutination activity (expressed as HA titer) as shown in FIG. 2B.

Yield and hemagglutination activity were further assessed in VLPs comprising H1 A/Brisbane/02/2018 or H1 A/Brisbane/02/2018 Y91F (FIGS. 2C and 2D). Y91F mutation in VLPs of Influenza-A strain H1/Brisbane leads to loss of binding (loss of HA titer in Hemagglutination assay) with no effect on yield (depicted in terms of fold change measured by WES analysis on crude biomass extracts).

### H3 HA

The yield of VLP comprising modified HAs produced in a plant was similar or greater than the yield of the corresponding parent or non-modified HA for VLPs comprising modified comprising a series of modified H3 Kansas/14/2017 HAs (H3 Kansas Y98F; H3 Kansas Y98F, S136D; H3 Kansas Y98F, S136N; H3 Kansas Y98F, S137N; H3 Kansas Y98F, D190G; H3 Kansas Y98F, D190K, H3 Kansas Y98F, R222W; H3 Kansas Y98F, S228N; H3 Kansas Y98F, S228Q; FIG. 3A). However, the series of modified H3 HA (excluding H3 Kansas Y98F) exhibited a significant reduction in hemagglutination activity (expressed as HA titer) as shown in FIG. 3B.

Yield and hemagglutination activity were further assessed in a series of VLPs comprising modified H3 Kansas/14/2017 with single non-binding candidate mutations S136D, S136N, D190K, R222W, S228N, and S228Q (FIGS. 3C and 3D). Non-binding candidates of Influenza-A strain H3/Kansas lead to loss of binding (loss of HA titer in Hemagglutination assay), except for R222W, with no loss of yield (depicted in terms of fold change measured by WES analysis on crude biomass extracts). The R222W mutation, in absence of Y98F, leads to restoration of binding, which is consistent with data presented for H3/Aichi strain in Bradley et al., (2011, J. Virol 85:12387-12398) where a tryptophan (W) at residue 222 is present in the wild-type HA and binding was lost by introduction of the Y98F mutation.

### B HA

The yield of VLP comprising modified HAs produced in a plant was similar or greater than the yield of the corresponding parent or non-modified HA for VLPs comprising modified B Phuket/3073/2013 HAs (B Phu S140A; B Phu S142A; B Phu G138A; B Phu L203A; B Phu D195G; B Phu L203W; FIG. 4A). However the series of modified B-HAs exhibited a significant reduction in hemagglutination activity (expressed as HA titer) as shown in FIG. 4B.

Yield and hemagglutination activity were further assessed in a series of VLPs comprising non-modified or modified single mutation HA B Singapore-INFKK-16-0569-2016 (G138A, S140A, S142A, D195G, L203A, or L203W; FIGS. 4C and 4D, n=6), non-modified or modified single mutation HA B Maryland-15-2016 (G138A, S140A, S142A, D194G, L202A, or L202W; FIGS. 4E and 4F, n=6), non-modified or modified single mutation HA B Washington-02-2019 (G138A, S140A, S142A, D193G, L201A, or L201W; FIGS. 4G and 4H, n=6), non-modified or modified single mutation HA B Darwin-20-2019 (G138A, S140A, S142A, D193G, L201A, or L201W; FIGS. 41 and 4J, n=6), or non-modified or modified single mutation HA B Victoria-705-2018 (G138A, S140A, S142A, D193G, L201A, or L201W; FIGS. 4K and 4L, n=6). Non-binding candidates of HA B Singapore-INFKK-16-0569-2016, HA B Maryland-15-2016, HA B Washington-02-2019, HA B Darwin-20-2019, and HA B Victoria-705-2018 each lead to loss of binding (loss of HA titer in Hemagglutination assay) with no loss of yield (depicted in terms of fold change measured by WES analysis on crude biomass extracts).

### H5 HA

Hemagglutination activity was assessed for VLPs comprising either H5 A/Indonesia/5/05 or modified Y91F H5 A/Indonesia/5/05. The VLPs comprising modified Y91F H5 A/Indonesia/5/05 exhibited a significant reduction in hemagglutination activity (expressed as HA titer) as shown in FIG. 4M. Mice (n=10/group) were vaccinated with 3 μg VLP comprising H5 A/Indonesia/5/05 or modified Y91F H5 A/Indonesia/5/05 and boosted with 3 μg at 8 weeks. Sera were collected and HI titers were measured at weeks 4, 8 and 13. Both VLP comprising H5 A/Indonesia/5/05 or modified Y91F H5 A/Indonesia/5/05 result in similar total H5-specific IgG titers and there no differences in IgG avidity were observed.

### H7 HA

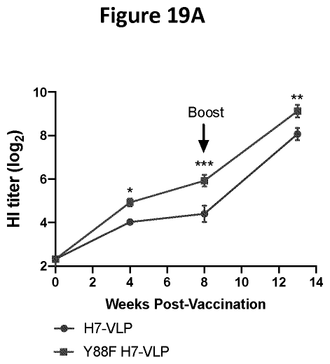

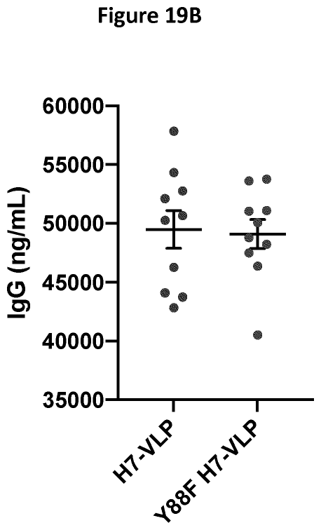

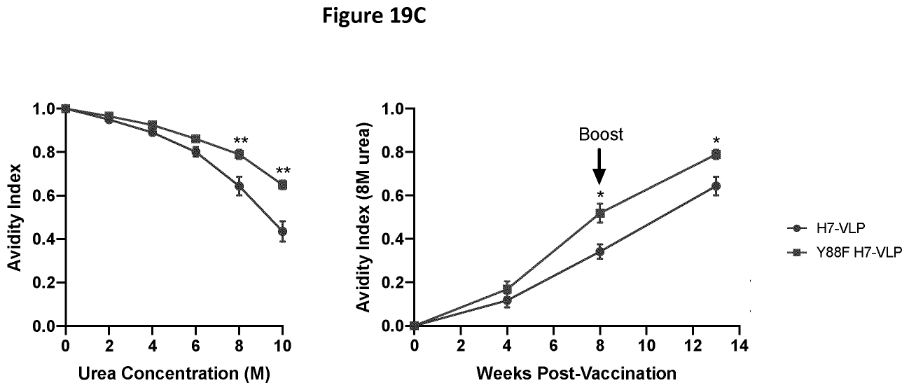

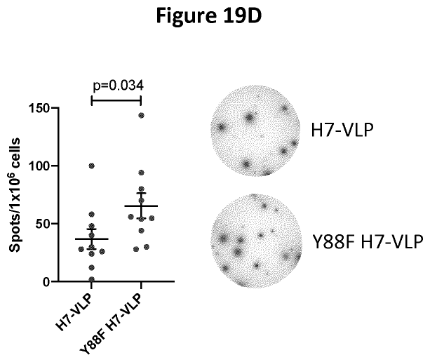

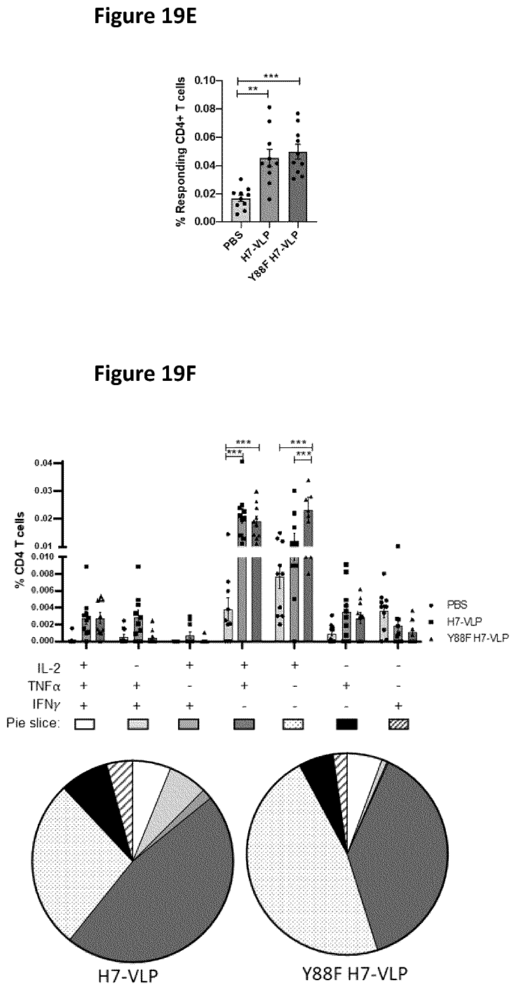

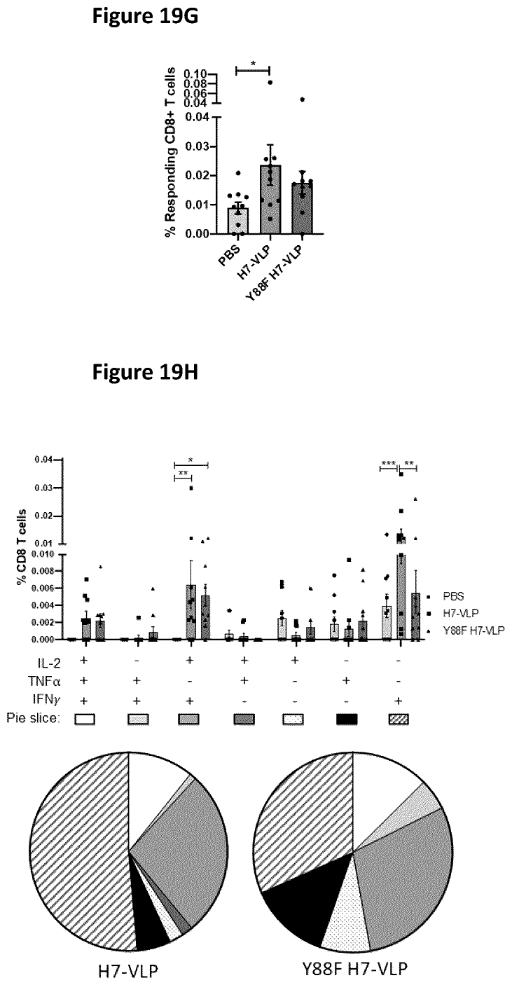

Hemagglutination activity was assessed for VLPs comprising either H7 A/Shanghai/2/2013 or modified Y88F H7 A/Shanghai/2/2013. The VLP comprising modified Y88F H7 A/Shanghai/2/2013 exhibited a significant reduction in hemagglutination activity (expressed as HA titer) as shown in FIG. 4N. The non-binding H7-VLP (Y88F) results in significantly higher hemagglutination inhibition (HI) titers at all time points measured, as shown in FIG. 19A. While the binding and non-binding (Y88F) H7-VLP result in similar total H7-specific IgG titers (FIG. 19B), non-binding H7-VLP results in enhanced IgG avidity maturation (FIG. 19C).

### Example 3: Materials & Methods

### Example 3.1: Human subjects and PBMC Isolation

Healthy adults aged 18-64 were recruited by the McGill Vaccine Study Centre and participants provided written consent prior to blood collection. This protocol was approved by the Research Ethics Board of the McGill University Health Centre.

Human PBMC were isolated from peripheral blood by differential-density gradient centrifugation within one hour of blood collection. Briefly, blood was diluted 1:1 in phosphate-buffered saline (PBS) (Wisent) at room temperature prior to layering over Lymphocyte Separation Medium (Ficoll) (Wisent). PBMC were collected from the Ficoll-PBS interface following centrifugation (650×g, 45 min, 22° C.) and washed 3 times in PBS (320×g, 10 min, 22° C.). Cells were resuspended in RPMI-1640 complete medium (Wisent) supplemented with 10% heat inactivated fetal bovine serum (Wisent), 10 mM HEPES (Wisent), and 1 mM penicillin/streptomycin (Wisent).

### Example 3.2. Hemagglutination Assay

Hemagglutination assay was based on a method described by Nayak and Reichl (2004, J. Viorl. Methods 122:9-15). Briefly, serial two-fold dilutions of the test samples (100 μL) were made in V-bottomed 96-well microtiter plates containing 100 μL PBS, leaving 100 μL of diluted sample per well. One hundred microliters of a 0.25% turkey (for H1) red blood cells suspension (Bio Link Inc., Syracuse, N.Y., or Lampire Biological Laboratories) were added to each well, and plates were incubated for 2-20h at room temperature. The reciprocal of the highest dilution showing complete hemagglutination was recorded as HA activity. In parallel, a recombinant HA standard was diluted in PBS and run as a control on each plate. Hemagglutination was indicated by the absence of a cell pellet after this period.

Where indicated, 1×10⁶ human PBMC were incubated for 30 min with 1-5 μg parent HA VLP (e.g. H1 HA) or modified HA VLP (e.g. Y91F H1 HA) and cell clustering was evaluated by light microscopy.

### Example 3.3: Surface Plasmon Resonance (SPR) Analysis

SPR is a label-free technology used to detect biomolecular interactions based on a collective electron oscillation happening at a metal/dielectric interface. Changes on the refractive index are measured on the surface of a sensor chip (mass change) which can deliver kinetics, equilibrium and concentration data. The SPR-based potency assay is an antibody independent receptor-binding SPR-based assay. The assay uses the Biacore™ T200 and 8K SPR instruments from GE Healthcare Life Sciences and quantifies the total amount of functionally active trimeric or oligomeric HA protein in the vaccine samples through binding to a biotinylated synthetic α-2,3 (avian) and α-2,6 (human) sialic acid glycan immobilized to a Streptavidin Sensor Chip as described in Khurana et. al. (Khurana S., et. al., 2014, Vaccine 32:2188-2197).

### Example 3.4: Mice and Vaccination

Female Balb/c mice were immunized by injection into the gastrocnemius muscle with 0.5-3 μg parent HA-VLP or modified HA VLP (50 μL total in PBS). Mice were vaccinated on day 0 and boosted on day 21 (when indicated). Blood was collected from the left lateral saphenous vein before vaccination and at D21 post-vaccination. Sera were obtained by centrifugation of blood in microtainer serum separator tubes (Beckton Dickinson) (8000×g, 10 min) and stored at −20° C. until further analysis.

To evaluate humoral and cell-mediated immune responses mice were euthanized on day 28 (one-dose) or day 49 (28d post-boost) by CO₂ asphyxiation. Blood was collected by cardiac puncture and cleared serum samples were obtained as described above. Spleens and bilateral femurs were harvested and splenocytes and bone marrow immune cells were isolated (Yam, K. K., et al., Front Immunol, 2015. 6: p. 207; Yam, K. K., et al., Hum Vaccin Immunother, 2017. 13(3): p. 561-571).

To evaluate vaccine efficacy, mice were challenged with 1.58×10³ times the median tissue culture infectious dose (TCID₅₀) of H1N1 A/California/07/09 (National Microbiology Laboratory, Public Health Agency of Canada). Mice were anesthetized using isoflurane and infected by intranasal instillation (25 μL/nare). Mice were monitored for weight loss for 12 days post-infection and were euthanized if they lost 20% of their pre-infection weight. On days 3 and 5 post-infection a subset of mice was sacrificed, and lungs were harvested for evaluation of viral load and inflammation. Lung homogenates were prepared as previously described (Hodgins, B., et al., Clin Vaccine Immunol, 2017. 24(12)) and stored at −80° C. until further analysis.

### Example 3.5: Antibody Titer Measurement

Neutralizing antibodies were evaluated by hemagglutination inhibition (HAI) assay (Zacour, M., et al., Clin Vaccine Immunol, 2016. 23(3): p. 236-42; WHO Global Influenza Surveillance Network. 2011. World Health Organization. ISBN 978 9241548090:43-62) and microneutralization (MN) assay (Yam, K. K., et al., Clin Vaccine Immunol, 2013. 20(4): p. 459-67). Titers are reported as the reciprocal of the highest dilution to inhibit hemagglutination (HAI) or cytopathic effects (MN). Samples below the limit of detection (<10) were assigned a value of 5 for statistical analysis.

HA-specific IgG was quantified by enzyme-linked immunosorbent assay (ELISA) as previously described (Hodgins, B., et al., Clin Vaccine Immunol, 2017. 24(12)) with the following modifications: plates were coated with 2 μg/mL recombinant HA (Immune Technologies) or HA-VLP (Medicago Inc.) and HA-specific IgG was detected using horseradish peroxidase (HRP)-conjugated anti-mouse IgG (Southern Biotech) diluted 1:20000 in blocking buffer. To evaluate the avidity of HA-specific IgG, wells containing bound antibody were incubated with urea (0M-8M) for 15 min and re-blocked for 1 h prior to detection. Avidity index (AI)=[IgG titer 2-8M urea/IgG titer 0M urea].

### Example 3.6: Antibody Secreting Cells (ASC)

HA-specific IgG ASC were quantified by ELISpot (Mouse IgG ELISpot^{BASIC}, Mabtech). Sterile PVDF membrane plates (Millipore) were coated with Anti-IgG capture antibody and blocked according to the manufacturer's guidelines. To quantify in vivo activated ASCs, wells were seeded with 250,000 (bone marrow) or 500,000 (splenocyte) freshly-isolated cells and incubated at 37° C., 5% CO₂ for 16-24h. HA-specific ASCs were detected according to the manufacturer's guidelines using 1 μg/mL biotinylated HA (immune tech, biotinylated using Sulfo-NHS-LC-Biotin). To evaluate memory ASCs, freshly isolated cells were polyclonally activated with 0.5 μg/mL R848 and 2.5 ng/mL recombinant mouse IL-2 (1.5×10⁶ cells/mL in 24-well plates) for 72h (37° C., 5% CO₂). Activated cells were re-counted and the assay was carried out as described above.

### Example 3.7: Splenocyte Proliferation

Splenocyte proliferation was measured by chemiluminescent bromodeoxyuridine (BrdU) incorporation ELISA (Sigma). Freshly isolated splenocytes were seeded in 96-well flat-bottom black plates (2.5×10⁵ cells/well). Cells were stimulated for 72h (37° C., 5% CO₂) with parent H1-VLP or peptide pools (BEI Resources) consisting of 15mer peptides overlapping by 11 amino acids spanning the complete HA sequences of parent H1/California/07/2009 (2.5 μg/mL). BrdU labelling reagent (10 μM) was added for the last 20h of incubation. BrdU was detected as described by the manufacturers. Proliferation is represented as a stimulation index compared to unstimulated samples.

### Example 3.8: Intracellular Cytokine Staining and Flow Cytometry

Freshly isolated splenocytes or bone marrow immune cells (1×10⁶/200 μL in a 96-well U-bottom plate) were stimulated with parent H1-VLP (2.5 μg/mL) or left unstimulated for 18h (37° C., 5% CO₂). After 12h, Golgi Stop and Golgi Plug (BD Biosciences) were added according to the manufacturer's instructions. Cells were washed 2× with PBS (320×g, 8 min, 4° C.) and labeled with Fixable Viability Dye eFluor 780 (eBioscience) (20 min, 4° C.). Cells were washed 3× followed by incubation with Fc Block (BD Biosciences) for 15 min at 4° C. Samples were incubated for an additional 30 min upon addition of the surface cocktail containing the following antibodies: anti-CD3 FITC (145-2C11, eBioscience), anti-CD4 V500 (RM4-5, BD Biosciences) anti-CD8 PerCP-Cy5.5 (53-6.7, BD Biosciences), anti-CD44 BUV395 (IM7, BD Biosciences) and anti-CD62L BUV373 (MEL-14, BD Biosciences). Cells were washed 3× and fixed (Fix/Perm solution, BD Biosciences) overnight. For detection of intracellular cytokines, fixed cells were washed 3× in perm/wash buffer (BD Biosciences) followed by intracellular staining with the following antibodies (30 min, 4° C.): anti-IL-2 APC (JES6-5H4, Biolegend), anti-IFNγ PE (XMG1.2, BD Biosciences) and anti-TNFα eFluor450 (MP6-XT22, Invitrogen). Cells were washed 3× in perm/wash buffer and then resuspended in PBS for acquisition using a BD LSRFortessa or BD LSRFortessa X20 cell analyzer. Data was analyzed using FlowJo software (Treestar, Ashland).

### Example 3.9: Lung Viral Load and Inflammation

Viral load was measured by TCID₅₀ in lung homogenates obtained at 3- and 5-days post infection (dpi). The assay was carried out and TCID₅₀ was calculated exactly as previously described (Hodgins, B., et al., Clin Vaccine Immunol, 2017, 24(12)). Lung homogenates were also evaluated in duplicate by multiplex ELISA (Quansys) according to the manufacturer's instructions.

### Example 4: Characterizing Modified, Non-Binding HA

VLPs Comprising Parent H1-HA or Modified H1-HA

Virus like particles comprising HA interact with human immune cells through binding to cell-surface SA (Hendin, H. E. et. al., 2017, Vaccine 35:2592-2599). Activation of human B cells following co-incubation with H1-VLP and VLPs bearing other mammalian HA proteins was also observed. However, VLPs targeting avian influenza strains such as H5N1 do not bind to or activate human B cells. Without wishing to be bound by theory, this lack of activation of B cells by H5N1 may be due to B cells not expressing terminal α(2,3)-linked SA.

A Y98F HA that does not bind to α(2,6)-linked SA (Whittle et al. (2014, J Virol, 88(8): p. 4047-57) was tested with the expectation that a VLP comprising Y98F HA would exhibit reduced humoral immune responses, since VLPs comprising Y98F HA would not be able to bind to or activate B cells through HA-SA interactions. However, as described below, modified H1 VLP (Y91F H1-VLP) elicited superior humoral responses and improved viral clearance compared to the native H1-VL.

Absence of Cell Clustering:

Incubation of human PBMC with the parent H1-VLP results in rapid cell clustering as a result of HA-SA interactions (Hendin, H. E., et al., Vaccine, 2017. 35(19): p. 2592-2599). However, PBMC incubated with the Y91F H1-VLP do not form clusters, even when the concentration of VLP is increased 5-fold. As shown in FIG. 5A, cell clustering was observed following incubation of human PBMC with VLPs comprising wild type H1 A/Calf (center panel). However, no cell clustering was observed when human PBMC was incubated in RPMI complete medium (cRPMI, control; left panel), or with VLPs comprising Y98F-H1 A/Calf (right panel).

Undetectable Hemagglutination:

The hemagglutination assay is a rapid method to estimate the amount of VLP or influenza virus in any given sample. The parent H1-VLP readily hemagglutinates tRBC and results in an HA titer of 48000. However, when this assay was conducted with an equivalent protein concentration of Y91F H1-VLP, the HA titer was <10 (FIG. 5B).

SPR Results:

The results shown in FIG. 5C (obtained using SPR) demonstrate that the relative binding of Y91F H1 A/Cal was below limit of quantification (BLQ), and greatly reduced when compared with the binding observed using parent (wild type) H1 A/Calf (control; set to 100%).

VLPs Comprising Parent H3-HA or Modified H3-HA

In contrast with the results observed noted above for Y91F H1 HA, VLPs comprising Y98F H3 A/Kansas HA were observed to hemagglutinate tRBCs (FIG. 3B), suggesting that Y98F H3 A/Kansas is able to bind SA. Sialic acid binding with VLPs comprising parent H3 A/Kansas or Y98F H3 A/Kansas HA was confirmed using SPR. VLPs comprising Y98F H3 A/Kansas exhibited approximately 80% of the amount of biding as VLPs comprising parent H3 HA ((FIG. 5D; Control; set to 100%). These results are to be contrasted with those reported for Y98F H3 A/Aichi which was shown to not bind SA (Bradley et al., 2011, J. Virol 85:12387-12398).

Additional modifications to H3 HA resulted in a significant reduction of HA titer (FIG. 3B). Examples of modifications to H3 HA that reduced H3 HA hemagglutination titer, include the Y98F in combination with any of S136D, S136N, S137N, D190G, D190K, R222W, S228N, S228Q.

The SA binding or non-binding properties for modified H3 HA comprising the following single mutations S136D, S136N, D190K, R222W, S228N, and S228Q were also evaluated (FIG. 3D). Mutations S136D, S136N, D190K, S228N, and S228Q in H3 HA lead to a loss of binding, as indicated by the reduced HA titer. The R222W mutation, in absence of Y98F, leads to restoration of binding, which is consistent with data presented for H3/Aichi strain in Bradley et al., (2011, J. Virol 85:12387-12398) where a tryptophan (W) at residue 222 is present in the wild-type HA and binding was lost by introduction of the Y98F mutation.

### Example 4.1: Activation of Human Immune Cells In Vitro

Human PBMC were stimulated with 1 μg parent H1-VLP or Y91F H1-VLP for 6h in vitro and cell activation was evaluated on the basis of CD69 expression.

Reduced B Cell Activation:

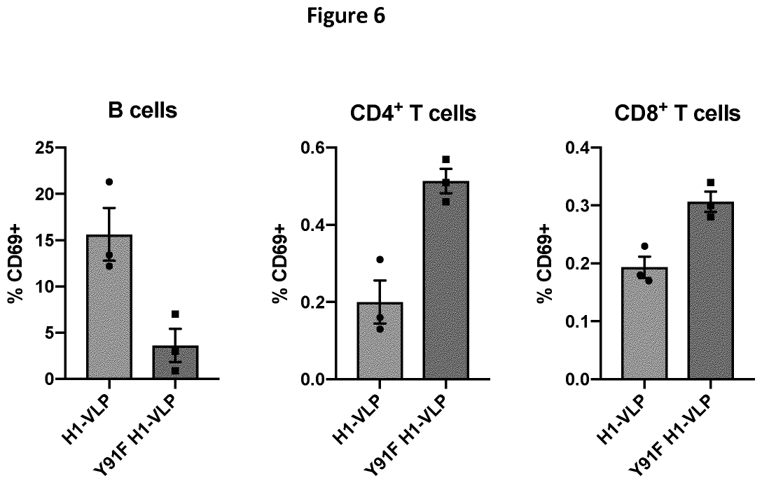

VLPs comprising wild type H1 resulted in activation of 15.6±2.9% of B cells compared to only 3.6±1.8% with VLPs comprising the modified HA (Y91F H1-VLP; FIG. 6, “B cells”). Activation of antigen-specific B cells is essential for a successful humoral immune response to vaccination. However, these cells typically make up <1% of total B cells (Kodituwakku, A. P., et al., Cell Biol, 2003. 81(3): p. 163-70). Without wishing to be bound be theory, HA-SA interactions between wild type (parent) H1-VLP and B cells likely facilitate activation of B cells that cannot produce HA-specific antibodies.

Increased T Cell Activation:

VLPs comprising modified HA (Y91F H1-VLP) resulted in increased activation of CD4⁺ and CD8⁺ T cells compared to VLPs comprising parent (wild type) HA (H1-VLP). The Y91F H1-VLP elicited activation of 0.2±0.06% of CD4⁺ T cells (FIG. 6, “CD4⁺ T cells”) and 0.19±0.02% of CD8⁺ T cells (FIG. 6, “CD8⁺ T cells”), compared to 0.5±0.03% of CD4⁺ T cells and 0.3±0.02% of CD8⁺ T cells with the parent H1-VLP.

### Example 4.2: Animal Study Results

Improved Humoral Immune Responses:

To establish whether HA-SA interactions influence the humoral immune response to vaccination in mice, neutralizing antibodies against H1N1 (A/California/07/2009) were measured in the serum 21 days post-vaccination with 3 μg parent H1-VLP or Y91F H1-VLP. Neutralizing antibodies were measured using hemagglutination inhibition (HAI) assay to measure antibodies that block the binding of live virus to turkey erythrocytes (Cooper, C., et al., HIV Clin Trials, 2012. 13(1): p. 23-32) and the microneutralization (MN) assay to measure antibodies that prevent infection of Madin-Darby Canine Kidney (MDCK) cells (Zacour, M., et al., Clin Vaccine Immunol, 2016. 23(3): p. 236-42; Yam, K. K., et al., Clin Vaccine Immunol, 2013. 20(4): p. 459-67).

Vaccination with the Y91F H1-VLP resulted in a statistically significant increase in HAI and MN titers compared to parent H1-VLP-vaccinated mice (FIG. 7A). Similar trends were observed when sera were evaluated at 2-week intervals for 8 weeks post-vaccination. Mice that received the Y91F H1-VLP had marginally higher H1-specific IgG titers at all timepoints, with the largest separation occurring at 8 weeks post-vaccination (FIG. 7B). At 8 weeks post-vaccination, the avidity of H1-specific IgG in Y91F H1-VLP-vaccinated mice was significantly higher than the parent H1-VLP vaccinated mice (P<0.033; FIG. 7C), and the increase in avidity was maintained over a 7 month period (FIG. 7F). The non-binding Y91F H1-VLP resulted in higher HI and MN titers at 7 months post-vaccination and improved durability of HI titers (FIGS. 7G and 7H). Mice (n=7-8/group) were vaccinated (IM) with H1-VLP or Y91F H1-VLP (3 μg/dose). Sera were collected on a monthly basis to measure HI titers (FIG. 7G) and MN titers (FIG. 7H). Statistical significance was determined by multiple t tests corrected for multiple comparisons using the Holm-Sidak method (*p<0.033, **p<0.01).

Similar titers were achieved by week 12, however, the Y91F H1-VLP treatment resulted in a more rapid increase over weeks 2-4, compared with vaccination using the corresponding wild type (parent) H1-VLP. High HAI titers at early time points may be associated with maintenance of titers at 28-weeks post vaccination. At week 28, only 3 out of 8 parent H1-VLP vaccinated mice had an HAI titer ≥40 compared to 6 out of 7 vaccinated mice in the Y91F H1-VLP group.

Hemagglutination inhibition (HI) titers were also increased following vaccination with VLP comprising Y91F H1-A/Idaho/07/2018 but narrowly failed to achieve statistical significance (FIG. 7I). Mice (n=8/group) were vaccinated with 1 μg binding or non-binding (Y91F) H1-VLP (A/Idaho/07/2018) and boosted with 1 μg at day 21. Sera were collected and HI titers were measured 21d post-boost. Statistical significance was evaluated using the Mann-Whitney test. The non-binding H1-VLP derived from A/Idaho/07/2018 results in a slight increase in H1-specific IgG following a single vaccine dose (FIG. 7J, left panel) but this difference is lost post-boost (FIG. 7J, right panel).

Vaccination with VLP comprising non-binding H1 A/Brisbane/02/2018 resulted in higher H1-specific IgG titers at day 21 and day 21 post-boost (day 42) and higher avidity (FIGS. 7K and 7L). Mice (n=18/group) were vaccinated with 0.5 μg binding or non-binding recombinant H1 (A/Brisbane/02/2018) and boosted with 0.5 μg at day 21. Sera were collected and H1-specific IgG was measured by ELISA 21d post-prime and 21d post-boost (d42). IgG avidity was assessed using an avidity ELISA. Bound serum samples were treated with 4-6M Urea and the avidity index represents the proportion of IgG that remains bound after the urea incubation ([IgG titer 2-10M urea]/[IgG titer 0M urea]). Statistical significance was determined by Mann-Whitney test (*p<0.033, ***p<0.001)

Vaccination with Y88F H7-VLP resulted in a statistically significant increase in HAI titers compared to parent H7-VLP-vaccinated mice, up to two months post vaccination (FIG. 7E).

In contrast to VLPs comprising non-binding H1 and H7, there was no change in hemagglutination inhibition (HI) titers following vaccination with VLP comprising non-binding (NB) D195G B/Phuket/3073/2013 (FIG. 7M, left panel). Mice (n=7-8/group) were vaccinated with 1 μg binding or non-binding (NB) B-VLP (D195G B/Phuket/3073/2013) and boosted with 1 μg at day 21. Sera were collected and HI titers were measured 21d post-boost. Microneutralization (MN) titers were lower following vaccination with NB B-VLP but the difference was not statistically significant (FIG. 7M, right panel). Vaccination with VLP comprising non-binding (NB) D195G B/Phuket/3073/2013 results in similar amounts of HA-specific IgG at day 21 and day 21 post-boost (day 42) (FIG. 7N) but there is a slight increase in IgG avidity (FIG. 7O). Sera were collected and H1-specific IgG was measured by ELISA 21d post-prime and 21d post-boost (d42). IgG avidity was assessed using an avidity ELISA. Bound serum samples were treated with 4-6M Urea and the avidity index represents the proportion of IgG that remains bound after the urea incubation ([IgG titer 2-10M urea]/[IgG titer 0M urea]). Differences in avidity were not statistically significant.

To further characterize the B cell response, memory B cells and in vivo activated antibody secreting cells (ASCs) were quantified in the spleen and bone marrow by enzyme-linked immune absorbent spot (ELISpot) assay. Mice were vaccinated twice (3 weeks apart) with 3 μg or 0.5 μg VLP and ASCs were evaluated 4 weeks post-boost. Similar levels of memory B cells were observed in the spleen regardless of vaccine or dose, but there was a trend towards an increase in the bone marrow of Y91F H1-VLP-vaccinated mice (FIG. 8A). In vivo activated ASCs were only evaluated in mice that received 0.5 μg VLP. In these mice, vaccination with Y91F H1-VLP resulted in an increase in ASCs in both spleen and bone marrow (FIG. 8B). In the bone marrow, ASCs from Y91F H1-VLP-vaccinated mice also produced more IgG on a per-cell basis as measured by spot size (FIG. 8C). Vaccination with Y91F H1-VLP results in slightly increased bone marrow plasma cells (BMPC) at 7 months post-vaccination and correlates with maintenance of MN titers (FIG. 8D). Mice (n=7-8/group) were vaccinated (IM) with H1-VLP or Y98F H1-VLP (3 μg/dose). Mice were euthanized at 7 mpv and BM was collected to quantify H1-specific plasma cells (PC) in the bone marrow by ELISpot. Representative wells from each group are shown on the right. All mice that had >10 BMPC/1×10⁶ cells maintained their MN titers between 3 and 7 months post-vaccination. All mice with <10 BMPC/1×10⁶ cells had a decline in MN titers after 3 months.

Strong Cell-Mediated Immune Responses:

The enhanced cell-mediated immunity (CMI) elicited by plant-derived HA-VLPs is one of the key features that distinguishes these vaccines from other formulations. Therefore, maintenance of cellular responses in mice vaccinated with Y91F H1-VLP was examined. CMI was evaluated on the basis of proliferative responses and cytokine profiles of memory T cells.

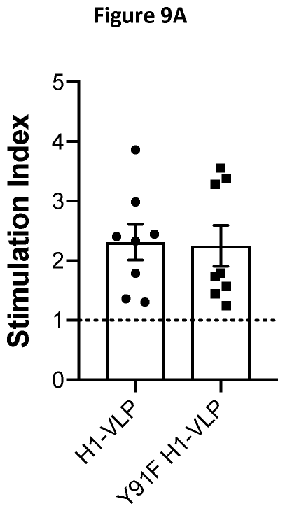

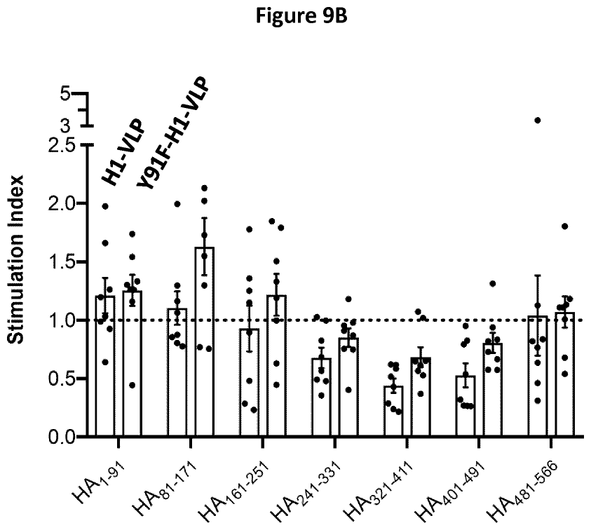

Proliferation was quantified by measuring incorporation of the synthetic thymidine analog bromodeoxyuridine (BrdU) in splenocytes upon re-stimulation with H1 antigens. Re-stimulation with parent H1-VLP (2 μg/mL) resulted in similar stimulation indices in mice vaccinated with parent H1-VLP or Y91F H1-VLP (FIG. 8A). However, unique proliferation profiles were observed when splenocytes were stimulated with peptide pools corresponding to different parts of the HA sequence. Pools designed for antigen-specific T cell stimulation were composed of 20 overlapping peptides (15aa each) and spanned the entire parent H1 A/California/07/09 sequence. Peptide pools spanning amino acids 81-251 elicited higher levels of proliferation in mice vaccinated with the Y91F H1-VLP compared with proliferation observed using the corresponding parent H1 HA peptides (FIG. 9B). Peptides pools spanning amino acids 81-251 encode a section of the HA protein found within the globular head.

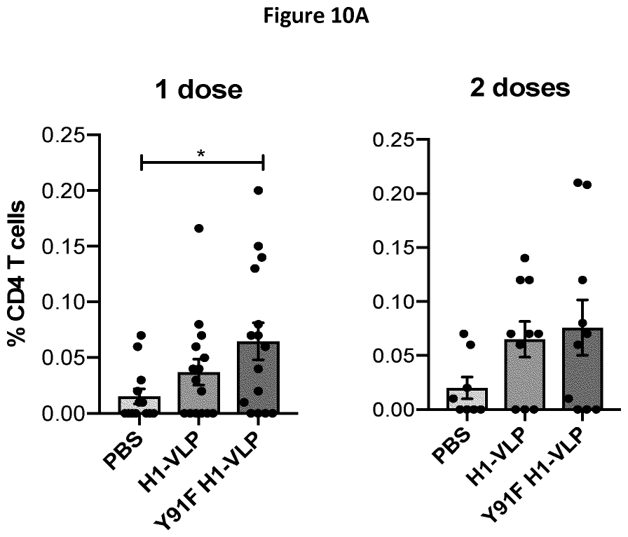

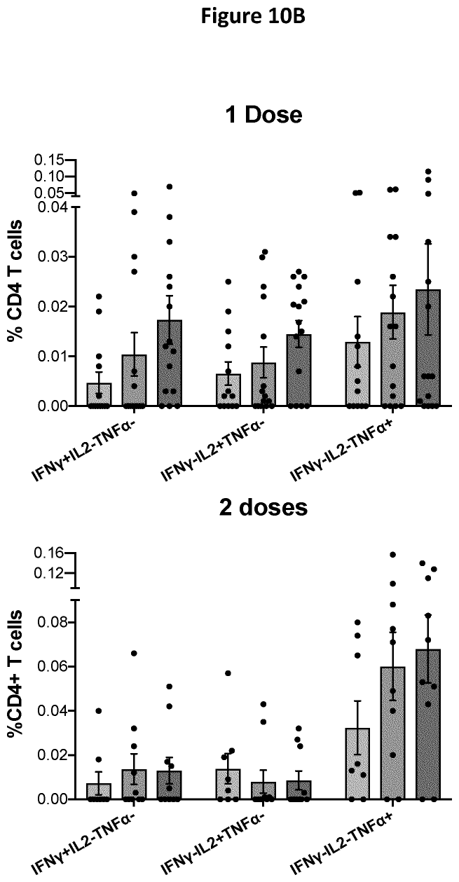

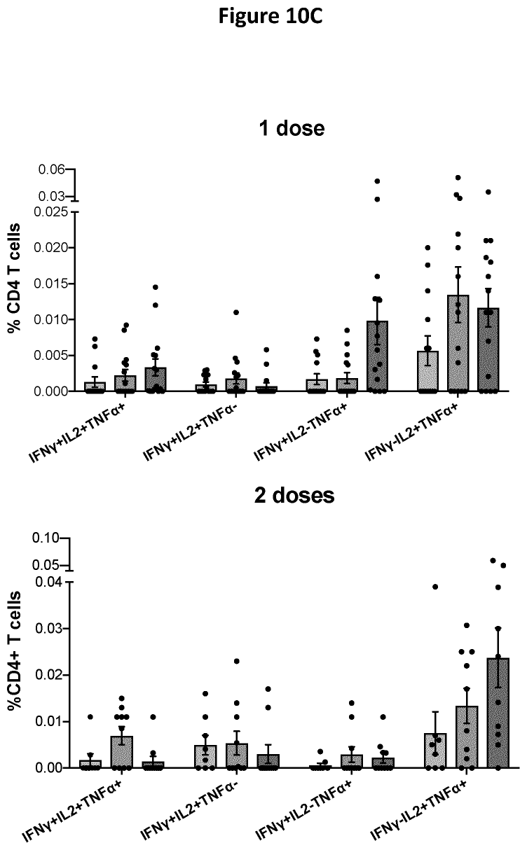

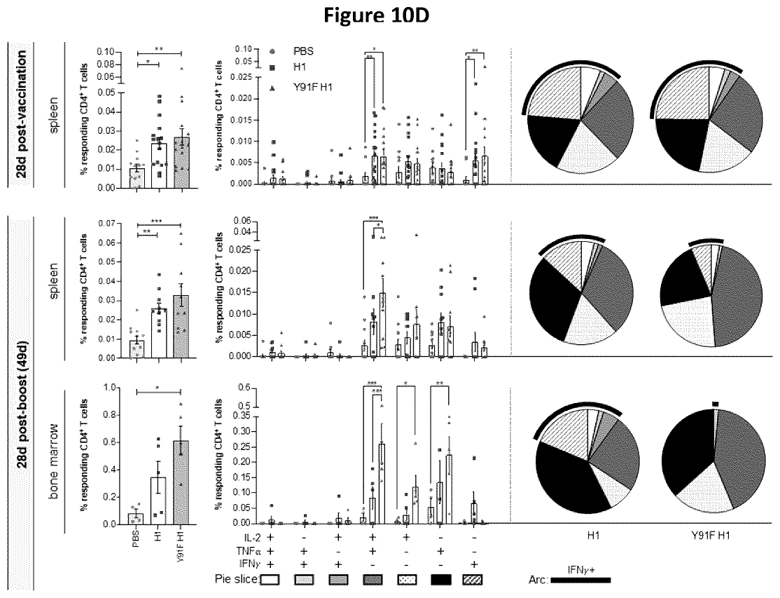

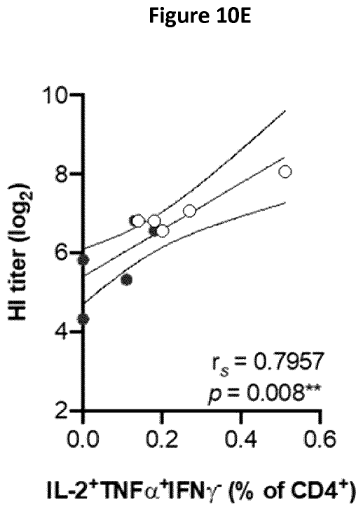

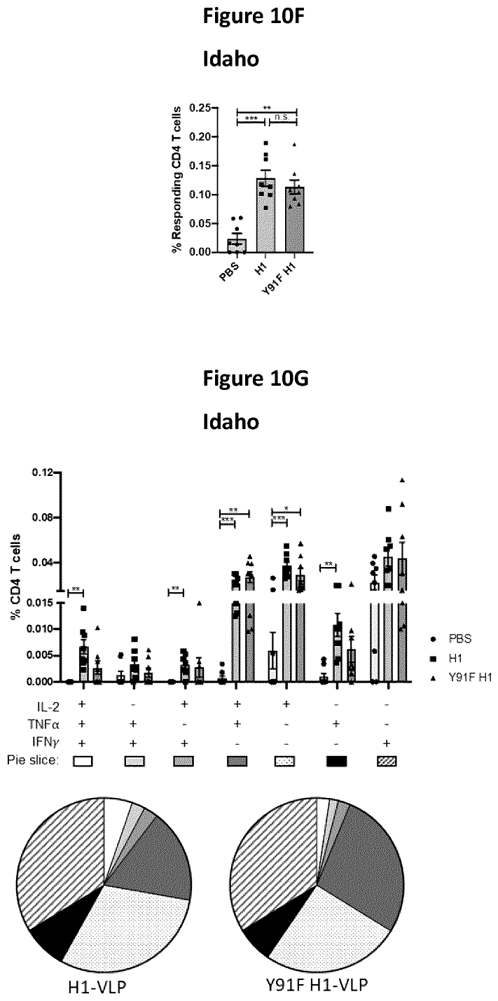

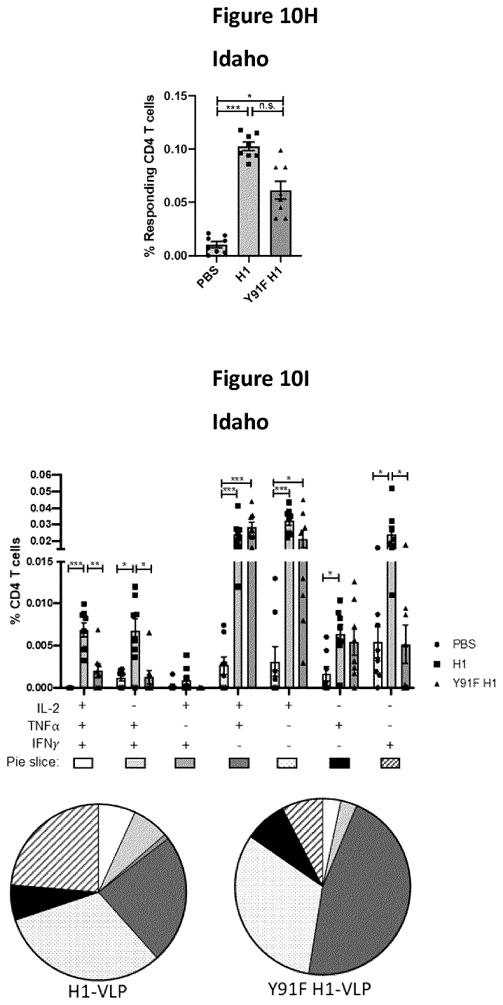

Cytokine production by splenocytes was measured using flow cytometry. Antigen-specific T cells were identified on the basis of IL-2, TNFα, or IFNγ production, following re-stimulation with parent H1-VLP or Y91F H1-VLP (both at 2.5 μg/mL) for 18h. Both the parent H1-VLP and Y91F H1-VLP resulted in an increase in H1-specific CD4⁺ T cells 28 days post-vaccination, however, this increase was only statistically significant in the Y91F H1-VLP group (FIG. 10A). Within this antigen-specific population, Boolean analysis was applied to evaluate the various populations of single-, double-, and triple-positive CD4⁺ T cells. Both vaccines (parent H1-VLP and Y91F H1-VLP) elicited a slight increase in each of the single-positive populations and the triple-positive population. However, only the Y91F H1-VLP elicited a substantial increase in the IFNγ⁺IL-2-TNFα⁺ population (FIG. 10B-C).

Splenocytes and bone marrow immune cells were further analyzed for the frequency of CD4⁺ T cells expressing CD44 (antigen specific) and at least one of IL-2, TNFα or IFNγ (FIG. 10D, left). At indicated time points following vaccination (28d post-vaccination and 28d post boost, i.e. 49d), mice were euthanized and splenocytes/bone marrow immune cells were isolated. Cells were stimulated for 18h with 2.5 μg/mL H1-VLP. Flow cytometry was used to quantify H1-specific CD4⁺ T cells. Statistical significance was determined by Kruskal-Wallis test with Dunn's multiple comparisons (total response) or two-way ANOVA with tukey's multiple comparisons (cytokine signatures) (*p<0.033, **p<0.01, ***p<0.001). Background values obtained from non-stimulated samples were subtracted from values obtained following stimulation with H1-VLP. Individual cytokine signatures for each mouse obtained by Boolean analysis were comparatively analyzed between the indicated time points and cell types. Background values obtained from non-stimulated samples were subtracted from values obtained following stimulation with H1-VLP. The bar graph shows the frequency of each of the populations and the pie charts show the prevalence of each responding population among total responding cells. After one dose (28d post-vaccination, FIG. 10D, top panel) there is no difference in the magnitude or cytokine signatures of the splenic CD4+ T cell response. Following the second dose (28d post-boost, FIG. 10D, middle panel) the magnitude of the splenic CD4+ T cell responses are similar, however, the non-binding H1-VLP results in a decreased proportion of cells expressing of IFNγ and an increase in the IL-2⁺TNFα*IFNγ⁻ CD4+ T cell population. These cytokine signatures were mirrored in the bone marrow (FIG. 10D, bottom panel), however, the frequency of H1-specific CD4 T cells was increased in the bone marrow of mice vaccinated with the non-binding H1-VLP. Bone marrow CD4+ T cells tend to be long-lived and may contribute to improved durability of antibody responses that we observed.

It was further observed that the frequency of IL-2⁺TNFα⁺IFNγ⁻ CD4⁺ T cells in the bone marrow correlate with HI titer (FIG. 10E). Mice vaccinated with the non-binding H1-VLP had a significant increase in the frequency of IL-2⁺TNFα⁺IFNγ⁻ CD4⁺ T cells in the BM (FIG. 10D, bottom panel) which correlated with increased HI titers in these mice (FIG. 10E). Rank correlation technique was applied to evaluate the relationship between the frequency of IL-2⁺TNFα⁺IFNγ⁻CD4⁺ T cells in the BM and HAI titer. Mice vaccinated with Y91F H1-VLP are shown in white circle and H1-VLP are shown in solid dark.

Total splenic CD4 T cell responses were similarly maintained following vaccination with VLP comprising non-binding (Y91F) H1-A/Idaho/07/2018 (1 week post-boost). Mice (n=8/group) were vaccinated with 1 μg VLP comprising binding H1 A/Idaho/07/2018 or non-binding (Y91F) H1 A/Idaho/07/2018 and boosted with 1 μg at day 21. Mice were euthanized 1 week post-boost and spleens were harvested to measure antigen-specific (CD44+) CD4 T cells by flow cytometry. Both vaccines resulted in similar frequencies of responding cells (FIG. 10F) with similar frequencies of polyfunctional CD4 T cells (FIG. 10G). However fewer CD4 T cells expressing IFNγ were observed upon vaccination with VLP comprising non-binding H1 A/Idaho/07/2018 3 weeks post-boost (FIG. 10H). Mice were euthanized 3 weeks post-boost and spleens were harvested to measure antigen-specific (CD44+) CD4 T cells by flow cytometry. The frequency of total responding CD4 T cells was reduced following vaccination with Y91F H1-VLP 3 weeks post-boost but this difference was not significant (FIG. 10H). Similar to mice vaccinated with VLP comprising H1 California, the IL-2⁺TNFα⁺IFNγ⁻ population dominated the response to Y91F H1-VLP 3 weeks post boost (FIG. 10I). Most IFNγ⁺ populations were reduced in mice vaccinated with Y91F H1-VLP. Statistical significance was determined by Kruskal-Wallis test with Dunn's multiple comparisons (10F and 10H) or two-way ANOVA with Tukey's multiple comparisons (10G and 10I). *p<0.033, **p<0.01, ***p<0.001

Since CMI responses in naïve animals are generally weak after the first dose and previous studies evaluating CMI in response to HA-VLPs were conducted following a two-dose vaccine schedule, CMI was also evaluated in mice vaccinated with 2 doses of VLP. By 28d post-boost only the TNFα single-positive population (IFNγ+) was increased compared to the PBS (control) group and there was no difference between the two vaccines (FIG. 10B). The IFNγ⁻IL-2⁺TNFα⁺ population, which was present in both vaccine groups after one dose, continued to expand following a second dose of Y91F H1-VLP but not the parent (wild type) H1-VLP (FIG. 10C). These cells (the IFNγ⁻IL-2⁺TNFα⁺ population) have previously been described as a population of primed but uncommitted memory T helper cells known as primed precursor T helper (Thpp) cells (Pillet, S., et al., NPJ Vaccines, 2018. 3: p. 3; Deng, N., J. M. Weaver, and T. R. Mosmann, PLoS One, 2014. 9(5):p. e95986). Without wishing to be bound by theory, Thpp cells are thought to serve as a reservoir of memory CD4⁺ T cells with effector potential. While vaccines elicit Thpp cells in a naïve individual, these cells normally become IFNγ⁺ upon subsequent exposure. Since the cells become IFNγ⁺ with subsequent exposure, this may explain the decrease in the Thpp population, and increase in the triple-positive population (IFNγ⁺IL-2⁺TNFα⁺) upon boosting with H1-VLP. Expansion of the Thpp population upon boosting with Y91F H1-VLP suggests that this vaccine behaves similarly to other protein vaccines which have been shown to elicit stronger and more durable antibody responses than influenza vaccines (e.g. protein vaccines tetanus, diptherea; Deng, N., J. M. Weaver, and T. R. Mosmann, PLoS One, 2014. 9(5):p. e95986).

Reduced Viral Load:

Mice were challenged with 1.58×10³ times the median tissue culture infectious dose (TCID₅₀) of parent (wild type) H1N1 (A/California/07/09) 28 days post-vaccination with 3 μg VLP. This resulted in substantial weight loss and 69% mortality in the control group (PBS), however, all mice vaccinated with parent H1-VLP or Y91F H1-VLP survived (FIG. 11A). In addition, there was no significant difference in post-infection weight loss between the vaccinated groups (FIG. 11B).

A subset of the infected mice were sacrificed 3 dpi (days post-infection) and 5 dpi to quantify viral titers in the lung as previously described (Hodgins, B., et al., Clin Vaccine Immunol, 2017. 24(12)). Consistent with survival and weight loss trends, a decrease in viral titer in mice vaccinated with either parent H1-VLP or Y91F H1-VLP was observed, compared to the PBS control group at 3 dpi. However, this difference is only statistically significant in the Y91F H1-VLP group (P<0.002). By 5 dpi, mice vaccinated with the Y91F H1-VLP had a 2-log reduction in viral titers compared to the PBS group (P<0.001), and significantly lower titers than the parent H1-VLP group (P<0.033; FIG. 11C).

Lung homogenates from 3 dpi and 5 dpi were also evaluated by multiplex ELISA (FIG. 11D). Mice were challenged with 1.6×10³ TCID₅₀ of H1N1 (A/California/07/09) 28 days post-vaccination and a subset of mice were mock infected with an equivalent volume of media. A subset of the mice (n=9/group/time point) were euthanized at 3 (FIG. 11D, left panel) and 5 (FIG. 11D, right panel) days post infection (dpi) to evaluate pulmonary inflammation. Concentrations of cytokines and chemokines in the supernatant of lung homogenates were measured by multiplex ELISA (Quansys). At 3 dpi the both vaccine groups had reduced inflammatory cytokines compared to the placebo group but there were no differences between vaccines. By 5 dpi the lungs of mice vaccinated with the non-binding H1-VLP had markedly less inflammatory cytokines typically associated with lung pathology. IFNγ neared baseline levels in these mice, suggesting that the Y91F H1-VLP results in enhanced protection from influenza-induced lung pathology compared to the parent H1-VLP. A subset of the mice was euthanized at 4 days post infection (dpi) to evaluate lung pathology (FIG. 11E). Mice vaccinated with Y91F H1-VLP had decreased pulmonary inflammation compared to H1-VLP-vaccinated mice and more closely resembled the mock-infected mice.

Immune Response Following Vaccination with VLP Comprising Modified H5:

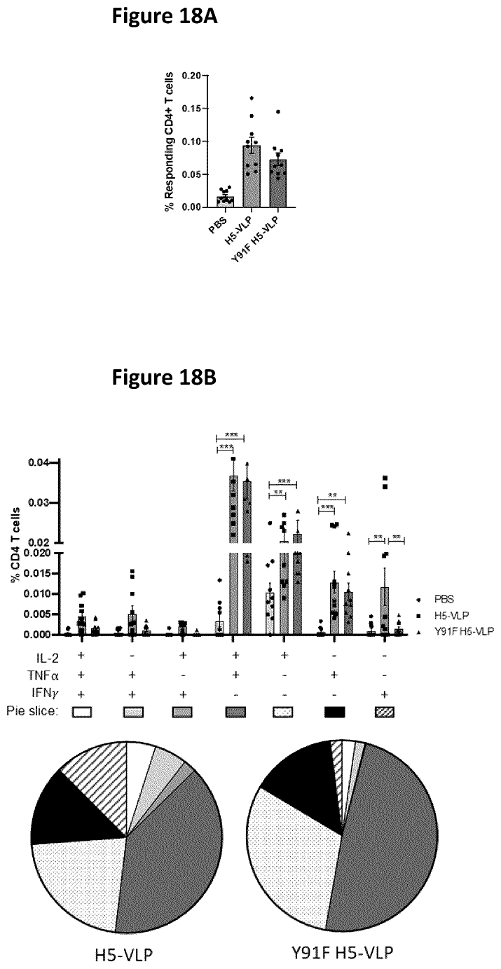

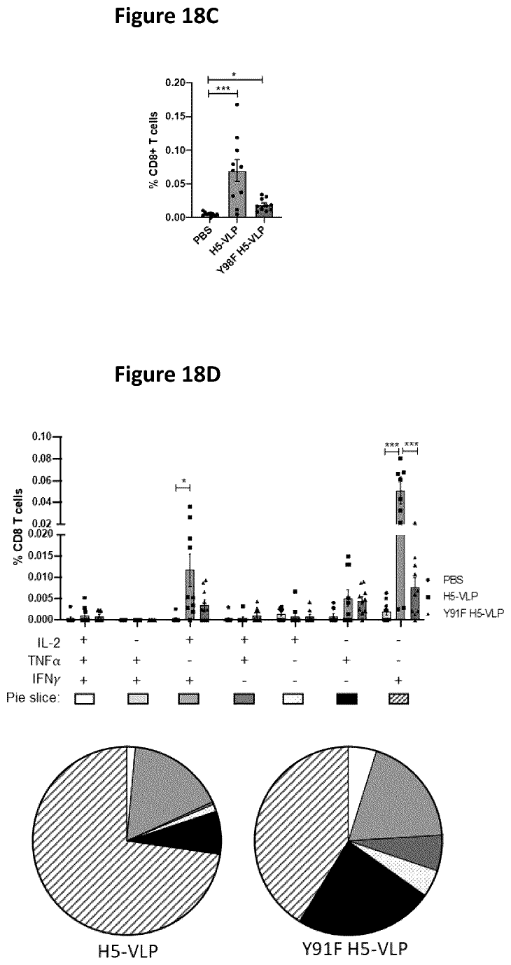

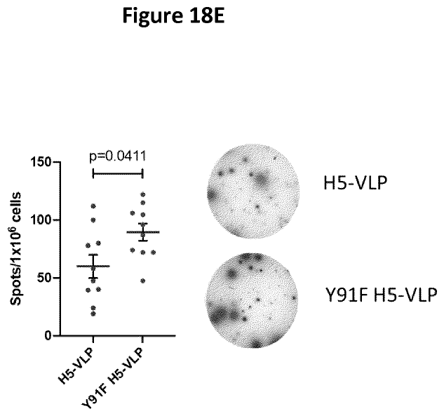

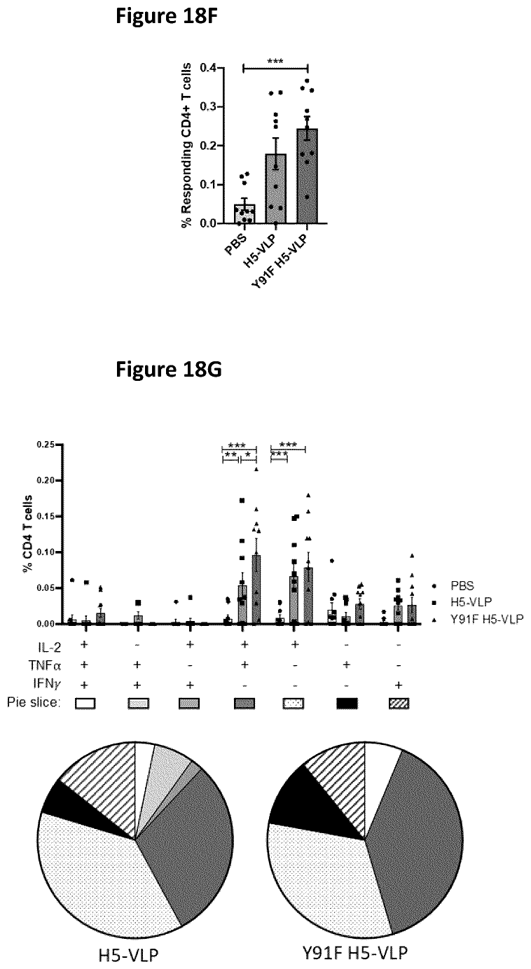

Total splenic CD4 T cell responses were maintained upon introduction of the Y91F mutation (FIGS. 18A and 18B). Mice (n=10/group) were vaccinated with 3 μg binding or non-binding (modified, Y91F) H5-VLP and boosted with 3 μg at 8 weeks. Mice were euthanized 5 weeks post-boost and spleens were harvested to measure antigen-specific (CD44+) CD4 T cells by flow cytometry. Both VLP comprising H5 A/Indonesia/5/05 or modified H5 A/Indonesia/5/05 resulted in similar frequencies of responding cells (FIG. 18A) with similar frequencies of polyfunctional CD4 T cells (FIG. 18B). However, Y91F H5-VLP resulted in fewer IFNγ single positive cells. (triple positive) CD4 T cells (FIG. 18B). In contrast to splenic CD4 T cell response following vaccination with VLP comprising modified H5 A/Indonesia/5/05, splenic CD8 T cell responses were reduced upon introduction of the non-binding mutation. Mice were euthanized 5 weeks post-boost and spleens were harvested to measure antigen-specific (CD44+) CD8 T cells by flow cytometry. Both VLPs resulted in a significant increase in total responding cells compared to the placebo group but the response was considerably stronger in mice that received the parent H5-VLP (FIG. 18C). This increase was driven by an increase in IFNγ single-positive cells and IL-2⁺IFNγ⁺ cells (FIG. 18D). Statistical significance was determined by Kruskal-Wallis test with Dunn's multiple comparisons (left) or two-way ANOVA with Tukey's multiple comparisons (FIG. 18D). *p<0.033, **p<0.01, ***p<0.001.

Notably, non-binding H5-VLP results in increased H5-specific bone marrow plasma cells (BMPC) (FIG. 18E). Mice were euthanized 5 weeks post-boost and bone marrow (BM) was harvested to measure H5-specific BMPC by ELISpot assay. Images of representative wells are shown on the right. Statistical significance was evaluated using the Mann-Whitney test. In contrast to splenic CD4 T cell frequency, non-binding H5-VLP results in increased antigen-specific CD4 T cells in the bone marrow (BM) (FIG. 18F). Mice were euthanized 5 weeks post-boost and BM harvested to measure antigen-specific (CD44+) CD4 T cells by flow cytometry.

Among evaluated VLPs comprising modified HA, non-binding H1, H5 and H7 VLP resulted in a significant increase in responding CD4 T cells when compared to the placebo group (see FIGS. 10D (H1) and 18F (H5), data for H7 not shown). The pattern of immunity seen with H5 VLP is similar to the pattern observed for H1 VLP. As shown in FIG. 18G, Y91F H1-VLP also resulted in a significant increase in IL-2⁺TNFα⁺IFNγ⁻ CD4 T cells compared to the parent H5. Statistical significance was determined by Kruskal-Wallis test with Dunn's multiple comparisons (FIG. 18F) or two-way ANOVA with Tukey's multiple comparisons (FIG. 18G). *p<0.033, **p<0.01, ***p<0.001.

Immune Response Following Vaccination with VLP Comprising Modified H7:

Non-binding H7-VLP results in significantly higher hemagglutination inhibition (HI) titers up to 14 weeks post-vaccination as compared to VLP with parent H7 (FIG. 19A). Mice (n=10/group) were vaccinated with 3 μg binding or non-binding (Y88F) H7-VLP and boosted with 3 μg at 8 weeks. Sera were collected and HI titers were measured at weeks 4, 8 and 13. Statistical significance was determined by multiple T-tests with Holm-Sidak's multiple comparisons. *p<0.033, **p<0.01, ***p<0.001. Both vaccines result in similar total H7-specific IgG titers (FIG. 19B). However, the non-binding H7-VLP results in enhanced IgG avidity maturation (FIG. 19C). Sera were collected and IgG avidity was measured at weeks 4, 8 and 13. IgG avidity was assessed using an avidity ELISA. Bound serum samples were treated with 0-10M Urea and the avidity index represents the proportion of IgG that remains bound after the urea incubation ([IgG titer 2-10M urea]/[IgG titer 0M urea]). The left panel of FIG. 19C shows avidity indices at week 13. The right panel of FIG. 19C shows changes in avidity over time (8M urea). Statistical significance was determined by multiple T-tests with Holm-Sidak's multiple comparisons. *p<0.033, **p<0.01. Non-binding H7-VLP results in increased H7-specific bone marrow plasma cells (BMPC) (FIG. 19D). Mice were euthanized 5 weeks post-boost and bone marrow (BM) was harvested to measure H7-specific BMPC by ELISpot assay. Images of representative wells are shown on the right. Statistical significance was evaluated using the Mann-Whitney test.

Splenic CD4 T cell responses were maintained upon introduction of the non-binding H7 mutation. Mice were euthanized 5 weeks post-boost and spleens were harvested to measure antigen-specific (CD44+) CD4 T cells by flow cytometry. Both vaccines resulted in similar frequencies of responding cells (FIG. 19E) with similar frequencies of IL-2⁺TNFα⁺IFNγ⁺ (triple positive) CD4 T cells (FIG. 19F). The Y88F H7-VLP resulted in increased IL-2 single positive cells. Statistical significance was determined by Kruskal-Wallis test with Dunn's multiple comparisons (FIG. 19E) or two-way ANOVA with Tukey's multiple comparisons (FIG. 19F). *p<0.033, **p<0.01, ***p<0.001. Splenic CD8 T cell responses were similar between vaccine groups. Mice were euthanized 5 weeks post-boost and spleens were harvested to measure antigen-specific (CD44+) CD8 T cells by flow cytometry. In general, CD8 T cell responses were weak. Only the WT H7-VLP resulted in a significant increase in total responding cells (FIG. 19G), driven by an increase in IFNγ single-positive cells (FIG. 19H). Polyfunctional CD8 T cell signatures were similar in both vaccine groups with a significant increase in IL-2⁺IFNγ⁺ cells.

Immune Response Following Vaccination with VLP Comprising Modified B HA:

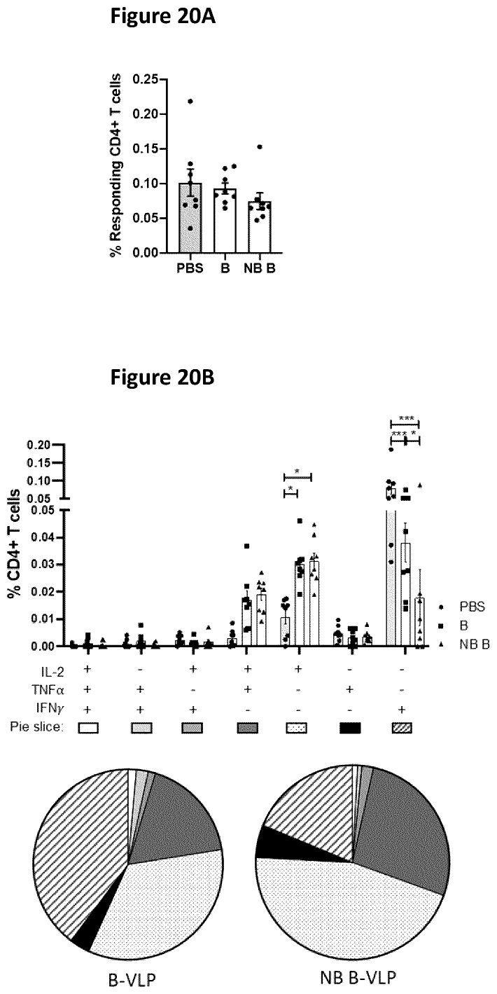

Fewer CD4 T cells expressing IFNγ were observed upon vaccination with non-binding B-VLP (3 weeks post-boost). Mice (n=8/group) were vaccinated with 1 μg binding or non-binding (NB) B-VLP (D195G B/Phuket/3073/2013) and boosted with 1 μg at day 21. Mice were euthanized 3 weeks post-boost and spleens were harvested to measure antigen-specific (CD44+) CD4 T cells by flow cytometry. The frequency of total responding CD4 T cells was similar between vaccine groups (FIG. 20A). Similar to other non-binding VLPs, the IL-2⁺ populations dominated the response to the NB B-VLP (FIG. 20B). However, IFNγ⁺ cells were reduced in mice vaccinated with NB B-VLP. Statistical significance was determined by Kruskal-Wallis test with Dunn's multiple comparisons (20A) or two-way ANOVA with Tukey's multiple comparisons (20B). *p<0.033, **p<0.01, ***p<0.001.

All citations are hereby incorporated by reference.

The present invention has been described with regard to one or more embodiments. However, it will be apparent to persons skilled in the art that a number of variations and modifications can be made without departing from the scope of the invention as defined in the claims.

What is claimed is:

1. A suprastructure comprising modified influenza hemagglutinin (HA), the modified HA comprising one or more than one alteration that reduces non-cognate interaction of the modified HA to sialic acid (SA) of a protein on the surface of a cell, while maintaining cognate interaction with the cell.

2. The suprastructure of claim 1 wherein the non-cognate interaction is binding of the modified HA to sialic acid (SA) of the protein on the surface of the cell.

3. The suprastructure of claim 1 or 2 wherein, the alteration comprises a substitution, deletion or insertion of one or more amino acids within the modified HA.

4. The suprastructure of claim 1 wherein the cell is a B cell.

5. The suprastructure of claim 1, wherein the protein on the surface of the cell is a B cell surface receptor.

6. The suprastructure of claim 1 wherein the suprastructure is a virus like particle (VLP).

7. A composition comprising the VLP of claim 6 and a pharmaceutically acceptable carrier.

8. A vaccine comprising the composition of claim 7.

9. A vaccine comprising the composition as defined in claim 7 and an adjuvant.

10. A plant or portion of a plant comprising the VLP of claim 6.

11. A nucleic acid encoding the modified HA of claim 1.

12. A plant or portion of a plant comprising the nucleic acid of claim 11.

13. A method of inducing immunity to influenza virus infection in an animal or subject in need thereof, comprising administering the vaccine as defined in claim 8 to the animal or subject.

14. The method of claim 13, wherein the vaccine is administered to the animal or the subject orally, intradermally, intranasally, intramuscularly, intraperitoneally, intravenously, or subcutaneously.

15. A use of the vaccine of claim 9 for inducing immunity to influenza virus infection in an animal or subject in need thereof.

16. A method of increasing an immunological response in an first animal or a subject in response to an antigen challenge comprising, administering a first vaccine, the first vaccine comprising the vaccine of claim 8 to the animal or subject and determining the immunological response, wherein the immunological response is a cellular immunological response, a humoral immunological response, or both the cellular immunological response and the humoral immunological response, and wherein the immunological response is increased when compared with a second immunological response obtained following administration of a second vaccine comprising virus like particles comprising a corresponding parent HA to a second animal or subject.

17. A method of producing a virus like particle (VLP) comprising, expressing the nucleic acid of claim 11 within a host under conditions that result in the expression of the nucleic acid and production of the VLP.

18. The method of claim 17, wherein the host is harvested and the VLP is purified.

19. A method of producing a suprastructure comprising modified HA in a plant or portion of a plant comprising, introducing the nucleic acid of claim 11 within the plant or portion of the plant, and growing the plant or portion of the plant under conditions that result in the expression of the nucleic acid and production of the suprastructure.

20. The method of claim 19, wherein the suprastructure is a virus like particle (VLP).

21. The method of claim 20, wherein the plant or portion of the plant is harvested and the VLP is purified.

22. A method of producing a suprastructure comprising modified HA in a plant or portion of a plant comprising, growing a plant, or portion of a plant that comprises the nucleic acid as defined in claim 11, under conditions that result in the expression of the nucleic acid and production of the suprastructure.

23. The method of claim 22, wherein the suprastructure is a virus like particle (VLP).

24. The method of claim 23, wherein the plant or portion of the plant is harvested and the VLP is purified.

25. A composition comprising the suprastructure of claim 1 or 2 and a pharmaceutically acceptable carrier.

26. A composition comprising one or more than one VLP as defined in claim 6.

27. The composition of claim 26, wherein at least one of the one or more than one VLP is selected from a VLP comprising the modified HA:
i) wherein the modified HA is H1 HA, and wherein the alteration that reduces binding of the modified HA to SA is Y91F; wherein the numbering of the alteration corresponds to the position of reference sequence with SEQ ID NO: 203;
ii) wherein the modified HA is H3 HA, and wherein the alteration that reduces binding of the modified HA to SA is selected from Y98F, S136D; Y98F, S136N; Y98F, S137N; Y98F, D190G; Y98F, D190K; Y98F, R222W; Y98F, S228N; Y98F, S228Q; S136D; S136N; D190K; S228N; or S228Q; wherein the numbering of the alteration corresponds to position of reference sequence with SEQ ID NO: 204.
iii) wherein the modified HA is H5 HA, and wherein the alteration that reduces binding of the modified HA to SA is Y91F; wherein the numbering of the alteration corresponds to position of reference sequence with SEQ ID NO: 205.
iv) wherein the modified HA is H7 HA, and wherein the alteration that reduces binding of the modified HA to SA is Y88F; wherein the numbering of the alteration corresponds to position of reference sequence with SEQ ID NO: 206;
v) wherein the modified HA is B HA, and wherein the alteration that reduces binding of the modified HA to SA is selected from S140A; S142A; G138A; L203A; D195G; or L203W; wherein the numbering of the alteration corresponds to position of reference sequence with SEQ ID NO: 207; or
vi) a combination thereof.

28. A modified influenza H1 hemagglutinin (HA) comprising one or more than one alteration that reduces binding of the modified H1 HA to sialic acid (SA) of a protein on the surface of a cell, while maintaining cognate interaction with the cell.

29. The modified influenza H1 HA of claim 28, wherein the cell is a B cell.

30. The modified influenza H1 HA of claim 28, wherein the protein on the surface of the cell is a B cell surface receptor.

31. The modified H1 HA of claim 27, wherein the modified H1 HA comprises plant-specific N-glycans or modified N-glycans.

32. A virus like particle (VLP) comprising the modified H1 HA of claim 28.

33. The VLP of claim 32 further comprising one or more than one lipid derived from a plant.

34. A modified influenza H3 hemagglutinin (HA) comprising one or more than one alteration that reduces binding of the modified H3 HA to sialic acid (SA) of a protein on the surface of a cell, while maintaining cognate interaction, with the cell.

35. The modified influenza H3 HA of claim 34, wherein the cell is a B cell.

36. The modified influenza H3 HA of claim 34, wherein the protein on the surface of the cell is a B cell surface receptor.

37. The modified H3 HA of claim 33, wherein the modified H3 HA comprises plant-specific N-glycans or modified N-glycans.

38. A virus like particle (VLP) comprising the modified H3 HA of claim 33.

39. The VLP of claim 38, further comprising one or more than one lipid derived from a plant.

40. A modified influenza H7 hemagglutinin (HA) comprising one or more than one alteration that reduces binding of the modified H7 HA to sialic acid (SA) of a protein on the surface of a cell, while maintaining cognate interaction, with the cell.

41. The modified influenza H7 HA of claim 40, wherein the cell is a B cell.

42. The modified influenza H7 HA of claim 40, wherein the protein on the surface of the cell is a B cell surface receptor.

43. The modified H7 HA of claim 40, wherein the modified H7 HA comprises plant-specific N-glycans or modified N-glycans.

44. A virus like particle (VLP) comprising the modified H7 HA of claim 40.

45. The VLP of claim 41 further comprising one or more than one lipid derived from a plant.

46. A modified influenza H5 hemagglutinin (HA) comprising one or more than one alteration that reduces binding of the modified H7 HA to sialic acid (SA) of a protein on the surface of a cell, while maintaining cognate interaction, with the cell.

47. The modified influenza H5 HA of claim 46, wherein the cell is a B cell.

48. The modified influenza H5 HA of claim 47, wherein the protein on the surface of the cell is a B cell surface receptor.

49. The modified H5 HA of claim 46, wherein the modified H5 HA comprises plant-specific N-glycans or modified N-glycans.

50. A virus like particle (VLP) comprising the modified H5 HA of claim 46.

51. The VLP of claim 50 further comprising one or more than one lipid derived from a plant.

52. A modified influenza B hemagglutinin (HA) comprising one or more than one alteration that reduces binding of the modified B HA to sialic acid (SA) of a protein on the surface of a cell, while maintaining cognate interaction, with the cell.

53. The modified influenza B HA of claim 52, wherein the cell is a B cell.

54. The modified influenza B HA of claim 52, wherein the protein on the surface of the cell is a B cell surface receptor.

55. The modified B HA of claim 48, wherein the modified B HA comprises plant-specific N-glycans or modified N-glycans.

56. A virus like particle (VLP) comprising the modified B HA of claim 52.

57. The VLP of claim 56 further comprising one or more than one lipid derived from a plant.

58. A suprastructure comprising modified influenza hemagglutinin (HA), the modified HA comprising one or more than one alteration, the modified HA being selected from:
i) a modified H1 HA, wherein the one or more than one alteration is Y91F; wherein the numbering of the alteration corresponds to the position of reference sequence with SEQ ID NO: 203;
ii) a modified H3 HA, wherein the one or more than one alteration is selected from Y98F, S136D; Y98F, S136N; Y98F, S137N; Y98F, D190G; Y98F, D190K; Y98F, R222W; Y98F, S228N; Y98F, S228Q; S136D; S136N; D190K; S228N; and S228Q; wherein the numbering of the alteration corresponds to position of reference sequence with SEQ ID NO: 204.
iii) a modified H5 HA, wherein the one or more than one alteration is Y91F; wherein the numbering of the alteration corresponds to position of reference sequence with SEQ ID NO: 205.
iv) a modified H7 HA, wherein the one or more than one alteration is Y88F; wherein the numbering of the alteration corresponds to position of reference sequence with SEQ ID NO: 206;
v) a modified B HA, wherein the one or more than one alteration is selected from S140A; S142A; G138A; L203A; D195G; and L203W; wherein the numbering of the alteration corresponds to position of reference sequence with SEQ ID NO: 207; or
vi) a combination thereof.

59. The suprastructure of claim 58, wherein the modified HA reduces non-cognate interaction of the modified HA to sialic acid (SA) of a protein on the surface of a cell, while maintaining cognate interaction, with the cell.

60. The suprastructure of claim 58, wherein the modified HA increases an immunological response of an animal or a subject in response to an antigen challenge.

61. A vaccine comprising the suprastructure of claim 58 and a pharmaceutically acceptable carrier.

62. A method of increasing an immunological response of an animal or a subject in response to an antigen challenge comprising, administering the vaccine of claim 61 to the animal or subject and determining the immunological response, wherein the immunological response is a cellular immunological response, a humoral immunological response, or both a cellular immunological response and a humoral immunological response, and wherein the immunological response is increased when compared with an immunological response obtained following administration of a vaccine comprising a suprastructure comprising influenza HA that do not comprise the one or more than one alteration.


SEQUENCE LISTING (209 sequences)

SEQ ID NO: 1 - DNA, 1722 bp, Artificial Sequence
  PDI-H1A/California/7/2009
atggcgaaaaacgttgcgattttcggcttattgttttctcttcttgtgttggttccttctcagatcttcgctgacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggtcatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggactatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaa

SEQ ID NO: 2 - protein, 573 aa, Artificial Sequence
  PDI-H1 A/ California/7/2009
MAKNVAIFGLLFSLLVLVPSQIFADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 3 - DNA, 49 bp, Artificial Sequence
  Primer IF-CPMV(fl5′UTR)_SpPDI.c
tcgtgcttcggcaccagtacaatggcgaaaaacgttgcgattttcggct

SEQ ID NO: 4 - DNA, 46 bp, Artificial Sequence
  Primer IF-H1cTMCT.S1-4r
actaaagaaaataggcctttaaatacatattctacactgtagagac

SEQ ID NO: 5 - DNA, 4540 bp, Artificial Sequence
  Cloning vector 1190 from left to right T-DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccgcggatggcgaaaaacgttgcgattttcggcttattgttttctcttcttgtgttggttccttctcagatcttcgcctgcaggctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagcgatcgctcaccatcaccatcaccatcaccatcaccattaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 6 - DNA, 3105 bp, Artificial Sequence
  Construct 1314 from 2X35S prom to NOS term
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgctgacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggtcatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggactatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 7 - DNA, 49 bp, Artificial Sequence
  Primer H1_Cal(Y91F).r
aaatctcctgggaaacacgttccattgtctgaactaggtgtttccacaa

SEQ ID NO: 8 - DNA, 49 bp, Artificial Sequence
  Primer H1_Cal(Y91F).c
agacaatggaacgtgtttcccaggagatttcatcgattatgaggagcta

SEQ ID NO: 9 - DNA, 4486 bp, Artificial Sequence
  Cloning vector 3637 from left to right T-DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccgcggatggcgaaaaacgttgcgattttcggcttattgttttctcttcttgtgttggttccttctcagatcttcgcctgcaggctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagcgatcgctcaccatcaccatcaccatcaccatcaccattaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 10 - DNA, 3051 bp, Artificial Sequence
  Construct 6100 from 2X35S prom to NOS term
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgctgacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgtttcccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggtcatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggactatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 11 - DNA, 1722 bp, Artificial Sequence
  PDI-H1 A/ California/7/2009 Y91F
atggcgaaaaacgttgcgattttcggcttattgttttctcttcttgtgttggttccttctcagatcttcgctgacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgtttcccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggtcatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggactatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaa

SEQ ID NO: 12 - protein, 573 aa, Artificial Sequence
  PDI-H1 A/ California/7/2009 Y91F
MAKNVAIFGLLFSLLVLVPSQIFADTLCIGYHANNSTDTVDTVLEKNVTVTHSVNLLEDKHNGKLCKLRGVAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 13 - protein, 550 aa, Artificial Sequence
  A/Minnesota/41/19 (H3N2)
QKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWAGVTQNGKSFSCIRGSSSSFFSRLNWLTHLNYTYPALNVTMPNKEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 14 - protein, 569 aa, Artificial Sequence
  B/Singapore/INFKK-16-0569/16 (Yamagata)
DRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 15 - protein, 568 aa, Artificial Sequence
  B/Maryland/15/16 (Victoria)
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGGPYKIGTSGSCPNITNGNGFFATMAWAVPDKNKTATNPLTIEVPYVCTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFSLPTFDSLNITAASLNDDGLDNHTILLYYSTAASSLAVTLMIAIFVVYMVSRDNVSCSICL

SEQ ID NO: 16 - protein, 567 aa, Artificial Sequence
  B/Victoria/705/18 (Victoria)
DRICTGITSSNSPHVVKTATQGEVNVTGVIPLTTTPTKSHFANLKGTETRGKLCPKCLNCTDLDVALSRPKCTGKIPSARVSILHEVRPVTSGCFPIMHDRTKIRQLPNLLRGYEHVRLSTHNVINAEDAPGGPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDSVSCSICL

SEQ ID NO: 17 - protein, 567 aa, Artificial Sequence
  B/Washington/12/19 (Victoria)
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 18 - protein, 567 aa, Artificial Sequence
  B/Darwin/8/19 (Victoria)
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNEAQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 19 - protein, 567 aa, Artificial Sequence
  B/Darwin/20/19 (Victoria)
DRICTGITSSNSPHVVKTATQGEVNVTGVIPLTTTPTKSHFANLKGTKTRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNEIQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 20 - DNA, 1701 bp, Artificial Sequence
  PDI H7 A/Shanghai/2/2013
atggcgaaaaacgttgcgattttcggcttattgttttctcttcttgtgttggttccttctcagatcttcgcggacaaaatctgcctcggacatcatgccgtgtcaaacggaaccaaagtaaacacattaactgaaagaggagtggaagtcgtcaatgcaactgaaacagtggaacgaacaaacatccccaggatctgctcaaaagggaaaaggacagttgacctcggtcaatgtggactcctggggacaatcactggaccacctcaatgtgaccaattcctagaattttcagccgatttaattattgagaggcgagaaggaagtgatgtctgttatcctgggaaattcgtgaatgaagaagctctgaggcaaattctcagagaatcaggcggaattgacaaggaagcaatgggattcacatacagtggaataagaactaatggagcaaccagtgcatgtaggagatcaggatcttcattctatgcagaaatgaaatggctcctgtcaaacacagataatgctgcattcccgcagatgactaagtcatataaaaatacaagaaaaagcccagctctaatagtatgggggatccatcattccgtatcaactgcagagcaaaccaagctatatgggagtggaaacaaactggtgacagttgggagttctaattatcaacaatcttttgtaccgagtccaggagcgagaccacaagttaatggtctgtctggaagaattgactttcattggctaatgctaaatcccaatgatacagtcactttcagtttcaatggggctttcatagctccagaccgtgcaagcttcctgagaggaaaatctatgggaatccagagtggagtacaggttgatgccaattgtgaaggggactgctatcatagtggagggacaataataagtaacttgccatttcagaacatagatagcagggcagttggaaaatgtccgagatatgttaagcaaaggagtctgctgctagcaacagggatgaagaatgttcctgagattccaaagggaagaggcctatttggtgctatagcgggtttcattgaaaatggatgggaaggcctaattgatggttggtatggtttcagacaccagaatgcacagggagagggaactgctgcagattacaaaagcactcaatcggcaattgatcaaataacaggaaaattaaaccggcttatagaaaaaaccaaccaacaatttgagttgatcgacaatgaattcaatgaggtagagaagcaaatcggtaatgtgataaattggaccagagattctataacagaagtgtggtcatacaatgctgaactcttggtagcaatggagaaccagcatacaattgatctggctgattcagaaatggacaaactgtacgaacgagtgaaaagacagctgagagagaatgctgaagaagatggcactggttgctttgaaatatttcacaagtgtgatgatgactgtatggccagtattagaaataacacctatgatcacagcaaatacagggaagaggcaatgcaaaatagaatacagattgacccagtcaaactaagcagcggctacaaagatgtgatactttggtttagcttcggggcatcatgtttcatacttctagccattgtaatgggccttgtcttcatatgtgtaaagaatggaaacatgcggtgcactatttgtatataa

SEQ ID NO: 21 - protein, 566 aa, Artificial Sequence
  PDI H7 A/Shanghai/2/2013
MAKNVAIFGLLFSLLVLVPSQIFADKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTNGATSACRRSGSSFYAEMKWLLSNTDNAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 22 - DNA, 50 bp, Artificial Sequence
  Primer IF-H7Shang.r
actaaagaaaataggcctttatatacaaatagtgcaccgcatgtttccat

SEQ ID NO: 23 - DNA, 50 bp, Artificial Sequence
  Primer H7Shang(Y88F).c
aggaagtgatgtctgtttccctgggaaattcgtgaatgaagaagctctga

SEQ ID NO: 24 - DNA, 50 bp, Artificial Sequence
  Primer H7Shang(Y88F).r
acgaatttcccagggaaacagacatcacttccttctcgcctctcaataat

SEQ ID NO: 25 - DNA, 1701 bp, Artificial Sequence
  PDI H7 A/Shanghai/2/2013 Y88F
atggcgaaaaacgttgcgattttcggcttattgttttctcttcttgtgttggttccttctcagatcttcgcggacaaaatctgcctcggacatcatgccgtgtcaaacggaaccaaagtaaacacattaactgaaagaggagtggaagtcgtcaatgcaactgaaacagtggaacgaacaaacatccccaggatctgctcaaaagggaaaaggacagttgacctcggtcaatgtggactcctggggacaatcactggaccacctcaatgtgaccaattcctagaattttcagccgatttaattattgagaggcgagaaggaagtgatgtctgtttccctgggaaattcgtgaatgaagaagctctgaggcaaattctcagagaatcaggcggaattgacaaggaagcaatgggattcacatacagtggaataagaactaatggagcaaccagtgcatgtaggagatcaggatcttcattctatgcagaaatgaaatggctcctgtcaaacacagataatgctgcattcccgcagatgactaagtcatataaaaatacaagaaaaagcccagctctaatagtatgggggatccatcattccgtatcaactgcagagcaaaccaagctatatgggagtggaaacaaactggtgacagttgggagttctaattatcaacaatcttttgtaccgagtccaggagcgagaccacaagttaatggtctgtctggaagaattgactttcattggctaatgctaaatcccaatgatacagtcactttcagtttcaatggggctttcatagctccagaccgtgcaagcttcctgagaggaaaatctatgggaatccagagtggagtacaggttgatgccaattgtgaaggggactgctatcatagtggagggacaataataagtaacttgccatttcagaacatagatagcagggcagttggaaaatgtccgagatatgttaagcaaaggagtctgctgctagcaacagggatgaagaatgttcctgagattccaaagggaagaggcctatttggtgctatagcgggtttcattgaaaatggatgggaaggcctaattgatggttggtatggtttcagacaccagaatgcacagggagagggaactgctgcagattacaaaagcactcaatcggcaattgatcaaataacaggaaaattaaaccggcttatagaaaaaaccaaccaacaatttgagttgatcgacaatgaattcaatgaggtagagaagcaaatcggtaatgtgataaattggaccagagattctataacagaagtgtggtcatacaatgctgaactcttggtagcaatggagaaccagcatacaattgatctggctgattcagaaatggacaaactgtacgaacgagtgaaaagacagctgagagagaatgctgaagaagatggcactggttgctttgaaatatttcacaagtgtgatgatgactgtatggccagtattagaaataacacctatgatcacagcaaatacagggaagaggcaatgcaaaatagaatacagattgacccagtcaaactaagcagcggctacaaagatgtgatactttggtttagcttcggggcatcatgtttcatacttctagccattgtaatgggccttgtcttcatatgtgtaaagaatggaaacatgcggtgcactatttgtatataa

SEQ ID NO: 26 - protein, 566 aa, Artificial Sequence
  PDI H7 A/Shanghai/2/2013 Y88F
MAKNVAIFGLLFSLLVLVPSQIFADKICLGHHAVSNGTKVNTLTERGVEVVNATETVERTNIPRICSKGKRTVDLGQCGLLGTITGPPQCDQFLEFSADLIIERREGSDVCFPGKFVNEEALRQILRESGGIDKEAMGFTYSGIRTNGATSACRRSGSSFYAEMKWLLSNTDNAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 27 - DNA, 1731 bp, Artificial Sequence
  PDI B/Phuket/3073/2013
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccactaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatgatgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 28 - protein, 576 aa, Artificial Sequence
  PDI B/Phuket/3073/2013)
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 29 - DNA, 52 bp, Artificial Sequence
  Primer IF.HBPhu3073.c
tctcagatcttcgcggatcgaatctgcactgggataacatcttcaaactcac

SEQ ID NO: 30 - DNA, 59 bp, Artificial Sequence
  Primer B_Phuket(S140A).c
gaccctacagacttggaaccgccggatcttgccctaacgctaccagtaaaatcggattt

SEQ ID NO: 31 - DNA, 57 bp, Artificial Sequence
  Primer B_Phuket(S140A).r
cgttagggcaagatccggcggttccaagtctgtagggtcctcctggtgctttttctg

SEQ ID NO: 32 - DNA, 1731 bp, Artificial Sequence
  PDI B/Phuket/3073/2013 S140A
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaaccgccggatcttgccctaacgctaccagtaaaatcggattttttgcaacaatggcttgggctgtcccaaaggacaactacaaaaatgcaacgaacccactaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatgatgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 33 - protein, 576 aa, Artificial Sequence
  PDI B/Phuket/3073/2013 S140A
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTAGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 34 - DNA, 58 bp, Artificial Sequence
  Primer B_Phuket(S142A).c
tggaacctcaggagcctgccctaacgctaccagtaaaatcggattttttgcaacaatg

SEQ ID NO: 35 - DNA, 48 bp, Artificial Sequence
  Primer B_Phuket(S142A).r
tggtagcgttagggcaggctcctgaggttccaagtctgtagggtcctc

SEQ ID NO: 36 - DNA, 1731 bp, Artificial Sequence
  PDI B/Phuket/3073/2013 S142A
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggagcctgccctaacgctaccagtaaaatcggattttttgcaacaatggcttgggctgtcccaaaggacaactacaaaaatgcaacgaacccactaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatgatgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 37 - protein, 576 aa, Artificial Sequence
  PDI B/Phuket/3073/2013 S142A
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGACPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 38 - DNA, 49 bp, Artificial Sequence
  Primer B_Phuket(G138A).c
gaccctacagacttgccacctcaggatcttgccctaacgctaccagtaa

SEQ ID NO: 39 - DNA, 51 bp, Artificial Sequence
  Primer B_Phuket(G138A).r
ggcaagatcctgaggtggcaagtctgtagggtcctcctggtgctttttctg

SEQ ID NO: 40 - DNA, 1731 bp, Artificial Sequence
  PDI B/Phuket/3073/2013 G138A
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttgccacctcaggatcttgccctaacgctaccagtaaaatcggattttttgcaacaatggcttgggctgtcccaaaggacaactacaaaaatgcaacgaacccactaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatgatgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 41 - protein, 576 aa, Artificial Sequence
  PDI B/Phuket/3073/2013 G138A
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLATSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 42 - DNA, 49 bp, Artificial Sequence
  Primer B_Phuket(L203A).c
cccaaatgaagagcgcctatggagactcaaatcctcaaaagttcacctc

SEQ ID NO: 43 - DNA, 48 bp, Artificial Sequence
  Primer B_Phuket(L203A).r
gatttgagtctccataggcgctcttcatttgggttttgttatccgaat

SEQ ID NO: 44 - DNA, 1731 bp, Artificial Sequence
  PDI-B Phu (L203A, Prl-)
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccactaacagtggaagtaccatacatttgtacagaaggggaagaccaaattactgtttgggggttccattcggataacaaaacccaaatgaagagcgcctatggagactcaaatcctcaaaagttcacctcatctgctaatggagtaaccacgcattatgtttctcagattggcgacttcccagatcaaacagaagacggaggactaccacaaagcggcagaattgttgttgattacatgatgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 45 - protein, 576 aa, Artificial Sequence
  PDI-B Phu (L203A, Prl-)
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSAYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 46 - DNA, 49 bp, Artificial Sequence
  Primer B_Phuket(D195G).c
gggggttccattcgggcaacaaaacccaaatgaagagcctctatggaga

SEQ ID NO: 47 - DNA, 48 bp, Artificial Sequence
  Primer B_Phuket(D195G).r
tcatttgggttttgttgcccgaatggaacccccaaacagtaatttggt

SEQ ID NO: 48 - DNA, 1731 bp, Artificial Sequence
  PDI-B Phu (D195G, Prl-)
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccactaacagtggaagtaccatacatttgtacagaaggggaagaccaaattactgtttgggggttccattcgggcaacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatgatgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 49 - protein, 576 aa, Artificial Sequence
  PDI-B Phu (D195G, Prl-)
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSGNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 50 - DNA, 49 bp, Artificial Sequence
  Primer B_Phuket(L203W).c
cccaaatgaagagctggtatggagactcaaatcctcaaaagttcacctc

SEQ ID NO: 51 - DNA, 48 bp, Artificial Sequence
  Primer B_Phuket(L203W).r
gatttgagtctccataccagctcttcatttgggttttgttatccgaat

SEQ ID NO: 52 - DNA, 1731 bp, Artificial Sequence
  PDI-B Phu (L203W, Prl-)
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccactaacagtggaagtaccatacatttgtacagaaggggaagaccaaattactgtttgggggttccattcggataacaaaacccaaatgaagagctggtatggagactcaaatcctcaaaagttcacctcatctgctaatggagtaaccacgcattatgtttctcagattggcgacttcccagatcaaacagaagacggaggactaccacaaagcggcagaattgttgttgattacatgatgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 53 - protein, 576 aa, Artificial Sequence
  PDI-B Phu (L203W, Prl-)
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSWYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 54 - DNA, 6443 bp, Artificial Sequence
  Cloning vector 2530 from left to right T-DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacacgcgtggcgcgccctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatgagtcttctaaccgaggtcgaaacgcctatcagaaacgaatgggggtgcagatgcaacgattcaagtgatcctcttgttgttgccgcaagtataattgggattgtgcacctgatattgtggattattgatcgccttttttccaaaagcatttatcgtatctttaaacacggtttaaaaagagggccttctacggaaggagtaccagagtctatgagggaagaatatcgagaggaacagcagaatgctgtggatgctgacgatggtcattttgtcagcatagagctggagtaagagctctaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggaactaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcgacgtcactcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagacgtccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 55 - DNA, 3114 bp, Artificial Sequence
  Construct 2835 from 2X35S prom to NOS term
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccactaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatgatgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 56 - DNA, 6389 bp, Artificial Sequence
  Cloning vector 4499 from left to right T-DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacacgcgtggcgcgccctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatgagtcttctaaccgaggtcgaaacgcctatcagaaacgaatgggggtgcagatgcaacgattcaagtgatcctcttgttgttgccgcaagtataattgggattgtgcacctgatattgtggattattgatcgccttttttccaaaagcatttatcgtatctttaaacacggtttaaaaagagggccttctacggaaggagtaccagagtctatgagggaagaatatcgagaggaacagcagaatgctgtggatgctgacgatggtcattttgtcagcatagagctggagtaagagctctaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggaactaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcgacgtcactcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagacgtccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 57 - DNA, 3060 bp, Artificial Sequence
  Construct 8352 from 2X35S prom to NOS term
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaaccgccggatcttgccctaacgctaccagtaaaatcggattttttgcaacaatggcttgggctgtcccaaaggacaactacaaaaatgcaacgaacccactaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatgatgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 58 - DNA, 3054 bp, Artificial Sequence
  Construct 7281 from 2X35S prom to NOS term
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 59 - DNA, 3054 bp, Artificial Sequence
  Construct 8179 from 2X35S prom to NOS term
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgtttcccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 60 - DNA, 1725 bp, Artificial Sequence
  PDI-H3 A/Kansas/14/2017
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 61 - protein, 574 aa, Artificial Sequence
  PDI-H3 A/Kansas/14/2017
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 62 - DNA, 57 bp, Artificial Sequence
  Primer IF-H3NewJer.c
tctcagatcttcgcgcaaaaaatccctggaaatgacaatagcacggcaacgctgtgc

SEQ ID NO: 63 - DNA, 51 bp, Artificial Sequence
  Primer IF-H3_Swi_13.r
actaaagaaaataggccttcaaatgcaaatgttgcacctaatgttgccctt

SEQ ID NO: 64 - DNA, 1725 bp, Artificial Sequence
  PDI-H3 A/Kansas/14/2017 Y98F
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgtttcccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 65 - protein, 574 aa, Artificial Sequence
  PDI-H3 A/Kansas/14/2017 Y98F
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCFPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 66 - DNA, 48 bp, Artificial Sequence
  Primer H3_Kansas(Y98F).c
cctacagcaactgtttcccttatgatgtgccggattatgcctccctta

SEQ ID NO: 67 - DNA, 50 bp, Artificial Sequence
  Primer H3_Kansas(Y98F).r
ccggcacatcataagggaaacagttgctgtaggctttgtttcgttcaaca

SEQ ID NO: 68 - DNA, 1725 bp, Artificial Sequence
  PDI-H3 A/Kansas/14/2017 Y98F, S136D
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgtttcccttatgatgtgccggattatgcctcccttaggtcactagttgcctcatccggcacactggagtttaacaatgaaagcttcaattgggctggagtcactcaaaacggaacagactcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 69 - protein, 574 aa, Artificial Sequence
  PDI-H3 A/Kansas/14/2017 Y98F, S136D
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCFPYDVPDYASLRSLVASSGTLEFNNESFNWAGVTQNGTDSSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 70 - DNA, 48 bp, Artificial Sequence
  Primer H3Kansas(S136D).c
aaacggaacagactcttcttgcataaggggatctaagagtagtttctt

SEQ ID NO: 71 - DNA, 50 bp, Artificial Sequence
  Primer H3Kansas(S136D).r
caagaagagtctgttccgttttgagtgactccagcccaattgaagctttc

SEQ ID NO: 72 - DNA, 1725 bp, Artificial Sequence
  PDI-H3 A/Kansas/14/2017 Y98F, S136N
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgtttcccttatgatgtgccggattatgcctcccttaggtcactagttgcctcatccggcacactggagtttaacaatgaaagcttcaattgggctggagtcactcaaaacggaacaaactcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 73 - protein, 574 aa, Artificial Sequence
  PDI-H3 A/Kansas/14/2017 Y98F, S136N
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCFPYDVPDYASLRSLVASSGTLEFNNESFNWAGVTQNGTNSSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 74 - DNA, 48 bp, Artificial Sequence
  Primer H3Kansas(S136N).c
aaacggaacaaactcttcttgcataaggggatctaagagtagtttctt

SEQ ID NO: 75 - DNA, 50 bp, Artificial Sequence
  Primer H3Kansas(S136N).r
caagaagagtttgttccgttttgagtgactccagcccaattgaagctttc

SEQ ID NO: 76 - DNA, 1725 bp, Artificial Sequence
  PDI-H3 A/Kansas/14/2017 Y98F, S137N
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgtttcccttatgatgtgccggattatgcctcccttaggtcactagttgcctcatccggcacactggagtttaacaatgaaagcttcaattgggctggagtcactcaaaacggaacaagtaactcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 77 - protein, 574 aa, Artificial Sequence
  PDI-H3 A/Kansas/14/2017 Y98F, S137N
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCFPYDVPDYASLRSLVASSGTLEFNNESFNWAGVTQNGTSNSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 78 - DNA, 51 bp, Artificial Sequence
  Primer H3Kansas(S137N).c
cggaacaagtaactcttgcataaggggatctaagagtagtttctttagtag

SEQ ID NO: 79 - DNA, 55 bp, Artificial Sequence
  Primer H3Kansas(S137N).r
atgcaagagttacttgttccgttttgagtgactccagcccaattgaagctttcat

SEQ ID NO: 80 - DNA, 1725 bp, Artificial Sequence
  PDI-H3 A/Kansas/14/2017 Y98F, D190G
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgtttcccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaagggccaaatcagcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 81 - protein, 574 aa, Artificial Sequence
  PDI-H3 A/Kansas/14/2017 Y98F, D190G
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCFPYDVPDYASLRSLVASSGTLEFNNESFNWAGVTQNGTSSSCIRGSKSSFFSRLNWLTHLNSKYPALNVTMPNNEQFDKLYIWGVHHPGTDKGQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 82 - DNA, 49 bp, Artificial Sequence
  Primer H3Kansas(D190G).c
tacggacaagggccaaatcagcctgtatgcacaatcatcaggaagaatc

SEQ ID NO: 83 - DNA, 48 bp, Artificial Sequence
  Primer H3Kansas(D190G).r
ctgatttggcccttgtccgtacccgggtggtgaaccccccaaatgtac

SEQ ID NO: 84 - DNA, 1725 bp, Artificial Sequence
  PDI-H3 A/Kansas/14/2017 Y98F, D190K
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgtttcccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaagaagcaaatcagcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 85 - protein, 574 aa, Artificial Sequence
  PDI-H3 A/Kansas/14/2017 Y98F, D190K
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCFPYDVPDYASLRSLVASSGTLEFNNESFNWAGVTQNGTSSSCIRGSKSSFFSRLNWLTHLNSKYPALNVTMPNNEQFDKLYIWGVHHPGTDKKQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 86 - DNA, 49 bp, Artificial Sequence
  Primer H3Kansas(D190K).c
tacggacaagaagcaaatcagcctgtatgcacaatcatcaggaagaatc

SEQ ID NO: 87 - DNA, 48 bp, Artificial Sequence
  Primer H3Kansas(D190K).r
ctgatttgcttcttgtccgtacccgggtggtgaaccccccaaatgtac

SEQ ID NO: 88 - DNA, 1725 bp, Artificial Sequence
  PDI-H3 A/Kansas/14/2017 Y98F, R222W
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgtttcccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagaccctggataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 89 - protein, 574 aa, Artificial Sequence
  PDI-H3 A/Kansas/14/2017 Y98F, R222W
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCFPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPWIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 90 - DNA, 50 bp, Artificial Sequence
  Primer H3Kansas(R222W).c
atctagaccctggataagggatatccctagcagaataagcatctattgga

SEQ ID NO: 91 - DNA, 49 bp, Artificial Sequence
  Primer H3Kansas(R222W).r
tcccttatccagggtctagatccgatattcgggattacagcttgttggc

SEQ ID NO: 92 - DNA, 1725 bp, Artificial Sequence
  PDI-H3 A/Kansas/14/2017 Y98F, S228N
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgtttcccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctaacagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 93 - protein, 574 aa, Artificial Sequence
  PDI-H3 A/Kansas/14/2017 Y98F, S228N
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCFPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPRIRDIPNRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 94 - DNA, 51 bp, Artificial Sequence
  Primer H3Kansas(S228N).c
ggatatccctaacagaataagcatctattggacaatagtaaaaccgggaga

SEQ ID NO: 95 - DNA, 51 bp, Artificial Sequence
  Primer H3Kansas(S228N).r
cttattctgttagggatatcccttattctgggtctagatccgatattcggg

SEQ ID NO: 96 - DNA, 1725 bp, Artificial Sequence
  PDI-H3 A/Kansas/14/2017 Y98F, S228Q
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgtttcccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctcagagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 97 - protein, 574 aa, Artificial Sequence
  PDI-H3 A/Kansas/14/2017 Y98F, S228Q
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCFPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPRIRDIPQRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 98 - DNA, 55 bp, Artificial Sequence
  Primer H3Kansas(S228Q).c
ggatatccctcagagaataagcatctattggacaatagtaaaaccgggagacata

SEQ ID NO: 99 - DNA, 51 bp, Artificial Sequence
  Primer H3Kansas(S228Q).r
cttattctctgagggatatcccttattctgggtctagatccgatattcggg

SEQ ID NO: 100 - DNA, 1722 bp, Artificial Sequence
  PDI-H1 A/Idaho/7/18
atggcgaaaaacgttgcgattttcggcttattgttttctcttcttgtgttggttccttctcagatcttcgcggacacattatgtataggttatcatgcgaacaattcaacagacactgtagacacagtactagaaaagaatgtaacagtaacacactctgttaaccttctggaagacaagcataacggaaaactatgcaaactaagaggggtagccccattgcatttgggtaaatgtaacattgctggctggatcctgggaaatccagagtgtgaatcactctccacagcaagatcatggtcctacattgtggaaacatctaattcagacaatggaacgtgttacccaggagatttcatcaattatgaggagctaagagagcaattgagctcagtgtcatcatttgaaaggtttgagatattccccaagacaagttcatggcccaatcatgactcgaacaaaggtgtaacggcagcatgtcctcacgctggagcaaaaagcttctacaaaaacttgatatggctagttaaaaaaggaaattcatacccaaagcttaaccaaacctacattaatgataaagggaaagaagtcctcgtgctgtggggcattcaccatccacccactactgctgaccaacaaagtctctatcagaatgcagatgcatatgtttttgtggggacatcaagatacagcaagaagttcaagccggaaatagcaacaagacccaaagtgagggatcaagaagggagaatgaactattactggacactagtagagccgggagacaaaataacattcgaagcaactggaaatctagtggtaccgagatatgcattcacaatggaaagaaatgctggatctggtattatcatttcagatacaccagtccacgattgcaatacaacttgtcagacacccgagggtgctataaacaccagcctcccatttcagaatgtgcatccgatcacaattggaaaatgtccaaagtatgtaaaaagcacaaaattgagactggccacaggattgaggaatgttccgtctattcaatctagaggcctattcggggccattgccggcttcattgaaggggggtggacagggatggtagatggatggtacggttatcaccatcaaaatgagcaggggtcaggatatgcagccgacctgaagagcacacaaaatgccattgacaagattactaacaaagtaaattctgttattgaaaagatgaatacacaggacacagcagtgggtaaagagttcaaccacctggaaaaaagaatagagaatctaaataaaaaagttgatgatggtttcctggacatttggacttacaatgccgaactgttggttctaatggaaaatgaaagaactttggactatcacgattcaaatgtgaagaacttgtatgaaaaagtaagaaaccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaaaaaatagatggggtaaagctggaatcaacaaggatttaccagattttggcgatctattcaactgtcgccagttcattggtactggtagtctccctgggggcaatcagcttctggatgtgctctaatgggtctctacagtgtagaatatgtatttaa

SEQ ID NO: 101 - protein, 573 aa, Artificial Sequence
  PDI- H1 A/Idaho/7/18
MAKNVAIFGLLFSLLVLVPSQIFADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQDTAVGKEFNHLEKRIENLNKKVDDGFLDIWTYNAELLVLMENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 102 - DNA, 48 bp, Artificial Sequence
  Primer IF-H1_Cal-7-09.c
tctcagatcttcgcggacacattatgtataggttatcatgcgaacaat

SEQ ID NO: 103 - DNA, 46 bp, Artificial Sequence; identical to SEQ ID NO: 4
  Primer IF-H1cTMCT.s1-4r

SEQ ID NO: 104 - DNA, 1722 bp, Artificial Sequence
  PDI-H1 A/Idaho/7/18 Y91F
atggcgaaaaacgttgcgattttcggcttattgttttctcttcttgtgttggttccttctcagatcttcgcggacacattatgtataggttatcatgcgaacaattcaacagacactgtagacacagtactagaaaagaatgtaacagtaacacactctgttaaccttctggaagacaagcataacggaaaactatgcaaactaagaggggtagccccattgcatttgggtaaatgtaacattgctggctggatcctgggaaatccagagtgtgaatcactctccacagcaagatcatggtcctacattgtggaaacatctaattcagacaatggaacgtgtttcccaggagatttcatcaattatgaggagctaagagagcaattgagctcagtgtcatcatttgaaaggtttgagatattccccaagacaagttcatggcccaatcatgactcgaacaaaggtgtaacggcagcatgtcctcacgctggagcaaaaagcttctacaaaaacttgatatggctagttaaaaaaggaaattcatacccaaagcttaaccaaacctacattaatgataaagggaaagaagtcctcgtgctgtggggcattcaccatccacccactactgctgaccaacaaagtctctatcagaatgcagatgcatatgtttttgtggggacatcaagatacagcaagaagttcaagccggaaatagcaacaagacccaaagtgagggatcaagaagggagaatgaactattactggacactagtagagccgggagacaaaataacattcgaagcaactggaaatctagtggtaccgagatatgcattcacaatggaaagaaatgctggatctggtattatcatttcagatacaccagtccacgattgcaatacaacttgtcagacacccgagggtgctataaacaccagcctcccatttcagaatgtgcatccgatcacaattggaaaatgtccaaagtatgtaaaaagcacaaaattgagactggccacaggattgaggaatgttccgtctattcaatctagaggcctattcggggccattgccggcttcattgaaggggggtggacagggatggtagatggatggtacggttatcaccatcaaaatgagcaggggtcaggatatgcagccgacctgaagagcacacaaaatgccattgacaagattactaacaaagtaaattctgttattgaaaagatgaatacacaggacacagcagtgggtaaagagttcaaccacctggaaaaaagaatagagaatctaaataaaaaagttgatgatggtttcctggacatttggacttacaatgccgaactgttggttctaatggaaaatgaaagaactttggactatcacgattcaaatgtgaagaacttgtatgaaaaagtaagaaaccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaaaaaatagatggggtaaagctggaatcaacaaggatttaccagattttggcgatctattcaactgtcgccagttcattggtactggtagtctccctgggggcaatcagcttctggatgtgctctaatgggtctctacagtgtagaatatgtatttaa

SEQ ID NO: 105 - protein, 573 aa, Artificial Sequence
  PDI- H1 A/Idaho/7/18 Y91F
MAKNVAIFGLLFSLLVLVPSQIFADTLCIGYHANNSTDTVDTVLEKNVTVTHSVNLLEDKHNGKLCKLRGVAPLHLGKCNIAGWILGNPECESLSTARSWSYIVETSNSDNGTCFPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQDTAVGKEFNHLEKRIENLNKKVDDGFLDIWTYNAELLVLMENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 106 - DNA, 48 bp, Artificial Sequence
  H1_Idaho(Y91F).c
acaatggaacgtgtttcccaggagatttcatcaattatgaggagctaa

SEQ ID NO: 107 - DNA, 48 bp, Artificial Sequence
  H1_Idaho(Y91F).r
tgatgaaatctcctgggaaacacgttccattgtctgaattagatgttt

SEQ ID NO: 108 - protein, 551 aa, Artificial Sequence
  A/Egypt/NO4915/14 (H5N1)
DQICIGYHANNSTEQVDTIMEKNVTVTHAQDILEKTHNGKLCNLDGVKPLILRDCSVAGWLLGNPMCDEFLNVPEWSYIVEKINPANDLCYPGNFNDYEELKHLLSRINHFEKIQIIPKDSWSDHEASGVSSACPYQGRSSFFRNVVWLTKKNDAYPTIKKSYNNTNQEDLLVLWGIHHPNDAAEQTRLYQNPTTYISVGTSTLNQRLVPKIATRSKVNGQSGRMEFFWTILKSNDAINFESNGNFIAPENAYKIVKKGDSTIMKSELEYSNCNTKCQTPIGAINSSMPFHNIHPLTIGECPKYVKSNRLVLATGLRNSPQGEKRRKKRGLFGAIAGFIEGGWQGMVDGWYGYHHSNEQGSGYAADKESTQRAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCRICI

SEQ ID NO: 109 - protein, 542 aa, Artificial Sequence
  A/Hangzhou/1/13 (H7N9)
DKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTNGATSACRRSGSSFYAEMKWLLSNTDNAAFPQMTKSYKNTRKSPALIVWGIHHSVSTAEQTKLYGSGNKLVTVGSSNYQQSFVPSPGARPQVNGISGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 110 - protein, 29 aa, Artificial Sequence
  Consensus amino acid sequence for transmembrane domain
  misc_feature (3)..(3): Xaa can be any naturally occurring amino acid
  misc_feature (15)..(15): Xaa can be any naturally occurring amino acid
  misc_feature (17)..(18): Xaa can be any naturally occurring amino acid
  misc_feature (23)..(23): Xaa can be any naturally occurring amino acid
  misc_feature (25)..(25): Xaa can be any naturally occurring amino acid
ILXIYYSTVAISSLXLXXMLAGXSXWMCS

SEQ ID NO: 111 - DNA, 1725 bp, Artificial Sequence
  PDI-H3 Kan-S136D DNA
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgttacccttatgatgtgccggattatgcctcccttaggtcactagttgcctcatccggcacactggagtttaacaatgaaagcttcaattgggctggagtcactcaaaacggaacagactcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 112 - protein, 574 aa, Artificial Sequence
  PDI-H3 Kan-S136D AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTDSSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 113 - DNA, 1725 bp, Artificial Sequence
  PDI-H3 Kan-S136N DNA
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgttacccttatgatgtgccggattatgcctcccttaggtcactagttgcctcatccggcacactggagtttaacaatgaaagcttcaattgggctggagtcactcaaaacggaacaaactcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 114 - protein, 574 aa, Artificial Sequence
  PDI-H3 Kan-S136N AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTNSSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 115 - DNA, 1725 bp, Artificial Sequence
  PDI-H3 Kan-D190K DNA
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaagaagcaaatcagcctgtatgcacaatcatcaggaagaatcacagtatctaccaaaagaagccaacaagctgtaatcccgaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 116 - protein, 574 aa, Artificial Sequence
  PDI-H3 Kan-D190K AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTSSSCIRGSKSSFFSRLNWLTHLNSKYPALNVTMPNNEQFDKLYIWGVHHPGTDKKQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 117 - DNA, 1725 bp, Artificial Sequence
  PDI-H3 Kan-R222W DNA
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagaccctggataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 118 - protein, 574 aa, Artificial Sequence
  PDI-H3 Kan-R222W AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPWIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 119 - DNA, 1725 bp, Artificial Sequence
  PDI-H3 Kan-S228N DNA
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctaacagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 120 - protein, 574 aa, Artificial Sequence
  PDI-H3 Kan-S228N AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPRIRDIPNRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 121 - DNA, 1725 bp, Artificial Sequence
  PDI-H3 Kan-S228Q DNA
atggcgaaaaacgttgcgattttcggcttattgttttctcttcttgtgttggttccttctcagatcttcgcgcaaaaaatccctggaaatgacaatagcacggcaacgctgtgccttgggcaccatgcagtaccaaacggaacgatagtgaaaacaatcacgaatgaccgaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaaacaaagcctacagcaactgttacccttatgatgtgccggattatgcctcccttaggtcactagttgcctcatccggcacactggagtttaacaatgaaagcttcaattgggctggagtcactcaaaacggaacaagttcttcttgcataaggggatctaagagtagtttctttagtagattaaattggttgacccacttaaactccaaatacccagcattaaacgtgactatgccaaacaatgaacaatttgacaaattgtacatttggggggttcaccacccgggtacggacaaggaccaaatcagcctgtatgcacaatcatcaggaagaatcacagtatctaccaaaagaagccaacaagctgtaatcccgaatatcggatctagacccagaataagggatatccctcagagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagagacaaactagaggcatatttggcgcaatagcgggtttcatagaaaatggttgggagggaatggtggatggttggtacggcttcaggcatcaaaattctgagggaagaggacaagcagcagatctcaaaagcactcaagcagcaatcgatcaaatcaatgggaagctggctcgagttatcgggaaaaccaacgagaaattccatcagattgaaaaagaattctcagaagtagaagggagaattcaggaccttgagaaatatgttgaggacacaaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcatgggatcaatcagaaatggaacttatgaccacaacgtatacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattaggtgcaacatttgcatttga

SEQ ID NO: 122 - protein, 574 aa, Artificial Sequence
  PDI-H3 Kan-S228Q AA
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPRIRDIPQRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLARVIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISSLVLLVALLGFIMWACQKGNIRCNICI

SEQ ID NO: 123 - DNA, 1731 bp, Artificial Sequence
  PDI-B Sing DNA
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccacaaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 124 - protein, 576 aa, Artificial Sequence
  PDI-B Sing AA
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 125 - DNA, 1731 bp, Artificial Sequence
  PDI-B Sing-G138A DNA
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttgccacctcaggatcttgccctaacgctaccagtaaaatcggattttttgcaacaatggcttgggctgtcccaaaggacaactacaaaaatgcaacgaacccacaaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 126 - protein, 576 aa, Artificial Sequence
  PDI-B Sing-G138A AA
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLATSGSCPNATSKIGFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 127 - DNA, 1731 bp, Artificial Sequence
  PDI-B Sing-S140A DNA
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaaccgccggatcttgccctaacgctaccagtaaaatcggattttttgcaacaatggcttgggctgtcccaaaggacaactacaaaaatgcaacgaacccacaaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 128 - protein, 576 aa, Artificial Sequence
  PDI-B Sing-S140A AA
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTAGSCPNATSKIGFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 129 - DNA, 1731 bp, Artificial Sequence
  PDI-B Sing-S142A DNA
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggagcctgccctaacgctaccagtaaaatcggattttttgcaacaatggcttgggctgtcccaaaggacaactacaaaaatgcaacgaacccacaaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 130 - protein, 576 aa, Artificial Sequence
  PDI-B Sing-S142A AA
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGACPNATSKIGFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 131 - DNA, 1731 bp, Artificial Sequence
  PDI-B Sing-D195G DNA
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccacaaacagtggaagtaccatacatttgtacagaaggggaagaccaaattactgtttgggggttccattcgggcaacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 132 - protein, 576 aa, Artificial Sequence
  PDI-B Sing-D195G AA
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPQTVEVPYICTEGEDQITVWGFHSGNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 133 - DNA, 1731 bp, Artificial Sequence
  PDI-B Sing-L203A DNA
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccacaaacagtggaagtaccatacatttgtacagaaggggaagaccaaattactgtttgggggttccattcggataacaaaacccaaatgaagagcgcctatggagactcaaatcctcaaaagttcacctcatctgctaatggagtaaccacgcattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 134 - protein, 576 aa, Artificial Sequence
  PDI-B Sing-L203A AA
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPQTVEVPYICTEGEDQITVWGFHSDNKTQMKSAYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 135 - DNA, 1731 bp, Artificial Sequence
  PDI-B Sing-L203W DNA
atggcgaaaaacgttgcgattttcggcttattgttttctcttcttgtgttggttccttctcagatcttcgcggatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccacaaacagtggaagtaccatacatttgtacagaaggggaagaccaaattactgtttgggggttccattcggataacaaaacccaaatgaagagctggtatggagactcaaatcctcaaaagttcacctcatctgctaatggagtaaccacgcattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctggtggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataactaccagattttggcgatctattcaactgtcgccagttcattggtactggtagtctccctgggggcaatcagtttctggatgtgctctaatgggtctctacagtgtagaatatgtatttaa

SEQ ID NO: 136 - protein, 576 aa, Artificial Sequence
  PDI-B Sing-L203W AA
MAKNVAIFGLLFSLLVLVPSQIFADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPQTVEVPYICTEGEDQITVWGFHSDNKTQMKSWYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNYQILAIYSTVASSLVLVVSLGAISFWMCSNGSLQCRICI

SEQ ID NO: 137 - DNA, 1728 bp, Artificial Sequence
  PDI-B Mary DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagacgcaccaggaggaccctacaaaattggaacctcagggtcttgccctaacattaccaatggaaacggattcttcgcaacaatggcttgggccgtcccagacaaaaacaaaacagcaacaaatccattaacaatagaagtaccatacgtgtgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacaagatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 138 - protein, 575 aa, Artificial Sequence
  PDI-B Mary AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGGPYKIGTSGSCPNITNGNGFFATMAWAVPDKNKTATNPLTIEVPYVCTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFSLPTFDSLNITAASLNDDGLDNYQILAIYSTVASSLVLVVSLGAISFWMCSNGSLQCRICI

SEQ ID NO: 139 - DNA, 50 bp, Artificial Sequence
  IF-B-Bris(nat).c
tctcagatcttcgcggatcgaatctgcactgggataacatcgtcaaactc

SEQ ID NO: 140 - DNA, 1728 bp, Artificial Sequence
  PDI-B Mary-G138A DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagacgcaccaggaggaccctacaaaattgccacctcagggtcttgccctaacattaccaatggaaacggattcttcgcaacaatggcttgggccgtcccagacaaaaacaaaacagcaacaaatccattaacaatagaagtaccatacgtgtgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacaagatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 141 - protein, 575 aa, Artificial Sequence
  PDI-B Mary-G138A AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGGPYKIATSGSCPNITNGNGFFATMAWAVPDKNKTATNPLTIEVPYVCTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFSLPTFDSLNITAASLNDDGLDNYQILAIYSTVASSLVLVVSLGAISFWMCSNGSLQCRICI

SEQ ID NO: 142 - DNA, 1728 bp, Artificial Sequence
  PDI-B Mary-S140A DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagacgcaccaggaggaccctacaaaattggaaccgccgggtcttgccctaacattaccaatggaaacggattcttcgcaacaatggcttgggccgtcccagacaaaaacaaaacagcaacaaatccattaacaatagaagtaccatacgtgtgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacaagatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 143 - protein, 575 aa, Artificial Sequence
  PDI-B Mary-S140A AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGGPYKIGTAGSCPNITNGNGFFATMAWAVPDKNKTATNPLTIEVPYVCTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFSLPTFDSLNITAASLNDDGLDNYQILAIYSTVASSLVLVVSLGAISFWMCSNGSLQCRICI

SEQ ID NO: 144 - DNA, 1728 bp, Artificial Sequence
  PDI-B Mary-S142A DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagacgcaccaggaggaccctacaaaattggaacctcaggggcctgccctaacattaccaatggaaacggattcttcgcaacaatggcttgggccgtcccagacaaaaacaaaacagcaacaaatccattaacaatagaagtaccatacgtgtgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacaagatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 145 - protein, 575 aa, Artificial Sequence
  PDI-B Mary-S142A AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGGPYKIGTSGACPNITNGNGFFATMAWAVPDKNKTATNPLTIEVPYVCTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFSLPTFDSLNITAASLNDDGLDNYQILAIYSTVASSLVLVVSLGAISFWMCSNGSLQCRICI

SEQ ID NO: 146 - DNA, 1728 bp, Artificial Sequence
  PDI-B Mary-D194G DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagacgcaccaggaggaccctacaaaattggaacctcagggtcttgccctaacattaccaatggaaacggattcttcgcaacaatggcttgggccgtcccagacaaaaacaaaacagcaacaaatccattaacaatagaagtaccatacgtgtgtacagaaggagaagaccaaattaccgtttgggggttccactctggcaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacaagatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 147 - protein, 575 aa, Artificial Sequence
  PDI-B Mary-D194G AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGGPYKIGTSGSCPNITNGNGFFATMAWAVPDKNKTATNPLTIEVPYVCTEGEDQITVWGFHSGNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFSLPTFDSLNITAASLNDDGLDNYQILAIYSTVASSLVLVVSLGAISFWMCSNGSLQCRICI

SEQ ID NO: 148 - DNA, 1728 bp, Artificial Sequence
  PDI-B Mary-L202A DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagacgcaccaggaggaccctacaaaattggaacctcagggtcttgccctaacattaccaatggaaacggattcttcgcaacaatggcttgggccgtcccagacaaaaacaaaacagcaacaaatccattaacaatagaagtaccatacgtgtgtacagaaggagaagaccaaattaccgtttgggggttccactctgacaacgagacccaaatggcaaaggcctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacaagatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 149 - protein, 575 aa, Artificial Sequence
  PDI-B Mary-L202A AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGGPYKIGTSGSCPNITNGNGFFATMAWAVPDKNKTATNPLTIEVPYVCTEGEDQITVWGFHSDNETQMAKAYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFSLPTFDSLNITAASLNDDGLDNYQILAIYSTVASSLVLVVSLGAISFWMCSNGSLQCRICI

SEQ ID NO: 150 - DNA, 1728 bp, Artificial Sequence
  PDI-B Mary-L202W DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagacgcaccaggaggaccctacaaaattggaacctcagggtcttgccctaacattaccaatggaaacggattcttcgcaacaatggcttgggccgtcccagacaaaaacaaaacagcaacaaatccattaacaatagaagtaccatacgtgtgtacagaaggagaagaccaaattaccgtttgggggttccactctgacaacgagacccaaatggcaaagtggtatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacaagatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 151 - protein, 575 aa, Artificial Sequence
  PDI-B Mary-L202W AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGGPYKIGTSGSCPNITNGNGFFATMAWAVPDKNKTATNPLTIEVPYVCTEGEDQITVWGFHSDNETQMAKWYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFSLPTFDSLNITAASLNDDGLDNYQILAIYSTVASSLVLVVSLGAISFWMCSNGSLQCRICI

SEQ ID NO: 152 - DNA, 1725 bp, Artificial Sequence
  PDI-B Wash DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaagaccctacgagattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 153 - protein, 574 aa, Artificial Sequence
  PDI-B Wash AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 154 - DNA, 1725 bp, Artificial Sequence
  PDI-B Wash-G138A DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaagaccctacgagattgccacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 155 - protein, 574 aa, Artificial Sequence
  PDI-B Wash-G138A AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIATSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 156 - DNA, 1725 bp, Artificial Sequence
  PDI-B Wash-S140A DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaagaccctacgagattggaaccgccgggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 157 - protein, 574 aa, Artificial Sequence
  PDI-B Wash-S140A AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTAGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 158 - DNA, 1725 bp, Artificial Sequence
  PDI-B Wash-S142A DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaagaccctacgagattggaacctcaggggcctgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 159 - protein, 574 aa, Artificial Sequence
  PDI-B Wash-S142A AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGACPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 160 - DNA, 1725 bp, Artificial Sequence
  PDI-B Wash-D193G DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaagaccctacgagattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctggcaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 161 - protein, 574 aa, Artificial Sequence
  PDI-B Wash-D193G AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSGNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 162 - DNA, 1725 bp, Artificial Sequence
  PDI-B Wash-L201A DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaagaccctacgagattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaaggcctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 163 - protein, 574 aa, Artificial Sequence
  PDI-B Wash-L201A AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKAYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 164 - DNA, 1725 bp, Artificial Sequence
  PDI-B Wash-L201W DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaagaccctacgagattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagtggtatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 165 - protein, 574 aa, Artificial Sequence
  PDI-B Wash-L201W AA
MAKNVAIFGLLFSLLVLVPSQIFADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKWYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 166-179 - skipped (14 entries)

SEQ ID NO: 180 - DNA, 1725 bp, Artificial Sequence
  PDI-B Vic DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgagcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaggaccctacgagattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 181 - protein, 574 aa, Artificial Sequence
  PDI-B Vic AA
MAKNVAIFGLLFSLLVLVPSQIFADRICTGITSSNSPHVVKTATQGEVNVTGVIPLTTTPTKSHFANLKGTETRGKLCPKCLNCTDLDVALSRPKCTGKIPSARVSILHEVRPVTSGCFPIMHDRTKIRQLPNLLRGYEHVRLSTHNVINAEDAPGGPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 182 - DNA, 1725 bp, Artificial Sequence
  PDI-B Vic-G138A DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgagcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaggaccctacgagattgccacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 183 - protein, 574 aa, Artificial Sequence
  PDI-B Vic-G138A AA
MAKNVAIFGLLFSLLVLVPSQIFADRICTGITSSNSPHVVKTATQGEVNVTGVIPLTTTPTKSHFANLKGTETRGKLCPKCLNCTDLDVALSRPKCTGKIPSARVSILHEVRPVTSGCFPIMHDRTKIRQLPNLLRGYEHVRLSTHNVINAEDAPGGPYEIATSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 184 - DNA, 1725 bp, Artificial Sequence
  PDI-B Vic-S140A DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgagcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaggaccctacgagattggaaccgccgggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 185 - protein, 574 aa, Artificial Sequence
  PDI-B Vic-S140A AA
MAKNVAIFGLLFSLLVLVPSQIFADRICTGITSSNSPHVVKTATQGEVNVTGVIPLTTTPTKSHFANLKGTETRGKLCPKCLNCTDLDVALSRPKCTGKIPSARVSILHEVRPVTSGCFPIMHDRTKIRQLPNLLRGYEHVRLSTHNVINAEDAPGGPYEIGTAGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 186 - DNA, 1725 bp, Artificial Sequence
  PDI-B Vic-S142A DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgagcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaggaccctacgagattggaacctcaggggcctgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 187 - protein, 574 aa, Artificial Sequence
  PDI-B Vic-S142A AA
MAKNVAIFGLLFSLLVLVPSQIFADRICTGITSSNSPHVVKTATQGEVNVTGVIPLTTTPTKSHFANLKGTETRGKLCPKCLNCTDLDVALSRPKCTGKIPSARVSILHEVRPVTSGCFPIMHDRTKIRQLPNLLRGYEHVRLSTHNVINAEDAPGGPYEIGTSGACPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 188 - DNA, 1725 bp, Artificial Sequence
  PDI-B Vic-D193G DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgagcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaggaccctacgagattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctggcaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 189 - protein, 574 aa, Artificial Sequence
  PDI-B Vic-D193G AA
MAKNVAIFGLLFSLLVLVPSQIFADRICTGITSSNSPHVVKTATQGEVNVTGVIPLTTTPTKSHFANLKGTETRGKLCPKCLNCTDLDVALSRPKCTGKIPSARVSILHEVRPVTSGCFPIMHDRTKIRQLPNLLRGYEHVRLSTHNVINAEDAPGGPYEIGTSGSCPNITNGNGFFATMAWAVPKNKTATNPLTIEVPYICTEGEDQITVWGFHSGNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 190 - DNA, 1725 bp, Artificial Sequence
  PDI-B Vic-L201A DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgagcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaggaccctacgagattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaaggcctatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 191 - protein, 574 aa, Artificial Sequence
  PDI-B Vic-L201A AA
MAKNVAIFGLLFSLLVLVPSQIFADRICTGITSSNSPHVVKTATQGEVNVTGVIPLTTTPTKSHFANLKGTETRGKLCPKCLNCTDLDVALSRPKCTGKIPSARVSILHEVRPVTSGCFPIMHDRTKIRQLPNLLRGYEHVRLSTHNVINAEDAPGGPYEIGTSGSCPNITNGNGFFATMAWAVPKNKTATNPLTIEVPYICTEGEDQITVWGFHSDNETQMAKAYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 192 - DNA, 1725 bp, Artificial Sequence
  PDI-B Vic-L201W DNA
atggcgaaaaacgttgcgattttcggcttattgttttctcttcttgtgttggttccttctcagatcttcgcggatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgagcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaggaccctacgagattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagtggtatggggactcaaagccccagaagttcacctcatctgccaacggagtgaccacacattacgtttcacagattggtggcttcccaaatcaaacagaagacggaggactaccacaaagtggcagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctggtggaggatgggaaggaatgattgcaggttggcacggatacacatcccatggggcacatggagtagcggtggcagcagaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgatggattggataattaccagattttggcgatctattcaactgtcgccagttcattggtactggtagtctccctgggggcaatcagtttctggatgtgctctaatgggtctctacagtgtagaatatgtatttaa

SEQ ID NO: 193 - protein, 574 aa, Artificial Sequence
  PDI-B Vic-L201W AA
MAKNVAIFGLLFSLLVLVPSQIFADRICTGITSSNSPHVVKTATQGEVNVTGVIPLTTTPTKSHFANLKGTETRGKLCPKCLNCTDLDVALSRPKCTGKIPSARVSILHEVRPVTSGCFPIMHDRTKIRQLPNLLRGYEHVRLSTHNVINAEDAPGGPYEIGTSGSCPNITNGNGFFATMAWAVPKNKTATNPLTIEVPYICTEGEDQITVWGFHSDNETQMAKWYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPGGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNYQILAIYSTVASSLVLVVSLGAISFWMCSNGSLQCRICI

SEQ ID NO: 194 - DNA, 1722 bp, Artificial Sequence
  PDI-H1 Bris DNA
atggcgaaaaacgttgcgattttcggcttattgttttctcttcttgtgttggttccttctcagatcttcgcggacacattatgtataggttatcatgcgaacaattcaacagacactgtagacacagtactagaaaagaatgtaacagtaacacactctgttaaccttctggaagacaagcataacggaaaactatgcaaactaggcggggtagccccattgcatttgggtaaatgtaacattgctggctggatcctgggaaatccagagtgtgaatcactctccacagcaagatcatggtcctacattgtggaaacatctaattcagacaatggaacgtgttacccaggagatttcatcaattatgaggagctaagagagcaattgagctcagtgtcatcatttgaaaggtttgagatattccccaagacaagttcatggcccaatcatgactcgaacaaaggtgtaacggcagcatgtcctcacgctggagcaaaaagcttctacaaaaacttgatatggctagttaaaaaaggaaattcatacccaaagcttaaccaaacctacattaatgataaagggaaagaagtcctcgtgctgtggggcattcaccatccacccactactgctgaccaacaaagtctctatcagaatgcagatgcatatgtttttgtggggacatcaagatacagcaagaagttcaagccggaaatagcaacaagacccaaagtgagggatcaagaagggagaatgaactattactggacactagtagagccgggagacaaaataacattcgaagcaactggaaatctagtggtaccgagatatgcattcacaatggaaagaaatgctggatctggtattatcatttcagatacaccagtccacgattgcaatacaacttgtcagacagccgagggtgctataaacaccagcctcccatttcagaatgtgcatccggtgacaattggaaaatgtccaaagtatgtaaaaagcacaaaattgagactggccacaggattgaggaatgttccgtctattcaatctagaggcctattcggggccattgccggcttcattgaaggggggtggacagggatggtagatggatggtacggttatcaccatcaaaatgagcaggggtcaggatatgcagccgacctgaagagcacacaaaatgccattgacaagattactaacaaagtaaattctgttattgaaaagatgaatacacaggacacagcagtgggtaaagagttcaaccacctggaaaaaagaatagagaatctaaataaaaaagttgatgatggtttcctggacatttggacttacaatgccgaactgttggttctaatggaaaatgaaagaactttggactatcacgattcaaatgtgaagaacttgtatgaaaaagtaagaaaccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaaaaaatagatggggtaaagctggaatcaacaaggatttaccagattttggcgatctattcaactgtcgccagttcattggtactggtagtctccctgggggcaatcagcttctggatgtgctctaatgggtctctacagtgtagaatatgtatttaa

SEQ ID NO: 195 - protein, 573 aa, Artificial Sequence
  PDI-H1 Bris AA
MAKNVAIFGLLFSLLVLVPSQIFADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNKVNSVIEKMNTQDTAVGKEFNHLEKRIENLNKKVDDGFLDIWTYNAELLVLMENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 196 - DNA, 1722 bp, Artificial Sequence
  PDI-H1 Bris-Y98F DNA
atggcgaaaaacgttgcgattttcggcttattgttttctcttcttgtgttggttccttctcagatcttcgcggacacattatgtataggttatcatgcgaacaattcaacagacactgtagacacagtactagaaaagaatgtaacagtaacacactctgttaaccttctggaagacaagcataacggaaaactatgcaaactaggcggggtagccccattgcatttgggtaaatgtaacattgctggctggatcctgggaaatccagagtgtgaatcactctccacagcaagatcatggtcctacattgtggaaacatctaattcagacaatggaacgtgtttcccaggagatttcatcaattatgaggagctaagagagcaattgagctcagtgtcatcatttgaaaggtttgagatattccccaagacaagttcatggcccaatcatgactcgaacaaaggtgtaacggcagcatgtcctcacgctggagcaaaaagcttctacaaaaacttgatatggctagttaaaaaaggaaattcatacccaaagcttaaccaaacctacattaatgataaagggaaagaagtcctcgtgctgtggggcattcaccatccacccactactgctgaccaacaaagtctctatcagaatgcagatgcatatgtttttgtggggacatcaagatacagcaagaagttcaagccggaaatagcaacaagacccaaagtgagggatcaagaagggagaatgaactattactggacactagtagagccgggagacaaaataacattcgaagcaactggaaatctagtggtaccgagatatgcattcacaatggaaagaaatgctggatctggtattatcatttcagatacaccagtccacgattgcaatacaacttgtcagacagccgagggtgctataaacaccagcctcccatttcagaatgtgcatccggtgacaattggaaaatgtccaaagtatgtaaaaagcacaaaattgagactggccacaggattgaggaatgttccgtctattcaatctagaggcctattcggggccattgccggcttcattgaaggggggtggacagggatggtagatggatggtacggttatcaccatcaaaatgagcaggggtcaggatatgcagccgacctgaagagcacacaaaatgccattgacaagattactaacaaagtaaattctgttattgaaaagatgaatacacaggacacagcagtgggtaaagagttcaaccacctggaaaaaagaatagagaatctaaataaaaaagttgatgatggtttcctggacatttggacttacaatgccgaactgttggttctaatggaaaatgaaagaactttggactatcacgattcaaatgtgaagaacttgtatgaaaaagtaagaaaccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaaaaaatagatggggtaaagctggaatcaacaaggatttaccagattttggcgatctattcaactgtcgccagttcattggtactggtagtctccctgggggcaatcagcttctggatgtgctctaatgggtctctacagtgtagaatatgtatttaa

SEQ ID NO: 197 - protein, 573 aa, Artificial Sequence
  PDI-H1 Bris-Y98F AA
MAKNVAIFGLLFSLLVLVPSQIFADTLCIGYHANNSTDTVDTVLEKNVTVTHSVNLLEDKHNGKLCKLGGVAPLHLGKCNIAGWILGNPECESLSTARSWSYIVETSNSDNGTCFPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNKVNSVIEKMNTQDTAVGKEFNHLEKRIENLNKKVDDGFLDIWTYNAELLVLMENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 198 - DNA, 1731 bp, Artificial Sequence
  PDI-H5 Indo DNA
atggcgaaaaacgttgcgattttcggcttattgttttctcttcttgtgttggttccttctcagatcttcgccgatcagatttgcattggttaccatgcaaacaattcaacagagcaggttgacacaatcatggaaaagaacgttactgttacacatgcccaagacatactggaaaagacacacaacgggaagctctgcgatctagatggagtgaagcctctaattttaagagattgtagtgtagctggatggctcctcgggaacccaatgtgtgacgaattcatcaatgtaccggaatggtcttacatagtggagaaggccaatccaaccaatgacctctgttacccagggagtttcaacgactatgaagaactgaaacacctattgagcagaataaaccattttgagaaaattcaaatcatccccaaaagttcttggtccgatcatgaagcctcatcaggagttagctcagcatgtccatacctgggaagtccctccttttttagaaatgtggtatggcttatcaaaaagaacagtacatacccaacaataaagaaaagctacaataataccaaccaagaggatcttttggtactgtggggaattcaccatcctaatgatgcggcagagcagacaaggctatatcaaaacccaaccacctatatttccattgggacatcaacactaaaccagagattggtaccaaaaatagctactagatccaaagtaaacgggcaaagtggaaggatggagttcttctggacaattttaaaacctaatgatgcaatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcaacagggctcagaaatagccctcaaagagagagcagaagaaaaaagagaggactatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcaatcattgacaaaatgaacactcagtttgaggccgttggaagggaatttaataacttagaaaggagaatagagaatttaaacaagaagatggaagacgggtttctagatgtctggacttataatgccgaacttctggttctcatggaaaatgagagaactctagactttcatgactcaaatgttaagaacctctacgacaaggtccgactacagcttagggataatgcaaaggagctgggtaacggttgtttcgagttctatcacaaatgtgataatgaatgtatggaaagtataagaaacggaacgtacaactatccgcagtattcagaagaagcaagattaaaaagagaggaaataagtggggtaaaattggaatcaataggaacttaccaaatactgtcaatttattcaacagtggcgagttccctagcactggcaatcatgatggctggtctatctttatggatgtgctccaatggatcgttacaatgcagaatttgcatttaa

SEQ ID NO: 199 - protein, 576 aa, Artificial Sequence
  PDI-H5 Indo AA
MAKNVAIFGLLFSLLVLVPSQIFADQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 200 - DNA, 50 bp, Artificial Sequence
  IF-H5ITMCT.s1-4r
actaaagaaaataggcctttaaatgcaaattctgcattgtaacgatccat

SEQ ID NO: 201 - DNA, 1731 bp, Artificial Sequence
  PDI-H5 Indo-Y91F DNA
atggcgaaaaacgttgcgattttcggcttattgttttctcttcttgtgttggttccttctcagatcttcgccgatcagatttgcattggttaccatgcaaacaattcaacagagcaggttgacacaatcatggaaaagaacgttactgttacacatgcccaagacatactggaaaagacacacaacgggaagctctgcgatctagatggagtgaagcctctaattttaagagattgtagtgtagctggatggctcctcgggaacccaatgtgtgacgaattcatcaatgtaccggaatggtcttacatagtggagaaggccaatccaaccaatgacctctgtttcccagggagtttcaacgactatgaagaactgaaacacctattgagcagaataaaccattttgagaaaattcaaatcatccccaaaagttcttggtccgatcatgaagcctcatcaggagttagctcagcatgtccatacctgggaagtccctccttttttagaaatgtggtatggcttatcaaaaagaacagtacatacccaacaataaagaaaagctacaataataccaaccaagaggatcttttggtactgtggggaattcaccatcctaatgatgcggcagagcagacaaggctatatcaaaacccaaccacctatatttccattgggacatcaacactaaaccagagattggtaccaaaaatagctactagatccaaagtaaacgggcaaagtggaaggatggagttcttctggacaattttaaaacctaatgatgcaatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcaacagggctcagaaatagccctcaaagagagagcagaagaaaaaagagaggactatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcaatcattgacaaaatgaacactcagtttgaggccgttggaagggaatttaataacttagaaaggagaatagagaatttaaacaagaagatggaagacgggtttctagatgtctggacttataatgccgaacttctggttctcatggaaaatgagagaactctagactttcatgactcaaatgttaagaacctctacgacaaggtccgactacagcttagggataatgcaaaggagctgggtaacggttgtttcgagttctatcacaaatgtgataatgaatgtatggaaagtataagaaacggaacgtacaactatccgcagtattcagaagaagcaagattaaaaagagaggaaataagtggggtaaaattggaatcaataggaacttaccaaatactgtcaatttattcaacagtggcgagttccctagcactggcaatcatgatggctggtctatctttatggatgtgctccaatggatcgttacaatgcagaatttgcatttaa

SEQ ID NO: 202 - protein, 576 aa, Artificial Sequence
  PDI-H5 Indo-Y91F AA
MAKNVAIFGLLFSLLVLVPSQIFADQICIGYHANNSTEQVDTIMEKNVTVTHAQDILEKTHNGKLCDLDGVKPLILRDCSVAGWLLGNPMCDEFINVPEWSYIVEKANPTNDLCFPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 203 - protein, 549 aa, Influenza Virus
DTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 204 - protein, 550 aa, Influenza Virus
QKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISCFLLCVALLGFIMWACQKGNIRCNICI

SEQ ID NO: 205 - protein, 552 aa, Influenza Virus
DQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 206 - protein, 542 aa, Influenza Virus
DKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTNGATSACRRSGSSFYAEMKWLLSNTDNAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 207 - protein, 569 aa, Influenza Virus
DRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 208 - protein, 568 aa, Influenza Virus; identical to SEQ ID NO: 15

SEQ ID NO: 209 - protein, 567 aa, Influenza Virus; identical to SEQ ID NO: 16